(19) United States
(12) Patent Application Publication
(10) Pub. No.: US 2026/0216314 A1
(43) Pub. Date: Jul. 30, 2026

(54) MOSAIC INFLUENZA VIRUS HEMAGGLUTININ POLYPEPTIDES AND USES THEREOF

(71) Applicant: ICAHN SCHOOL OF MEDICINE AT MOUNT SINAI, New York, NY (US)

(72) Inventors: Adolfo GARCIA-SASTRE, New York, NY (US); Peter PALESE, New York, NY (US); Florian KRAMMER, New York, NY (US); Felix BRÖCKER, Berlin (DE); Weina SUN, New York, NY (US)

(73) Assignee: ICAHN SCHOOL OF MEDICINE AT MOUNT SINAI, New York, NY (US)

(21) Appl. No.: 19/237,764

(22) Filed: Jun. 13, 2025

Related U.S. Application Data
(62) Division of application No. 17/252,638, filed on Jun. 20, 2019, now Pat. No. 12,364,746.
(60) Provisional application No. 62/688,329, filed on Jun. 21, 2018.

(51) Int. Cl. A61K 39/145; A61P 31/16; A61P 37/04; C07K 14/005; C12N 7/00; G01N 33/50

(52) U.S. Cl. CPC A61K 39/145; A61P 31/16; A61P 37/04; C07K 14/005; C12N 7/00; G01N 33/5091; C12N 2760/16121; C12N 2760/16122; C12N 2760/16123; C12N 2760/16134; C12N 2760/16171

(57) ABSTRACT

In one aspect, provided herein is a mosaic influenza virus hemagglutinin (HA) polypeptide comprising an influenza A virus HA ectodomain of an influenza A virus strain HA, wherein the HA ectodomain comprises an HA stem domain of the influenza A virus strain HA and an HA globular head domain of the influenza A virus strain HA, wherein the HA globular head domain of the influenza A virus strain HA has been engineered to comprise one or more amino acid substitutions in one, two, three, four or all of the antigenic sites. In another aspect, provided herein are influenza A viruses comprising such a mosaic influenza virus HA polypeptide. In another aspect, provided herein are immunogenic compositions comprising such a mosaic influenza virus HA polypeptide or an influenza A virus comprising such a mosaic influenza virus HA polypeptide, and optionally an adjuvant. In yet another aspect, provided herein are methods for immunizing a subject against an influenza A virus, or preventing an influenza A virus infection in a subject comprising administering such an immunogenic composition to the subject.

This application is a divisional of U.S. patent application Ser. No. 17/252,638, which is a U.S. National Stage Application under 35 U.S.C. § 371 of International Patent Application No. PCT/US2019/038178, filed Jun. 20, 2019, which claims the benefit of U.S. Provisional Patent Application No. 62/688,329, filed Jun. 21, 2018, the disclosure of each of which is incorporated by reference herein in its entirety.

This invention was made with government support under P01AI097092, U19 AI109946, HHSN272201400008C, and 5T32AI007647-18, awarded by NIH. The government has certain rights in the invention.

This application contains an electronic Sequence Listing which has been submitted in XML file format via Patent Center, the entire content of which is incorporated by reference herein in its entirety. The Sequence Listing XML file submitted via Patent Center is entitled “06923-448-999_SEQ_LISTING.xml”, was created on Jun. 12, 2025, and is 223,642 bytes in size.

### 1. INTRODUCTION

In one aspect, provided herein is a mosaic influenza A virus hemagglutinin (HA) polypeptide comprising an influenza A virus HA ectodomain of an influenza A virus strain HA, wherein the HA ectodomain comprises an HA stem domain of the influenza A virus strain HA and an HA globular head domain of the influenza A virus strain HA, wherein the HA globular head domain of the influenza A virus strain HA has been engineered to comprise one or more amino acid substitutions in one, two, three, four or all of the antigenic sites. In another aspect, provided herein are influenza A viruses comprising such a mosaic influenza A virus HA polypeptide. In another aspect, provided herein are immunogenic compositions comprising such a mosaic influenza A virus HA polypeptide or an influenza A virus comprising such a mosaic influenza A virus HA polypeptide, and optionally an adjuvant. In yet another aspect, provided herein are methods for immunizing a subject against an influenza A virus, or preventing an influenza A virus infection in a subject comprising administering such an immunogenic composition to the subject.

### 2. BACKGROUND

Influenza viruses are enveloped RNA viruses that belong to the family of Orthomyxoviridae (Palese and Shaw (2007) Orthomyxoviridae: The Viruses and Their Replication, 5th ed. Fields' Virology, edited by B. N. Fields, D. M. Knipe and P. M. Howley. Wolters Kluwer Health/Lippincott Williams & Wilkins, Philadelphia, USA, p 1647-1689). The natural host of influenza A viruses are mainly avians, but influenza A viruses (including those of avian origin) also can infect and cause illness in humans and other animal hosts (bats, canines, pigs, horses, sea mammals, and mustelids). For example, the H5N1 avian influenza A virus circulating in Asia has been found in pigs in China and Indonesia and has also expanded its host range to include cats, leopards, and tigers, which generally have not been considered susceptible to influenza A (CIDRAP—Avian Influenza: Agricultural and Wildlife Considerations). The occurrence of influenza virus infections in animals could potentially give rise to human pandemic influenza strains.

Influenza A and B viruses are major human pathogens, causing a respiratory disease that ranges in severity from sub-clinical infection to primary viral pneumonia which can result in death. The clinical effects of infection vary with the virulence of the influenza strain and the exposure, history, age, and immune status of the host. The cumulative morbidity and mortality caused by seasonal influenza is substantial due to the relatively high attack rate. In a normal season, influenza can cause between 3-5 million cases of severe illness and up to 500,000 deaths worldwide (World Health Organization (2003) Influenza: Overview; March 2003). In the United States, influenza viruses infect an estimated 10-15% of the population (Glezen and Couch R B (1978) Interpandemic influenza in the Houston area, 1974-76. N Engl J Med 298:587-592; Fox et al. (1982) Influenza virus infections in Seattle families, 1975-1979. II. Pattern of infection in invaded households and relation of age and prior antibody to occurrence of infection and related illness. Am J Epidemiol 116:228-242) and are associated with approximately 30,000 deaths each year (Thompson W W et al. (2003) Mortality Associated with Influenza and Respiratory Syncytial Virus in the United States. JAMA 289:179-186; Belshe (2007) Translational research on vaccines: influenza as an example. Clin Pharmacol Ther 82:745-749).

In addition to annual epidemics, influenza viruses are the cause of infrequent pandemics. For example, influenza A viruses can cause pandemics such as those that occurred in 1918, 1957, 1968, and 2009. Due to the lack of pre-formed immunity against the major viral antigen, hemagglutinin (HA), pandemic influenza can affect greater than 50% of the population in a single year and often causes more severe disease than epidemic influenza. A stark example is the pandemic of 1918, in which an estimated 50-100 million people were killed (Johnson and Mueller (2002) Updating the Accounts: Global Mortality of the 1918-1920 “Spanish” Influenza Pandemic Bulletin of the History of Medicine 76:105-115). Since the emergence of the highly pathogenic avian H5N1 influenza virus in the late 1990s (Claas et al. (1998) Human influenza A H5N1 virus related to a highly pathogenic avian influenza virus. Lancet 351:472-7), there have been concerns that it may be the next pandemic virus. Further, H7, H9 and H10 strains are candidates for new pandemics since these strains infect humans on occasion.

Seasonal vaccination is currently the most effective intervention against influenza (Gross et al., Ann Intern Med, 1995, 123(7): p. 518-27; Ogburn et al., J Reprod Med, 2007, 52(9): p. 753-6; Jefferson et al., Lancet, 2005. 366(9492): p. 1165-74; Beyer et al., Vaccine, 2013, 31(50): p. 6030-3; Nichol et al., N Engl J Med, 1995. 333(14): p. 889-93; Jefferson et al., Lancet, 2005. 365(9461): p. 773-80), yet overall vaccine effectiveness was only 36% in the recent 2017-2018 season (Flannery et al., MMWR Morb Mortal Wkly Rep, 2018. 67(6): p. 180-185). However, current vaccination approaches rely on achieving a good match between circulating strains and the isolates included in the vaccine. Such a match is often difficult to attain due to a combination of factors. First, influenza viruses are constantly undergoing change: every 3-5 years the predominant strain of influenza A virus is replaced by a variant that has undergone sufficient antigenic drift to evade existing antibody responses. Isolates to be included in vaccine preparations must therefore be selected each year based on the intensive surveillance efforts of the World Health Organization (WHO) collaborating centers. Second, to allow sufficient time for vaccine manufacture and distribution, strains must be selected approximately six months prior to the initiation of the influenza season. Often, the predictions of the vaccine strain selection committee are inaccurate, resulting in a substantial drop in the efficacy of vaccination.

The possibility of a novel subtype of influenza A virus entering the human population also presents a significant challenge to current vaccination strategies. Since it is impossible to predict what subtype and strain of influenza virus will cause the next pandemic, current, strain-specific approaches cannot be used to prepare a pandemic influenza vaccine in advance of a pandemic. Thus, there is a need for vaccines that cross-protect subjects against different strains and/or subtypes of influenza virus.

### 3. SUMMARY

In one aspect, provided herein is a mosaic influenza virus hemagglutinin (HA) polypeptide comprising an influenza A virus HA ectodomain, wherein the HA ectodomain comprises an HA stem domain of the group 1 influenza A virus strain HA and an HA globular head domain of the group 1 influenza A virus strain HA, wherein the HA globular head domain of the group 1 influenza A virus strain HA has been engineered to comprise 1, 2, 3, 4, 5, 6, 7, 8, 9, 10, 11, 12 or more amino acid substitutions within one, two, three, four or more antigenic sites of the HA globular head domain. In a specific embodiment, provided herein is a mosaic influenza virus hemagglutinin (HA) polypeptide comprising an influenza A virus HA ectodomain, wherein the HA ectodomain comprises an HA stem domain of the group 1 influenza A virus strain HA and an HA globular head domain of the group 1 influenza A virus strain HA, wherein the HA globular head domain of the group 1 influenza A virus strain HA has been engineered to comprise 1, 2, 3, 4, 5, 6, 7, 8, 9, 10, 11, 12 or more amino acid substitutions within each of one, two, three, four or more hypervariable antigenic sites of the globular head domain of the HA globular head domain. In a specific embodiment, the mosaic influenza virus HA polypeptide further comprises the transmembrane and cytoplasmic domains of the influenza A virus strain HA. In some embodiments, the amino acid substitutions are random amino acid substitutions that do not change the conformation of the HA. For example, amino acid residues in an antigenic site of the globular head domain of an influenza A virus HA may be substituted with alanines or other amino acid residues so long as the substitution does not change the conformation of the HA. In certain embodiments, the amino acid substitutions are amino acid residues substitutions with residues found in a corresponding hypervariable antigenic site of the globular head domain of a HA of another influenza A virus strain or subtype.

In another aspect, provided herein is a mosaic influenza virus hemagglutinin (HA) polypeptide comprising an influenza A virus HA ectodomain of a group 1 influenza A virus strain HA, wherein the HA ectodomain comprises an HA stem domain of the group 1 influenza A virus strain HA and an HA globular head domain of the group 1 influenza A virus strain HA, wherein the HA globular head domain of the group 1 influenza A virus strain HA has been engineered to comprise one, two, three, four or all of the following: (a) 1, 2, 3, 4, 5, 6, 7, 8, 9, 10, 11, 12 or more amino acid substitutions within the Sa antigenic site or a corresponding hypervariable antigenic site of the globular head domain of the group 1 influenza A virus strain HA; (b) 1, 2, 3, 4, 5, 6, 7, 8, 9, 10, 11 or more amino acid substitutions within the Sb antigenic site or a corresponding hypervariable antigenic site of the globular head domain of the group 1 influenza A virus strain HA; (c) 1, 2, 3, 4, 5, 6, 7, 8, 9, 10 or more amino acid substitutions within the Ca1 antigenic site or a corresponding hypervariable antigenic site of the globular head domain of the group 1 influenza A virus strain HA; (d) 1, 2, 3, 4, 5, 6, 7 or more amino acid substitutions within the Ca2 antigenic site or a corresponding hypervariable antigenic site of the globular head domain of the group 1 influenza A virus strain HA; and (e) 1, 2, 3, 4, 5 or more amino acid substitutions within the Cb antigenic site or a corresponding hypervariable antigenic site of the globular head domain of the group 1 influenza A virus strain HA. In another specific embodiment, the mosaic influenza virus HA polypeptide further comprises the transmembrane and cytoplasmic domains of the group 1 influenza A virus strain HA. In another specific embodiment, the group 1 influenza A virus is an H1 subtype (e.g., influenza A/Michigan/45/2015 virus).

In another aspect, provided herein is a mosaic influenza virus hemagglutinin (HA) polypeptide comprising an HA ectodomain of a first group 1 influenza A virus strain HA, wherein the HA ectodomain comprises an HA stem domain of the first group 1 influenza A virus strain HA and an HA globular head domain of the first group 1 influenza A virus strain HA, wherein the HA globular head domain of the first group 1 influenza A virus strain has been engineered to comprise one, two, three, four or all of the following: (a) 1, 2, 3, 4, 5, 6, 7, 8, 9, 10, 11, 12 or more amino acid substitutions within the Sa antigenic site or a corresponding hypervariable antigenic site of the globular head domain of the first group 1 influenza A virus strain HA, wherein the amino acid substitutions substitute 1, 2, 3, 4, 5, 6, 7, 8, 9, 10, 11, 12 or more amino acid residues within the Sa antigenic site or the corresponding hypervariable antigenic site of the globular head domain of the first group 1 influenza A virus strain HA with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; (b) 1, 2, 3, 4, 5, 6, 7, 8, 9, 10, 11 or more amino acid substitutions within the Sb antigenic site or a corresponding hypervariable antigenic site of the globular head domain of the first group 1 influenza A virus strain HA, wherein the amino acid substitutions substitute 1, 2, 3, 4, 5, 6, 7, 8, 9, 10, 11 or more amino acid residues within the Sb antigenic site or the corresponding hypervariable antigenic site of the globular head domain of the first group 1 influenza A virus strain HA with amino acid residues found in a corresponding region of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; (c) 1, 2, 3, 4, 5, 6, 7, 8, 9, 10 or more amino acid substitutions within the Ca1 antigenic site or a corresponding hypervariable antigenic site of the globular head domain of the first group 1 influenza A virus, strain HA, wherein the amino acid substitutions substitute 1, 2, 3, 4, 5, 6, 7, 8, 9, 10 or more amino acid residues within the Ca1 antigenic site or the corresponding hypervariable antigenic site of the globular head domain of the first group 1 influenza A virus strain HA with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; (d) 1, 2, 3, 4, 5, 6, 7 or more amino acid substitutions within the Ca2 antigenic site or a corresponding hypervariable antigenic site of the globular head domain of the first group 1 influenza A virus strain HA, wherein the amino acid substitutions substitute 1, 2, 3, 4, 5, 6, 7 or more amino acid residues within the Ca2 antigenic site or the corresponding hypervariable antigenic site of the globular head domain of the first group 1 influenza A virus strain HA with amino acid residues found in a corresponding region of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; and (e) 1, 2, 3, 4, 5 or more amino acid substitutions within the Cb antigenic site or a corresponding hypervariable antigenic site of the globular head domain of the first group 1 influenza A virus strain, wherein the amino acid substitutions substitute 1, 2, 3, 4, 5 or more amino acid residues within the Cb antigenic site or the corresponding hypervariable antigenic site of the globular head domain of the first group 1 influenza A virus strain with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain. In a specific embodiment, the corresponding region of the HA globular head domain is of either: (1) a group 1 influenza A virus HA of a different subtype than the first group 1 influenza A virus strain (e.g., an H5 subtype (such as, e. g., influenza A/Vietnam/1203/2004 virus) or an H13 subtype (such as, e g., A/black headed gull/Sweden/1/1999 virus); (2) or a combination of group 1 influenza A virus HAs of different subtypes than the first group 1 influenza A virus strain (e.g., a combination of an H5 subtype (such as, e g., influenza A/Vietnam/1203/2004 virus) and an H13 subtype (such as, e g., A/black headed gull/Sweden/1/1999 virus). In another specific embodiment, the mosaic influenza virus HA polypeptide further comprises the transmembrane and cytoplasmic domains of the first group 1 influenza A virus strain HA. In another specific embodiment, the first group 1 influenza A virus is an H1 subtype (e.g., influenza A/Michigan/45/2015 virus).

In another aspect, provided here is a mosaic influenza virus HA polypeptide comprising an HA ectodomain of influenza A/Michigan/45/2015 virus HA, wherein the HA ectodomain comprises the influenza A/Michigan/45/2015 virus HA stem domain and HA globular head domain, wherein the globular head domain has been engineered to comprise one, two, three, four or all of the following amino acid sequence substitutions: (a) the amino acid sequences PN, KKGNS (SEQ ID NO: 1), and PKLNQS (SEQ ID NO: 2) in the HA globular head domain Sa antigenic site of influenza A/Michigan/45/2015 virus HA have been substituted with the following amino acid sequences PS, KKNST (SEQ ID NO: 3), and PTIKRS (SEQ ID NO: 4), respectively; (b) the amino acid sequence TTADQQSLYQNA (SEQ ID NO: 5) in the HA globular head domain Sb antigenic site of influenza A/Michigan/45/2015 virus HA has been substituted with the following amino acid sequence DAAEQTKLYQNP (SEQ ID NO: 6); (c) the amino acid sequences INDKG (SEQ ID NO: 7), TSR, and EPG in the HA globular head domain Ca1 antigenic site of influenza A/Michigan/45/2015 virus HA have been substituted with the following amino acid sequences NNTTG (SEQ ID NO: 8), TSS, and HPG, respectively; (d) the amino acid sequences PHAGAK (SEQ ID NO: 9) and RD in the HA globular head domain Ca2 antigenic site of influenza A/Michigan/45/2015 virus HA have been substituted with the following amino acid sequences PYQGKS (SEQ ID NO:10) and ND, respectively; and (e) the amino acid sequence LSTASS (SEQ ID NO: 11) in the HA globular head domain Cb antigenic site of influenza A/Michigan/45/2015 virus HA have been substituted with the following amino acid sequence LNVPE (SEQ ID NO: 12). In a specific embodiment, the mosaic influenza virus HA polypeptide further comprises the transmembrane and cytoplasmic domains of the influenza A/Michigan/45/2015 virus HA.

In another aspect, provided herein a mosaic influenza virus HA polypeptide comprising an HA ectodomain of a first group 2 influenza A virus strain HA, wherein the HA ectodomain comprises an HA stem domain of the first group 2 influenza A virus strain HA and an HA globular head domain of the first group 2 influenza A virus strain HA, wherein the HA globular head domain of the first group 2 influenza A virus strain HA has been engineered to comprise one, two, three, four or all of the following: (a) 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the first group 2 influenza A virus strain HA; (b) 1, 2, 3, 4, 5, 6, 7, 8, 9, 10, 11, 12, 13, 14, 15, 16, 17 or more amino acid substitutions within the B antigenic site or a corresponding hypervariable antigenic site of the globular head domain of the first group 2 influenza A virus strain HA; (c) 1, 2, 3, 4, 5, 6, 7, 8, 9, 10, 11, 12, 13, 14, 15, 16, 17, 18 or more amino acid substitutions within the C antigenic site or a corresponding hypervariable antigenic site of the globular head domain of the first group 2 influenza A virus strain HA; (d) 1, 2, 3, 4, 5, 6, 7, 8, 9, 10, 11, 12, 13, 14, 15, 16, 17, 18 or more amino acid substitutions within the D antigenic site or a corresponding hypervariable antigenic site of the globular head domain of the first group 2 influenza A virus strain HA; and (e) 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the first group 2 influenza A virus strain HA. In a specific embodiment, the mosaic influenza virus HA polypeptide further comprises the transmembrane and cytoplasmic domains of the first group 2 influenza A virus strain HA. In another specific embodiment, the first group 2 influenza virus strain is A/Hong Kong/4801/2014.

In another aspect, provided herein is a mosaic influenza virus HA polypeptide comprising an HA ectodomain of a first group 2 influenza A virus strain HA, wherein the HA ectodomain comprises an HA stem domain of the first group 2 influenza A virus strain HA and an HA globular head domain of the first group 2 influenza A virus HA strain HA, wherein the HA globular head domain of the first group 2 influenza A virus strain HA has been engineered to comprise one, two, three, four or all of the following: (a) 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18, 19, 20, 21, 22, 23, 24, 25, 26 or more amino acid residues within the A antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; (b) 1, 2, 3, 4, 5, 6, 7, 8, 9, 10, 11, 12, 13, 14, 15, 16, 17 or more amino acid substitutions within the B antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17 or more amino acid residues within the B antigenic site or the corresponding hypervariable antigenic site of the globular head domain of the group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; (c) 1, 2, 3, 4, 5, 6, 7, 8, 9, 10, 11, 12, 13, 14, 15, 16, 17, 18 or more amino acid substitutions within the C antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18 or more amino acid residues within the C antigenic site or the corresponding hypervariable antigenic site of the globular head domain of the first group 2 influenza A virus strain HA with amino acid residues found in the corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; (d) 1, 2, 3, 4, 5, 6, 7, 8, 9, 10, 11, 12, 13, 14, 15, 16, 17, 18 or more amino acid substitutions within the D antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18, or more amino acid residues within the D antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; and (e) 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the group 2 influenza A virus strain HA, wherein the amino acid substitutions substitute 1, 2, 3, 4, 5, 6, 7, 8, 9, 10, 11, 12, 13, 14, 15, 16, 17, 18, 19, 20, 21, 22, 23, 24, 25, 26 or more amino acid residues within the E antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain. In a specific embodiment, the mosaic influenza virus HA polypeptide further comprises the transmembrane and cytoplasmic domains of the first group 2 influenza A virus strain HA. In another specific embodiment, the corresponding region of the HA globular head domain is of a group 2 influenza A virus HA of a different subtype than the first influenza A virus group 2 strain (e.g., an H10 subtype, such as, e.g., influenza A/Jiangxi-Donghu/346-1/2013 virus or A/mallard/Gurjev/263/1982). In another specific embodiment, the first group 2 influenza virus strain is A/Hong Kong/4801/2014.

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise one, two, three, four or all of the following amino acid sequence substitutions: (a) the amino acid sequence NNESFNWT-GVTQNGTSSACIRRSSSS (SEQ ID NO: 13) in the HA globular head domain A antigenic site of influenza A/Hong Kong/4801/2014 virus HA has been substituted with the following amino acid sequence NNESFNWTGVTQNGTSSACMRNGGNS (SEQ ID NO: 14); (b) the amino acid sequences THL-NYK (SEQ ID NO: 15) and GTDKDQIFLYAQ (SEQ ID NO: 16) in the HA globular head domain B antigenic site of influenza A/Hong Kong/4801/2014 virus HA have been substituted with the following amino acid sequences THL-NQK (SEQ ID NO: 17) and GTNQDQIFLYAQ (SEQ ID NO: 18), respectively; (c) the amino acid sequences QNSSIGEICDS (SEQ ID NO: 19) and PIG-KCKSE (SEQ ID NO: 20) in the HA globular head domain C antigenic site of influenza A/Hong Kong/4801/2014 virus HA have been substituted with the following amino acid sequences ESTGINRLCMK (SEQ ID NO: 21) and PIDNNCESK (SEQ ID NO: 22), respectively; (d) the amino acid sequence RITVSTKRSQQAVIPNIGS (SEQ ID NO: 23) in the HA globular head domain D antigenic site of influenza A/Hong Kong/4801/2014 virus HA has been substituted with the following amino acid sequence RITVSTSTYQQAVIPNIGS (SEQ ID NO: 25); and (e) the amino acid sequences ENCT (SEQ ID NO: 124), GFQNKKWDLFVERSKAY (SEQ ID NO: 27) and IRSGKS (SEQ ID NO: 28) in the HA globular head domain E antigenic site of influenza A/Hong Kong/4801/2014 virus HA have been substituted with the following amino acid sequences GNCH (SEQ ID NO: 125), GFQNKMWDLFVERSKAY (SEQ ID NO: 29) and LRIGRS (SEQ ID NO: 24), respectively. In a specific embodiment, the mosaic influenza virus HA polypeptide further comprises the transmembrane and cytoplasmic domains of the influenza A/Hong Kong/4801/2014 virus HA.

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise one, two, three, four or all of the following amino acid sequence substitutions: (a) the amino acid sequence IRRSSSS (SEQ ID NO: 127) in the HA globular head domain A antigenic site of influenza A/Hong Kong/4801/2014 virus HA has been substituted with the following amino acid sequence MRNGGNS (SEQ ID NO: 128); (b) the amino acid sequences THLNYK (SEQ ID NO: 15) and TDKDQIFPYA (SEQ ID NO: 130) the HA globular head domain B antigenic site of influenza A/Hong Kong/4801/2014 virus HA have been substituted with the following amino acid sequences THLNQK (SEQ ID NO: 17) and TDQDQIFPYA (SEQ ID NO: 131), respectively; (c) the amino acid sequences QNSSIGEICDS (SEQ ID NO: 19) and G-KCKSE (SEQ ID NO: 132) in the HA globular head domain C antigenic site of influenza A/Hong Kong/4801/2014 virus HA have been substituted with the following amino acid sequences ESTGTNRLCMK (SEQ ID NO: 133) and DNNCESK (SEQ ID NO: 134), respectively; (d) the amino acid sequence KRSQQA (SEQ ID NO: 135) in the HA globular head domain D antigenic site of influenza A/Hong Kong/4801/2014 virus HA has been substituted with the following amino acid sequence STYQQT (SEQ ID NO: 136); and (e) the amino acid sequences ENCT (SEQ ID NO: 124), K and IRSGK (SEQ ID NO: 137) the HA globular head domain E antigenic site of influenza A/Hong Kong/4801/2014 virus HA have been substituted with the following amino acid sequences GNCH (SEQ ID NO: 125), M, and LRIGR (SEQ ID NO: 138), respectively. In a specific embodiment, the mosaic influenza virus HA polypeptide further comprises the transmembrane and cytoplasmic domains of the influenza A/Hong Kong/4801/2014 virus HA.

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise one, two, three, four or all of the following amino acid sequence substitutions: (a) the amino acid sequence NNESFNWT-GVTQNGTSSACIRRSSSS (SEQ ID NO: 13) in the HA globular head domain A antigenic site of influenza A/Hong Kong/4801/2014 virus HA has been substituted with the following amino acid sequence NNESFNWT-GVTQNGTSSACLRGGRNS (SEQ ID NO: 139); (b) the amino acid sequences THL-NYK (SEQ ID NO: 15) and GTDKDQIFLYAQ (SEQ ID NO: 16) in the HA globular head domain B antigenic site of influenza A/Hong Kong/4801/2014 virus HA have been substituted with the following amino acid sequences THL-NGK (SEQ ID NO: 140) and GTDNDQIFLYAQ (SEQ ID NO: 141), respectively; (c) the amino acid sequences QNSSIGEICDS (SEQ ID NO: 19) and PIG-KCKSE (SEQ ID NO: 20) in the HA globular head domain C antigenic site of influenza A/Hong Kong/4801/2014 virus HA have been substituted with the following amino acid sequences ETTNHTDECPK (SEQ ID NO: 142) and PIGKSCTSP (SEQ ID NO: 143), respectively; (d) the amino acid sequence RITVSTKRSQQAVIPNIGS (SEQ ID NO: 23) in the HA globular head domain D antigenic site of influenza A/Hong Kong/4801/2014 virus HA has been substituted with the following amino acid sequence RITVSTRSDQQTVIPNIGS (SEQ ID NO: 144); and (e) the amino acid sequences ENCT (SEQ ID NO: 124), GFQNKKWDLFVERSKAY (SEQ ID NO: 27) and IRSGKS (SEQ ID NO: 28) in the HA globular head domain E antigenic site of influenza A/Hong Kong/4801/2014 virus HA have been substituted with the following amino acid sequences QNCD (SEQ ID NO: 145), GFQNKTWDLFVERSKAY (SEQ ID NO: 146) and IRKGRS (SEQ ID NO: 147), respectively. In a specific embodiment, the mosaic influenza virus HA polypeptide further comprises the transmembrane and cytoplasmic domains of the influenza A/Hong Kong/4801/2014 virus HA.

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise one, two, three, four or all of the following amino acid sequence substitutions: (a) the amino acid sequence IRRSSSS (SEQ ID NO: 127) in the HA globular head domain A antigenic site of influenza A/Hong Kong/4801/2014 virus HA has been substituted with the following amino acid sequence LRGGRNS (SEQ ID NO: 148); (b) the amino acid sequences THLNYK (SEQ ID NO: 15) and TDKDQIFPYA (SEQ ID NO: 130) the HA globular head domain B antigenic site of influenza A/Hong Kong/4801/2014 virus HA have been substituted with the following amino acid sequences THLNGK (SEQ ID NO: 140) and TDNDQIFPYA (SEQ ID NO: 149), respectively; (c) the amino acid sequences QNSSIGEICDS (SEQ ID NO: 19) and G-KCKSE (SEQ ID NO: 132) in the HA globular head domain C antigenic site of influenza A/Hong Kong/4801/2014 virus HA have been substituted with the following amino acid sequences ETNHTDELCPS (SEQ ID NO: 150) and G-SCTSP (SEQ ID NO: 151), respectively; (d) the amino acid sequence KRSQQA (SEQ ID NO: 135) in the HA globular head domain D antigenic site of influenza A/Hong Kong/4801/2014 virus HA has been substituted with the following amino acid sequence RSDQQT (SEQ ID NO: 152); and (e) the amino acid sequences ENCT (SEQ ID NO: 124), K and IRSGK (SEQ ID NO: 137) the HA globular head domain E antigenic site of influenza A/Hong Kong/4801/2014 virus HA have been substituted with the following amino acid sequences QNCD (SEQ ID NO: 145), T, and IRKGK (SEQ ID NO: 153), respectively. In a specific embodiment, the mosaic influenza virus HA polypeptide further comprises the transmembrane and cytoplasmic domains of the influenza A/Hong Kong/4801/2014 virus HA.

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise the amino acid sequence substitutions in one, two, three, four or all of the following: (a) the amino acid substitutions in the HA globular head domain A antigenic site of influenza A/Hong Kong/4801/2014 virus HA set forth in FIG. 9B under H3-AA; (b) the amino acid substitutions in the HA globular head domain B antigenic site of influenza A/Hong Kong/4801/2014 virus HA set forth in FIG. 9B under H3-AA; (c) the amino acid substitutions in the HA globular head domain C antigenic site of influenza A/Hong Kong/4801/2014 virus HA set forth in FIG. 9B under H3-AA; (d) the amino acid substitutions in the HA globular head domain D antigenic site of influenza A/Hong Kong/4801/2014 virus HA set forth in FIG. 9B under H3-AA; and the amino acid substitutions in the HA globular head domain E antigenic site of influenza A/Hong Kong/4801/2014 virus HA set forth in FIG. 9B under H3-AA.

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise the amino acid sequence substitutions in one, two, three, four or all of the following: (a) the amino acid substitutions in the HA globular head domain A antigenic site of influenza A/Hong Kong/4801/2014 virus HA set forth in FIG. 14B under mH10/3; (b) the amino acid substitutions in the HA globular head domain B antigenic site of influenza A/Hong Kong/4801/2014 virus HA set forth in FIG. 14B under mH10/3; (c) the amino acid substitutions in the HA globular head domain C antigenic site of influenza A/Hong Kong/4801/2014 virus HA set forth in FIG. 14B under mH10/3; (d) the amino acid substitutions in the HA globular head domain D antigenic site of influenza A/Hong Kong/4801/2014 virus HA set forth in FIG. 14B under mH10/3; and the amino acid substitutions in the HA globular head domain E antigenic site of influenza A/Hong Kong/4801/2014 virus HA set forth in FIG. 14B under mH10/3.

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise the amino acid sequence substitutions in one, two, three, four or all of the following: (a) the amino acid substitutions in the HA globular head domain A antigenic site of influenza A/Hong Kong/4801/2014 virus HA set forth in FIG. 14B under mH14/3; (b) the amino acid substitutions in the HA globular head domain B antigenic site of influenza A/Hong Kong/4801/2014 virus HA set forth in FIG. 14B under mH14/3; (c) the amino acid substitutions in the HA globular head domain C antigenic site of influenza A/Hong Kong/4801/2014 virus HA set forth in FIG. 14B under mH14/3; (d) the amino acid substitutions in the HA globular head domain D antigenic site of influenza A/Hong Kong/4801/2014 virus HA set forth in FIG. 14B under mH14/3; and the amino acid substitutions in the HA globular head domain E antigenic site of influenza A/Hong Kong/4801/2014 virus HA set forth in FIG. 14B under mH14/3.

In a specific embodiment, provided herein is a mosaic influenza virus HA polypeptide comprising the amino acid sequence set forth in SEQ ID NO: 30. In another specific embodiment, provided herein is a mosaic influenza virus HA polypeptide comprising the amino acid sequence set forth in SEQ ID NO: 31.

In another specific embodiment, provided herein is a mosaic influenza virus HA polypeptide comprising the amino acid sequence set forth in FIG. 18A under mH10/3 (SEQ ID NO: 173). In another specific embodiment, provided herein is a mosaic influenza virus HA polypeptide comprising the amino acid sequence set forth in FIG. 18B under mH14/3 (SEQ ID NO: 175).

In another aspect, provided herein is a nucleic acid sequence comprising the nucleotide sequence encoding a mosaic influenza virus HA polypeptide described herein. In a specific embodiment, the nucleic acid sequence further comprises a nucleotide sequence encoding an influenza A virus signal sequence. In some embodiments, the nucleic acid sequence further comprises a nucleotide sequence comprising the 5′ and 3′ non-coding regions of an influenza A virus. In a specific embodiment, the nucleic acid sequence is isolated.

In a specific embodiment, provided herein is a nucleic acid sequence comprising the nucleotide sequence set forth in FIG. 18A under mH10/3 (SEQ ID NO: 172). In another specific embodiment, provided herein is a nucleic acid sequence comprising the nucleotide sequence set forth in FIGS. 18A-18B under mH14/3 (SEQ ID NO: 174). In a specific embodiment, the nucleic acid sequence further comprises a nucleotide sequence encoding an influenza A virus signal sequence. In some embodiments, the nucleic acid sequence further comprises a nucleotide sequence comprising the 5′ and 3′ non-coding regions of an influenza A virus. In a specific embodiment, the nucleic acid sequence is isolated.

In another aspect, provided herein is an expression vector or viral vector comprising a nucleic acid sequence described herein. In a specific embodiment, provided herein is an expression vector or viral vector comprising a nucleic acid sequence, wherein the nucleic acid sequence comprises a nucleotide sequence encoding a mosaic influenza virus HA polypeptide. In another aspect, provided herein is a viral vector comprising a mosaic influenza virus HA polypeptide described herein.

In another aspect, provided herein is an influenza A virus comprising a mosaic influenza virus HA polypeptide described herein. In another aspect, provided herein influenza A virus engineered to express a mosaic influenza virus HA polypeptide described herein. In another aspect, provided herein is an influenza A virus engineered to express and comprise a mosaic influenza virus HA polypeptide described herein. In a specific embodiment, the influenza A virus is A/Puerto Rico/8/34 or an influenza A virus lacking the NS1 protein. In another specific embodiment, the influenza A virus is cold-adapted influenza A virus (e.g., influenza A/Ann Arbor/6/60 virus or influenza A/Leningrad/134/17/57 virus). In some embodiment, such an influenza A virus which is attenuated. In certain embodiments, such an influenza A virus is inactivated.

In another aspect, provided herein is a virus-like particle comprising a mosaic influenza virus HA polypeptide described herein. In another aspect, provided herein is a cell (including, e.g., a population of cells) or cell line expressing a mosaic influenza virus HA polypeptide. See, e.g., Section 5.3, infra for examples of cells. In a specific embodiment, the cell is ex vivo or in vitro. In another specific embodiment, the cell is isolated.

In another aspect, provided herein is a cell or cell line comprising an influenza A virus described herein. See, e.g., Section 5.3, infra for examples of cells. In a specific embodiment, provided herein is a cell (including, e.g., a population of cells) or cell line comprising an influenza A virus, wherein the influenza A virus is engineered to express or contain a mosaic influenza virus HA polypeptide, or the influenza A virus is engineered to express and comprise a mosaic influenza virus HA polypeptide. In a specific embodiment, the cell is ex vivo or in vitro. In another specific embodiment, the cell is isolated.

In another aspect, provided herein is an immunogenic composition comprising a mosaic influenza virus HA polypeptide described herein. In some embodiments, the immunogenic composition further comprises an adjuvant. In a specific embodiment, provide herein is a subunit vaccine comprising a mosaic influenza virus HA polypeptide described herein. In some embodiments, the subunit vaccine further comprises an adjuvant. In another specific embodiment, a split vaccine comprising a mosaic influenza virus HA polypeptide described herein. In some embodiments, the split vaccine further comprises an adjuvant.

In another aspect, provided herein is an immunogenic composition comprising a viral vector described herein. In another aspect, provided herein is an immunogenic composition comprising an influenza A virus described herein. In a specific embodiment, provided herein is an immunogenic composition comprising an influenza A virus, wherein the influenza A virus is engineered to express or contain a mosaic influenza virus HA polypeptide, or the influenza A virus is engineered to express and comprise a mosaic influenza virus HA polypeptide. In some embodiments, the immunogenic composition further comprises an adjuvant.

In another aspect, provided herein is an immunogenic composition comprising a nucleic acid sequence described herein. In a specific embodiment, provided herein is an immunogenic composition comprising a nucleic acid sequence (e.g., an RNA sequence), wherein the nucleic acid sequence comprises a nucleotide sequence encoding a mosaic influenza virus HA polypeptide described herein. In certain embodiments, the immunogenic composition further comprises a second nucleic acid sequence comprising a second nucleotide sequence encoding an influenza A virus neuraminidase (NA). In some embodiments, the immunogenic composition further comprises a second nucleic acid sequence comprising a second nucleotide sequence encoding an influenza A virus nucleoprotein (NP). In certain embodiments, the immunogenic composition further comprises (1) a second nucleic acid sequence comprising a second nucleotide sequence encoding an influenza A virus neuraminidase (NA); and (2) a third nucleic acid sequence comprising a third nucleotide sequence encoding an influenza A virus nucleoprotein (NP). In specific embodiments, the second nucleic acid sequence, third nucleic acid sequence or both is/are RNA sequences. In some embodiments, the immunogenic composition further comprises an adjuvant.

In another aspect, provided herein is an immunogenic composition comprising a virus-like particle described herein. In some embodiments, the immunogenic composition further comprises an adjuvant.

In another aspect, provided herein are methods for inducing an immune response against influenza A virus to a subject (e.g., a human subject) using a mosaic influenza virus HA polypeptide, or a composition thereof. In a specific embodiment, provided herein is a method for inducing an immune response against influenza A virus in a subject (e.g., a human subject) comprising administering to the subject an immunogenic composition described herein (e.g., a subunit vaccine, a split virus vaccine, or a live attenuated virus).

In another aspect, provided herein are methods for immunizing a subject (e.g., a human subject) against influenza A virus using a mosaic influenza virus HA polypeptide, or a composition thereof. In a specific embodiment, provided herein is a method for immunizing a subject (e.g., a human subject) against influenza A virus comprising administering to the subject an immunogenic composition described herein (e.g., a subunit vaccine or a split virus vaccine).

In another aspect, provided herein are methods for preventing an influenza virus disease in a subject (e.g., a human subject) using a mosaic influenza virus HA polypeptide, or a composition thereof. In a specific embodiment, provided herein is a method for preventing an influenza virus disease in a subject (e.g., a human subject) comprising administering to the subject an immunogenic composition described herein (e.g., a subunit vaccine or a split virus vaccine).

In another aspect, provided herein is a method of determining a change in a subject's (e.g., a human subject's) immune response using a mosaic influenza virus HA polypeptide described herein. In a specific embodiment, provided herein is a method of determining a change in a subject's (e.g., a human subject's) immune response to a first influenza A virus, comprising: (a) measuring hemagglutination inhibition in a series of wells containing red blood cells and either inactivated plasma or sera from the subject from a first time point or inactivated plasma or sera from the subject from a second time point, wherein each of the series of wells contains a different influenza A virus, wherein each of the different influenza A viruses comprises a different mosaic influenza virus HA polypeptide, wherein each mosaic influenza virus HA polypeptide comprises an HA ectodomain of the first influenza A virus HA, wherein the HA ectodomain comprises an HA stem domain of the first influenza A virus HA and an HA globular head domain of the first influenza A virus HA, and wherein the HA globular head domain of the first influenza A virus HA has been engineered to comprise amino acid substitutions in one, two, three, four or more of the antigenic sites; and (b) comparing the hemagglutination inhibition in each of the wells, wherein a difference in the inhibition of the hemagglutination in wells containing the plasma or sera from the first time point relative to the inhibition of hemagglutination in wells containing the plasma or sera from the second time point indicates a change in the subject's immune response to the first influenza A virus. In a specific embodiment, the first time point is prior to vaccination with an influenza virus vaccine and the second time point is post-vaccination. In another specific embodiment, the first time point is 6 months, 1 year, 2 years or more before the second time point. In certain embodiments, the difference is an increase in inhibition of hemagglutination using inactivated plasma or sera from the second time point relative to the inhibition of hemagglutinin using inactivated plasma or sera from the first time point. In some embodiments, the change in the subject's immune response to the first influenza A virus is an improvement.

### 3.1 Terminology

As used herein, the term “A antigenic site” refers to an antigenic region in an influenza A virus group 2 HA. In a specific embodiment, the term “A antigenic site” refers to amino acid residues 121-146 of the HA1 domain of influenza A virus A/Hong Kong/4801/2014 or amino acid residues in the HA1 domain of an influenza A virus other than A/Hong Kong/4801/2014 that correspond to amino acid residues 121-146 of the HA1 domain of influenza A virus A/Hong Kong/4801/2014 (wherein the amino acid residues 121-146 correspond to the numbered positions of the influenza A virus A/Hong Kong/4801/2014 not including the signal peptide, i.e., the numbering of the mature HA). In another specific embodiment, the term “A antigenic site” refers to the antigenic region defined by Webster et al., 1980, Virology 104:139-148; Wiley and Skehel, 1987, Annu. Rev. Biochem. 56:365-394; Lee et al., 2014, Nat. Commun. 5:3614; Wilson et al., 1981, Nature 289:366; or Wiley et al., 1981, Nature 289:373 as the A antigenic site or the equivalent thereof in other influenza A viruses.

As used herein, the term “B antigenic site” refers to an antigenic region in an influenza A virus group 2 HA. In a specific embodiment, the term “B antigenic site” refers to amino acid residues 155-160 and 186-197 of the HAI domain of influenza A virus A/Hong Kong/4801/2014 or amino acid residues in the HAI domain of an influenza A virus other than A/Hong Kong/4801/2014 that correspond to amino acid residues 155-160 and 186-197 of the HA1 domain of influenza A virus A/Hong Kong/4801/2014 (wherein the amino acid residues 150-160 and 186-197 correspond to the numbered positions of the influenza A virus A/Hong Kong/4801/2014 not including the signal peptide, i.e., the numbering of the mature HA). In another specific embodiment, the term “B antigenic site” refers to the antigenic region defined by Webster et al., 1980, Virology 104:139-148; Wiley and Skehel, 1987, Annu. Rev. Biochem. 56:365-394; Lee et al., 2014, Nat. Commun. 5:3614; Wilson et al., 1981, Nature 289:366; or Wiley et al., 1981, Nature 289:373 as the B antigenic site or the equivalent thereof in other influenza A viruses.

As used herein, the term “C antigenic site” refers to an antigenic region in an influenza A virus group 2 HA. In a specific embodiment, the term “C antigenic site” refers to amino acid residues 44-54 and 273-280 of the HA1 domain of influenza A virus A/Hong Kong/4801/2014 or amino acid residues in the HAI domain of an influenza A virus other than A/Hong Kong/4801/2014 that correspond to amino acid residues 44-54 and 273-280 of the HA1 domain of influenza A virus A/Hong Kong/4801/2014 (wherein the amino acid residues 44-54 and 273-280 correspond to the numbered positions of the influenza A virus A/Hong Kong/4801/2014 not including the signal peptide, i.e., the numbering of the mature HA). In another specific embodiment, the term “C antigenic site” refers to the antigenic region defined by Webster et al., 1980, Virology 104:139-148; Wiley and Skehel, 1987, Annu. Rev. Biochem. 56:365-394; Lee et al., 2014, Nat. Commun. 5:3614; Wilson et al., 1981, Nature 289:366; or Wiley et al., 1981, Nature 289:373 as the C antigenic site or the equivalent thereof in other influenza A viruses.

As used herein, the term “D antigenic site” refers to an antigenic region in an influenza A virus group 2 HA. In a specific embodiment, the term “D antigenic site” refers to amino acid residues 201-219 of the HAI domain of influenza A virus A/Hong Kong/4801/2014 or amino acid residues in the HAI domain of an influenza A virus other than A/Hong Kong/4801/2014 that correspond to amino acid residues 201-219 of the HA1 domain of influenza A virus A/Hong Kong/4801/2014 (wherein the amino acid residues 201-219 correspond to the numbered positions of the influenza A virus A/Hong Kong/4801/2014 not including the signal peptide, i.e., the numbering of the mature HA). In another specific embodiment, the term “D antigenic site” refers to the antigenic region defined by Webster et al., 1980, Virology 104:139-148; Wiley and Skehel, 1987, Annu. Rev. Biochem. 56:365-394; Lee et al., 2014, Nat. Commun. 5:3614; Wilson et al., 1981, Nature 289:366; or Wiley et al., 1981, Nature 289:373 as the D antigenic site or the equivalent thereof in other influenza A viruses.

As used herein, the term “E antigenic site” refers to an antigenic region in an influenza A virus group 2 HA. In a specific embodiment, the term “E antigenic site” refers to amino acid residues 62-65, 78-94, and 260-265 of the HA1 domain of influenza A virus A/Hong Kong/4801/2014 or amino acid residues in the HA1 domain of an influenza A virus other than A/Hong Kong/4801/2014 that correspond to amino acid residues 62-65, 78-94, and 260-265 of the HA1 domain of influenza A virus A/Hong Kong/4801/2014 (wherein the amino acid residues 62-65, 78-94, and 260-265 correspond to the numbered positions of the influenza A virus A/Hong Kong/4801/2014 not including the signal peptide, i.e., the numbering of the mature HA). In another specific embodiment, the term “E antigenic site” refers to the antigenic region defined by Webster et al., 1980, Virology 104:139-148; Wiley and Skehel, 1987, Annu. Rev. Biochem. 56:365-394; Lee et al., 2014, Nat. Commun. 5:3614; Wilson et al., 1981, Nature 289:366; or Wiley et al., 1981, Nature 289:373 as the E antigenic site or the equivalent thereof in other influenza A viruses.

As used herein, the term “Ca1 antigenic site” refers to an antigenic region in an influenza A virus group 1 HA. In a specific embodiment, the term “Ca1 antigenic site” refers to amino acid residues 166-170, 203-205, and 235-237 of the HAI domain of influenza A virus A/Michigan/45/2015 or amino acid residues in the HA1 domain of an influenza A virus other than A/Michigan/45/2015 that correspond to amino acid residues 166-170, 203-205, and 235-237 of the HAI domain of influenza A virus A/Michigan/45/2015 (wherein the amino acid residues 166-170, 203-205, and 235-237 correspond to the numbered positions of the influenza A virus A/Michigan/45/2015 not including the signal peptide, i.e., the numbering of the mature HA). In another specific embodiment, the term “Ca1 antigenic site” refers to the antigenic region defined by Caton et al., 1982, Cell 31:417-427; or Zhang et al., 2010, Protein Cell 1:549 as the Ca1 antigenic site or the equivalent thereof in other influenza A viruses.

As used herein, the term “Ca2 antigenic site” refers to an antigenic region in an influenza A virus group 1 HA. In a specific embodiment, the term “Ca2 antigenic site” refers to amino acid residues 137-142, 221, and 222 of the HAI domain of influenza A virus A/Michigan/45/2015 or amino acid residues in the HAI domain of an influenza A virus other than A/Michigan/45/2015 that correspond to amino acid residues 137-142, 221, and 222 of the HAI domain of influenza A virus A/Michigan/45/2015 (wherein the amino acid residues 137-142, 221, and 222 correspond to the numbered positions of the influenza A virus A/Michigan/45/2015 not including the signal peptide, i.e., the numbering of the mature HA). In another specific embodiment, the term “Ca2 antigenic site” refers to the antigenic region defined by Caton et al., 1982, Cell 31:417-427; or Zhang et al., 2010, Protein Cell 1:549 as the Ca2 antigenic site or the equivalent thereof in other influenza A viruses.

As used herein, the term “Cb antigenic site” refers to an antigenic region in an influenza A virus group 1 HA. In a specific embodiment, the term “Cb antigenic site” refers to amino acid residues 70-75 of the HA1 domain of influenza A virus A/Michigan/45/2015 or amino acid residues in the HAI domain of an influenza A virus other than A/Michigan/45/2015 that correspond to amino acid residues 70-75 of the HAI domain of influenza A virus A/Michigan/45/2015 (wherein the amino acid residues 70-75 correspond to the numbered positions of the influenza A virus A/Michigan/45/2015 not including the signal peptide, i.e., the numbering of the mature HA). In another specific embodiment, the term “Cb antigenic site” refers to the antigenic region defined by Caton et al., 1982, Cell 31:417-427; or Zhang et al., 2010, Protein Cell 1:549 as the Cb antigenic site or the equivalent thereof in other influenza A viruses.

As used herein, the term “Sa antigenic site” refers to an antigenic region in an influenza A virus group 1 HA. In a specific embodiment, the term “Sa antigenic site” refers to amino acid residues 123, 124, 153-157, and 159-164 of the HA1 domain of influenza A virus A/Michigan/45/2015 or amino acid residues in the HAI domain of an influenza A virus other than A/Michigan/45/2015 that correspond to amino acid residues 123, 124, 153-157, and 159-164 of the HA1 domain of influenza A virus A/Michigan/45/2015 (wherein the amino acid residues 123, 124, 153-157, and 159-164 correspond to the numbered positions of the influenza A virus A/Michigan/45/2015 not including the signal peptide, i.e., the numbering of the mature HA). In another specific embodiment, the term “Sa antigenic site” refers to the antigenic region defined by Caton et al., 1982, Cell 31:417-427; or Zhang et al., 2010, Protein Cell 1:549 as the Sa antigenic site or the equivalent thereof in other influenza A viruses.

As used herein, the term “Sb antigenic site” refers to an antigenic region in an influenza A virus group 1 HA. In a specific embodiment, the term “Sb antigenic site” refers to amino acid residues 184-195 of the HA1 domain of influenza A virus A/Michigan/45/2015 or amino acid residues in the HAI domain of an influenza A virus other than A/Michigan/45/2015 that correspond to amino acid residues 184-195 of the HAI domain of influenza A virus A/Michigan/45/2015 (wherein the amino acid residues 184-195 correspond to the numbered positions of the influenza A virus A/Michigan/45/2015 not including the signal peptide, i.e., the numbering of the mature HA). In another specific embodiment, the term “Sb antigenic site” refers to the antigenic region defined by Caton et al., 1982, Cell 31:417-427; or Zhang et al., 2010, Protein Cell 1:549 as the Sb antigenic site or the equivalent thereof in other influenza A viruses.

The terms “about” or “approximate,” when used in reference to an amino acid position refer to the particular amino acid position in a sequence or any amino acid that is within five, four, three, two, or one residues of that amino acid position, either in an N-terminal direction or a C-terminal direction.

As used herein, the term “about” or “approximately” when used in conjunction with a number refers to any number within 1, 5 or 10% of the referenced number. In certain embodiments, the term “about” encompasses the exact number recited.

The term “amino acid sequence identity” has the meaning understood to a person skilled in the art. The term “amino acid identity” generally refers to the degree of identity or similarity between a pair of aligned amino acid sequences, usually expressed as a percentage. Percent identity is the percentage of amino acid residues in a candidate sequence that are identical (i.e., the amino acid residues at a given position in the alignment are the same residue) or similar (i.e., the amino acid substitution at a given position in the alignment is a conservative substitution, as discussed below), to the corresponding amino acid residue in the peptide after aligning the sequences and introducing gaps, if necessary, to achieve the maximum percent sequence homology. Sequence homology, including percentages of sequence identity and similarity, may be determined using sequence alignment techniques well-known in the art, preferably computer algorithms designed for this purpose, using the default parameters of said computer algorithms or the software packages containing them. Non-limiting examples of computer algorithms and software packages incorporating such algorithms include the following. The BLAST family of programs exemplify a particular, non-limiting example of a mathematical algorithm utilized for the comparison of two sequences (e.g., Karlin & Altschul, 1990, Proc. Natl. Acad. Sci. USA 87:2264-2268 (modified as in Karlin & Altschul, 1993, Proc. Natl. Acad. Sci. USA 90:5873-5877), Altschul et al., 1990, J. Mol. Biol. 215:403-410, (describing NBLAST and XBLAST), Altschul et al., 1997, Nucleic Acids Res. 25:3389-3402 (describing Gapped BLAST, and PSI-Blast). Another particular example is the algorithm of Myers and Miller (1988 CABIOS 4:11-17) which is incorporated into the ALIGN program (version 2.0) and is available as part of the GCG sequence alignment software package. Also particular is the FASTA program (Pearson W. R. and Lipman D. J., Proc. Nat. Acad. Sci. USA, 85:2444-2448, 1988), available as part of the Wisconsin Sequence Analysis Package. Additional examples include BESTFIT, which uses the “local homology” algorithm of Smith and Waterman (Advances in Applied Mathematics, 2:482-489, 1981) to find best single region of similarity between two sequences, and which is preferable where the two sequences being compared are dissimilar in length; and GAP, which aligns two sequences by finding a “maximum similarity” according to the algorithm of Neddleman and Wunsch (J. Mol. Biol. 48:443-354, 1970), and is preferable where the two sequences are approximately the same length and an alignment is expected over the entire length.

“Conservative substitution” refers to replacement of an amino acid of one class is with another amino acid of the same class. In particular embodiments, a conservative substitution does not alter the structure or function, or both, of a polypeptide. Classes of amino acids for the purposes of conservative substitution include hydrophobic (Met, Ala, Val, Leu, Ile), neutral hydrophilic (Cys, Ser, Thr), acidic (Asp, Glu), basic (Asn, Gln, His, Lys, Arg), conformation disrupters (Gly, Pro) and aromatic (Trp, Tyr, Phe).

As described herein, the term “ectodomain” in reference to an influenza A virus HA polypeptide would be understood by one of skill in the art. Typically, the ectodomain of an influenza A virus HA comprises the globular head domain and stem domain of an influenza virus HA. See, e.g., Table 1, Table 2, Table 3, Table 4, and Table 5 below, for exemplary influenza A virus ectodomain sequences and locations. In certain embodiments, the ectodomain of an influenza A virus HA polypeptide is a region of the influenza A virus HA polypeptide that aligns with the ectodomain of influenza A/Hong Kong/4801/2014 virus HA ectodomain set forth in Table 1, below.

In some embodiments, the ectodomain of an influenza A virus HA polypeptide is a region of the influenza A virus HA polypeptide that aligns with the ectodomain of influenza A/Jiangxi-Donghu/346-1/2013 virus HA ectodomain set forth in Table 2, below. In certain embodiments, the ectodomain of an influenza A virus HA polypeptide is a region of the influenza A virus HA polypeptide that aligns with the ectodomain of influenza A/Michigan/45/2015 virus HA ectodomain set forth in Table 3, below. In some embodiments, the ectodomain of an influenza A virus HA polypeptide is a region of the influenza A virus HA polypeptide that aligns with the ectodomain of influenza virus A/Vietnam/1203/2004 virus HA ectodomain set forth in Table 4, below. In certain embodiments, the ectodomain of an influenza A virus HA polypeptide is a region of the influenza A virus HA polypeptide that aligns with the ectodomain of influenza virus A/black headed gull/Sweden/1/1999 virus HA ectodomain set forth in Table 5, below. In some embodiments, the ectodomain of an influenza A virus HA polypeptide is a region of the influenza A virus HA polypeptide that aligns with the ectodomain of influenza virus A/mallard/Gurjev/263/1982.

TABLE 1
Exemplary domains for influenza A/Hong Kong/4801/2014 HA.

| | Amino Acid | | |
|---|---|---|---|
| | residue | | |
| | numbers | | |
| | using | | |
| | immature | | |
| | numbering | Residue | |
| | (positions in | numbers | |
| | influenza | using mature | |
| | virus A/Hong | numbering | |
| | Kong/4801/ | (positions in | |
| | 2014 HA, | influenza | |
| | inclusive of | virus A/Hong | |
| | the signal | Kong/4801/ | |
| Domain for | peptide with | 2014 HA, not | |
| influenza virus | exception of | including the | |
| A/Hong | loops; | signal | |
| Kong/4801/2014 | immature | peptide; | |
| HA | HA) | mature HA) | Amino Acid Sequence |
| Signal Sequence | 1-16 | | MKTIIALSYILCLVFA (SEQ ID NO: 40) |
| Ectodomain | 1-531 | 1-515 | QKIPGNDNSTATLCLGHHAVPNGTIVKTITNDRIEVT |
| | | | NATELVQNSSIGEICDSPHQILDGENCTLIDALLGDP |
| | | | QCDGFQNKKWDLFVERSKAYSNCYPYDVPDYASL |
| | | | RSLVASSGTLEFNNESFNWTGVTQNGTSSACIRRSSS |
| | | | SFFSRLNWLTHLNYTYPALNVTMPNNEQFDKLYIW |
| | | | GVHHPGTDKDQIFLYAQSSGRITVSTKRSQQAVIPNI |
| | | | GSRPRIRDIPSRISIYWTIVKPGDILLINSTGNLIAPRG |
| | | | YFKIRSGKSSIMRSDAPIGKCKSECITPNGSIPNDKPF |
| | | | QNVNRITYGACPRYVKHSTLKLATGMRNVPEKQTR |
| | | | GIFGAIAGFIENGWEGMVDGWYGFRHQNSEGRGQA |
| | | | ADLKSTQAAIDQINGKLNRLIGKTNEKFHQIEKEFSE |
| | | | VEGRIQDLEKYVEDTKIDLWSYNAELLVALENQHTI |
| | | | DLTDSEMNKLFEKTKKQLRENAEDMGNGCFKIYHK |
| | | | CDNACIGSIRNGTYDHNVYRDEALNNRFQIKGVELK |
| | | | SGYKDWI (SEQ ID NO: 41) |
| HA1 Domain | 17-345 | 1-329 | QKIPGNDNSTATLCLGHHAVPNGTIVKTITNDRIEVT |
| | | | NATELVQNSSIGEICDSPHQILDGENCTLIDALLGDP |
| | | | QCDGFQNKKWDLFVERSKAYSNCYPYDVPDYASL |
| | | | RSLVASSGTLEFNNESFNWTGVTQNGTSSACIRRSSS |
| | | | SFFSRLNWLTHLNYTYPALNVTMPNNEQFDKLYIW |
| | | | GVHHPGTDKDQIFLYAQSSGRITVSTKRSQQAVIPNI |
| | | | GSRPRIRDIPSRISIYWTIVKPGDILLINSTGNLIAPRG |
| | | | YFKIRSGKSSIMRSDAPIGKCKSECITPNGSIPNDKPF |
| | | | QNVNRITYGACPRYVKHSTLKLATGMRNVPEKQTR |
| | | | (SEQ ID NO: 42) |
| HA2 Domain¹ | 346-531 | 330-515 | GIFGAIAGFIENGWEGMVDGWYGFRHQNSEGRGQA |
| | | | ADLKSTQAAIDQINGKLNRLIGKTNEKFHQIEKEFSE |
| | | | VEGRIQDLEKYVEDTKIDLWSYNAELLVALENQHTI |
| | | | DLTDSEMNKLFEKTKKQLRENAEDMGNGCFKIYHK |
| | | | CDNACIGSIRNGTYDHNVYRDEALNNRFQIKGVELK |
| | | | SGYKDWI (SEQ ID NO: 71) |
| Stem Domain | 17-68; | 1-52; | QKIPGNDNSTATLCLGHHAVPNGTIVKTITNDRIEVT |
| | 293-531 | 277-515 | NATELVQNSSIGEIC; |
| | | | CKSECITPNGSIPNDKPFQNVNRITYGACPRYVKHST |
| | | | LKLATGMRNVPEKQTRGIFGAIAGFIENGWEGMVD |
| | | | GWYGFRHQNSEGRGQAADLKSTQAAIDQINGKLNR |
| | | | LIGKTNEKFHQIEKEFSEVEGRIQDLEKYVEDTKIDL |
| | | | WSYNAELLVALENQHTIDLTDSEMNKLFEKTKKQL |
| | | | RENAEDMGNGCFKIYHKCDNACIGSIRNGTYDHNV |
| | | | YRDEALNNRFQIKGVELKSGYKDWI (SEQ ID NO: |
| | | | 44) |
| HA1 C-Terminal | 293-345 | 277-329 | CKSECITPNGSIPNDKPFQNVNRITYGACPRYVKHST |
| Stem Segment | | | LKLATGMRNVPEKQTR (SEQ ID NO: 63) |
| HA1 N-Terminal | 17-68 | 1-52 | QKIPGNDNSTATLCLGHHAVPNGTIVKTITNDRIEVT |
| Stem Segment | | | NATELVQNSSIGEIC (SEQ ID NO: 64) |
| Globular Head | 69-294 | 53-276 | DSPHQILDGENCTLIDALLGDPQCDGFQNKKWDLF |
| Domain | | | VERSKAYSNCYPYDVPDYASLRSL VASSGTLEFNNE |
| | | | SFNWTGVTQNGTSSACIRRSSSSFFSRLNWLTHLNY |
| | | | TYPALNVTMPNNEQFDKLYIWGVHHPGTDKDQIFL |
| | | | YAQSSGRITVSTKRSQQAVIPNIGSRPRIRDIPSRISIY |
| | | | WTIVKPGDILLINSTGNLIAPRGYFKIRSGKSSIMRSD |
| | | | APIGK (SEQ ID NO: 45) |
| Transmembrane | 532-552 | 516-536 | LWISFAISCFLLCVALLGFIM (SEQ ID NO: 46) |
| Domain² | | | |
| Cytoplasmic | 553-566 | 537-550 | WACQKGNIRCNICI (SEQ ID NO: 47) |
| Domain | | | |

¹HA2 was defined here to not include the transmembrane domain (TMD) or cytoplasmic domain (CD).
²http://octopus.cbr.su.se/ was used to determine the transmembrane domain

TABLE 2
Exemplary domains for influenza viruse A/Jiangxi-Donghu/346-1/2013
HA.

| | Amino Acid | | |
|---|---|---|---|
| | residue | | |
| | numbers | | |
| | using | | |
| | immature | | |
| | numbering | Residue | |
| | (positions in | numbers | |
| | influenza | using mature | |
| | virus | numbering | |
| | A/Jiangxi- | (positions in | |
| | Donghu/346- | influenza | |
| | 1/2013 HA, | virus | |
| | inclusive of | A/Jiangxi- | |
| Domain for | the signal | Donghu/346- | |
| influenza | peptide with | 1/2013 HA, | |
| virus | exception of | not including | |
| A/Jiangxi- | loops; | the signal | |
| Donghu/346- | immature | peptide; | |
| 1/2013 HA | HA) | mature HA) | Amino Acid Sequence |
| Signal Sequence | 1-16 | | MYKIVVIIALLGAVKG (SEQ ID NO: 48) |
| Ectodomain | 17-526 | 1-510 | LDKICLGHHAVANGTIVKTLTNEQEEVTNATETVES |
| | | | TGINRLCMKGRKHKDLGNCHPIGMLIGTPACDLHLT |
| | | | GMWDTLIERENAIAYCYPGATVNVEALRQKIMESG |
| | | | GINKISTGFTYGSSINSAGTTRACMRNGGNSFYAEL |
| | | | KWLVSKSKGQNFPQTTNTYRNTDTAEHLIMWGIHH |
| | | | PSSTQEKNDLYGTQSLSISVGSSTYRNNFVPVVGAR |
| | | | PQVNGQSGRIDFHWTLVQPGDNITFSHNGGLIAPSR |
| | | | VSKLIGRGLGIQSDAPIDNNCESKCFWRGGSINTRLP |
| | | | FQNLSPRTVGQCPKYVNRRSLMLATGMRNVPELIQ |
| | | | GRGLFGAIAGFLENGWEGMVDGWYGFRHQNAQGT |
| | | | GQAADYKSTQAAIDQITGKLNRLVEKTNTEFESIESE |
| | | | FSEIEHQIGNVINWTKDSITDIWTYQAELLVAMENQ |
| | | | HTIDMADSEMLNLYERVRKQLRQNAEEDGKGCFEI |
| | | | YHACDDSCMESIRNNTYDHSQYREEALLNRLNINPV |
| | | | TLSSGYKDII (SEQ ID NO: 49) |
| HA1 Domain | 17-340 | 1-324 | LDKICLGHHAVANGTIVKTLTNEQEEVTNATETVES |
| | | | TGINRLCMKGRKHKDLGNCHPIGMLIGTPACDLHLT |
| | | | GMWDTLIERENAIAYCYPGATVNVEALRQKIMESG |
| | | | GINKISTGFTYGSSINSAGTTRACMRNGGNSFYAEL |
| | | | KWLVSKSKGQNFPQTTNTYRNTDTAEHLIMWGIHH |
| | | | PSSTQEKNDLYGTQSLSISVGSSTYRNNFVPVVGAR |
| | | | PQVNGQSGRIDFHWTLVQPGDNITFSHNGGLIAPSR |
| | | | VSKLIGRGLGIQSDAPIDNNCESKCFWRGGSINTRLP |
| | | | FQNLSPRTVGQCPKYVNRRSLMLATGMRNVPELIQ |
| | | | GR (SEQ ID NO: 50) |
| HA2 Domain¹ | 341-526 | 325-510 | GLFGAIAGFLENGWEGMVDGWYGFRHQNAQGTGQ |
| | | | AADYKSTQAAIDQITGKLNRLVEKTNTEFESIESEFS |
| | | | EIEHQIGNVINWTKDSITDIWTYQAELLVAMENQHT |
| | | | IDMADSEMLNLYERVRKQLRQNAEEDGKGCFEIYH |
| | | | ACDDSCMESIRNNTYDHSQYREEALLNRLNINPVTL |
| | | | SSGYKDII (SEQ ID NO: 72) |
| Stem Domain | 17-59; | 1-43; | LDKICLGHHAVANGTIVKTLTNEQEEVTNATETVES |
| | 287-526 | 271-510 | TGINRLC; |
| | | | CESKCFWRGGSINTRLPFQNLSPRTVGQCPKYVNRR |
| | | | SLMLATGMRNVPELIQGRGLFGAIAGFLENGWEGM |
| | | | VDGWYGFRHQNAQGTGQAADYKSTQAAIDQITGK |
| | | | LNRLVEKTNTEFESIESEFSEIEHQIGNVINWTKDSIT |
| | | | DIWTYQAELLVAMENQHTIDMADSEMLNLYERVR |
| | | | KQLRQNAEEDGKGCFEIYHACDDSCMESIRNNTYD |
| | | | HSQYREEALLNRLNINPVTLSSGYKDII (SEQ ID |
| | | | NO: 52) |
| HA1 C-Terminal | 287-340 | 271-324 | CESKCFWRGGSINTRLPFQNLSPRTVGQCPKYVNRR |
| Stem Segment | | | SLMLATGMRNVPELIQGR (SEQ ID NO: 65) |
| HA1 N-Terminal | 17-59 | 1-43 | LDKICLGHHAVANGTIVKTLTNEQEEVTNATETVES |
| Stem Segment | | | TGINRLC (SEQ ID NO: 66) |
| Globular Head | 60-286 | 44-270 | MKGRKHKDLGNCHPIGMLIGTPACDLHLTGMWDT |
| Domain | | | LIERENAIAYCYPGATVNVEALRQKIMESGGINKIST |
| | | | GFTYGSSINSAGTTRACMRNGGNSFYAELKWLVSK |
| | | | SKGQNFPQTTNTYRNTDTAEHLIMWGIHHPSSTQEK |
| | | | NDLYGTQSLSISVGSSTYRNNFVPVVGARPQVNGQS |
| | | | GRIDFHWTLVQPGDNITFSHNGGLIAPSRVSKLIGRG |
| | | | LGIQSDAPIDNN (SEQ ID NO: 53) |
| Transmembrane | 527-547 | 511-531 | LWFSFGASCFVLLAVVMGLFF (SEQ ID NO: 54) |
| Domain² | | | |
| Cytoplasmic | 548-561 | 532-545 | FCLKNGNMRCTICI (SEQ ID NO: 126) |
| Domain | | | |

¹HA2 was defined here to not include the TMD or CD.
²http://octopus.cbr.su.se/ was used to determine the transmembrane domain

TABLE 3
Exemplary domains for influenza A/Michigan/45/2015 HA.

| | Amino Acid | | |
|---|---|---|---|
| | residue | | |
| | numbers | | |
| | using | | |
| | immature | | |
| | numbering | Residue | |
| | (positions in | numbers | |
| | influenza | using mature | |
| | virus | numbering | |
| | A/Michigan/ | (positions in | |
| | 45/2015 HA, | influenza | |
| | inclusive of | virus | |
| | the signal | A/Michigan/ | |
| | peptide with | 45/2015 HA, | |
| Domain for | exception of | not including | |
| influenza virus | loops; | the signal | |
| A/Michigan/45/ | immature | peptide; | |
| 2015 HA | HA) | mature HA) | Amino Acid Sequence |
| Signal Sequence | 1-17 | | MKAILVVLLYTFTTANA (SEQ ID NO: 32) |
| Ectodomain | 18-531 | 1-514 | DTLCIGYHANNSTDTVDTVLEKNVTVTHSVNLLED |
| | | | KHNGKLCKLRGVAPLHLGKCNIAGWILGNPECESLS |
| | | | TASSWSYIVETSNSDNGTCYPGDFINYEELREQLSSV |
| | | | SSFERFEIFPKTSSWPNHDSNKGVTAACPHAGAKSF |
| | | | YKNLIWLVKKGNSYPKLNQSYINDKGKEVLVLWGI |
| | | | HHPSTTADQQSLYQNADAYVFVGTSRYSKKFKPEIA |
| | | | TRPKVRDQEGRMNYYWTLVEPGDKITFEATGNLVV |
| | | | PRYAFTMERNAGSGIIISDTPVHDCNTTCQTPEGAIN |
| | | | TSLPFQNIHPITIGKCPKYVKSTKLRLATGLRNVPSIQ |
| | | | SRGLFGAIAGFIEGGWTGMVDGWYGYHHQNEQGS |
| | | | GYAADLKSTQNAIDKITNKVNSVIEKMNTQFTAVG |
| | | | KEFNHLEKRIENLNKKVDDGFLDIWTYNAELLVLLE |
| | | | NERTLDYHDSNVKNLYEKVRNQLKNNAKEIGNGCF |
| | | | EFYHKCDNTCMESVKNGTYDYPKYSEEAKLNREKI |
| | | | DGVKLESTRIYQIL (SEQ ID NO: 33) |
| HA1 Domain | 18-344 | 1-327 | DTLCIGYHANNSTDTVDTVLEKNVTVTHSVNLLED |
| | | | KHNGKLCKLRGVAPLHLGKCNIAGWILGNPECESLS |
| | | | TASSWSYIVETSNSDNGTCYPGDFINYEELREQLSSV |
| | | | SSFERFEIFPKTSSWPNHDSNKGVTAACPHAGAKSF |
| | | | YKNLIWLVKKGNSYPKLNQSYINDKGKEVLVLWGI |
| | | | HHPSTTADQQSLYQNADAYVFVGTSRYSKKFKPEIA |
| | | | TRPKVRDQEGRMNYYWTLVEPGDKITFEATGNLVV |
| | | | PRYAFTMERNAGSGIIISDTPVHDCNTTCQTPEGAIN |
| | | | TSLPFQNIHPITIGKCPKYVKSTKLRLATGLRNVPSIQ |
| | | | SR (SEQ ID NO: 34) |
| HA2 Domain¹ | 345-531 | 328-514 | GLFGAIAGFIEGGWTGMVDGWYGYHHQNEQGSGY |
| | | | AADLKSTQNAIDKITNKVNSVIEKMNTQFTAVGKEF |
| | | | NHLEKRIENLNKKVDDGFLDIWTYNAELLVLLENE |
| | | | RTLDYHDSNVKNLYEK VRNQLKNNAKEIGNGCFEF |
| | | | YHKCDNTCMESVKNGTYDYPKYSEEAKLNREKIDG |
| | | | VKLESTRIYQIL (SEQ ID NO: 73) |
| Stem Domain | 18-59; | 1-42; | DTLCIGYHANNSTDTVDTVLEKNVTVTHSVNLLED |
| | 292-531 | 275-514 | KHNGKLC; |
| | | | CNTTCQTPEGAINTSLPFQNIHPITIGKCPKYVKSTKL |
| | | | RLATGLRNVPSIQSRGLFGAIAGFIEGGWTGMVDG |
| | | | WYGYHHQNEQGSGYAADLKSTQNAIDKITNKVNS |
| | | | VIEKMNTQFTAVGKEFNHLEKRIENLNKKVDDGFL |
| | | | DIWTYNAELLVLLENERTLDYHDSNVKNLYEKVRN |
| | | | QLKNNAKEIGNGCFEFYHKCDNTCMESVKNGTYD |
| | | | YPKYSEEAKLNREKIDGVKLESTRIYQIL (SEQ ID |
| | | | NO: 36) |
| HA1 C-Terminal | 292-344 | 275-327 | CNTTCQTPEGAINTSLPFQNIHPITIGKCPKYVKSTKL |
| Stem Segment | | | RLATGLRNVPSIQSR (SEQ ID NO: 67) |
| HA1 N-Terminal | 18-59 | 1-42 | DTLCIGYHANNSTDTVDTVLEKNVTVTHSVNLLED |
| Stem Segment | | | KHNGKLC (SEQ ID NO: 68) |
| Globular Head | 60-291 | 43-274 | KLRGVAPLHLGKCNIAGWILGNPECESLSTASSWSY |
| Domain | | | IVETSNSDNGTCYPGDFINYEELREQLSSVSSFERFEI |
| | | | FPKTSSWPNHDSNKGVTAACPHAGAKSFYKNLIWL |
| | | | VKKGNSYPKLNQSYINDKGKEVLVLWGIHHPSTTA |
| | | | DQQSLYQNADAYVFVGTSRYSKKFKPEIATRPKVR |
| | | | DQEGRMNYYWTLVEPGDKITFEATGNL VVPRYAFT |
| | | | MERNAGSGIIISDTPVHD (SEQ ID NO: 37) |
| Transmembrane | 532-552 | 515-535 | AIYSTVASSLVLVVSLGAISF (SEQ ID NO: 38) |
| Domain² | | | |
| Cytoplasmic | 553-566 | 536-549 | WMCSNGSLQCRICI (SEQ ID NO: 39) |
| Domain | | | |

¹HA2 was defined here to not include the TMD or CD
²http://octopus.cbr.su.se/ was used to determine the transmembrane domain

TABLE 4
Exemplary domains for influenza A/Vietnam/1203/2004 HA.

| | Amino Acid | | |
|---|---|---|---|
| | residue | | |
| | numbers | | |
| | using | | |
| | immature | | |
| | numbering | | |
| | (positions | Residue | |
| | in | numbers | |
| | influenza | using mature | |
| | virus | numbering | |
| | A/Vietnam/12 | (positions in | |
| | 03/04 HA, | influenza | |
| | inclusive of | virus | |
| | the signal | A/Vietnam/12 | |
| | peptide with | 03/04 HA, not | |
| Domain for | exception of | including the | |
| influenza virus | loops; | signal | |
| A/Vietnam/1203/ | immature | peptide; | |
| 04 HA | HA) | mature HA) | Amino Acid Sequence |
| Signal Sequence | 1-16 | | MEKIVLLFAIVSLVKS (SEQ ID NO: 55) |
| Ectodomain | 17-533 | 1-517 | DQICIGYHANNSTEQVDTIMEKNVTVTHAQDILEKK |
| | | | HNGKLCDLDGVKPLILRDCSVAGWLLGNPMCDEFI |
| | | | NVPEWSYIVEKANPVNDLCYPGDFNDYEELKHLLS |
| | | | RINHFEKIQIIPKSSWSSHEASLGVSSACPYQGKSSFF |
| | | | RNVVWLIKKNSTYPTIKRSYNNTNQEDLLVLWGIH |
| | | | HPNDAAEQTKLYQNPTTYISVGTSTLNQRLVPRIAT |
| | | | RSKVNGQSGRMEFFWTILKPNDAINFESNGNFIAPE |
| | | | YAYKIVKKGDSTIMKSELEYGNCNTKCQTPMGAIN |
| | | | SSMPFHNIHPLTIGECPKYVKSNRLVLATGLRNSPQR |
| | | | ERRRKKRGLFGAIAGFIEGGWQGMVDGWYGYHHS |
| | | | NEQGSGYAADKESTQKAIDGVTNKVNSIIDKMNTQ |
| | | | FEAVGREFNNLERRIENLNKKMEDGFLDVWTYNAE |
| | | | LLVLMENERTLDFHDSNVKNLYDKVRLQLRDNAK |
| | | | ELGNGCFEFYHKCDNECMESVRNGTYDYPQYSEEA |
| | | | RLKREEISGVKLESIGIYQIL (SEQ ID NO: 56) |
| HA1 Domain | 17-346 | 1-330 | DQICIGYHANNSTEQVDTIMEKNVTVTHAQDILEKK |
| | | | HNGKLCDLDGVKPLILRDCSVAGWLLGNPMCDEFI |
| | | | NVPEWSYIVEKANPVNDLCYPGDFNDYEELKHLLS |
| | | | RINHFEKIQIIPKSSWSSHEASLGVSSACPYQGKSSFF |
| | | | RNVVWLIKKNSTYPTIKRSYNNTNQEDLLVLWGIH |
| | | | HPNDAAEQTKLYQNPTTYISVGTSTLNQRLVPRIAT |
| | | | RSKVNGQSGRMEFFWTILKPNDAINFESNGNFIAPE |
| | | | YAYKIVKKGDSTIMKSELEYGNCNTKCQTPMGAIN |
| | | | SSMPFHNIHPLTIGECPKYVKSNRLVLATGLRNSPQR |
| | | | ERRRKKR (SEQ ID NO: 57) |
| HA2 Domain¹ | 347-533 | 331-517 | GLFGAIAGFIEGGWQGMVDGWYGYHHSNEQGSGY |
| | | | AADKESTQKAIDGVTNKVNSIIDKMNTQFEAVGREF |
| | | | NNLERRIENLNKKMEDGFLDVWTYNAELLVLMEN |
| | | | ERTLDFHDSNVKNLYDKVRLQLRDNAKELGNGCFE |
| | | | FYHKCDNECMESVRNGTYDYPQYSEEARLKREEIS |
| | | | GVKLESIGIYQIL (SEQ ID NO: 74) |
| Stem Domain | 17-58; | 1-42; | DQICIGYHANNSTEQVDTIMEKNVTVTHAQDILEKK |
| | 290-533 | 274-517 | HNGKLC; |
| | | | CNTKCQTPMGAINSSMPFHNIHPLTIGECPKYVKSN |
| | | | RLVLATGLRNSPQRERRRKKRGLFGAIAGFIEGGWQ |
| | | | GMVDGWYGYHHSNEQGSGYAADKESTQKAIDGVT |
| | | | NKVNSIIDKMNTQFEAVGREFNNLERRIENLNKKME |
| | | | DGFLDVWTYNAELLVLMENERTLDFHDSNVKNLY |
| | | | DKVRLQLRDNAKELGNGCFEFYHKCDNECMESVR |
| | | | NGTYDYPQYSEEARLKREEISGVKLESIGIYQIL |
| | | | (SEQ ID NO: 59) |
| HA1 C-Terminal | 290-346 | 274-330 | CNTKCQTPMGAINSSMPFHNIHPLTIGECPKYVKSN |
| Stem Segment | | | RLVLATGLRNSPQRERRRKKR (SEQ ID NO: 69) |
| HA1 N-Terminal | 17-58 | 1-42 | DQICIGYHANNSTEQVDTIMEKNVTVTHAQDILEKK |
| Stem Segment | | | HNGKLC (SEQ ID NO: 70) |
| Globular Head | 59-289 | 43-273 | DLDGVKPLILRDCSVAGWLLGNPMCDEFINVPEWS |
| Domain | | | YIVEKANPVNDLCYPGDFNDYEELKHLLSRINHFEK |
| | | | IQIIPKSSWSSHEASLGVSSACPYQGKSSFFRNVVWL |
| | | | IKKNSTYPTIKRSYNNTNQEDLLVLWGIHHPNDAAE |
| | | | QTKLYQNPTTYISVGTSTLNQRLVPRIATRSKVNGQ |
| | | | SGRMEFFWTILKPNDAINFESNGNFIAPEYAYKIVKK |
| | | | GDSTIMKSELEYGN (SEQ ID NO: 60) |
| Transmembrane | 534-554 | 518-538 | SIYSTVASSLALAIMVAGLSL (SEQ ID NO: 61) |
| Domain² | | | |
| Cytoplasmic | 555-568 | 539-552 | WMCSNGSLQCRICI (SEQ ID NO: 62) |
| Domain | | | |

¹HA2 was defined here to not include the TMD or CD
²http://octopus.cbr.su.se/ was used to determine the transmembrane domain

TABLE 5
Exemplary domains for influenza virus A/black headed
gull/Sweden/1/1999 HA.

| | Amino Acid | | |
|---|---|---|---|
| | residue | | |
| | numbers | | |
| | using | | |
| | immature | | |
| | numbering | Residue | |
| | (positions in | numbers | |
| | influenza | using mature | |
| | virus A/black | numbering | |
| | headed | (positions in | |
| | gull/Sweden/ | influenza | |
| | 1/1999 HA, | virus A/black | |
| | inclusive of | headed | |
| | the signal | gull/Sweden/ | |
| Domain for | peptide with | 1/1999 HA, | |
| influenza virus | exception of | not including | |
| A/black headed | loops; | the signal | |
| gull/Sweden/1/ | immature | peptide; | |
| 1999 HA | HA) | mature HA) | Amino Acid Sequence |
| Signal Sequence | 1-18 | | MDIPVVAFLILTSTCVQA (SEQ ID NO: 75) |
| Ectodomain | 19-530 | 1-512 | DRICVGYLSTNSSEKVDTLLENDVPVTSSVDLVETN |
| | | | HTGTYCSLGGISPVHLGDCSFEGWIVGNPACASNLG |
| | | | IREWSYLIEDPSAPHGLCYPGELDNNGELRHLFSGIR |
| | | | SFSRTELIAPTSWGAVNDGVSSACPDKGASSFYRNL |
| | | | VWFVKRGNQYPVIRGTYNNTTGRDVLVIWGIHHPV |
| | | | STDEAKQLYVNNNPYTLVSTSSWSRKYNLETGTRP |
| | | | GYNGQKSWMKIYWYLMHPGESISFESNGGLLAPRY |
| | | | GYIIEEYGKGRIFQSRIRIAKCNTKCQTSVGGINTNKT |
| | | | FQNIERNALGDCPKYIKSGQLKLATGLRNVPAISNR |
| | | | GLFGAIAGFIEGGWPGLINGWYGFQHQNEQGVGMA |
| | | | ADKESTQKAIDQITTKINNIIEKMNGNYDSIRGEFNQ |
| | | | VEQRINMLADRIDDAVTDVWSYNAKLLVLLENDKT |
| | | | LDMHDANVRNLHDQVRRALKTNAIDEGNGCFELL |
| | | | HKCNDSCMETIRNGTYNHTEYEEESKLKRQEIEGIK |
| | | | LKSDDSVYKAL (SEQ ID NO: 76) |
| HA1 Domain | 19-342 | 1-324 | DRICVGYLSTNSSEKVDTLLENDVPVTSSVDLVETN |
| | | | HTGTYCSLGGISPVHLGDCSFEGWIVGNPACASNLG |
| | | | IREWSYLIEDPSAPHGLCYPGELDNNGELRHLFSGIR |
| | | | SFSRTELIAPTSWGAVNDGVSSACPDKGASSFYRNL |
| | | | VWFVKRGNQYPVIRGTYNNTTGRDVLVIWGIHHPV |
| | | | STDEAKQLYVNNNPYTLVSTSSWSRKYNLETGTRP |
| | | | GYNGQKSWMKIYWYLMHPGESISFESNGGLLAPRY |
| | | | GYIIEEYGKGRIFQSRIRIAKCNTKCQTSVGGINTNKT |
| | | | FQNIERNALGDCPKYIKSGQLKLATGLRNVPAISNR |
| | | | (SEQ ID NO: 77) |
| HA2 Domain¹ | 343-530 | 325-512 | GLFGAIAGFIEGGWPGLINGWYGFQHQNEQGVGMA |
| | | | ADKESTQKAIDQITTKINNIIEKMNGNYDSIRGEFNQ |
| | | | VEQRINMLADRIDDAVTDVWSYNAKLLVLLENDKT |
| | | | LDMHDANVRNLHDQVRRALKTNAIDEGNGCFELL |
| | | | HKCNDSCMETIRNGTYNHTEYEEESKLKRQEIEGIK |
| | | | LKSDDSVYKAL (SEQ ID NO: 88) |
| Stem Domain | 19-60; | 1-42; | DRICVGYLSTNSSEKVDTLLENDVPVTSSVDLVETN |
| | 290-530 | 272-512 | HTGTYC; |
| | | | CNTKCQTSVGGINTNKTFQNIERNALGDCPKYIKSG |
| | | | QLKLATGLRNVPAISNRGLFGAIAGFIEGGWPGLING |
| | | | WYGFQHQNEQGVGMAADKESTQKAIDQITTKINNII |
| | | | EKMNGNYDSIRGEFNQVEQRINMLADRIDDAVTDV |
| | | | WSYNAKLLVLLENDKTLDMHDANVRNLHDQVRR |
| | | | ALKTNAIDEGNGCFELLHKCNDSCMETIRNGTYNH |
| | | | TEYEEESKLKRQEIEGIKLKSDDSVYKAL (SEQ ID |
| | | | NO: 79) |
| HA1 C-Terminal | 290-342 | 272-324 | CNTKCQTSVGGINTNKTFQNIERNALGDCPKYIKSG |
| Stem Segment | | | QLKLATGLRNVPAISNR (SEQ ID NO: 80) |
| HA1 N-Terminal | 19-60 | 1-42 | DRICVGYLSTNSSEKVDTLLENDVPVTSSVDLVETN |
| Stem Segment | | | HTGTYC (SEQ ID NO: 81) |
| Globular Head | 61-289 | 43-271 | SLGGISPVHLGDCSFEGWIVGNPACASNLGIREWSY |
| Domain | | | LIEDPSAPHGLCYPGELDNNGELRHLFSGIRSFSRTE |
| | | | LIAPTSWGAVNDGVSSACPDKGASSFYRNLVWFVK |
| | | | RGNQYPVIRGTYNNTTGRDVLVIWGIHHPVSTDEA |
| | | | KQLYVNNNPYTLVSTSSWSRKYNLETGTRPGYNGQ |
| | | | KSWMKIYWYLMHPGESISFESNGGLLAPRYGYIIEE |
| | | | YGKGRIFQSRIRIAK (SEQ ID NO: 82) |
| Transmembrane | 531-551 | 512-533 | SIYSCIASSIVLVGLILTFIM (SEQ ID NO: 83) |
| Domain² | | | |
| Cytoplasmic | 552-565 | 534-547 | WACSSGNCRFNICI (SEQ ID NO: 84) |
| Domain | | | |

¹HA2 was defined here to not include the TMD or CD
²http://octopus.cbr.su.se/ was used to determine the transmembrane domain

The term “fragment” in the context of a nucleic acid sequence refers to a nucleotide sequence comprising a portion of consecutive nucleotides from a parent sequence. In a specific embodiment, the term refers to a nucleotide sequence of 5 to 15, 5 to 25, 10 to 30, 15 to 30, 10 to 60, 25 to 100, 50 to 100, 75 to 100, 150 to 300 or more consecutive nucleotides from a parent sequence. In another embodiment, the term refers to a nucleotide sequence of at least 5, 10, 15, 20, 25, 30, 35, 40, 45, 50, 55, 60, 65, 70, 75, 80, 85, 90, 95, 100, 110, 125, 150, 175, 200, 250, 275, 300, 325, 350, 375, 400, 425, 450 or 475 consecutive nucleotides of a parent sequence.

The term “fragment” in the context of an amino acid sequence refers to an amino acid sequence comprising a portion of consecutive amino acid residues from a parent sequence. In a specific embodiment, the term refers to an amino acid sequence of 8 to 15, 10 to 20, 2 to 30, 5 to 30, 10 to 60, 25 to 100, 50 to 100, 75 to 100, 150 to 300 or more consecutive amino acid residues from a parent sequence. In another embodiment, the term refers to an amino acid sequence of at least 5, 10, 15, 20, 25, 30, 35, 40, 45, 50, 55, 60, 65, 70, 75, 80, 85, 90, 95, 100, 110, 125, 150, 175, or 200 consecutive amino acid residues of a parent sequence.

“HA” and “hemagglutinin” refer to any influenza virus hemagglutinin known to those of skill in the art or a derivative thereof. In specific embodiments, the hemagglutinin is an influenza A hemagglutinin. A typical hemagglutinin comprises domains known to those of skill in the art including a signal peptide (optional herein), a stem domain, a globular head domain, a transmembrane domain (optional herein) and a cytoplasmic domain (optional herein). In certain embodiments, a hemagglutinin consists of a single polypeptide chain, such as HA0. In certain embodiments, a hemagglutinin consists of more than one polypeptide chain in quaternary association, e.g. HAI and HA2. Those of skill in the art will recognize that an immature HA0 might be cleaved to release a signal peptide (generally approximately 15-20 amino acids) yielding a mature hemagglutinin HA0 (i.e., HA0 without a signal peptide). In the context of an influenza A virus, a mature hemagglutinin HA0 might be cleaved at another site to yield HA1 polypeptide (approximately 320 amino acids, including the globular head domain and a portion of the stem domain) and HA2 polypeptide (approximately 220 amino acids, including the remainder of the stem domain, a luminal domain, a transmembrane domain and a cytoplasmic domain). Those of skill in the art will recognize that the delineation of the domains of an influenza A virus HA may be determined from, e.g., crystal structure and/or by using structure prediction software (for example, the website for the Center for Biological Sequence Analysis, Technical University of Denmark DTU, or Pymol) in conjunction with protein alignments. Thus, in one aspect, one skilled in the art will recognize that the delineation of the domains of influenza A/Hong Kong/4801/2014 virus HA. See, e.g., Table 2, above, for exemplary domains for influenza A/Hong Kong/4801/2014 virus HA are as set forth in Table 1, above.

In another aspect, one skilled in the art will recognize the delineation of domains of the influenza A/Jiangxi-Donghu/346-1/2013 virus HA. See, e.g., Table 2, above, for exemplary domains for influenza A/Jiangxi-Donghu/346-1/2013 virus HA. In another aspect, one skilled in the art will recognize the delineation of domains of the influenza A/Michigan/45/2015 virus HA. See, e.g., Table 3, above, for exemplary domains of the influenza A/Michigan/45/2015 virus HA. In another aspect, one skilled in the art will recognize the delineation of domains of the influenza A/Vietnam/1203/2004 virus HA. See, e.g., Table 4, above, for exemplary domains of the influenza A/Vietnam/1203/2004 virus HA virus HA. In another aspect, one skilled in the art will recognize the delineation of domains of the influenza s A/black headed gull/Sweden/1/1999 virus HA. See, e.g., Table 5, above, for exemplary domains of the influenza A/black headed gull/Sweden/1/1999 virus HA. In another aspect, one skilled in the art will recognize the delineation of domains of the influenza A/mallard/Gurjev/263/1982. In certain embodiments, a hemagglutinin comprises a signal peptide, a transmembrane domain and a cytoplasmic domain. In certain embodiments, a hemagglutinin lacks a signal peptide, i.e. the hemagglutinin is a mature hemagglutinin. In certain embodiments, a hemagglutinin lacks a transmembrane domain or cytoplasmic domain, or both. As used herein, the terms “hemagglutinin” and “HA” encompass hemagglutinin polypeptides that are modified by post-translational processing such as signal peptide cleavage, disulfide bond formation, glycosylation (e.g., N-linked glycosylation), protease cleavage and lipid modification (e.g. S-palmitoylation).

“HA2” refers to a polypeptide domain that corresponds to the HA2 domain of an influenza hemagglutinin polypeptide known to those of skill in the art. Typically, an HA2 domain includes a stem domain, a transmembrane domain and a cytoplasmic domain of an HA polypeptide. See, e.g., SEQ ID Nos: 71, 72, 73, 74 and 88 for exemplary HA2 domains.

The term “HA1 C-terminal stem segment” refers to a polypeptide segment that corresponds to the carboxy-terminal portion of the stem domain of an influenza hemagglutinin HA1 polypeptide. In the context of an influenza A virus, in certain embodiments, an HA1 C-terminal stem segment consists of amino acid residues corresponding approximately to amino acids A_{q} through A_{C term} of an HA1 domain. A_{q} is the cysteine residue in the HA1 C-terminal stem segment that forms or is capable of forming a disulfide bond with a cysteine residue in an influenza A virus HA1 N-terminal stem segment. A_{C term} or otherwise referred to herein as HA1C-term is the C-terminal amino acid of the HA1 domain as recognized by those of skill in the art. Residue A_{q} is identified in influenza A hemagglutinin polypeptides in FIG. 1B (i.e., A_{q} is Cys at amino acid position 277 of an HA1 domain according to H3 numbering). Exemplary HA1 C-terminal stem segments are described herein and in International Publication Nos. WO 2010/117786, WO 2011/123495, WO 2013/043729, and WO 2014/099931, U.S. Publication Nos. 2010/0297174, 2013/0129761, 2014/0328875, and U.S. application Ser. No. 14/345,816, which published as U.S. Patent Publication No. 2015/0132330, which are incorporated herein by reference in their entirety. In the context of an influenza A virus, in certain embodiments, an HA1 C-terminal stem segment consists of amino acid residues corresponding approximately to amino acids 277-329 of HA1 according to H3 numbering. Note that, in this numbering system, 1 refers to the N-terminal amino acid of the mature HA0 protein, from which the signal peptide has been removed. See Tables 1-5 above for exemplary HA1 C-terminal stem segments. Those of skill in the art will readily be able to recognize the amino acid residues that correspond to the HA1 C-terminal stem segment of other influenza HA polypeptides, e.g., the amino acid residues that correspond to the HA1 C-terminal stem segment of HA1 from an H1 hemagglutinin (see, e.g., FIGS. 1A-1D).

The term “HA1 N-terminal stem segment” refers to a polypeptide segment that corresponds to the amino-terminal portion of the stem domain of an influenza virus hemagglutinin HA1 polypeptide. In the context of an influenza A virus, in certain embodiments, an HA1 N-terminal stem segment consists of amino acid residues corresponding approximately to amino acids A_{N-term} through Ap of an HA1 domain. A_{N-term} otherwise referred to herein as HAIN-term is the N-terminal amino acid of HA1 as recognized by those of skill in the art. Aₚ is the cysteine residue in the HA1 N-terminal stem segment that forms or is capable of forming a disulfide bond with a cysteine residue in an influenza A virus HA1 C-terminal stem segment. Residue Aₚ is identified in influenza A hemagglutinin polypeptides in FIG. 1A (i.e., Aₚ is Cys at amino acid position 52 of an HA1 domain according to H3 numbering). Exemplary HA1 N-terminal stem segments are described herein or in International Publication Nos. WO 2010/117786, WO 2011/123495, WO 2013/043729, and WO 2014/099931, U.S. Publication Nos. 2010/0297174, 2013/0129761, 2014/0328875, and U.S. application Ser. No. 14/345,816, which published as U.S. Patent Publication No. 2015/0132330, which are incorporated herein by reference in their entirety. In certain embodiments, an HA1 N-terminal stem segment consists of amino acid residues corresponding approximately to amino acids 1-52 of HA1 according to H3 numbering. Note that, in this numbering system, 1 refers to the N-terminal amino acid of the mature HA0 protein, from which the signal peptide has been removed. See Tables 1-5 above for exemplary HA1 N-terminal stem segments. Those of skill in the art will readily be able to recognize the amino acid residues that correspond to the HA1 N-terminal stem segment of other influenza HA polypeptides, e.g., the amino acid residues that correspond to the HA1 N-terminal stem segment of HA1 from an H1 hemagglutinin (see, e.g., FIG. 1A-1D).

As used herein, the term “heterologous” in the context of a polypeptide, nucleic acid or virus refers to a polypeptide, nucleic acid or virus, respectively, that is not normally found in nature or not normally associated in nature with a polypeptide, nucleic acid or virus of interest. For example, a heterologous polypeptide may refer to a polypeptide derived from a different virus, e.g., a different influenza strain or subtype, or an unrelated virus or different species.

As used herein, the term “in combination,” in the context of the administration of two or more therapies to a subject, refers to the use of more than one therapy (e.g., more than one prophylactic agent and/or therapeutic agent). The use of the term “in combination” does not restrict the order in which therapies are administered to a subject. For example, a first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

As used herein, the term “infection” has the meaning understood to a person skilled in the art. Generally, the term “infection” means the invasion by, multiplication and/or presence of a virus in a cell or a subject.

As used herein, the term “influenza virus disease” has the meaning understood to a person skilled in the art. Generally, the term “influenza virus disease” refers to the pathological state resulting from the presence of an influenza (e.g., influenza A virus) virus in a cell or subject or the invasion of a cell or subject by an influenza virus. In specific embodiments, the term refers to a respiratory illness caused by an influenza virus.

As used herein, the terms “influenza virus hemagglutinin head domain polypeptide,” “influenza virus hemagglutinin head domain,” “HA globular head domain,” and “HA head domain” refer to the globular head domain of an influenza hemagglutinin polypeptide known to those of skill in the art or a derivative thereof. An influenza virus hemagglutinin head domain polypeptide or influenza virus hemagglutinin head domain may comprise or consist of a known (e.g., wild-type) influenza virus hemagglutinin head domain or may comprise or consist of a derivative, e.g. an engineered derivative, of a known (e.g., wild-type) influenza virus hemagglutinin head domain. Those of skill in the art will recognize that an influenza A virus HA globular head domain typically comprises the amino acid residues intervening Cys that corresponds to amino acid position 52 of an influenza virus hemagglutinin HA1 domain according to H3 numbering and Cys that corresponds to amino acid position 277 of an influenza virus hemagglutinin HA1 domain according to H3 numbering, e.g., Aₚ and A_{q} of FIGS. 1A-1B, respectively. See Tables 1-5 above for exemplary HA globular head domains.

As used herein, the phrases “IFN deficient system” or “IFN-deficient substrate” refer to systems, e.g., cells, cell lines and animals, such as pigs, mice, chickens, turkeys, rabbits, rats, etc., which do not produce interferon (IFN) or produce low levels of IFN (i.e., a reduction in IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to IFN, and/or are deficient in the activity of one or more antiviral genes induced by IFN.

As used herein, the numeric term “log” refers to log₁₀.

As used herein, the terms “mosaic influenza A virus hemagglutinin polypeptide,” mosaic influenza virus hemagglutinin polypeptide,” “mosaic influenza virus HA polypeptide’ “mosaic influenza A virus HA polypeptide,” “mosaic hemagglutinin polypeptide,” “mosaic HA,” “mosaic hemagglutinin,” and “mosaic influenza A hemagglutinin polypeptide,” unless otherwise indicated, refer to an influenza A virus hemagglutinin that comprises the ectodomain of an influenza A virus, wherein the ectodomain comprises the influenza A virus hemagglutinin stem domain and the influenza A virus hemagglutinin globular head domain, and wherein the influenza A virus hemagglutinin globular head domain comprises one or more amino acid substitutions in one or more antigenic sites. In a specific embodiment, a mosaic influenza virus HA polypeptide comprises an influenza A virus hemagglutinin that comprises the ectodomain, transmembrane and cytoplasmic domains of an influenza A virus, wherein the ectodomain comprises the influenza A virus hemagglutinin stem domain and the influenza A virus hemagglutinin globular head domain, and wherein the influenza A virus hemagglutinin globular head domain comprises one or more amino acid substitutions in one or more antigenic sites. See, e.g., Sections 5.1 below, for a discussion of mosaic influenza virus HA polypeptides. In certain embodiments, in the context of the mosaic influenza virus hemagglutinin polypeptides described herein, a mosaic influenza virus hemagglutinin head domain refers to an influenza virus hemagglutinin head domain that is between 1% to 5%, 0.5% to 5%, 1% to 4.5%, 0.5% to 4.5%, 1% to 4%, 0.5% to 4%, 1% to 3.5%, 0.5% to 3.5%, 1% to 3%, 0.5% to 3%, 1% to 2.5%, 0.5% to 2.5%, 1% to 2% or 0.5% to 2% different from the homologous head (i.e., the head domain that would normally be associated with the stem domain of the mosaic influenza virus hemagglutinin polypeptide). In certain embodiments, in the context of a mosaic influenza virus hemagglutinin polypeptide described herein, a mosaic influenza virus hemagglutinin head domain refers to an influenza virus head domain that is between 10% to 25%, 10% to 15%, 10% to 30%, 15% to 30%, or 20% to 30% different from the homologous head domain. Those of skill in the art will recognize that such a difference can be measured using approaches known in the art and described herein, e.g., comparing sequence identity or sequence homology of the head domains. In certain embodiments, in the context of the mosaic influenza virus hemagglutinin polypeptides described herein, a mosaic influenza virus hemagglutinin head domain refers to an influenza virus hemagglutinin head that, in a hemagglutination inhibition assay, results in antisera with at least 2, at least 3, at least 4, at least 5, or at least 6 times less hemagglutination inhibition titers relative to the hemagglutination inhibition titers of the antisera raised against the homologous heads (i.e., the head domain that would normally be associated with the stem domain of the mosaic influenza virus hemagglutinin polypeptide). Those of skill in the art will recognize that such a difference can be measured using approaches known in the art and described herein (see, e.g., Sections 5.7 below). In a specific embodiment, a mosaic influenza virus HA polypeptide is not a naturally occurring influenza A virus HA. In other words, the hand of man has been used to engineer the mosaic influenza virus HA. In a specific embodiment, a mosaic influenza virus HA is not the result of antigenic drift.

As used herein, the phrase “multiplicity of infection” or “MOI” has the meaning understood to a person skilled in the art. Generally, phrase “multiplicity of infection” or “MOI” is the average number of infectious virus particles per infected cell. The MOI is determined by dividing the number of infectious virus particles added (ml added x PFU/ml) by the number of cells added (ml added x cells/ml).

As used herein, the term “nucleic acid” is intended to include DNA molecules (e.g., cDNA or genomic DNA) and RNA molecules (e.g., mRNA) and analogs of the DNA or RNA generated using nucleotide analogs. The nucleic acid can be single-stranded or double-stranded.

“Polypeptide” refers to a polymer of amino acids linked by amide bonds as is known to those of skill in the art. As used herein, the term can refer to a single polypeptide chain linked by covalent amide bonds. The term can also refer to multiple polypeptide chains associated by non-covalent interactions such as ionic contacts, hydrogen bonds, Van der Waals contacts and hydrophobic contacts. Those of skill in the art will recognize that the term includes polypeptides that have been modified, for example by post-translational processing such as signal peptide cleavage, disulfide bond formation, glycosylation (e.g., N-linked glycosylation), protease cleavage and lipid modification (e.g. S-palmitoylation).

As used herein, the terms “purified” and “isolated” when used in the context of a polypeptide (including an antibody) that is obtained from a natural source, e.g., cells, refers to a polypeptide which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide (including an antibody) that is chemically synthesized refers to a polypeptide which is substantially free of chemical precursors or other chemicals which are involved in the syntheses of the polypeptide. In a specific embodiment, a mosaic HA polypeptide is chemically synthesized. In another specific embodiment, a mosaic influenza hemagglutinin polypeptide is isolated.

As used herein, the terms “replication,” “viral replication” and “virus replication” in the context of a virus refer to one or more, or all, of the stages of a viral life cycle which result in the propagation of virus. The steps of a viral life cycle include, but are not limited to, virus attachment to the host cell surface, penetration or entry of the host cell (e.g., through receptor mediated endocytosis or membrane fusion), uncoating (the process whereby the viral capsid is removed and degraded by viral enzymes or host enzymes thus releasing the viral genomic nucleic acid), genome replication, synthesis of viral messenger RNA (mRNA), viral protein synthesis, and assembly of viral ribonucleoprotein complexes for genome replication, assembly of virus particles, post-translational modification of the viral proteins, and release from the host cell by lysis or budding and acquisition of a phospholipid envelope which contains embedded viral glycoproteins. In some embodiments, the terms “replication,” “viral replication” and “virus replication” refer to the replication of the viral genome. In other embodiments, the terms “replication,” “viral replication” and “virus replication” refer to the synthesis of viral proteins.

As used herein, the terms “stem domain polypeptide,” “stem domain,” “influenza virus hemagglutinin stem domain polypeptide,” “HA stem domain,” “stalk domain” and “stalk” refer to any influenza virus hemagglutinin stem domain known to those of skill in the art or a derivative thereof, e.g. an engineered derivative, that comprises one or more polypeptide chains that make up a stem domain of hemagglutinin. A stem domain polypeptide might be a single polypeptide chain, two polypeptide chains or more polypeptide chains. Typically, a stem domain polypeptide is a single polypeptide chain (i.e. corresponding to the stem domain of a hemagglutinin HA0 polypeptide) or two polypeptide chains (i.e. corresponding to the stem domain of a hemagglutinin HA1 polypeptide in association with a hemagglutinin HA2 polypeptide). In a particular embodiment, a stem domain comprises an N-terminal HA1 stem segment, a C-terminal HA1 stem segment, and a portion of an HA2 domain (e.g., a portion of an HA2 domain that does not include the transmembrane and cytoplasmic domains). One skilled in the art will understand that the exact location of the C-terminus of the HA stem domain is determined according to the hydrophobicity of the HA2 domain of the particular influenza virus HA strain and can be identified using programs such as, e.g., the TMHMM server (www.cbs.dtu.dk/services/TMHMM/; see, e.g., Cuthbertson et al., 2005, Protein Eng Des Sel, 18(6):295-308) hydrophobicity prediction, or uniprot. See, e.g., Tables 1-5 for exemplary HA stem domains.

As used herein, the term “HA2 stem domain” refers to the portion of the HA2 domain that does not include the transmembrane and cytoplasmic domains. The portion of the HA2 domain that corresponds to the HA2 stem domain may be determined using the TMHMM server (www.cbs.dtu.dk/services/TMHMM/; see, e.g., Cuthbertson et al., 2005, Protein Eng Des Sel, 18(6):295-308) hydrophobicity prediction. Exemplary HA2 stem domains are provided below:

HA2 Stem Domain of A/Hong Kong/4801/2014 HA
(SEQ ID NO: 71)
GIFGAIAGFIENGWEGMVDGWYGFRHQNSEGRGQAADLKSTQAAIDQIN
GKLNRLIGKTNEKFHQIEKEFSEVEGRIQDLEKYVEDTKIDLWSYNAEL
LVALENQHTIDLTDSEMNKLFEKTKKQLRENAEDMGNGCFKIYHKCDNA
CIGSIRNGTYDHNVYRDEALNNRFQIKGVELKSGYKDWI
HA2 Stem Domain of A/Jiangxi-Donghu/346-1/2013 HA
(SEQ ID NO: 72)
GLFGAIAGFLENGWEGMVDGWYGFRHQNAQGTGQAADYKSTQAAIDQIT
GKLNRLVEKTNTEFESIESEFSEIEHQIGNVINWTKDSITDIWTYQAEL
LVAMENQHTIDMADSEMLNLYERVRKQLRQNAEEDGKGCFEIYHACDDS
CMESIRNNTYDHSQYREEALLNRLNINPVTLSSGYKDII
HA2 Stem Domain of A/Michigan/45/2015 HA
(SEQ ID NO: 73)
GLFGAIAGFIEGGWTGMVDGWYGYHHQNEQGSGYAADLKSTQNAIDKIT
NKVNSVIEKMNTQFTAVGKEFNHLEKRIENLNKKVDDGFLDIWTYNAEL
LVLLENERTLDYHDSNVKNLYEKVRNQLKNNAKEIGNGCFEFYHKCDNT
CMESVKNGTYDYPKYSEEAKLNREKIDGVKLESTRIYQIL
HA2 Stem Domain of A/Vietnam/1203/2004 HA
(SEQ ID NO: 74)
GLFGAIAGFIEGGWQGMVDGWYGYHHSNEQGSGYAADKESTQKAIDGVT
NKVNSIIDKMNTQFEAVGREFNNLERRIENLNKKMEDGFLDVWTYNAEL
LVLMENERTLDFHDSNVKNLYDKVRLQLRDNAKELGNGCFEFYHKCDNE
CMESVRNGTYDYPQYSEEARLKREEISGVKLESIGIYQIL
HA2 Stem Domain of A/black headed gull/
Sweden/1/1999 HA
(SEQ ID NO: 88)
GLFGAIAGFIEGGWPGLINGWYGFQHQNEQGVGMAADKESTQKAIDQIT
TKINNIIEKMNGNYDSIRGEFNQVEQRINMLADRIDDAVTDVWSYNAKL
LVLLENDKTLDMHDANVRNLHDQVRRALKTNAIDEGNGCFELLHKCNDS
CMETIRNGTYNHTEYEEESKLKRQEIEGIKLKSDDSVYKAL

As used herein, terms “subject” and “patient” are used interchangeably to refer to an animal (e.g., birds, reptiles, and mammals). In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human. In another embodiment, a subject is a human infant. In another embodiment, a subject is a human child. In another embodiment, a subject is a human adult. In another embodiment, a subject is an elderly human. In another embodiment, a subject is a premature human infant. As used herein, the term “premature human infant” refers to a human infant born at less than 37 weeks of gestational age.

As used herein, the term “seasonal influenza virus strain” refers to a strain of influenza virus to which a subject population is exposed to on a seasonal basis. In specific embodiments, the term seasonal influenza virus strain refers to a strain of influenza A virus. In specific embodiments, the term seasonal influenza virus strain refers to a strain of influenza virus that belongs to the H1 or the H3 subtype, i.e., the two subtypes of influenza A virus that presently persist in the human subject population. In other embodiments, the term seasonal influenza virus strain refers to a strain of influenza B virus. In specific embodiments, the term seasonal influenza virus strain refers to a strain of influenza B virus. In specific embodiments, the term seasonal influenza virus strain refers to a strain of influenza virus that belongs to the Yamagata or the Victoria lineages, i.e., the two influenza B virus lineages that presently persist in the human subject population.

The terms “tertiary structure” and “quaternary structure” have the meanings understood by those of skill in the art. Tertiary structure refers to the three-dimensional structure of a single polypeptide chain. Quaternary structure refers to the three dimensional structure of a polypeptide having multiple polypeptide chains.

As used herein, in some embodiments, the phrase “wild-type” in the context of a viral polypeptide refers to a viral polypeptide that is found in nature and is associated with a naturally occurring virus.

As used herein, in some embodiments, the phrase “wild-type” in the context of a virus refers to the types of a virus that are prevalent, circulating naturally and producing typical outbreaks of disease. In other embodiments, the term “wild-type” in the context of a virus refers to a parental virus.

### 4. BRIEF DESCRIPTION OF THE DRAWINGS

FIGS. 1A-FIG. 1D. Sequence alignment by CLUSTALW of representative sequences of 17 subtypes of influenza virus A hemagglutinin (SEQ ID NOS: 103-119, H1-H17, respectively). The residue designated Ap is the cysteine residue in the HA1 N-terminal stem segment that forms or is capable of forming a disulfide bond with the residue designated Aq, a cysteine residue in an HA1 C-terminal stem segment. Due to size limitations, the sequence alignment is split between FIG. 1A, FIG. 1B, FIG. 1C and FIG. 1D.

FIGS. 2A-FIG. 2B. Head domain epitopes of pandemic-like H1 hemagglutinin and amino acid sequences of mutant epitope substitutions. (FIG. 2A) Crystal structure of pandemic H1 HA trimer (PDB:3UBE) (Xu et al., J. Virol., 2012, 86(2): p. 982-90.) (top view and side view, one monomer in white and two monomers in gray) with classically defined antigenic sites Sa, Sb, Ca1, Ca2, and Cb. A sialic acid molecule is present in the receptor binding pocket of the white HA monomer. (FIG. 2B) Amino acid sequences of the antigenic sites of pandemic-like H1 strain, A/Michigan/45/2015, Sa, Sb, Ca1, Ca2, and Cb, are included here (SEQ ID NOs: 1-12). Amino acid sequences of heterologous epitopes for the mutant virus panel are listed below the respective pandemic H1 sites. Amino acids bolded in black represent substituted residues. Amino acids in gray are unchanged.

FIGS. 3A-FIG. 3C. Hemagglutination inhibition (HI) profiles for the antisera of mice, guinea pigs, and ferrets. Hemagglutination inhibition titers of mouse (n=10) (FIG. 3A), guinea pig (n=4) (FIG. 3B), and ferret (n=5) (FIG. 3C) antisera were measured against a panel of mutant viruses (see FIGS. 2A and 2B). Naïve animals were intranasally infected with 1×10⁵ PFU of pandemic-like H1N1 virus, A/Michigan/45/2015, and antisera were harvested at 4 weeks post infection (except for two ferret antisera which were harvested at 3 weeks post infection). The HI profiles for each species are listed in which statistically significant reductions are in black and minimal reductions are in gray. Statistical significance was determined between the mutant virus to the wt-H1 virus using Kruskal-Wallis One-way analysis of variance (ANOVA) of the mean HI titers (*P≤0.05, **P≤0.01, **P≤0.001, ****P≤0.0001).

FIGS. 4A-FIG. 4B. Hemagglutination inhibition (HI) profiles for adult humans pre- and post-2017-18 seasonal vaccination. (FIG. 4A) HI activities of plasma samples (n=36) collected from 18 adult donors pre- and post-seasonal vaccination (left and right, respectively) measured against a panel of mutant viruses (see FIGS. 2A-2B). The human HI profile is listed in which statistically significant reductions are in black and minimal reductions are in gray. (FIG. 4B) An HI dominance index was calculated for individual samples against each mutant virus (from A). The HI dominance index represents a fold reduction of HI titer in a mutant virus versus its respective wild-type H1 virus. Single individuals are represented by dotted lines. Averaged HI dominance indices for pre- and post-vaccination are plotted in solid lines. Statistical significance was determined between the mutant virus to the respective wt-H1 virus data set (pre or post vaccination) using Dunn's-corrected Kruskal-Wallis one-way analysis of variance (ANOVA) of the mean HI titers (*P≤0.05, ***P≤0.001, ****P≤0.0001).

FIGS. 5A-FIG. 5R. Diversity of human HI profiles. HI profiles showing HI dominance indices of mutant reagents were plotted for each antiserum (pre- and post-vaccination).

FIGS. 6A-FIG. 6R. Hemagglutination inhibition (HI) titers of antisera from human volunteers. HI titers were measured for antisera from human donors (pre- and post-vaccination: white and black, respectively) tested against wild-type H1 virus and the panel of mutant reagents.

FIG. 7A-FIG. 7B. Species-specific hemagglutination inhibition (HI) profiles. ((A) HI dominance indices of post-vaccination human plasma (●, taken for comparison from FIG. 4B) and antisera of infected mice (⋄), ferrets (□) and guinea pigs (◯) were plotted. (B) Absolute HI titers of post-vaccination human plasma and antisera of infected mice, ferrets, and guinea pigs were mapped by antigenic cartography (Smith et al., Science, 2004. 305(5682): p. 371-6). Plots were created from a re-analysis of the datasets shown in FIGS. 3A-3C and 4A-4B.

FIGS. 8A-FIG. 8C. Comparison of the H3 and H10 proteins. (FIG. 8A) Phylogenetic tree of influenza A and B virus HA proteins. The scale bar represents 7% change at the amino acid level. Figure adapted from Krammer et al, Biotechnol J, 10:690-701. (FIG. 8B) Models of the H3 (left) and the H10 (right) HA monomers. The head domains are shown in dark gray and the stalk domains in light gray. The H3 model is based on the crystal structure of A/Victoria/361/2011 (H3N2) influenza virus HA (PDB: 4O5N) (Lee et al., Nat Commun, 5:3614) and the H10 model is based on the crystal structure of A/Jiangxi-Donghu/346-1/2013 (H10N8) influenza virus HA (PDB: 4XQO) (Zhang et al., Cell Host Microbe, 17:377-384). The models were visualized with UCSF Chimera (Pettersen et. al., J Comput Chem, 25:1605-1612). (FIG. 8C) The amino acid sequences of the H3 and H10 HAs are aligned. Only regions including the H3 antigenic sites are shown. Conserved amino acids are marked with asterisks. The indicated amino acid numbers are according to H3 numbering (A) Phylogenetic tree of influenza A and B virus HA proteins. The scale bar represents 7% change at the amino acid level. Figure adapted from Krammer et al, Biotechnol J, 10:690-701. (FIG. 8B) Models of the H3 (left) and the H10 (right) HA monomers. The head domains are shown in dark gray and the stalk domains in light gray. The H3 model is based on the crystal structure of A/Victoria/361/2011 (H3N2) influenza virus HA (PDB: 4O5N) (Lee et al., Nat Commun, 5:3614) and the H10 model is based on the crystal structure of A/Jiangxi-Donghu/346-1/2013 (H10N8) influenza virus HA (PDB: 4XQO) (Zhang et al., Cell Host Microbe, 17:377-384). The models were visualized with UCSF Chimera (Pettersen et. al., J Comput Chem, 25:1605-1612). (FIG. 8C) The amino acid sequences of the H3 (SEQ ID NO: 159) and H10 (SEQ ID NO: 160) HAs are aligned. Only regions including the H3 antigenic sites are shown. Conserved amino acids are marked with asterisks. The indicated amino acid numbers are according to H3 numbering.

FIGS. 9A-FIG. 9G. Recombinant influenza viruses expressing H3 HA protein with mutated antigenic sites. (FIG. 9A) Model of the H3 HA trimer. Residues that were mutated are highlighted according to the antigenic sites, as indicated. The three monomers are shown in three shades of gray. The three-dimensional model is based on the crystal structure of A/Victoria/361/2011 influenza virus (H3N2) HA (PDB: 4O5N) (Krammer et al, Biotechnol J, 10:690-701) and was visualized using UCSF Chimera (Pettersen et. al., J Comput Chem, 25:1605-1612). (FIG. 9B) Amino acid sequences of the antigenic sites of HK2014 HA (top sequences, H3 numbering) (SEQ ID NOs: 127, 15, 130, 19, 132, 135, 124, and 137) are aligned with the corresponding sequences of the H10 HA (bottom sequences) (SEQ ID NOs: 128, 161, 163, 164, 134, 165, 125, and 166). Amino acids that have been exchanged in the mutant HAs of the H3-AA through H3-AE (SEQ ID NOs: 128, 17, 162, 133, 134, 136, 125, and 138) viruses for each antigenic site of FIG. 9A are shown here. (FIG. 9C) and (D) Bars represent the mean of viral titers expressed as plaque-forming units (PFU) per milliliter of allantoic fluid (C) or HA titer per 50 μL of allantoic fluid (FIG. 9D) after the virus has been grown in eggs for 48 h at 37° C. (FIG. 9E) Representative images of MDCK cells infected with the indicated viruses for 16 h obtained by immunofluorescence microscopy. Surface staining with mAb 9H10 (top) or polyclonal mouse antiserum (bottom) is shown. (FIG. 9F) Results from whole cell ELISA of MDCK cells infected with the indicated viruses for 16 h. Bars show mean±SD. (FIG. 9G) Antibody response in polyclonal mouse antiserum, as measured by ELISA. ELISA plates were coated with recombinant HA proteins. H3 is the full-length HA of the HK2014 virus, cH14/3 is a chimeric HA with an H14 head and the HK2014 stalk, and H14 is the full length H14 HA protein. Data points show the mean±SD of three replicates. Positive control mAbs are 9H10 (for H3) and 2F11, an in-house produced mAb against H14. n.d., not detectable.

FIGS. 10A-FIG. 10E. HI titers of mouse antisera. (FIG. 10A) Immunization regime. Mice received two doses of PR8-H3N2 (HK2014) (6:2) virus either intranasally (i.n.) at a dose of 10⁷ PFU, or intraperitoneally (i.p.) at a dose of 4×10⁶ PFU at the indicated time points. This virus expresses the surface glycoproteins of the HK2014 H3N2 virus in the PR8 backbone. Serum drawn four weeks after the second immunization was used for ELISA and HI assays. (FIG. 10B) IgG response measured by ELISA. ELISA plates were coated with whole H3-wt virus. The different routes of immunization and mouse strains are indicated to the right of the graphs. Data points show the mean of two replicates. (FIG. 10C) HI titers to the indicated viruses are shown. Symbols representing individual mice are shown similar to FIG. 10B, bars show the mean value of each group. Statistical significance compared to H3-wt was inferred by Newman-Keuls corrected One-way analysis of variance (ANOVA) of the log 2-transformed HI titers with *P≤0.05, **P≤0.01 and ***P≤0.001. Data points represent individual mice except for the first subpanel that shows pooled serum of five mice measured in triplicate. (FIG. 10D) This panel shows the same data as in FIG. 10C, but for each serum sample the HI titer against the H3-AA through H3-AE viruses was normalized to the respective HI titer obtained for the H3-wt virus. Individual serum samples are shown in light gray dotted lines, many of which are overlapping. The mean value of all samples is shown as a solid black line. Statistical significance compared to H3-wt was inferred by performing a Dunn-corrected Kruskal-Wallis tests with ^{##}P≤0.01 and ^{###}P≤0.001. (FIG. 10E) This figure shows the same data as in FIG. 10C but plotted as an antigenic map (Koel et al., Science 342:976-979). The viruses (H3-wt and H3-ΔA through H3-ΔE) are shown as black data points, whereby the data point for H3-ΔD is hidden. Sera are shown as indicated to the right of the map. The spacing between grid lines corresponds to a factor 2 difference in HI titers. Numbers indicate overlapping data points, e.g., 2 indicates that the data point represents two sera with identical or near-identical HI profiles.

FIGS. 11A-FIG. 11C. HI titers of human plasma samples pre- and post-vaccination. (FIG. 11A) HI titers to the indicated viruses are shown. White circles represent individual plasma samples of 18 subjects. The bars show the geometric mean of each group pre-vaccination and four to eight weeks post-vaccination. (FIG. 11B) This figure shows the same data as in FIG. 11A, but for each plasma sample the HI titer against the H3-ΔA through H3-ΔE viruses was normalized to the HI titer obtained for H3-wt virus. Individual plasma samples (n=36) are shown in dotted lines, the mean values are shown as solid lines. (FIG. 11C) This figure shows the same data as in FIG. 11A but plotted as antigenic map (Koel et al., 2013, Science 342:976). The viruses (H3-wt and H3-ΔA through H3-ΔE) are shown as data points. Plasma samples pre- and post-vaccination are indicated to the right of the map. The spacing between grid lines corresponds to a factor 2 difference in HI titers. Numbers indicate overlapping data points, e.g., 2 indicates that the data point represents two sera with identical or near-identical HI profiles. In FIG. 10A, statistical significance pre-vs. post-vaccination was determined by paired Student's t-tests of the log 2-transformed HI titers with ⁺P≤0.05 and ⁺⁺P≤0.01. Statistical significance compared to H3-wt was inferred by Newman-Keuls corrected one-way analysis of variance (ANOVA) of the log 2-transformed HI titers with **P≤0.01 and ***P≤0.001, whereby pre- and post-vaccination groups were analyzed separately. Statistical significance in FIG. 10B was inferred by Dunn-corrected Kruskal-Wallis tests with ***P≤0.001 with pre- and post-vaccination groups separately analyzed. The normalized HI titers for H3-AB in FIG. 10B are significantly lower compared to H3-wt both pre- and post-vaccination.

FIGS. 12A-12B. Sequence alignment of mosaic influenza virus H1 (SEQ ID NO: 30), influenza virus A/Michigan/45/2015 (H1) (SEQ ID NO: 169), A/Vietnam/1203/2004 (H5) (SEQ ID NO: 168), and A/black headed gull/Sweden/1/1999 (H13) (SEQ ID NO: 167). Antigenic sites are indicated as Sa, Sb, Ca1, Ca2 and Cb and they are underlined and bolded for H1 and mH1. Light gray indicates H5 and H13 sequences not used in the mH1 construct.

FIGS. 13A-13B. Sequence alignment of mosaic influenza virus H3 (SEQ ID NO: 31), influenza virus A/Hong Kong/4801/2014 (H3) (SEQ ID NO: 171), and A/Jiangxi-Donghu/346-1/2013 (H10) (SEQ ID NO: 170). Antigenic sites are indicated as A, B, C, D and E and they are underlined and bolded for H3 and mH3. Light gray indicates H10 sequences not used in the mH3 construct.

FIGS. 14A-14E. Rescue and characterization of recombinant influenza viruses expressing mosaic hemagglutinin proteins. FIG. 14A: Model of the H3 hemagglutinin trimer. Residues that were mutated are indicated according to the major antigenic sites. The model is based on the published crystal structure of the hemagglutinin of A/Victoria/361/2011 (H3N2) 26, PDB accession no. 405N, and was visualized with the UCSF Chimera software 27. FIG. 14B: Amino acid sequences of parts of the major antigenic sites of HK2014 hemagglutinin (H3 numbering) (SEQ ID NOs: 127, 15, 130, 19, 132, 135, 124, and 137) are aligned with the corresponding sequences of the mosaic mH10/3 (SEQ ID NOs: 128, 17, 131, 133, 134, 136, 125, and 138) and mH14/3 (SEQ ID NOs: 148, 140, 149, 150, 151, 152, 145, and 153) hemagglutinins. FIG. 14C: Representative scans of HA assays with influenza viruses (allantoic fluids) carrying wildtype (wt), mosaic (mH10/3 and mH14/3) or chimeric (cH10/3 and cH14/3) hemagglutinins. FIG. 14D: Representative images of MDCK cells infected with the indicated viruses for 16 hours obtained by immunofluorescence microscopy. Surface staining with mAb 9H10 is shown. The scale bar indicates 100 μm. FIG. 14E: HI assays using the indicated viruses and antisera of two ferrets raised against HK2014 virus are shown, with both antisera measured in triplicates.

FIGS. 15A-15E. Serum antibody responses of vaccinated mice determined by ELISA. FIG. 15A: Immunization regime. FIG. 15 Overview of the mouse groups (QIV, quadrivalent influenza vaccine). FIGS. 15C-E: Serum IgG responses against the indicated recombinant trimeric hemagglutinin proteins depicted as area under the curve (AUC). Data points represent sera of individual mice (15 per group), horizontal bars the geometric mean values. The dashed lines indicate the limit of detection (AUC=100), signals below this threshold were set to 100. Statistical significance was determined using Bonferroni-corrected ANOVA with *P≤0.05, **P≤0.01, ***P≤0.001.

FIGS. 16A-16E. Functional analyses of murine antisera. FIGS. 16A, 16B: Hemagglutination inhibition (HI) titers against the indicated H3N2 viruses carrying HA and NA of A/Hong Kong/4801/2014 (HK2014), A/Perth/16/2009 (Perth 2009), A/Philippines/2/1982 (Phi 1982), A/Hong Kong/1/1968 (HK1968). FIG. 16A shows data for pooled sera from 15 mice measured in triplicates, FIG. 16B shows data for individual sera. The horizontal bars show the geometric mean values and the dashed lines the limit of detection. Statistical significance in FIG. 16B was inferred by performing ANOVA with the Newman-Keuls posttest on log-transformed values with ***P≤0.001. FIG. 16C: Microneutralization (MNT) endpoint titers determined with HK2014 virus using pooled sera (n=15 mice) measured in triplicates. The horizontal bars show the geometric mean values and the dashed lines the limit of detection. FIGS. 16D, 16E: In vitro antibody-dependent cellular cytotoxicity (ADCC) activity using MDCK cells infected with HK2014 virus (FIG. 16D) or with HK1968 virus (FIG. 16E). Data points represent mean±SD of pooled sera from 15 mice measured in triplicates.

FIGS. 17A-17E. Virus challenge studies in mice. FIG. 17A: Mice (n=4-5) received 200 μL of pooled sera intraperitoneally (i.p.) and were challenged intranasally (i.n.) with 5 mLD50 of X-31 (a reassortant virus with the HA and NA of A/Hong Kong/1/1968 and the internal proteins of PR8) or X-79 (a reassortant virus expressing HA and NA of A/Philippines/2/1982 and the internal proteins of PR8). Weight and survival were observed for 14 days post-infection. FIGS. 17B-17E: Weight curves (FIGS. 17 B and D) and survival curves (FIGS. 17C and E) of mice challenged with the indicated viruses. The weight curves show the mean with SD. In the survival plots, the proportion of surviving animals in each group is shown in parentheses and statistical significance was inferred by log rank Mantel-Cox tests against the Mock groups (DNA prime only or untreated) with *P≤0.05 and **P≤0.01.

FIGS. 18A-18C: HA DNA and protein sequences (SEQ ID NOs: 172-179). In the DNA sequences, 15 bp overhangs used for cloning are shown in bold lowercase letters. In the mosaic HAs, sequences that have been altered compared to the H3 backbone sequence are highlighted depending on the antigenic site; bold, site A; italics & bold, site B; italics, site C; underline, site D; bold and underlined, site E. Adaptive mutations that occurred after viral rescue of the mH10/3 virus are highlighted in grey and in lowercase; the same adaptive mutations except the one located in site C were incorporated into the mH14/3 sequence. In the chimeric HAs, sequences changed to H10 or H14 are shown in lowercase letters, and one adaptive mutation in cH10/3 is in italics and underlined.

FIGS. 19A-19C. Serum antibody responses of vaccinated mice determined by ELISA. FIG. 19A: Schematic representation of the HA protein. The globular head domain is located between residues C52 and C277 (H3 numbering). The stalk domain comprises the remaining portions of HA1 and HA2 subunits. SP, signal peptide; TM, transmembrane domain; CT, cytoplasmic tail. The HA1 proteins used in FIGS. 19B and 19C comprise the HA1 region without signal peptide and carry a C-terminal hexahistidine tag. FIGS. 19B, 19C: IgG responses against recombinant HA1 proteins from the A/Hong Kong/4801/2014 (FIG. 19B) and A/Aichi/2/1968 (FIG. 19C) H3N2 viruses depicted as area under the curve (AUC). Data points represent sera of individual mice (15 per group), horizontal bars the geometric mean values. The dashed lines indicate the limit of detection (AUC=100), signals below this threshold were set to 100. Statistical significance was determined using Bonferroni-corrected ANOVA with *P≤0.05, **P≤0.01, ***P≤0.001. The mouse groups and immunization regime are explained in FIG. 15 of the main text.

FIGS. 20A-20D. Detection of serum antibodies cross-reacting with group 2 HAs. FIGS. 20A, 20B: Immunofluorescence microscopy. 293T cells transfected with plasmids expressing H4 of A/duck/Czechoslovakia/1956 (H4N6) (FIG. 20A) or H7 of A/Hunan/02285/2017 (H7N9) (FIG. 20B) were incubated with pooled sera of 15 mice per group diluted 1:50 or monoclonal antibodies at 10 μg/mL. Binding was visualized with Alexa Fluor 488 labeled secondary antibodies. Anti-H4 and anti-H7 monoclonal antibodies were produced in-house and CR9114 is a pan anti-HA stalk antibody45,46. FIG. 20C: Antibody response of pooled sera against trimeric recombinant H15 from A/shearwater/West Australia/2576/1979 (H15N9), trimeric recombinant H10 from A/Jiangxi-Donghu/346-1/2013 (H10N8) and H14 from A/mallard/Gurjev/263/1982 (H14N5) as determined by ELISA. Data points represent the mean±SD of pooled sera from 15 mice measured in triplicates. FIG. 20D: Phylogenetic tree of influenza A virus HA proteins26. The scale bar represents a 5% change at the amino acid level.

### 5. DETAILED DESCRIPTION

### 5.1 Mosaic Influenza Virus Hemagglutinin Polypeptides

In one aspect, a mosaic influenza virus hemagglutinin (HA) polypeptide provided herein comprises an influenza A virus hemagglutinin (HA) ectodomain, wherein the HA ectodomain comprises an HA stem domain polypeptide and an HA globular head domain of the influenza A virus HA, and wherein the HA globular head domain comprises one or more amino acid substitutions in one or more antigenic sites. In certain embodiments, the mosaic influenza virus HA polypeptide further comprises the transmembrane and cytoplasmic domains of the same influenza A virus. The primary sequence of a mosaic influenza virus hemagglutinin polypeptide provided herein might be formed by a single polypeptide, or it might be formed by multiple polypeptides. Typically, a single polypeptide is expressed by any technique deemed suitable by one of skill in the art.

In another aspect, provided herein are mosaic hemagglutinin (HA) polypeptides comprising an HA ectodomain of a first influenza A virus comprising 2, 3, 4, 5, 6, 7, 8, 9, 10, 11, 12 or more amino acid substitutions within an antigenic site of the globular head domain of the influenza A virus HA (e.g., Ca1, Ca2, Cb, Sa and/or Sb antigenic site for an H1 or A, B, C, D and/or E antigenic site for an H3), wherein the amino acid substitutions substitute 2, 3, 4, 5, 6, 7, 8, 9, 10, 11, 12 or more amino acid residues in the antigenic site of the globular head domain of the influenza A virus HA with amino acid residues from a second different influenza A virus HA strain or subtype that do not affect the conformation/structure of the HA. In specific embodiment, provided herein are mosaic hemagglutinin (HA) polypeptides comprising an HA ectodomain of a first influenza A virus comprising 2, 3, 4, 5, 6, 7, 8, 9, 10, 11, 12 or more amino acid substitutions within an antigenic site of the globular head domain of the first influenza A virus HA, wherein the amino acid substitutions substitute 2, 3, 4, 5, 6, 7, 8, 9, 10, 11, 12 or more amino acid residues in the antigenic site of the globular head of the first influenza A virus HA with amino acid residues found in a corresponding region of the globular domain of a second different influenza A virus HA strain or subtype. In certain embodiments, the mosaic influenza virus HA polypeptide further comprises the transmembrane and cytoplasmic domains of the first influenza A virus HA.

In another aspect, provided herein are mosaic hemagglutinin (HA) polypeptides comprising an HA ectodomain of an influenza A virus comprising 2, 3, 4, 5, 6, 7, 8, 9, 10, 11, 12 or more amino acid substitutions within an antigenic site of the globular head domain of the influenza A virus HA (e.g., Ca1, Ca2, Cb, Sa and/or Sb antigenic site for an H1 or A, B, C, D and/or E antigenic site for an H3), wherein the amino acid substitutions substitute 2, 3, 4, 5, 6, 7, 8, 9, 10, 11, 12 or more amino acid residues in the antigenic site of the globular head domain of the influenza A virus HA with random amino acid residues that do not affect the conformation/structure of the HA. For example, amino acid residues in an antigenic site of the globular head domain of an influenza A virus HA may be substituted with alanines or other amino acid residues so long as the substitution does not change the conformation/structure of the HA so long as the substitution does not change the conformation/structure of the HA. The effect of amino acid substitutions on the conformation/structure of an influenza A virus HA may be determined by assays known in art or disclosed herein (e.g., in § 5.1 or 6), such as, e.g., structure programs, crystallography, or functional assays. In certain embodiments, the amino acid residues are not derived from influenza virus neuraminidase. In certain embodiments, the mosaic influenza virus HA polypeptide further comprises the transmembrane and cytoplasmic domains of the influenza A virus HA.

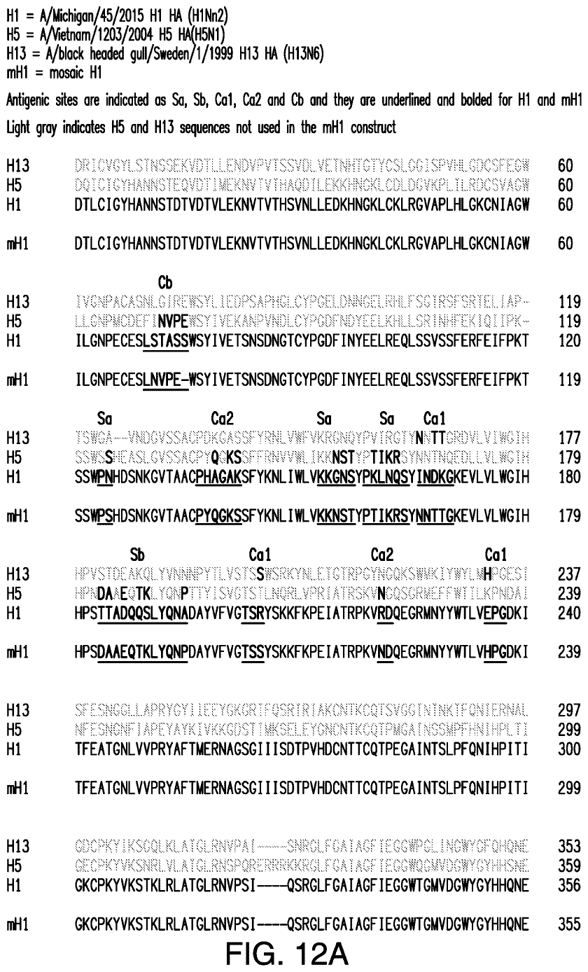

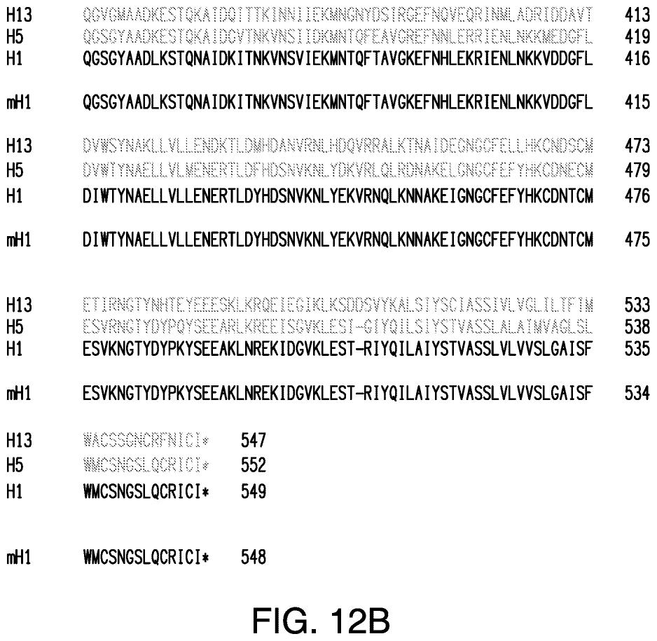

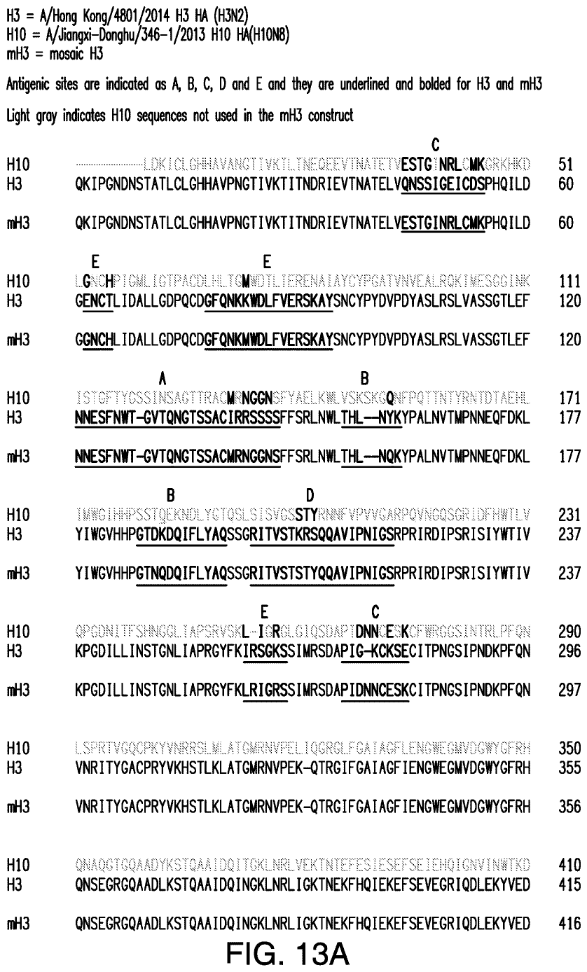

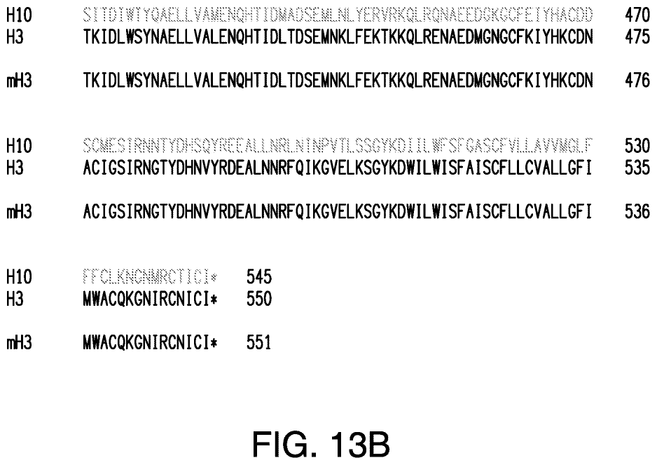

The amino acid residues in the globular head domain of an influenza A virus HA in a region corresponding to an antigenic site (e.g., Ca1, Ca2, Cb, Sa and/or Sb antigenic site for an H1 or A, B, C, D and/or E antigenic site for an H3) in the globular head domain of an influenza A virus HA may be identified using techniques known to one skilled in the art. In specific embodiments, the amino acid residues in the globular head domain of an influenza A virus HA in a region corresponding to an antigenic site (e.g., Ca1, Ca2, Cb, Sa and/or Sb antigenic site for an H1 or A, B, C, D and/or E antigenic site for an H3) in the globular head domain of an influenza A virus HA are identified by comparing the amino acid sequences and/or structural information (e.g., crystal structures) of influenza A viruses. In particular embodiments, alignments of the amino acid sequences of HA of influenza A viruses as well as assessing the viruses for structural similarity enables the skilled person in the art to select the amino acid residues in the influenza A virus HA antigenic site to substitute with amino acid residues from a corresponding region in the globular head domain of a different influenza A virus HA strain or subtype. See, e.g., the sequence alignments in FIGS. 12A-12B and 13A-13B. For example, one might want to refrain from substituting amino acid residues, such as cysteine, proline or both, in the influenza A virus HA antigenic site that may impact the folding of the mosaic HA with amino acid residues from a corresponding region in the globular head domain of a different influenza A virus HA strain or subtype. In addition, one might want to refrain from substituting amino acid residues in the influenza A virus HA antigenic site that impact the coding for N-linked glycosylation sites (N-X-S/T). In selecting the amino acid residues to substitute, care should be taken to maintain the conformation/structure of the HA. In some embodiments, amino acid residues that are highly conserved in an antigenic site of the globular head domain of an influenza A virus HA, one might want to refrain from substituting with amino acid residues from a corresponding region in the globular head domain of a different influenza A virus HA strain or subtype. For example, one of skill in the art may not want to substitute the methionine in antigenic sites of an influenza A virus HA with another amino acid residue. See, e.g., Section 6. In certain embodiments, with respect to amino acid residues such as proline found in an antigenic site of the globular head domain of an influenza A virus HA, one might want to refrain from substituting with amino acid residues from a corresponding region in the globular head domain of a different influenza A virus HA strain or subtype. In some embodiments, with respect to amino acid residues such as cysteine, proline or both found in an antigenic site of the globular head domain of an influenza A virus HA, one might want to refrain from substituting with amino acid residues from a corresponding region in the globular head domain of a different influenza A virus HA strain or subtype. In specific embodiments, the amino acid residues substituted in an antigenic site of the globular head domain of an influenza A virus are not consecutive amino acid residues. For example, amino acid residues that are found conformationally close to one another may be substituted for other amino acid residues. In other embodiments, the amino acid residues substituted in an antigenic site of the globular head domain of an influenza A virus are consecutive amino acid residues. In certain embodiments, an amino acid residue found in the antigenic site of an influenza A virus is substituted with a conservative amino acid residue (i.e., a conservative substitution). In a specific embodiment, the selection of amino acid residues in an antigenic site of an influenza A virus HA to substitute with other amino acid residues may be identified as described in Section 6, infra. The effect of amino acid substitutions on the conformation/structure may be determined by assays known to one of skill in the art, e.g., structure programs, crystallography, or functional assays. See, e.g., Section 5.9 below, and Section 6. In a particular embodiment, the mosaic HA polypeptides may be evaluated for antigenic conservation using a panel of monoclonal antibodies that bind to conserved epitopes in the globular head domain of HA and the stem domain of HA. In a specific embodiment, the methods described in Section 6 are used to evaluate antigenic conservation of the mosaic HA. In addition, the mosaic HA polypeptides described herein may be evaluated to determine whether the antigenic sites of the influenza A virus HA were mutated using techniques known to one of skill in the art or described herein (see, e.g., Section 6 including the HI assay described therein). In particular, the mosaic HA polypeptides described herein may be evaluated to determine if the amino acid substitutions in the antigenic site(s) of the influenza A virus HA result in loss of a variable region(s) of the influenza A virus HA using techniques known to one of skill in the art or described herein (see, e.g., Section 6 including the HI assay described therein). In a specific embodiment, the mosaic HA polypeptides described herein may be evaluated to determine if the amino acid substitutions in the antigenic site(s) of the influenza A virus HA reduce or eliminate the immunodominant epitopes of the influenza A virus HA using techniques known to one of skill in the art or described herein (see, e.g., Section 6, including the HI assay described therein). In a specific embodiment, a mosaic HA polypeptide described herein is assessed in an HI assay, such as described in Section 6 to evaluate the replacement of the antigenic site(s) in the influenza A virus HA.

In certain embodiments, a mosaic influenza virus hemagglutinin polypeptide provided herein comprises a signal peptide. Typically, the signal peptide is cleaved during or after polypeptide expression and translation to yield a mature mosaic influenza virus hemagglutinin polypeptide. In certain embodiments, also provided herein are mature mosaic influenza virus hemagglutinin polypeptides that lack a signal peptide. In embodiments where a mosaic influenza virus hemagglutinin polypeptide provided herein comprises a signal peptide, the signal peptide might be based on any influenza virus signal peptide known to those of skill in the art. In certain embodiments, the signal peptides are based on influenza A signal peptides. In certain embodiments, the signal peptides are based on the signal peptide of an influenza A hemagglutinin selected from the group consisting of H1, H2, H3, H4, H5, H6, H7, H8, H9, H10, H11, H12, H13, H14, H15, H16, H17, and H18. In certain embodiments, the signal peptide might be any signal peptide deemed useful to one of skill in the art. In certain embodiments, the signal peptide is selected from those in Tables 1-5, above, and Table 6, below. In a specific embodiment, the signal peptide is from the HA of the same influenza A virus strain as the HA ectodomain.

TABLE 6
Exemplary signal peptide sequences.

| Description | Sequence |
|---|---|
| Exemplary influenza A HA | MKANLLVLLCALAAADA |
| subtype H1 signal peptide | (SEQ ID NO: 85) |
| Exemplary influenza A HA | MAIIYLILLFTAVRG |
| subtype H2 signal peptide | (SEQ ID NO: 86) |
| Exemplary influenza A HA | MKTIIALSYIFCLALG |
| subtype H3 signal peptide | (SEQ ID NO: 87) |
| Exemplary influenza A HA | MLSIVILFLLIAENSS |
| subtype H4 signal peptide | (SEQ ID NO: 89) |
| Exemplary influenza A HA | MLSIVILFLLIAENSS |
| subtype H5 signal peptide | (SEQ ID NO: 89) |
| Exemplary influenza A HA | MIAIIVVAILATAGRS |
| subtype H6 signal peptide | (SEQ ID NO: 90) |
| Exemplary influenza A HA | MNTQILVFALVAVIPTNA |
| subtype H7 signal peptide | (SEQ ID NO: 91) |
| Exemplary influenza A HA | MEKFIAIATLASTNAY |
| subtype H8 signal peptide | (SEQ ID NO: 92) |
| Exemplary influenza A HA | METKAIIAALLMVTAA |
| subtype H9 signal peptide | (SEQ ID NO: 93) |
| Exemplary influenza A HA | MYKVVVIIALLGAVKG |
| subtype H10 signal peptide | (SEQ ID NO: 94) |
| Exemplary influenza A HA | MEKTLLFAAIFLCVKA |
| subtype H11 signal peptide | (SEQ ID NO: 95) |
| Exemplary influenza A HA | MEKFIILSTVLAASFAY |
| subtype H12 signal peptide | (SEQ ID NO: 96) |
| Exemplary influenza A HA | MALNVIATLTLISVCVHA |
| subtype H13 signal peptide | (SEQ ID NO: 97) |
| Exemplary influenza A HA | MIALILVALALSHTAYS |
| subtype H14 signal peptide | (SEQ ID NO: 98) |
| Exemplary influenza A HA | MNTQIIVILVLGLSMVKS |
| subtype H15 signal peptide | (SEQ ID NO: 99) |
| Exemplary influenza A HA | MMIKVLYFLIIVLGRYSKA |
| subtype H16 signal peptide | (SEQ ID NO: 100) |

In certain embodiments, the mosaic influenza virus hemagglutinin polypeptides provided herein further comprise one or more polypeptide domains. Useful polypeptide domains include domains that facilitate purification, folding and cleavage of portions of a polypeptide. For example, a His tag (His-His-His-His-His-His, SEQ ID NO: 120), FLAG epitope or other purification tag can facilitate purification of a mosaic influenza virus hemagglutinin polypeptide provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In specific embodiments, the mosaic influenza virus hemagglutinin polypeptides provided herein comprise a foldon, or trimerization, domain, such as from bacteriophage T4 fibritin. A foldon, or trimerization, domain from bacteriophage T4 fibritin can facilitate trimerization of polypeptides provided herein. In some embodiments, the trimerization domain comprises a wildtype GCN4pII trimerization heptad repeat or a modified GCN4pII trimerization heptad repeat that allows for the formation of trimeric or tetrameric coiled coils. See, e.g., Weldon et al., 2010, PLoSONE 5(9): e12466. The foldon domain can have any foldon sequence known to those of skill in the art (see, e.g., Papanikolopoulou et al., 2004, J. Biol. Chem. 279(10):8991-8998, the contents of which are hereby incorporated by reference in their entirety. Examples include GSGYIPEAPRDGQAYVRKDGEWVLLSTFL (SEQ ID NO: 121). A foldon domain can be useful to facilitate trimerization of soluble polypeptides provided herein. In specific embodiments, the mosaic influenza virus hemagglutinin polypeptides provided herein comprise a cleavage site. Cleavage sites can be used to facilitate cleavage of a portion of a polypeptide, for example cleavage of a purification tag or foldon domain or both. Useful cleavage sites include a thrombin cleavage site, for example one with the sequence LVPRGSP (SEQ ID NO:122). In certain embodiments, the cleavage site is a cleavage site recognized by Tobacco Etch Virus (TEV) protease (e.g., amino acid sequence Glu-Asn-Leu-Tyr-Phe-Gln-(Gly/Ser) (SEQ ID NO: 123).

In certain embodiments, a mosaic influenza virus hemagglutinin polypeptide provided herein is monomeric. In certain embodiments, a mosaic influenza virus hemagglutinin polypeptide provided herein is multimeric. In certain embodiments, a mosaic influenza virus hemagglutinin polypeptide provided herein is trimeric.

In a specific embodiment, an influenza A virus HA utilized in the generation of a mosaic HA polypeptide described herein is the HA from an influenza virus of the H1, H2, H3, H4, H5, H6, H7, H8, H9, H10, H11, H12, H13, H14, H15, H16, H17, or H18 subtype. In a specific embodiment, an influenza A virus HA utilized in the generation of a mosaic HA polypeptide described herein is the HA from an influenza virus of the H2, H3, H4, H5, H6, H7, H8, H9, H10, H11, H12, H13, H14, H15, H16, H17, or H18 subtype. In a specific embodiment, an influenza A virus HA utilized in the generation of a mosaic HA polypeptide described herein is the HA from an influenza virus of the H5, H8, H11, H12, or H13 subtype. In a specific embodiment, an influenza A virus HA utilized in the generation of a mosaic HA polypeptide described herein is the HA from an influenza virus of the H5 subtype. In a specific embodiment, an influenza A virus HA utilized in the generation of a mosaic HA polypeptide described herein is the HA from an influenza virus of the H10 subtype. In a specific embodiment, an influenza A virus HA utilized in the generation of a mosaic HA polypeptide described herein is the HA from an influenza virus of the H8 subtype. In a specific embodiment, an influenza A virus HA utilized in the generation of a mosaic HA polypeptide described herein is the HA from an influenza virus of the H11 subtype. In a specific embodiment, an influenza A virus HA utilized in the generation of a mosaic HA polypeptide described herein is the HA from an influenza virus of the H12 subtype. In a specific embodiment, an influenza A virus HA utilized in the generation of a mosaic HA polypeptide described herein is the HA from an influenza virus of the H13 subtype. In a specific embodiment, an influenza A virus HA utilized in the generation of a mosaic HA polypeptide described herein is the HA from an influenza virus of the H14 subtype. In a specific embodiment, an influenza A virus HA utilized in the generation of a mosaic HA polypeptide described herein is the HA from an avian influenza virus.

In a specific embodiment, an influenza A virus HA utilized in the generation of a mosaic HA polypeptide described herein is the HA from influenza A/mallard/Sweden/24/2002 virus (GenBank Accession No. CY060249.1; GenBank GI No. 294441479). In a specific embodiment, an influenza A virus HA utilized in the generation of a mosaic HA polypeptide described herein is the HA from influenza A/Vietnam/1203/04 virus (GenBank Accession No. EF541403.1; GenBank GI No. 145284465; see, also, Steel et al., 2009, Journal of Virology, 83(4):1742-1753 for the HA of influenza A/Vietnam/1203/04 (HALo) virus). In a specific embodiment, an influenza A virus HA utilized in the generation of a mosaic HA polypeptide described herein is the HA from influenza A/northern shoveler/Netherlands/18/99 virus (GenBank Accession No. CY060417.1; GenBank GI No. 294441876). In a specific embodiment, an influenza A virus HA utilized in the generation of a mosaic HA polypeptide described herein is the HA from influenza A_mallard_interior Alaska_7MP0167_2007 virus (GenBank Accession No. CY077198.1; GenBank GI No. 312652817). In a specific embodiment, an influenza A virus HA utilized in the generation of a mosaic HA polypeptide described herein is the HA from influenza A/Puerto Rico/8/34 virus (GenBank Accession No. AF389118.1; GenBank GI No. 21693168). In a specific embodiment, an influenza A virus HA utilized in the generation of a mosaic HA polypeptide described herein is the HA from influenza A/black headed gull/Sweden/1/99 (GenBank Accession No. AY684887.1). In a specific embodiment, an influenza A virus HA utilized in the generation of a mosaic HA polypeptide described herein is the HA from influenza A/mallared/Gurjev/263/1982 (e.g., Influenza Research Database Accession No. GQ247868). In a specific embodiment, an influenza A virus HA utilized in the generation of a mosaic HA polypeptide described herein is the HA from influenza A/Jiagi-Donghu/346-1/2013 (e.g., Global Research Database Accession No. EP1530526).

In a specific embodiment, an influenza A virus HA utilized in the generation of a mosaic HA polypeptide described herein is the HA from influenza A/OKLAHOMA/309/2006. In other embodiments, an influenza A virus utilized in the generation of a mosaic HA polypeptide described herein is not the HA from A/OKLAHOMA/309/2006. In a specific embodiment, an influenza A virus HA utilized in the generation of a mosaic HA polypeptide described herein is the HA from influenza A/WSN/33. In other embodiments, an influenza A virus utilized in the generation of a mosaic HA polypeptide described herein is not the HA from influenza A/WSN/33.

In a specific embodiment, an influenza A virus HA sequence utilized to generate a mosaic HA polypeptide described herein is the HA sequence from an influenza A virus described in Section 5.4 below. In a specific embodiment, the influenza A virus HA sequence utilized to generate a mosaic HA polypeptide described herein is the HA sequence from an influenza A virus described in Section 6. For example, the influenza A virus HA may be from a group 1 virus (e.g, H1, H2, H5, H6, H8, H9, H11, H12, H13, H16 or H17) or a group 2 virus (e.g., H3, H4, H7, H10, H14 or H15). In specific embodiments, the influenza A virus HA is from an H1, H2, H3, H4, H5, H6, H7, H8, H9, H10, H11, H12, H13, H14, H15, H16, or H17 influenza A virus.

In a specific embodiment, a mosaic HA polypeptide is a mosaic HA polypeptide described in Section 6. In a specific embodiment, a mosaic HA polypeptide comprises the amino acid sequence of the mosaic HA polypeptide in SEQ ID NO: 30. In another specific embodiment, a mosaic HA polypeptide comprises the amino acid sequence of the ectodomain of the mosaic HA polypeptide in SEQ ID NO: 31.

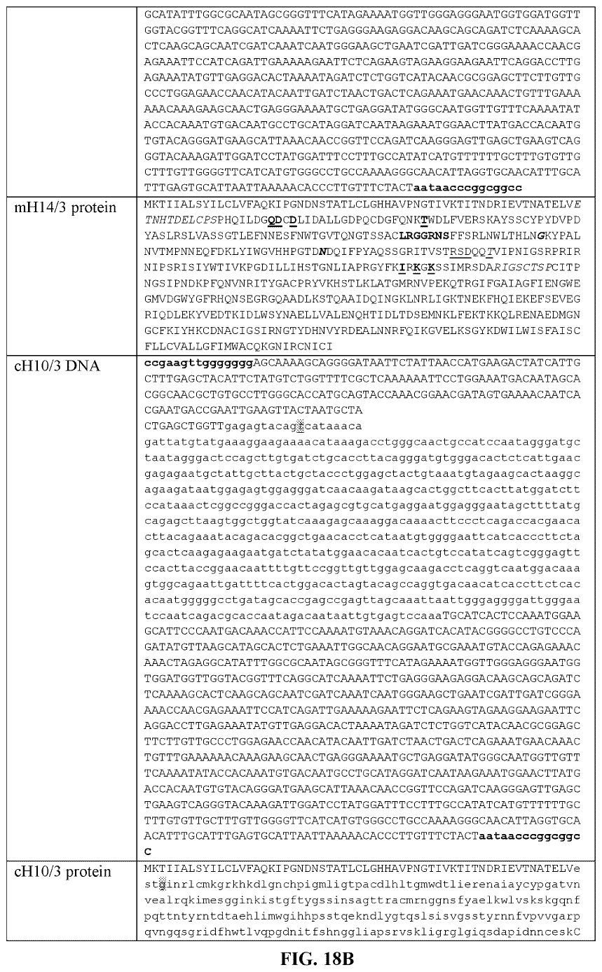

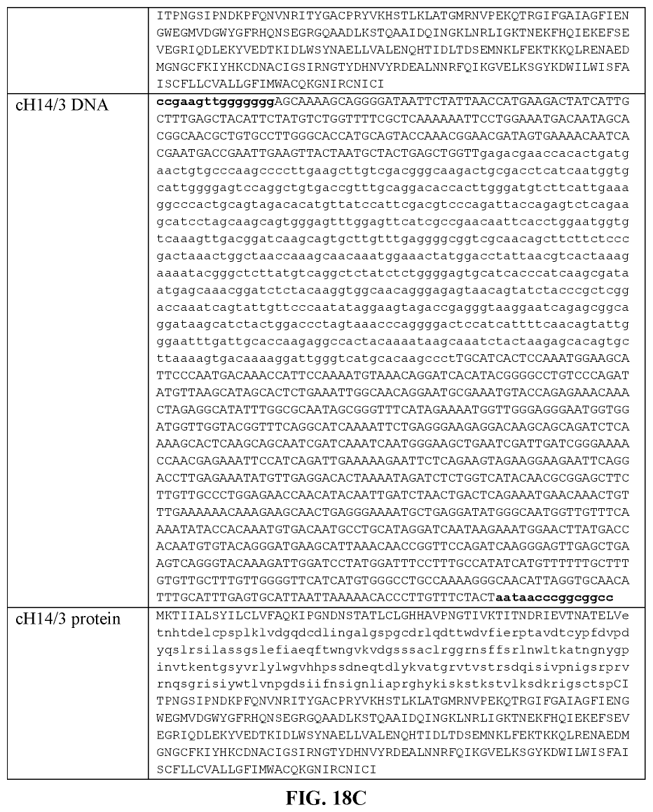

In another specific embodiment, a mosaic HA polypeptide is a mosaic influenza virus HA polypeptide comprising the amino acid sequence set forth in FIG. 18A under mH10/3 (SEQ ID NO: 173). In another specific embodiment, a mosaic HA polypeptide is a mosaic influenza virus HA polypeptide comprising the amino acid sequence set forth in FIG. 18B under mH14/3 (SEQ ID NO: 175).

In another specific embodiment, a mosaic HA polypeptide is a mosaic influenza HA polypeptide described in Section 6, infra. In another specific embodiment, a mosaic HA polypeptide is a mosaic influenza virus HA polypeptide comprising the amino acid sequence of the ectodomain of the mosaic HA polypeptide set forth in FIG. 18A under mH10/3 (SEQ ID NO: 172). In another specific embodiment, a mosaic HA polypeptide is a mosaic influenza virus HA polypeptide comprising the amino acid sequence of the ectodomain of the mosaic HA polypeptide set forth in FIG. 18B under mH14/3 (SEQ ID NO: 174).

In specific embodiments, the mosaic influenza virus hemagglutinin polypeptides provided herein are capable of forming a three dimensional structure that is similar to the three dimensional structure of a wild-type influenza hemagglutinin. Structural similarity might be evaluated based on any technique deemed suitable by those of skill in the art. For instance, reaction, e.g. under non-denaturing conditions, of a mosaic influenza virus hemagglutinin polypeptide with a neutralizing antibody or antiserum that recognizes a native influenza hemagglutinin might indicate structural similarity. Useful neutralizing antibodies or antisera are described in, e.g. Sui, et al., 2009, Nat. Struct. Mol. Biol. 16(3):265-273, Ekiert et al., Feb. 26, 2009, Science [DOI: 10.1126/science. 1171491], and Kashyap et al., 2008, Proc. Natl. Acad. Sci. USA 105(16):5986-5991, the contents of which are hereby incorporated by reference in their entireties. In certain embodiments, the antibody or antiserum is an antibody or antiserum that reacts with a non-contiguous epitope (i.e., not contiguous in primary sequence) that is formed by the tertiary or quaternary structure of a hemagglutinin.

In certain embodiments, a mosaic influenza hemagglutinin (HA) polypeptide described herein retains one, two, or more, or all of the functions of a wild-type influenza HA. Nonlimiting examples of functions of a wild-type influenza HA include fusogenic activity, receptor binding activity, budding, and particle formation. In a specific embodiment, a mosaic influenza hemagglutinin (HA) polypeptide described herein has fusogenic activity. Assays known to one skilled in the art can be utilized the assess the fusogenic activity of a mosaic influenza hemagglutinin (HA) polypeptide described herein, such as, for example, immunofluorescence assays and pseudotyped virus-like-particle assays.

It will be understood by those of skill in the art that the mosaic influenza virus hemagglutinin polypeptides provided herein can be prepared according to any technique known by and deemed suitable to those of skill in the art, including the techniques described herein. In certain embodiments, the mosaic influenza virus hemagglutinin polypeptides are isolated.

### 5.1.1 MOSAIC INFLUENZA A VIRUS—GROUP 1

In another aspect, provided herein are mosaic hemagglutinin (HA) polypeptides comprising an HA ectodomain of a group 1 influenza A virus strain comprising 2, 3, 4, 5, 6, 7, 8, 9, 10, 11, 12 or more amino acid substitutions within the Ca1, Ca2, Cb, Sa, or Sb antigenic site or a corresponding hypervariable antigenic site of the globular head domain of the group influenza A virus HA strain, wherein the amino acid substitutions substitute 2, 3, 4, 5, 6, 7, 8, 9, 10, 11, 12 or more amino acid residues in the Ca1, Ca2, Cb, Sa, or Sb antigenic site of the globular head of the group 1 influenza A virus strain HA with amino acid residues found in a corresponding region of the globular domain of a different group 1 influenza A virus HA strain or subtype, or a group 2 influenza A virus strain HA. In another embodiment, provided herein are mosaic hemagglutinin (HA) polypeptides comprising an HA ectodomain of a group 1 influenza A virus strain 1, 2, 3, 4, 5, 6, 7, 8, 9, 10 or more amino acid substitutions within the Ca1 antigenic site or a corresponding hypervariable antigenic site of the globular head domain of the group 1 influenza A virus strain HA, wherein the amino acid substitutions substitute 1, 2, 3, 4, 5, 6, 7, 8, 9, 10 or more amino acid substitutions within the Ca1 antigenic site or a corresponding hypervariable antigenic site of the globular head domain of the group 1 influenza A virus strain HA with amino acid residues found in a corresponding region of the globular domain of a different group 1 influenza A virus HA strain or subtype, or a group 2 influenza A virus strain HA. In another embodiment, provided herein are mosaic hemagglutinin (HA) polypeptides comprising an HA ectodomain of a group 1 influenza A virus strain 1, 2, 3, 4, 5, 6, 7 or more amino acid substitutions within the Ca2 antigenic site or a corresponding hypervariable antigenic site of the globular head domain of the group 1 influenza A virus strain HA, wherein the amino acid substitutions substitute 1, 2, 3, 4, 5, 6, 7 or more amino acid substitutions within the Ca2 antigenic site or a corresponding hypervariable antigenic site of the globular head domain of the group 1 influenza A virus strain HA with amino acid residues found in a corresponding region of the globular domain of a different group 1 influenza A virus HA strain or subtype, or a group 2 influenza A virus strain HA. In another embodiment, provided herein are mosaic hemagglutinin (HA) polypeptides comprising an HA ectodomain of a group 1 influenza A virus strain 1, 2, 3, 4, 5 or more amino acid substitutions within the Cb antigenic site or a corresponding hypervariable antigenic site of the globular head domain of the group 1 influenza A virus strain HA, wherein the amino acid substitutions substitute 1, 2, 3, 4, 5 or more amino acid substitutions within the Cb antigenic site or a corresponding hypervariable antigenic site of the globular head domain of the group 1 influenza A virus strain HA with amino acid residues found in a corresponding region of the globular domain of a different group 1 influenza A virus HA strain or subtype, or a group 2 influenza A virus strain HA. In another embodiment, provided herein are mosaic hemagglutinin (HA) polypeptides comprising an HA ectodomain of a group 1 influenza A virus strain 1, 2, 3, 4, 5, 6, 7, 8, 9, 10, 11, 12 or more amino acid substitutions within the Sa antigenic site or a corresponding hypervariable antigenic site of the globular head domain of the group 1 influenza A virus strain HA, wherein the amino acid substitutions substitute 1, 2, 3, 4, 5, 6, 7, 8, 9, 10, 11, 12 or more amino acid substitutions within the Sa antigenic site or a corresponding hypervariable antigenic site of the globular head domain of the group 1 influenza A virus strain HA with amino acid residues found in a corresponding region of the globular domain of a different group 1 influenza A virus HA strain or subtype, or a group 2 influenza A virus strain HA. In another embodiment, provided herein are mosaic hemagglutinin (HA) polypeptides comprising an HA ectodomain of a group 1 influenza A virus strain 1, 2, 3, 4, 5, 6, 7, 8, 9, 10, 11 or more amino acid substitutions within the Sb antigenic site or a corresponding hypervariable antigenic site of the globular head domain of the group 1 influenza A virus strain HA, wherein the amino acid substitutions substitute 1, 2, 3, 4, 5, 6, 7, 8, 9, 10, 11 or more amino acid substitutions within the Sb antigenic site or a corresponding hypervariable antigenic site of the globular head domain of the group 1 influenza A virus strain HA with amino acid residues found in a corresponding region of the globular domain of a different group 1 influenza A virus HA strain or subtype, or a group 2 influenza A virus strain HA. In some embodiments, the group 1 influenza A virus strain is an H1 virus. In some embodiments, the group 1 influenza A virus strain is an H2, H5, H6, H8, H9, H12, H13, H16 or H17. In specific embodiments, the influenza A virus from which the amino acid residues are derived for the amino acid substitutions in one, two, three or more of the antigenic sites is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99).

In specific embodiments, one or more of the following amino acid residues in the Ca1 antigenic site of the globular head domain of the group 1 influenza A virus strain HA may be substituted with amino acid residues found in a corresponding region of a group 1 influenza A virus HA of a different strain or subtype, or a group 2 influenza virus HA: INDKG (SEQ ID NO: 7), TSR, and EPG. In a specific embodiment, the amino acid sequences INDKG (SEQ ID NO: 7), TSR, and EPG in the Ca1 antigenic site of the globular head domain of the group 1 influenza A virus strain HA are substituted with the amino acid sequences in Table 7, infra.

In specific embodiments, one or more of the following amino acid residues in the Ca2 antigenic site of the globular head domain of the group 1 influenza A virus strain HA may be substituted with amino acid residues found in a corresponding region of a group 1 influenza A virus HA of a different strain or subtype, or a group 2 influenza virus HA: PHAGAK (SEQ ID NO: 9) and RD. In a specific embodiment, the amino acid sequences PHAGAK (SEQ ID NO: 9) and RD in the Ca2 antigenic site of the globular head domain of the group 1 influenza A virus strain HA are substituted with the amino acid sequences in Table 7, infra.

In specific embodiments, one or more of the following amino acid residues in the Cb antigenic site of the globular head domain of the group 1 influenza A virus strain HA may be substituted with amino acid residues found in a corresponding region of a group 1 influenza A virus HA of a different strain or subtype, or a group 2 influenza virus HA: LSTASS (SEQ ID NO: 11). In a specific embodiment, the amino acid sequence LSTASS (SEQ ID NO: 11) in the Cb antigenic site of the globular head domain of the group 1 influenza A virus strain HA are substituted with the amino acid sequences in Table 7, infra.

In specific embodiments, one or more of the following amino acid residues in the Sa antigenic site of the globular head domain of the group 1 influenza A virus strain HA may be substituted with amino acid residues found in a corresponding region of a group 1 influenza A virus HA of a different strain or subtype, or a group 2 influenza virus HA: PN, KKGNS (SEQ ID NO: 1) and PKLNQS (SEQ ID NO: 2). In a specific embodiment, the amino acid sequences PN, KKGNS (SEQ ID NO: 1) and PKLNQS (SEQ ID NO: 2) in the Sa antigenic site of the globular head domain of the group 1 influenza A virus strain HA are substituted with the amino acid sequences in Table 7, infra.

In specific embodiments, one or more of the following amino acid residues in the Sb antigenic site of the globular head domain of the group 1 influenza A virus strain HA may be substituted with amino acid residues found in a corresponding region of a group 1 influenza A virus HA of a different strain or subtype, or a group 2 influenza virus HA: TTADQQSLYQNA (SEQ ID NO: 5). In a specific embodiment, the amino acid sequence TTADQQSLYQNA (SEQ ID NO: 5) in the Sb antigenic site of the globular head domain of the group 1 influenza A virus strain HA are substituted with the amino acid sequences in Table 7, infra.

TABLE 7
Exemplary Antigenic Sites of Mosaic H1.

| | Original H1 sequence | |
|---|---|---|
| Antigenic | (A/Michigan/45/2015 | |
| Site | H1 HA) | Mosaic H1 sequence |
| Sa | PN (aa 123-124) | PS (aa 123-124) |
| Sa | KKGNS (aa 153-157) | KKNST (aa 152-156) |
| | (SEQ ID NO: 1) | (SEQ ID NO: 3) |
| Sa | PKLNQS (aa 159-164) | PTIKRS (aa 158-163) |
| | (SEQ ID NO: 2) | (SEQ ID NO: 4) |
| Sb | TTADQQSLYQNA | DAAEQTKLYQNP |
| | (aa 184-195) | (aa 183-194) |
| | (SEQ ID NO: 5) | (SEQ ID NO: 6) |
| Ca1 | INDKG (aa 166-170) | NNTTG (aa 165-169) |
| | (SEQ ID NO: 7) | (SEQ ID NO: 8) |
| Ca1 | TSR (aa 203-205) | TSS (aa 202-204) |
| Ca1 | EPG (aa 235-237) | HPG (aa 234-236) |
| Ca2 | PHAGAK (aa 137-142) | PYQGKS (aa 136-141) |
| | (SEQ ID NO: 9) | (SEQ ID NO: 10) |
| Ca2 | RD (aa 221-222) | ND (aa 220-221) |
| Cb | LSTASS (aa 70-75) | LNVPE (aa 70-74; |
| | (SEQ ID NO: 11) | gap not counted) |
| | | (SEQ ID NO: 12) |

Amino acid numbering is based on the mature HA, gaps are not counted.

In specific embodiments, a mosaic HA polypeptide described herein comprises the ectodomain of a group 1 influenza A virus HA, wherein the ectodomain comprises one, two, three, four or all of the Ca1, Ca2, Cb, Sa, and Sb antigenic sites or the corresponding hypervariable antigenic sites of the globular head domain of a group 1 influenza A virus strain HA with the amino acid sequences of the Ca1, Ca2, Cb, Sa, and Sb, respectively, set forth in Table 7. In some embodiments, the mosaic HA polypeptide may also comprise the transmembrane domain, and cytoplasmic tail domain from the group 1 influenza A virus strain HA. In some embodiments, the mosaic HA polypeptide may also comprise the signal peptide, transmembrane domain, and cytoplasmic tail domain from the group 1 influenza A virus strain HA. In other embodiments, the mosaic HA polypeptide comprises the signal peptide from the group 1 influenza A virus HA strain but lacks the transmembrane and cytoplasmic tail domains. In certain embodiment, the mosaic HA comprises the signal peptide of the HA of the influenza virus backbone of the mosaic HA. For example, if the mosaic HA is engineered for an influenza A virus backbone of a different influenza A virus strain or subtype than the group 1 influenza virus strain (e.g., the influenza A virus comprising or engineered to express the mosaic HA is an influenza A virus), then the mosaic HA may comprise the signal peptide of the influenza A virus backbone. In specific embodiments, the mosaic HA polypeptide is soluble. In certain embodiments, the mosaic HA polypeptides comprise 1, 2, 3, 4, 5 or more amino acid substitutions in the globular head domain of the group 1 influenza A virus strain HA which are outside of any or all of the Ca1, Ca2, Cb, Sa, and Sb antigenic sites or the corresponding hypervariable antigenic sites of the globular head domain of a group 1 influenza A virus strain HA.

In a specific embodiment, provided herein is a mosaic influenza virus hemagglutinin (HA) polypeptide comprising an influenza A virus HA ectodomain of a group 1 influenza A virus strain HA, wherein the HA ectodomain comprises an HA stem domain of the group 1 influenza A virus strain HA and an HA globular head domain of the group 1 influenza A virus strain HA, wherein the HA globular head domain of the group 1 influenza A virus strain HA has been engineered to comprise four or all of the following: a. 1, 2, 3, 4, 5, 6, 7, 8, 9, 10, 11, 12 or more amino acid substitutions within the Sa antigenic site or a corresponding hypervariable antigenic site of the globular head domain of the group 1 influenza A virus strain HA; b. 1, 2, 3, 4, 5, 6, 7, 8, 9, 10, 11 or more amino acid substitutions within the Sb antigenic site or a corresponding hypervariable antigenic site of the globular head domain of the group 1 influenza A virus strain HA; c. 1, 2, 3, 4, 5, 6, 7, 8, 9, 10 or more amino acid substitutions within the Ca1 antigenic site or a corresponding hypervariable antigenic site of the globular head domain of the group 1 influenza A virus strain HA; d. 1, 2, 3, 4, 5, 6, 7 or more amino acid substitutions within the Ca2 antigenic site or a corresponding hypervariable antigenic site of the globular head domain of the group 1 influenza A virus strain HA; and e. 1, 2, 3, 4, 5 or more amino acid substitutions within the Cb antigenic site or a corresponding hypervariable antigenic site of the globular head domain of the group 1 influenza A virus strain HA. In a specific embodiment, the mosaic influenza virus HA polypeptide further comprises the transmembrane and cytoplasmic domains of the first group 1 influenza A virus strain HA. In a preferred embodiment, the first group 1 influenza A virus is an H1 subtype (e.g., A/Michigan/45/2015). In some embodiments, the first group 1 influenza A virus is A/Michigan/45/2015 or A/California/7/2009 (H1N1)pdm09-like virus.

In a specific embodiment, provided herein is a mosaic influenza virus hemagglutinin (HA) polypeptide comprising an influenza A virus HA ectodomain of a group 1 influenza A virus strain HA, wherein the HA ectodomain comprises an HA stem domain of the group 1 influenza A virus strain HA and an HA globular head domain of the group 1 influenza A virus strain HA, wherein the HA globular head domain of the group 1 influenza A virus strain HA has been engineered to comprise four or all of the following: a. 4, 5, 6, 7, 8, 9, 10, 11, 12 or more amino acid substitutions within the Sa antigenic site or a corresponding hypervariable antigenic site of the globular head domain of the group 1 influenza A virus strain HA; b. 4, 5, 6, 7, 8, 9, 10, 11 or more amino acid substitutions within the Sb antigenic site or a corresponding hypervariable antigenic site of the globular head domain of the group 1 influenza A virus strain HA; c. 4, 5, 6, 7, 8, 9, 10 or more amino acid substitutions within the Ca1 antigenic site or a corresponding hypervariable antigenic site of the globular head domain of the group 1 influenza A virus strain HA; d. 1, 2, 3, 4, 5, 6, 7 or more amino acid substitutions within the Ca2 antigenic site or a corresponding hypervariable antigenic site of the globular head domain of the group 1 influenza A virus strain HA; and e. 4, 5 or more amino acid substitutions within the Cb antigenic site or a corresponding hypervariable antigenic site of the globular head domain of the group 1 influenza A virus strain HA. In a specific embodiment, the mosaic influenza virus HA polypeptide further comprises the transmembrane and cytoplasmic domains of the first group 1 influenza A virus strain HA. In a preferred embodiment, the first group 1 influenza A virus is an H1 subtype (e.g., A/Michigan/45/2015). In some embodiments, the first group 1 influenza A virus is A/Michigan/45/2015 or A/California/7/2009 (H1N1)pdm09-like virus.

In another specific embodiment, provided herein is a mosaic influenza virus hemagglutinin (HA) polypeptide comprising an HA ectodomain of a first group 1 influenza A virus strain HA, wherein the HA ectodomain comprises an HA stem domain of the first group 1 influenza A virus strain HA and an HA globular head domain of the first group 1 influenza A virus strain HA, wherein the HA globular head domain of the first group 1 influenza A virus strain has been engineered to comprise four or all of the following: a. 1, 2, 3, 4, 5, 6, 7, 8, 9, 10, 11, 12 or more amino acid substitutions within the Sa antigenic site or a corresponding hypervariable antigenic site of the globular head domain of the first group 1 influenza A virus strain HA, wherein the amino acid substitutions substitute 1, 2, 3, 4, 5, 6, 7, 8, 9, 10, 11, 12 or more amino acid residues within the Sa antigenic site or the corresponding hypervariable antigenic site of the globular head domain of the first group 1 influenza A virus strain HA with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; b. 1, 2, 3, 4, 5, 6, 7, 8, 9, 10, 11 or more amino acid substitutions within the Sb antigenic site or a corresponding hypervariable antigenic site of the globular head domain of the first group 1 influenza A virus strain HA, wherein the amino acid substitutions substitute 1, 2, 3, 4, 5, 6, 7, 8, 9, 10, 11 or more amino acid residues within the Sb antigenic site or the corresponding hypervariable antigenic site of the globular head domain of the first group 1 influenza A virus strain HA with amino acid residues found in a corresponding region of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; c. 1, 2, 3, 4, 5, 6, 7, 8, 9, 10 or more amino acid substitutions within the Ca1 antigenic site or a corresponding hypervariable antigenic site of the globular head domain of the first group 1 influenza A virus, strain HA, wherein the amino acid substitutions substitute 1, 2, 3, 4, 5, 6, 7, 8, 9, 10 or more amino acid residues within the Ca1 antigenic site or the corresponding hypervariable antigenic site of the globular head domain of the first group 1 influenza A virus strain HA with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; d. 1, 2, 3, 4, 5, 6, 7 or more amino acid substitutions within the Ca2 antigenic site or a corresponding hypervariable antigenic site of the globular head domain of the first group 1 influenza A virus strain HA, wherein the amino acid substitutions substitute 1, 2, 3, 4, 5, 6, 7 or more amino acid residues within the Ca2 antigenic site or the corresponding hypervariable antigenic site of the globular head domain of the first group 1 influenza A virus strain HA with amino acid residues found in a corresponding region of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; and e. 1, 2, 3, 4, 5 or more amino acid substitutions within the Cb antigenic site or a corresponding hypervariable antigenic site of the globular head domain of the first group 1 influenza A virus strain, wherein the amino acid substitutions substitute 1, 2, 3, 4, 5 or more amino acid residues within the Cb antigenic site or the corresponding hypervariable antigenic site of the globular head domain of the first group 1 influenza A virus strain with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain. In a particular embodiment, the corresponding region of the HA globular head domain is of a group 1 influenza A virus HA of a different subtype than the first group 1 influenza A virus strain. In a specific embodiment, wherein the different subtype is an H5 subtype (e.g., A/Vietnam/1203/2004) or an H13 subtype (e.g., A/black headed gull/Sweden/1/1999 H13 HA). In another embodiment, the corresponding region of the HA globular head domain is of a combination of group 1 influenza A virus HAs of different subtypes than the first group 1 influenza A virus strain. In a specific embodiment, the different subtypes are H5 and H13 subtypes (e.g., A/Vietnam/1203/2004 and A/black headed gull/Sweden/1/1999 H13 HA, respectively). In a specific embodiment, the mosaic influenza virus HA polypeptide further comprises the transmembrane and cytoplasmic domains of the first group 1 influenza A virus strain HA. In a preferred embodiment, the first group 1 influenza A virus is an H1 subtype (e.g., A/Michigan/45/2015). In some embodiments, the first group 1 influenza A virus is A/Michigan/45/2015 or A/California/7/2009.

In another embodiment, provided herein are mosaic hemagglutinin (HA) polypeptides comprising (i) a hemagglutinin ectodomain from a first group 1 influenza A virus strain with 2, 3, 4, 5, 6, 7, 8, 9, 10, 11, 12 or more amino acid substitutions within the Ca1, Ca2, Cb, Sa or Sb antigenic site or a corresponding hypervariable antigenic site of the globular head domain of the group 1 influenza A virus strain HA and (ii) a transmembrane domain and a cytoplasmic tail domain from the first group 1 influenza A virus strain HA, wherein the amino acid substitutions substitute 2, 3, 4, 5, 6, 7, 8, 9, 10, 11, 12 or more amino acid residues in the Ca1, Ca2, Cb, Sa or Sb or a corresponding hypervariable antigenic site of the globular head of the first group 1 influenza A virus strain HA with amino acid residues found in a corresponding region of the globular domain of an influenza A virus HA of a different strain or subtype than the first group 1 influenza A virus strain. In another embodiment, provided herein are mosaic hemagglutinin (HA) polypeptides comprising (i) a hemagglutinin ectodomain from a first group 1 influenza A virus strain with 2, 3, 4, 5, 6, 7, 8, 9, 10, 11, 12 or more amino acid substitutions within each of the Ca1, Ca2, Cb, Sa and Sb antigenic sites or corresponding hypervariable antigenic sites of the globular head domain of the group 1 influenza A virus strain HA and (ii) a transmembrane domain and a cytoplasmic tail domain from the first group 1 influenza A virus strain HA, wherein the amino acid substitutions substitute 2, 3, 4, 5, 6, 7, 8, 9, 10, 11, 12 or more amino acid residues in each of the Ca1, Ca2, Cb, Sa and Sb antigenic sites or corresponding hypervariable antigenic sites of the globular head of the first group 1 influenza A virus strain HA with amino acid residues found in corresponding regions of the globular domain of an influenza A virus HA of a different strain or subtype than the first group 1 influenza A virus strain. In specific embodiments, the first group 1 influenza A virus strain is an H1 (e.g., A/Michigan/45/2015). In a specific embodiment, the influenza A virus backbone of an influenza virus either comprising, containing, or both the mosaic HA is from a second influenza A virus, e.g., A/Puerto Rico/8/34, an influenza virus lacking the NS1 protein (e.g., a delNS1 virus, such as described, e.g., in U.S. Pat. No. 6,468,544; Garcia-Sastre et al., 1998, Virology 252:324; or Mössler et al., 2013, Vaccine 31:6194), or a cold-adapted influenza A virus (e.g., A/Ann Arbor/6/60 or A/Leningrad/134/17/57). In specific embodiments, the influenza A virus from which the amino acid residues are derived for the amino acid substitutions in one, two, three or more of the antigenic sites is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99), or a combination thereof. In specific embodiments, a mosaic HA polypeptide described herein comprises the Ca1, Ca2, Cb, Sa and/or Sb with the amino acid sequences of the Ca1, Ca2, Cb, Sa and/or Sb, respectively, set forth in Table 7. In certain embodiments, the mosaic HA polypeptides comprise 1, 2, 3, 4, 5 or more amino acid substitutions in the globular head domain of the group 2 influenza A virus strain HA which are outside of one, two, three, four or all the following antigenic sites: Ca1, Ca2, Cb, Sa and Sb, or corresponding hypervariable antigenic sites.

In another embodiment, provided herein are mosaic hemagglutinin (HA) polypeptides comprising (i) a hemagglutinin ectodomain from a first group 1 influenza A virus strain with 2, 3, 4, 5, 6, 7, 8, 9, 10, 11, 12 or more amino acid substitutions within the Ca1, Ca2, Cb, Sa or Sb or a corresponding hypervariable antigenic site of the globular head domain of the group 1 influenza A virus strain HA and (ii) a signal peptide, a transmembrane domain and a cytoplasmic tail domain from the first group 1 influenza A virus strain HA, wherein the amino acid substitutions substitute 2, 3, 4, 5, 6, 7, 8, 9, 10, 11, 12 or more amino acid residues in the Ca1, Ca2, Cb, Sa or Sb or a corresponding hypervariable antigenic site of the globular head of the first group 1 influenza A virus strain HA with amino acid residues found in a corresponding region of the globular domain of an influenza A virus HA of a different strain or subtype than the first group 1 influenza A virus strain. In another embodiment, provided herein are mosaic hemagglutinin (HA) polypeptides comprising (i) a hemagglutinin ectodomain from a first group 1 influenza A virus strain with 2, 3, 4, 5, 6, 7, 8, 9, 10, 11, 12 or more amino acid substitutions within each of the Ca1, Ca2, Cb, Sa and Sb antigenic sites or corresponding hypervariable antigenic sites of the globular head domain of the group 1 influenza A virus strain HA and (ii) a signal peptide, a transmembrane domain and a cytoplasmic tail domain from the first group 1 influenza A virus strain HA, wherein the amino acid substitutions substitute 2, 3, 4, 5, 6, 7, 8, 9, 10, 11, 12 or more amino acid residues in each of the Ca1, Ca2, Cb, Sa and Sb antigenic sites or corresponding hypervariable antigenic sites of the globular head of the first group 1 influenza A virus strain HA with amino acid residues found in corresponding regions of the globular domain of an influenza A virus HA of a different strain or subtype than the first group 1 influenza A virus strain. In specific embodiments, the first group 1 influenza A virus strain is an H1 (e.g., A/Michigan/45/2015). In a specific embodiment, the influenza A virus backbone of an influenza virus either comprising, containing, or both the mosaic HA is from a second influenza A virus, e.g., A/Puerto Rico/8/34, an influenza virus lacking the NS1 protein (e.g., a delNS1 virus, such as described, e.g., in U.S. Pat. No. 6,468,544; Garcia-Sastre et al., 1998, Virology 252:324; or Mössler et al., 2013, Vaccine 31:6194), or a cold-adapted influenza A virus (e.g., A/Ann Arbor/6/60 or A/Leningrad/134/17/57). In specific embodiments, the influenza A virus from which the amino acid residues are derived for the amino acid substitutions in one, two, three or more of the antigenic sites is an H5 (e.g., A/Vietnam/1203/04 (HALo)), H8 (e.g., A/mallard/Sweden/24/2002), H11 (e.g., A/northern shoveler/Netherlands/18/99), H12 strain (e.g., A_mallard_interior Alaska_7MP0167_2007), or H13 strain (e.g., A/black headed gull/Sweden/1/99). In specific embodiments, a mosaic HA polypeptide described herein comprises a Ca1, Ca2, Cb, Sa and/or Sb with the amino acid sequences of the Ca1, Ca2, Cb, Sa and/or Sb, respectively, set forth in Table 7. In certain embodiments, the mosaic HA polypeptides comprise 1, 2, 3, 4, 5 or more amino acid substitutions in the globular head domain of the group 1 influenza A virus strain HA which are outside of one, two, three, four or all the following antigenic sites: Ca1, Ca2, Cb, Sa and Sb, or corresponding hypervariable antigenic sites.

In another specific embodiment, provided herein is a mosaic influenza virus HA polypeptide comprising an HA ectodomain of influenza A/Michigan/45/2015 virus HA, wherein the HA ectodomain comprises the influenza A/Michigan/45/2015 virus HA stem domain and HA globular head domain, wherein the globular head domain has been engineered to comprises four or all of the following amino acid sequence substitutions: a. the amino acid sequences PN, KKGNS (SEQ ID NO: 1), and PKLNQS (SQ ID NO: 2) in the HA globular head domain Sa antigenic site of influenza A/Michigan/45/2015 virus HA have been substituted with the following amino acid sequences PS, KKNST (SEQ ID NO: 3), and PTIKRS (SEQ ID NO: 4), respectively; b. the amino acid sequence TTADQQSLYQNA (SEQ ID NO: 5) in the HA globular head domain Sb antigenic site of influenza A/Michigan/45/2015 virus HA has been substituted with the following amino acid sequence DAAEQTKLYQNP (SEQ ID NO: 6); c. the amino acid sequences INDKG (SEQ ID NO: 7), TSR, and EPG in the HA globular head domain Ca1 antigenic site of influenza A/Michigan/45/2015 virus HA have been substituted with the following amino acid sequences NNTTG (SEQ ID NO: 8), TSS, and HPG, respectively; d. the amino acid sequences PHAGAK (SEQ ID NO: 9) and RD in the HA globular head domain Ca2 antigenic site of influenza A/Michigan/45/2015 virus HA have been substituted with the following amino acid sequences PYQGKS (SEQ ID NO: 10) and ND, respectively; and e. the amino acid sequence LSTASS (SEQ ID NO: 11) in the HA globular head domain Cb antigenic site of influenza A/Michigan/45/2015 virus HA have been substituted with the following amino acid sequence LNVPE (SEQ ID NO: 12). In a specific embodiment, the mosaic influenza virus HA polypeptide further comprises the transmembrane and cytoplasmic domains of the influenza A/Michigan/45/2015 virus HA. In a specific embodiment, the influenza A virus backbone of an influenza virus either comprising, containing, or both the mosaic HA is from a second influenza A virus, e.g., A/Puerto Rico/8/34, an influenza virus lacking the NS1 protein (e.g., a delNS1 virus, such as described, e.g., in U.S. Pat. No. 6,468,544; Garcia-Sastre et al., 1998, Virology 252:324; or Mössler et al., 2013, Vaccine 31:6194), or a cold-adapted influenza A virus (e.g., A/Ann Arbor/6/60 or A/Leningrad/134/17/57).

In a specific embodiment, a mosaic influenza virus HA polypeptide described herein comprises the amino acid sequence set forth in SEQ ID NO: 30.

### 5.1.2 MOSAIC INFLUENZA VIRUS A—GROUP 2

In another aspect, provided herein are mosaic hemagglutinin (HA) polypeptides comprising an HA ectodomain of a group 2 influenza A virus strain comprising 2, 3, 4, 5, 6, 7, 8, 9, 10, 11, 12, 13, 14, 15, 16, 17, 18 or more amino acid substitutions within the A, B, C, D, or E antigenic site or a corresponding hypervariable antigenic site of the globular head domain of the group 2 influenza A virus HA strain, wherein the amino acid substitutions substitute 1, 2, 3, 4, 5, 6, 7, 8, 9, 10, 11, 12, 13, 14, 15, 16, 17, 18 or more amino acid substitutions within the A, B, C, D, or E antigenic site of the globular head of the group 2 influenza A virus strain HA with amino acid residues found in a corresponding region of the globular domain of a different influenza A virus HA strain or subtype, or a group 2 influenza A virus strain HA. In another embodiment, provided herein are mosaic hemagglutinin (HA) polypeptides comprising an HA ectodomain of a group 2 influenza A virus strain 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the group 2 influenza A virus strain HA, wherein the amino acid substitutions substitute 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the group 2 influenza A virus strain HA with amino acid residues found in a corresponding region of the globular domain of a different influenza A virus HA strain or subtype, or a group 1 influenza A virus strain HA. In another embodiment, provided herein are mosaic hemagglutinin (HA) polypeptides comprising an HA ectodomain of an influenza A virus strain 1, 2, 3, 4, 5, 6, 7, 8, 9, 10, 11, 12, 13, 14, 15, 16, 17 or more amino acid substitutions within the B antigenic site or a corresponding hypervariable antigenic site of the globular head domain of the group 2 influenza A virus strain HA, wherein the amino acid substitutions substitute 1, 2, 3, 4, 5, 6, 7, 8, 9, 10, 11, 12, 13, 14, 15, 16, 17 or more amino acid substitutions within the B antigenic site or a corresponding hypervariable antigenic site of the globular head domain of the group 2 influenza A virus strain HA with amino acid residues found in a corresponding region of the globular domain of a different group 2 influenza A virus HA strain or subtype, or a group 1 influenza A virus strain HA. In another embodiment, provided herein are mosaic hemagglutinin (HA) polypeptides comprising an HA ectodomain of a group 2 influenza A virus strain 1, 2, 3, 4, 5, 6, 7, 8, 9, 10, 11, 12, 13, 14, 15, 16, 17, 18 or more amino acid substitutions within the C antigenic site or a corresponding hypervariable antigenic site of the globular head domain of the group 2 influenza A virus strain HA, wherein the amino acid substitutions substitute 1, 2, 3, 4, 5, 6, 7, 8, 9, 10, 11, 12, 13, 14, 15, 16, 17, 18 or more amino acid substitutions within the C antigenic site or a corresponding hypervariable antigenic site of the globular head domain of the group 2 influenza A virus strain HA with amino acid residues found in a corresponding region of the globular domain of a different group 2 influenza A virus HA strain or subtype, or a group 1 influenza A virus strain HA. In another embodiment, provided herein are mosaic hemagglutinin (HA) polypeptides comprising an HA ectodomain of a group 2 influenza A virus strain 1, 2, 3, 4, 5, 6, 7, 8, 9, 10, 11, 12, 13, 14, 15, 16, 17, 18 or more amino acid substitutions within the D antigenic site or a corresponding hypervariable antigenic site of the globular head domain of the group 2 influenza A virus strain HA, wherein the amino acid substitutions substitute 1, 2, 3, 4, 5, 6, 7, 8, 9, 10, 11, 12, 13, 14, 15, 16, 17, 18 or more amino acid substitutions within the D antigenic site or a corresponding hypervariable antigenic site of the globular head domain of the group 2 influenza A virus strain HA with amino acid residues found in a corresponding region of the globular domain of a different group 2 influenza A virus HA strain or subtype, or a group 1 influenza A virus strain HA. In another embodiment, provided herein are mosaic hemagglutinin (HA) polypeptides comprising an HA ectodomain of a group 2 influenza A virus strain 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the group 2 influenza A virus strain HA, wherein the amino acid substitutions substitute 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the group 2 influenza A virus strain HA with amino acid residues found in a corresponding region of the globular domain of a different influenza A virus HA strain or subtype, or a group 2 influenza A virus strain HA. In some embodiments, the group 1 influenza A virus strain is an H3 virus. In some embodiments, the group 2 influenza A virus strain is an H4, H7, H10, H14, or H15. In specific embodiments, the influenza A virus from which the amino acid residues are derived for the amino acid substitutions in one, two, three or more of the antigenic sites is an H10 (e.g., A/Jiangxi-Donghu/346-1/2013). In specific embodiments, the influenza A virus from which the amino acid residues are derived for the amino acid substitutions in one, two, three or more of the antigenic sites is an H14 (e.g., A/mallard/Gurjev/263/1982).

In specific embodiments, one or more of the following amino acid residues in the A antigenic site of the globular head domain of the group 2 influenza A virus strain HA may be substituted with amino acid residues found in a corresponding region of a group 2 influenza A virus HA of a different strain or subtype, or a group 1 influenza A virus HA: NNESFNWT-GVTQNGTSSACIRRSSSS (SEQ ID NO: 13). In a specific embodiment, the amino acid sequence NNESFNWT-GVTQNGTSSACIRRSSSS (SEQ ID NO: 13) in the A antigenic site of the globular head domain of the group 2 influenza A virus strain HA are substituted with the amino acid sequence in Table 8, infra.

In specific embodiments, one or more of the following amino acid residues in the B antigenic site of the globular head domain of the group 2 influenza A virus strain HA may be substituted with amino acid residues found in a corresponding region of a group 2 influenza A virus HA of a different strain or subtype, or a group 1 influenza A virus HA: THL-NYK (SEQ ID NO: 15) and GTDKDQIFLYAQ (SEQ ID NO: 16). In a specific embodiment, the amino acid sequences THL-NYK (SEQ ID NO: 15) and GTDKDQIFLYAQ (SEQ ID NO:16) in the B antigenic site of the globular head domain of the group 2 influenza A virus strain HA are substituted with the amino acid sequences in Table 8, infra.

In specific embodiments, one or more of the following amino acid residues in the C antigenic site of the globular head domain of the group 2 influenza A virus strain HA may be substituted with amino acid residues found in a corresponding region of a group 2 influenza A virus HA of a different strain or subtype, or a group 1 influenza A virus HA: QNSSIGEICDS (SEQ ID NO: 19) and PIG-KCKSE (SEQ ID NO: 20). In a specific embodiment, the amino acid sequences QNSSIGEICDS (SEQ ID NO: 19) and PIG-KCKSE (SEQ ID NO: 20) in the C antigenic site of the globular head domain of the group 2 influenza A virus strain HA are substituted with the amino acid sequences in Table 8, infra.

In specific embodiments, one or more of the following amino acid residues in the D antigenic site of the globular head domain of the group 2 influenza A virus strain HA may be substituted with amino acid residues found in a corresponding region of a group 2 influenza D virus HA of a different strain or subtype, or a group 1 influenza A virus HA: RITVSTKRSQQAVIPNIGS (SEQ ID NO: 23). In a specific embodiment, the amino acid sequence RITVSTKRSQQAVIPNIGS (SEQ ID NO: 23) in the D antigenic site of the globular head domain of the group 2 influenza A virus strain HA are substituted with the amino acid sequence in Table 8, infra.

In specific embodiments, one or more of the following amino acid residues in the E antigenic site of the globular head domain of the group 2 influenza A virus strain HA may be substituted with amino acid residues found in a corresponding region of a group 2 influenza E virus HA of a different strain or subtype, or a group 1 influenza A virus HA: ENCT (SEQ ID NO: 124), GFQNKKWDLFVERSKAY (SEQ ID NO: 27) and IRSGKS (SEQ ID NO: 28). In a specific embodiment, the amino acid sequences ENCT (SEQ ID NO: 124), GFQNKKWDLFVERSKAY (SEQ ID NO: 27) and IRSGKS (SEQ ID NO: 28) in the E antigenic site of the globular head domain of the group 2 influenza A virus strain HA are substituted with the amino acid sequence in Table 8, infra.

TABLE 8
Exemplary Antigenic Sites of Mosaic H3.

| Anti- | Original H3 | |
|---|---|---|
| genic | sequence (A/Hong | |
| Site | Kong/4801/2014) | Mosaic H3 sequence |
| A | NNESFNWT- | NNESFNWT- |
| | GVTQNGTSSACIRRSSSS | GVTQNGTSSACMRNGGNS |
| | (aa 121-146) | (aa 121-146) |
| | (SEQ ID NO: 13) | (SEQ ID NO: 14) |
| B | THL--NYK | THL--NQK |
| | (aa 155-160) | (aa 155-160) |
| | (SEQ ID NO: 15) | (SEQ ID NO: 17) |
| B | GTDKDQIFLYAQ | GTNQDQIFLYAQ |
| | (aa 186-197) | (aa 186-197) |
| | (SEQ ID NO: 16) | (SEQ ID NO: 18) |
| C | QNSSIGEICDS | ESTGINRLCMK |
| | (aa 44-54) | (aa 44-54) |
| | (SEQ ID NO: 19) | (SEQ ID NO: 21) |
| C | PIG-KCKSE | PIDNNCESK |
| | (aa 273-280) | (aa 273-281) |
| | (SEQ ID NO: 20) | (SEQ ID NO: 22) |
| D | RITVSTKRSQQAVIPNIGS | RITVSTSTYQQAVIPNIGS |
| | (aa 201-219) | (aa 201-219) |
| | (SEQ ID NO: 23) | (SEQ ID NO: 25) |
| E | ENCT (aa 62-65) | GNCH (aa 62-65) |
| | (SEQ ID NO: 124) | (SEQ ID NO: 125) |
| E | GFQNKKWDLFVERSKAY | GFQNKMWDLFVERSKAY |
| | (aa 78-94) | (aa 78-94) |
| | (SEQ ID NO: 27) | (SEQ ID NO: 29) |
| E | IRSGKS (aa 260-265) | LRIGRS (aa 260-265) |
| | (SEQ ID NO: 28) | (SEQ ID NO: 24) |

Amino acid numbering is based on the mature HA, gaps are not counted.

In specific embodiments, a mosaic HA polypeptide described herein comprises the ectodomain of a group 2 influenza A virus HA, wherein the ectodomain comprises one, two, three, four or all of the A, B, C, D and E antigenic sites or the corresponding hypervariable antigenic sites of the globular head domain of a group 2 influenza A virus strain HA with the amino acid sequences of the A, B, C, D and E, respectively, set forth in Table 8. In some embodiments, the mosaic HA polypeptide may also comprise the transmembrane domain, and cytoplasmic tail domain from the group 2 influenza A virus strain HA. In some embodiments, the mosaic HA polypeptide may also comprise the signal peptide, transmembrane domain, and cytoplasmic tail domain from the group 2 influenza A virus strain HA. In other embodiments, the mosaic HA polypeptide comprises the signal peptide from the group 2 influenza A virus HA strain but lacks the transmembrane and cytoplasmic tail domains. In certain embodiment, the mosaic HA comprises the signal peptide of the HA of the influenza virus backbone of the mosaic HA. For example, if the mosaic HA is engineered for an influenza A virus backbone of a different influenza A virus strain or subtype than the group 2 influenza virus strain (e.g., the influenza A virus comprising or engineered to express the mosaic HA is an influenza A virus), then the mosaic HA may comprise the signal peptide of the influenza A virus backbone. In specific embodiments, the mosaic HA polypeptide is soluble. In certain embodiments, the mosaic HA polypeptides comprise 1, 2, 3, 4, 5 or more amino acid substitutions in the globular head domain of the group 2 influenza A virus strain HA which are outside of any or all of the A, B, C, D, and E antigenic sites or the corresponding hypervariable antigenic sites of the globular head domain of a group 2 influenza A virus strain HA.

In specific embodiments, provided herein is a mosaic influenza virus HA polypeptide comprising an HA ectodomain of a first group 2 influenza A virus strain HA, wherein the HA ectodomain comprises an HA stem domain of the first group 2 influenza A virus strain HA and an HA globular head domain of the first group 2 influenza A virus strain HA, wherein the HA globular head domain of the first group 2 influenza A virus strain HA has been engineered to comprise four or all of the following: a. 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the first group 2 influenza A virus strain HA; b. 1, 2, 3, 4, 5, 6, 7, 8, 9, 10, 11, 12, 13, 14, 15, 16, 17 or more amino acid substitutions within the B antigenic site or a corresponding hypervariable antigenic site of the globular head domain of the first group 2 influenza A virus strain HA; c. 1, 2, 3, 4, 5, 6, 7, 8, 9, 10, 11, 12, 13, 14, 15, 16, 17, 18 or more amino acid substitutions within the C antigenic site or a corresponding hypervariable antigenic site of the globular head domain of the first group 2 influenza A virus strain HA; d. 1, 2, 3, 4, 5, 6, 7, 8, 9, 10, 11, 12, 13, 14, 15, 16, 17, 18 or more amino acid substitutions within the D antigenic site or a corresponding hypervariable antigenic site of the globular head domain of the first group 2 influenza A virus strain HA; and e. 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the first group 2 influenza A virus strain. In specific embodiments, the mosaic influenza virus HA polypeptide further comprises the transmembrane and cytoplasmic domains of the first group 2 influenza A virus strain HA. In a preferred embodiment, the first group 2 influenza A virus strain is an H3 subtype (e.g., A/Hong Kong/4801/2014, A/Texas/50/2012 or A/Singapore/INFIMH-16-0019/2016). In a preferred embodiment, the first group 2 influenza A virus strain is A/Hong Kong/4801/2014.

In specific embodiments, provided herein is a mosaic influenza virus HA polypeptide comprising an HA ectodomain of a first group 2 influenza A virus strain HA, wherein the HA ectodomain comprises an HA stem domain of the first group 2 influenza A virus strain HA and an HA globular head domain of the first group 2 influenza A virus strain HA, wherein the HA globular head domain of the first group 2 influenza A virus strain HA has been engineered to comprise four or all of the following: a. 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the first group 2 influenza A virus strain HA; b. 4, 5, 6, 7, 8, 9, 10, 11, 12, 13, 14, 15, 16, 17 or more amino acid substitutions within the B antigenic site or a corresponding hypervariable antigenic site of the globular head domain of the first group 2 influenza A virus strain HA; c. 4, 5, 6, 7, 8, 9, 10, 11, 12, 13, 14, 15, 16, 17, 18 or more amino acid substitutions within the C antigenic site or a corresponding hypervariable antigenic site of the globular head domain of the first group 2 influenza A virus strain HA; d. 4, 5, 6, 7, 8, 9, 10, 11, 12, 13, 14, 15, 16, 17, 18 or more amino acid substitutions within the D antigenic site or a corresponding hypervariable antigenic site of the globular head domain of the first group 2 influenza A virus strain HA; and e. 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the first group 2 influenza A virus strain. In specific embodiments, the mosaic influenza virus HA polypeptide further comprises the transmembrane and cytoplasmic domains of the first group 2 influenza A virus strain HA. In a preferred embodiment, the first group 2 influenza A virus strain is an H3 subtype (e.g., A/Hong Kong/4801/2014, A/Texas/50/2012 or A/Singapore/INFIMH-16-0019/2016). In a preferred embodiment, the first group 2 influenza A virus strain is A/Hong Kong/4801/2014. In another embodiment, the first group 2 influenza A virus strain is A/Singapore/INFIMH-16-0019/2016.

In specific embodiments, provided herein is a mosaic influenza virus HA polypeptide comprising an HA ectodomain of a first group 2 influenza A virus strain HA, wherein the HA ectodomain comprises an HA stem domain of the first group 2 influenza A virus strain HA and an HA globular head domain of the first group 2 influenza A virus HA strain HA, wherein the HA globular head domain of the first group 2 influenza A virus strain HA has been engineered to comprise four or all of the following: a. 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18, 19, 20, 21, 22, 23, 24, 25, 26 or more amino acid residues within the A antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; b. 1, 2, 3, 4, 5, 6, 7, 8, 9, 10, 11, 12, 13, 14, 15, 16, 17 or more amino acid substitutions within the B antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17 or more amino acid residues within the B antigenic site or the corresponding hypervariable antigenic site of the globular head domain of the group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; c. 11, 2, 3, 4, 5, 6, 7, 8, 9, 10, 11, 12, 13, 14, 15, 16, 17, 18 or more amino acid substitutions within the C antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18 or more amino acid residues within the C antigenic site or the corresponding hypervariable antigenic site of the globular head domain of the first group 2 influenza A virus strain HA with amino acid residues found in the corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; d. 1, 2, 3, 4, 5, 6, 7, 8, 9, 10, 11, 12, 13, 14, 15, 16, 17, 18 or more amino acid substitutions within the D antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18, or more amino acid residues within the D antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; and e. 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the group 2 influenza A virus strain HA, wherein the amino acid substitutions substitute 1, 2, 3, 4, 5, 6, 7, 8, 9, 10, 11, 12, 13, 14, 15, 16, 17, 18, 19, 20, 21, 22, 23, 24, 25, 26 or more amino acid residues within the E antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain. In some embodiments, the corresponding region of the HA globular head domain is of a group 2 influenza A virus HA of a different subtype than the first influenza A virus group 2 strain. In a specific embodiment, the different subtype is an H10 subtype (e.g., A/Jiangxi-Donghu/346-1/2013). In specific embodiments, the mosaic influenza virus HA polypeptide further comprises the transmembrane and cytoplasmic domains of the first group 2 influenza A virus strain HA. In a preferred embodiment, the first group 2 influenza A virus strain is an H3 subtype (e.g., A/Hong Kong/4801/2014, A/Texas/50/2012 or A/Singapore/INFIMH-16-0019/2016). In a preferred embodiment, the first group 2 influenza A virus strain is A/Hong Kong/4801/2014. In some embodiments, the first group 2 influenza A virus strain is A/Singapore/INFIMH-16-0019/2016.

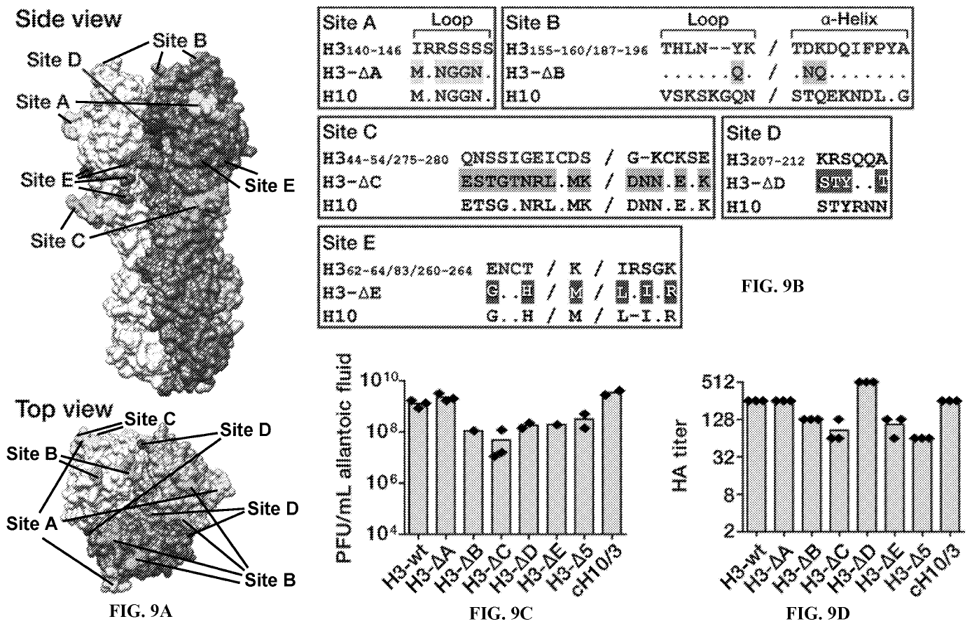

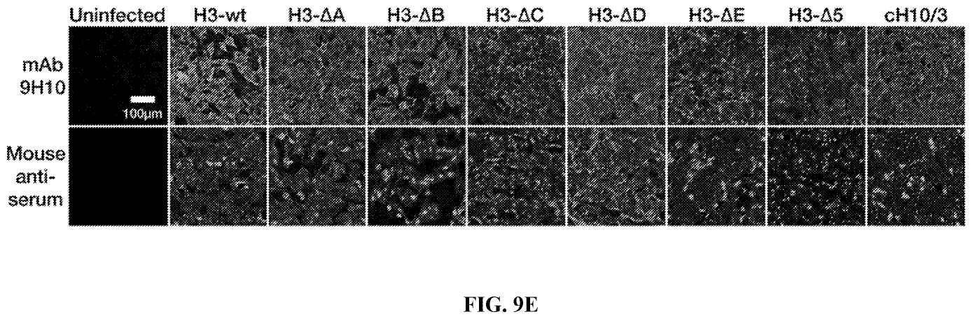

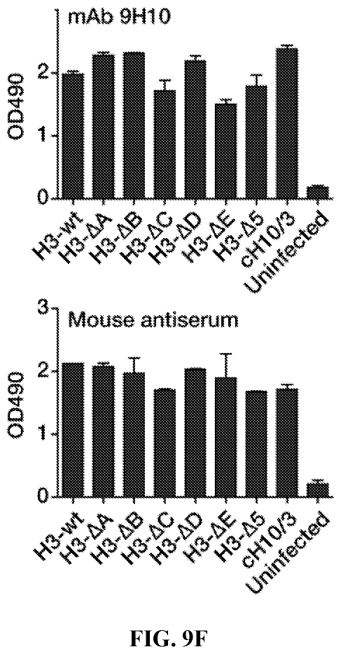

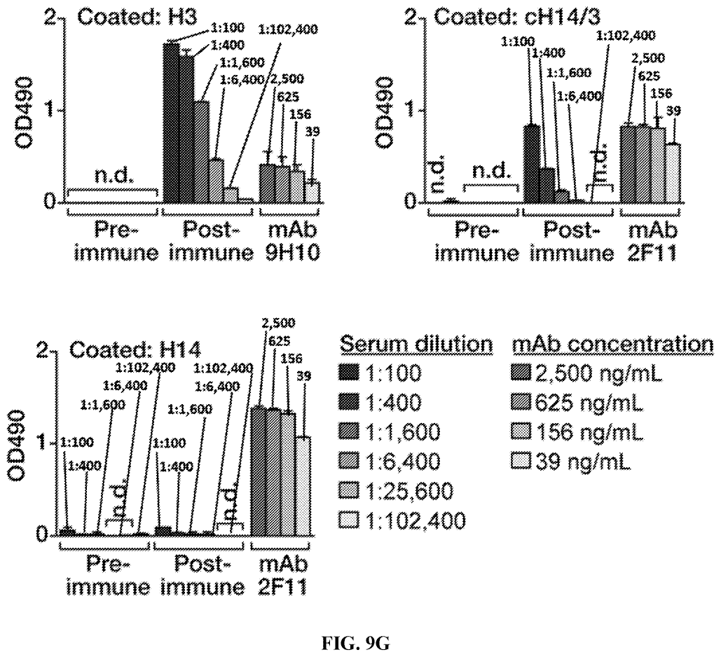

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise the amino acid sequence substitutions in one, two, three, four or all of the following: (a) the amino acid substitutions in the HA globular head domain A antigenic site of influenza A/Hong Kong/4801/2014 virus HA set forth in FIG. 9B under H3-ΔA; (b) the amino acid substitutions in the HA globular head domain B antigenic site of influenza A/Hong Kong/4801/2014 virus HA set forth in FIG. 9B under H3-ΔA; (c) the amino acid substitutions in the HA globular head domain C antigenic site of influenza A/Hong Kong/4801/2014 virus HA set forth in FIG. 9B under H3-ΔA; (d) the amino acid substitutions in the HA globular head domain D antigenic site of influenza A/Hong Kong/4801/2014 virus HA set forth in FIG. 9B under H3-ΔA; and the amino acid substitutions in the HA globular head domain E antigenic site of influenza A/Hong Kong/4801/2014 virus HA set forth in FIG. 9B under H3-ΔA.

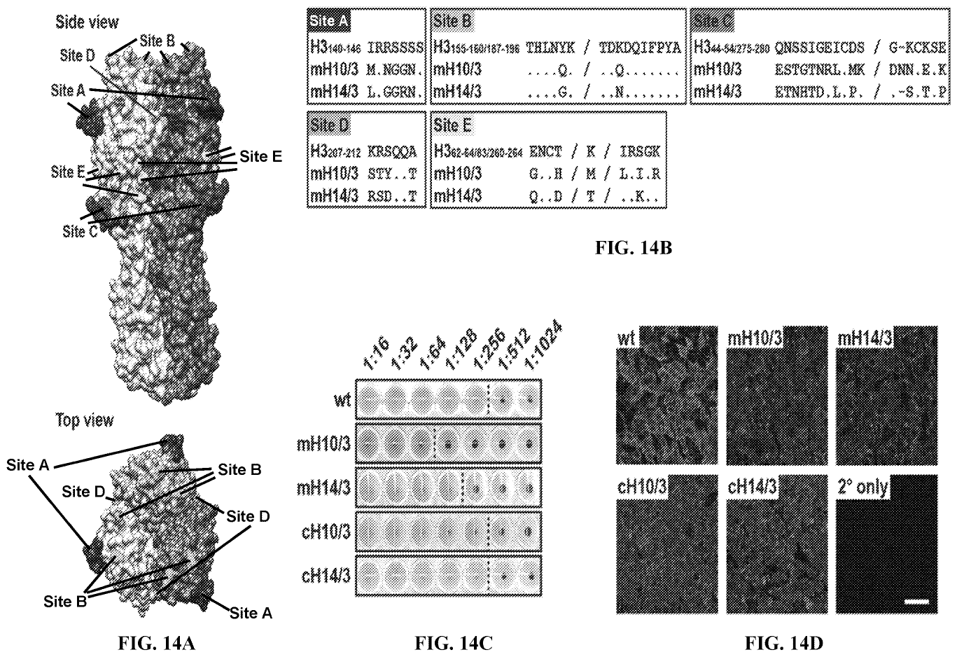

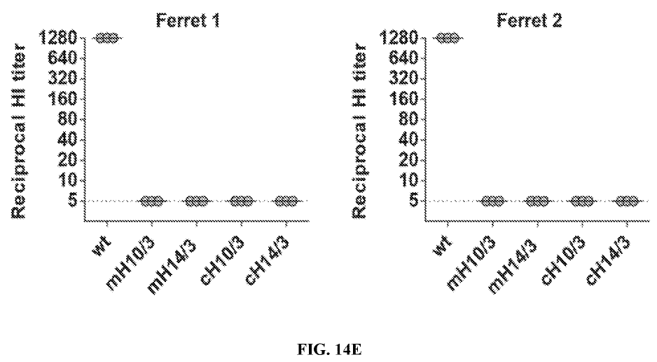

In another aspect, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise the amino acid sequence substitutions in one, two, three, four or all of the following: (a) the amino acid substitutions in the HA globular head domain A antigenic site of influenza A/Hong Kong/4801/2014 virus HA set forth in FIG. 14B under mH10/3; (b) the amino acid substitutions in the HA globular head domain B antigenic site of influenza A/Hong Kong/4801/2014 virus HA set forth in FIG. 14B under mH10/3; (c) the amino acid substitutions in the HA globular head domain C antigenic site of influenza A/Hong Kong/4801/2014 virus HA set forth in FIG. 14B under mH10/3; (d) the amino acid substitutions in the HA globular head domain D antigenic site of influenza A/Hong Kong/4801/2014 virus HA set forth in FIG. 14B under mH10/3; and the amino acid substitutions in the HA globular head domain E antigenic site of influenza A/Hong Kong/4801/2014 virus HA set forth in FIG. 14B under mH10/3.

In a specific embodiment, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise the amino acid sequence substitutions in one, two, three, four or all of the following: (a) the amino acid substitutions in the HA globular head domain A antigenic site of influenza A/Hong Kong/4801/2014 virus HA set forth in FIG. 14B under mH14/3; (b) the amino acid substitutions in the HA globular head domain B antigenic site of influenza A/Hong Kong/4801/2014 virus HA set forth in FIG. 14B under mH14/3; (c) the amino acid substitutions in the HA globular head domain C antigenic site of influenza A/Hong Kong/4801/2014 virus HA set forth in FIG. 14B under mH14/3; (d) the amino acid substitutions in the HA globular head domain D antigenic site of influenza A/Hong Kong/4801/2014 virus HA set forth in FIG. 14B under mH14/3; and the amino acid substitutions in the HA globular head domain E antigenic site of influenza A/Hong Kong/4801/2014 virus HA set forth in FIG. 14B under mH14/3.

In another specific embodiment, provided herein are mosaic hemagglutinin (HA) polypeptides comprising (i) a hemagglutinin ectodomain from a first group 2 influenza A virus strain with 2, 3, 4, 5, 6, 7, 8, 9, 10, 11, 12 or more amino acid substitutions within the A, B, C, D, or E antigenic site or a corresponding hypervariable antigenic site of the globular head domain of the group 2 influenza A virus strain HA and (ii) a transmembrane domain and a cytoplasmic tail domain from the first group 2 influenza A virus strain HA, wherein the amino acid substitutions substitute 2, 3, 4, 5, 6, 7, 8, 9, 10, 11, 12 or more amino acid residues in the A, B, C, D, or E or a corresponding hypervariable antigenic site of the globular head of the first group 2 influenza A virus strain HA with amino acid residues found in a corresponding region of the globular domain of an influenza A virus HA of a different strain or subtype than the first group 2 influenza A virus strain. In another embodiment, provided herein are mosaic hemagglutinin (HA) polypeptides comprising (i) a hemagglutinin ectodomain from a first group 2 influenza A virus strain with 2, 3, 4, 5, 6, 7, 8, 9, 10, 11, 12 or more amino acid substitutions within each of the A, B, C, D, and E antigenic sites or corresponding hypervariable antigenic sites of the globular head domain of the first group 2 influenza A virus strain HA and (ii) a transmembrane domain and a cytoplasmic tail domain from the first group 2 influenza A virus strain HA, wherein the amino acid substitutions substitute 2, 3, 4, 5, 6, 7, 8, 9, 10, 11, 12 or more amino acid residues in each of the A, B, C, D, and E antigenic sites or corresponding hypervariable antigenic sites of the globular head of the first group 2 influenza A virus strain HA with amino acid residues found in a corresponding region of the globular domain of an influenza A virus HA of a different strain or subtype than the first group 2 influenza A virus strain. In specific embodiments, the first group 2 influenza A virus strain is an H3 (e.g., A/Hong Kong/4801/2014). In a specific embodiment, the influenza A virus backbone of an influenza virus either comprising, containing, or both the mosaic HA is from a second influenza A virus, e.g., A/Puerto Rico/8/34, an influenza virus lacking the NS1 protein (e.g., a delNS1 virus, such as described, e.g., in U.S. Pat. No. 6,468,544; Garcia-Sastre et al., 1998, Virology 252:324; or Mössler et al., 2013, Vaccine 31:6194), or a cold-adapted influenza A virus (e.g., A/Ann Arbor/6/60 or A/Leningrad/134/17/57). In specific embodiments, the influenza A virus from which the amino acid residues are derived for the amino acid substitutions in one, two, three or more of the antigenic sites is an H10 strain (e.g., A/Jiangxi-Donghu/346-1/2013). In specific embodiments, the influenza A virus from which the amino acid residues are derived for the amino acid substitutions in one, two, three or more of the antigenic sites is an H14 strain (e.g., A/mallard/Gurjev/263/1982). In specific embodiments, a mosaic HA polypeptide described herein comprises the A, B, C, D and/or E with the amino acid sequences of the A, B, C, D and/or E, respectively, set forth in Table 8. In certain embodiments, the mosaic HA polypeptides comprise 1, 2, 3, 4, 5 or more amino acid substitutions in the globular head domain of the group 2 influenza A virus strain HA which are outside of one, two, three, four or all the following antigenic sites: A, B, C, D and E, or corresponding hypervariable antigenic sites.

In another embodiment, provided herein are mosaic hemagglutinin (HA) polypeptides comprising (i) a hemagglutinin ectodomain from a first group 2 influenza A virus strain with 2, 3, 4, 5, 6, 7, 8, 9, 10, 11, 12 or more amino acid substitutions within the A, B, C, D, or E antigenic site or a corresponding hypervariable antigenic site of the globular head domain of the group 2 influenza A virus strain HA and (ii) a signal peptide, a transmembrane domain and a cytoplasmic tail domain from the first group 2 influenza A virus strain HA, wherein the amino acid substitutions substitute 2, 3, 4, 5, 6, 7, 8, 9, 10, 11, 12 or more amino acid residues in the A, B, C, D, or E or a corresponding hypervariable antigenic site of the globular head of the first group 2 influenza A virus strain HA with amino acid residues found in a corresponding region of the globular domain of an influenza A virus HA of a different strain or subtype than the first group 2 influenza A virus strain. In another embodiment, provided herein are mosaic hemagglutinin (HA) polypeptides comprising (i) a hemagglutinin ectodomain from a first group 2 influenza A virus strain with 2, 3, 4, 5, 6, 7, 8, 9, 10, 11, 12 or more amino acid substitutions within each of the A, B, C, D, and E antigenic sites or corresponding hypervariable antigenic sites of the globular head domain of the first group 2 influenza A virus strain HA and (ii) a signal peptide, a transmembrane domain and a cytoplasmic tail domain from the first group 2 influenza A virus strain HA, wherein the amino acid substitutions substitute 2, 3, 4, 5, 6, 7, 8, 9, 10, 11, 12 or more amino acid residues in each of the A, B, C, D, and E antigenic sites or corresponding hypervariable antigenic sites of the globular head of the first group 2 influenza A virus strain HA with amino acid residues found in corresponding regions of the globular domain of an influenza A virus HA of a different strain or subtype than the first group 2 influenza A virus strain. In specific embodiments, the first group 2 influenza A virus strain is an H3 (e.g., A/Hong Kong/4801/2014). In a specific embodiment, the influenza A virus backbone of an influenza virus either comprising, containing, or both the mosaic HA is from a second influenza A virus, e.g., A/Puerto Rico/8/34, an influenza virus lacking the NS1 protein (e.g., a delNS1 virus, such as described, e.g., in U.S. Pat. No. 6,468,544; Garcia-Sastre et al., 1998, Virology 252:324; or Mössler et al., 2013, Vaccine 31:6194), or a cold-adapted influenza A virus (e.g., A/Ann Arbor/6/60 or A/Leningrad/134/17/57). In specific embodiments, the influenza A virus from which the amino acid residues are derived for the amino acid substitutions in one, two, three or more of the antigenic sites is an H10 strain (e.g., A/Jiangxi-Donghu/346-1/2013). In specific embodiments, a mosaic HA polypeptide described herein comprises the A, B, C, D and/or E with the amino acid sequences of the A, B, C, D and/or E, respectively, set forth in Table 8. In certain embodiments, the mosaic HA polypeptides comprise 1, 2, 3, 4, 5 or more amino acid substitutions in the globular head domain of the group 2 influenza A virus strain HA which are outside of one, two, three, four or all the following antigenic sites: A, B, C, D and E, or corresponding hypervariable antigenic sites.

In specific embodiments, provided herein is a mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprises four or all of the following amino acid sequence substitutions: a. the amino acid sequence NNESFNWT-GVTQNGTSSACIRRSSSS (SEQ ID NO: 13) in the HA globular head domain A antigenic site of influenza A/Hong Kong/4801/2014 virus HA has been substituted with the following amino acid sequence NNESFNWTGVTQNGTSSACMRNGGNS (SEQ ID NO: 14); b. the amino acid sequences THL-NYK (SEQ ID NO: 15) and GTDKDQIFLYAQ (SEQ ID NO: 16) in the HA globular head domain B antigenic site of influenza A/Hong Kong/4801/2014 virus HA have been substituted with the following amino acid sequences THL-NQK (SEQ ID NO: 17) and GTNQDQIFLYAQ (SEQ ID NO: 18), respectively; c. the amino acid sequences QNSSIGEICDS (SEQ ID NO: 19) and PIG-KCKSE (SEQ ID NO: 20) in the HA globular head domain C antigenic site of influenza A/Hong Kong/4801/2014 virus HA have been substituted with the following amino acid sequences ESTGINRLCMK (SEQ ID NO: 21) and PIDNNCESK (SEQ ID NO; 22), respectively; d. the amino acid sequence RITVSTKRSQQAVIPNIGS (SEQ ID NO: 23) in the HA globular head domain D antigenic site of influenza A/Hong Kong/4801/2014 virus HA has been substituted with the following amino acid sequence RITVSTSTYQQAVIPNIGS (SEQ ID NO: 25); and e. the amino acid sequences ENCT (SEQ ID NO: 124), GFQNKKWDLFVERSKAY (SEQ ID NO: 27) and IRSGKS (SEQ ID NO: 28) in the HA globular head domain E antigenic site of influenza A/Hong Kong/4801/2014 virus HA have been substituted with the following amino acid sequences GNCH (SEQ ID NO: 125), GFQNKMWDLFVERSKAY (SEQ ID NO: 29) and LRIGRS (SEQ ID NO: 24), respectively. In specific embodiments, the mosaic influenza virus HA polypeptide further comprises the transmembrane and cytoplasmic domains of the influenza A/Hong Kong/4801/2014 virus HA. In a specific embodiment, the influenza A virus backbone of an influenza virus either comprising, containing, or both the mosaic HA is from a second influenza A virus, e.g., A/Puerto Rico/8/34, an influenza virus lacking the NS1 protein (e.g., a delNS1 virus, such as described, e.g., in U.S. Pat. No. 6,468,544; Garcia-Sastre et al., 1998, Virology 252:324; or Mössler et al., 2013, Vaccine 31:6194), or a cold-adapted influenza A virus (e.g., A/Ann Arbor/6/60 or A/Leningrad/134/17/57).

In a specific embodiment, a mosaic influenza virus HA polypeptide described herein comprises the amino acid sequence set forth in SEQ ID NO: 31. In a specific embodiment, a mosaic influenza virus HA polypeptide described herein comprises the amino acid sequence of mH10/3 or mH14 set forth in FIGS. 18A-18B (SEQ ID NO: 173 or 175).

### 5.2 Nucleic Acid Sequences Encoding Mosaic Hemagglutinin (HA) Polypeptides

Provided herein are nucleic acid sequences that encode the mosaic influenza virus hemagglutinin polypeptides described herein. In specific embodiments, provided herein is a nucleic acid sequence comprising a nucleotide sequence encoding a mosaic influenza virus HA polypeptide (with or without the signal peptide). In a specific embodiment, a nucleic acid sequence comprises the nucleotide sequence of mH10/3 or mH14/3 set forth in FIGS. 18A-18B (SEQ ID NO: 172 or 174). In certain embodiment, the nucleotide sequence encoding the mosaic influenza virus HA polypeptide comprises a nucleotide sequence encoding a signal peptide (e.g., a signal peptide from the HA of the same influenza virus as the influenza virus engineered to express the mosaic HA polypeptide). In some embodiment, the nucleic acid sequence further comprises the 5′ non-coding region and 3′ non-coding region of an influenza virus HA (e.g., the 5′ non-coding region and 3′ non-coding region from the HA of the same influenza A virus as the influenza virus engineered to express the mosaic influenza virus HA polypeptide).

Due to the degeneracy of the genetic code, any nucleic acid sequence that encodes a mosaic hemagglutinin (HA) polypeptide described herein is encompassed herein. In certain embodiments, nucleic acid sequences corresponding to naturally occurring influenza A virus nucleic acid sequences encoding an HA globular head domain and HA stem domain are used to produce a mosaic influenza virus hemagglutinin polypeptide. In certain embodiments, a nucleic acid sequence corresponding to naturally occurring influenza A virus HA nucleic acid sequence comprising a nucleotide sequence encoding an HA globular head domain and a nucleotide sequence encoding HA stem domain is used to produce a mosaic influenza virus hemagglutinin polypeptide. In certain embodiments, a nucleic acid sequence corresponding to a naturally occurring influenza A virus HA nucleic acid sequence comprising a nucleotide sequence encoding an HA globular head domain, a nucleotide sequence encoding an HA stem domain, a nucleotide sequence encoding an HA transmembrane domain, and an HA cytoplasmic domain is used to produce a mosaic influenza virus hemagglutinin polypeptide. In accordance with such embodiments, the nucleotide sequence of the HA globular head domain may be engineered to include one, two or more amino acid substitutions in one, two, three, four or more antigenic sites. In some embodiments, the nucleic acid sequence further comprises a nucleotide sequence encoding an influenza virus HA signal peptide. In specific embodiments, the nucleic acid sequence(s) encoding a mosaic influenza virus HA polypeptide further comprises the 5′ non-coding region and 3′ non-coding region from an HA (e.g., the t′ non-coding region and 3′ non-coding region from the HA of the same influenza A virus as the influenza virus engineered to express the mosaic influenza virus HA polypeptide).

Also provided herein are nucleic acid sequences capable of hybridizing to a nucleic acid encoding a mosaic influenza virus hemagglutinin polypeptide. In certain embodiments, provided herein are nucleic acid sequences capable of hybridizing to a fragment of a nucleic acid sequence encoding a mosaic influenza virus hemagglutinin polypeptide. In other embodiments, provided herein are nucleic acid sequences capable of hybridizing to the full length of a nucleic acid sequence encoding a mosaic influenza virus hemagglutinin polypeptide. General parameters for hybridization conditions for nucleic acids are described in Sambrook et al., Molecular Cloning-A Laboratory Manual (2nd Ed.), Vols. 1-3, Cold Spring Harbor Laboratory, Cold Spring Harbor, New York (1989), and in Ausubel et al., Current Protocols in Molecular Biology, vol. 2, Current Protocols Publishing, New York (1994). Hybridization may be performed under high stringency conditions, medium stringency conditions, or low stringency conditions. Those of skill in the art will understand that low, medium and high stringency conditions are contingent upon multiple factors all of which interact and are also dependent upon the nucleic acids in question. For example, high stringency conditions may include temperatures within 5° C. melting temperature of the nucleic acid(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the nucleic acid(s), a high salt concentration (e.g., greater than 1000 mM) and the absence of co-solvents.

In some embodiments, a nucleic acid sequence comprising a nucleotide sequence encoding a mosaic influenza virus hemagglutinin polypeptide is isolated. In certain embodiments, an “isolated” nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences in which the nucleic acid sequence is separated from cellular components of the cells from which it is isolated or recombinantly produced. Thus, nucleic acid sequence that is substantially free of cellular material includes preparations of nucleic acid sequence having less than about 30%, 20%, 10%, or 5% (by dry weight) of other nucleic acids. The term “substantially free of culture medium” includes preparations of nucleic acid sequence in which the culture medium represents less than about 50%, 20%, 10%, or 5% of the volume of the preparation. The term “substantially free of chemical precursors or other chemicals” includes preparations in which the nucleic acid sequence is separated from chemical precursors or other chemicals which are involved in the synthesis of the nucleic acid sequence. In specific embodiments, such preparations of the nucleic acid sequence have less than about 50%, 30%, 20%, 10%, 5% (by dry weight) of chemical precursors or compounds other than the nucleic acid sequence of interest.

In addition, provided herein are nucleic acid sequences encoding the individual components of a mosaic influenza virus hemagglutinin polypeptide. In specific embodiments, nucleic acid sequences encoding the globular head domain and/or the stem domain of the mosaic influenza virus hemagglutinin polypeptide are provided. Nucleic acid sequences encoding components of a mosaic influenza virus hemagglutinin polypeptide may be assembled using standard molecular biology techniques known to one of skill in the art. In specific embodiments, the individual components of a mosaic influenza virus hemagglutinin polypeptide can be expressed by the same or different vector.

### 5.3 Expression of a Mosaic Hemagglutinin (HA) Polypeptide

Provided herein are vectors, including expression vectors, containing a nucleic acid sequence comprising a nucleotide sequence encoding a mosaic influenza virus hemagglutinin polypeptide described herein. In a specific embodiment, the vector is an expression vector that is capable of directing the expression of a nucleic acid sequence encoding a mosaic influenza virus hemagglutinin polypeptide. Non-limiting examples of expression vectors include, but are not limited to, plasmids and viral vectors, such as replication defective retroviruses, adenoviruses, vesicular stomatitis virus (VSV), Newcastle disease virus (NDV), Modified Vaccinia Ankara virus, adeno-associated viruses and baculoviruses. Techniques known to one of skill in the art may be used to engineer such viral vectors to express a mosaic influenza virus HA polypeptide described herein. Expression vectors also may include, without limitation, transgenic animals and non-mammalian cells/organisms, e.g., mammalian cells/organisms that have been engineered to perform mammalian N-linked glycosylation.

In some embodiments, provided herein are expression vectors encoding components of a mosaic hemagglutinin (HA) polypeptide (e.g., the stem domain and the head domain, or portions of either domain). Such vectors may be used to express the components in one or more host cells and the components may be isolated and conjugated together with a linker using techniques known to one of skill in the art.

An expression vector comprises a nucleic acid sequence comprising a nucleotide sequence encoding a mosaic hemagglutinin (HA) polypeptide described herein and in a form suitable for expression of the nucleic acid sequence in a host cell. In a specific embodiment, an expression vector includes one or more regulatory sequences, selected on the basis of the host cells to be used for expression, which is operably linked to the nucleic acid to be expressed. Within an expression vector, “operably linked” is intended to mean that a nucleic acid of interest is linked to the regulatory sequence(s) in a manner which allows for expression of the nucleic acid sequence (e.g., in an in vitro transcription/translation system or in a host cell when the vector is introduced into the host cell). Regulatory sequences include promoters, enhancers and other expression control elements (e.g., polyadenylation signals). Regulatory sequences include those which direct constitutive expression of a nucleic acid in many types of host cells, those which direct expression of the nucleic acid only in certain host cells (e.g., tissue-specific regulatory sequences), and those which direct the expression of the nucleic acid upon stimulation with a particular agent (e.g., inducible regulatory sequences). It will be appreciated by those skilled in the art that the design of the expression vector can depend on such factors as the choice of the host cell to be transformed, the level of expression of protein desired, etc. The term “host cell” is intended to include a particular subject cell transformed or transfected with a nucleic acid sequence and the progeny or potential progeny of such a cell. Progeny of such a cell may not be identical to the parent cell transformed or transfected with the nucleic acid sequence due to mutations or environmental influences that may occur in succeeding generations or integration of the nucleic acid sequence into the host cell genome. In specific embodiments, the host cell is a cell line.

Expression vectors can be designed for expression of a mosaic hemagglutinin (HA) polypeptide described herein using prokaryotic (e. g., E. coli) or eukaryotic cells (e.g., insect cells (using baculovirus expression vectors, see, e.g., Treanor et al., 2007, JAMA, 297(14):1577-1582 incorporated by reference herein in its entirety), yeast cells, plant cells, algae, avian, or mammalian cells). Examples of yeast host cells include, but are not limited to S. pombe and S. cerevisiae and examples, infra. An example of avian cells includes, but is not limited to EB66 cells. Examples of mammalian host cells include, but are not limited to, A549 cells, Crucell Per.C6 cells, Vero cells, CHO cells, VERO cells, BHK cells, HeLa cells, COS cells, MDCK cells, 293 cells, 3T3 cells or WI38 cells. In certain embodiments, the hosts cells are myeloma cells, e.g., NS0 cells, 45.6 TG1.7 cells, AF-2 clone 9B5 cells, AF-2 clone 9B5 cells, J558L cells, MOPC 315 cells, MPC-11 cells, NCI-H929 cells, NP cells, NS0/1 cells, P3 NS1 Ag4 cells, P3/NS1/1-Ag4-1 cells, P3Ul cells, P3X63Ag8 cells, P3X63Ag8.653 cells, P3X63Ag8U.1 cells, RPMI 8226 cells, Sp20-Ag14 cells, U266B1 cells, X63AG8.653 cells, Y3. Ag.1.2.3 cells, and YO cells. Non-limiting examples of insect cells include Sf9, S/21, Trichoplusia ni, Spodoptera frugiperda and Bombyx mori. In a particular embodiment, a mammalian cell culture system (e.g. Chinese hamster ovary or baby hamster kidney cells) is used for expression of a mosaic hemagglutinin (HA) polypeptide. In another embodiment, a plant cell culture system is used for expression of a mosaic hemagglutinin (HA) polypeptide. See, e.g., U.S. Pat. Nos. 7,504,560; 6,770,799; 6,551,820; 6,136,320; 6,034,298; 5,914,935; 5,612,487; and 5,484,719, and U.S. patent application publication Nos. 2009/0208477, 2009/0082548, 2009/0053762, 2008/0038232, 2007/0275014 and 2006/0204487 for plant cells and methods for the production of proteins utilizing plant cell culture systems. In specific embodiments, plant cell culture systems are not used for expression of a mosaic influenza virus hemagglutinin (HA) polypeptide. The host cells comprising the nucleic acids that encode the mosaic influenza virus hemagglutinin (HA) polypeptides described herein can be isolated, i.e., the cells are outside of the body of a subject. In certain embodiments, the cells are engineered to express nucleic acids that encode the mosaic influenza virus hemagglutinin polypeptides described herein. In specific embodiments, the host cells are cells from a cell line.

An expression vector can be introduced into host cells via conventional transformation or transfection techniques. Such techniques include, but are not limited to, calcium phosphate or calcium chloride co-precipitation, DEAE-dextran-mediated transfection, lipofection, and electroporation. Suitable methods for transforming or transfecting host cells can be found in Sambrook et al., 1989, Molecular Cloning-A Laboratory Manual, 2nd Edition, Cold Spring Harbor Press, New York, and other laboratory manuals. In certain embodiments, a host cell is transiently transfected with an expression vector containing a nucleic acid sequence encoding a mosaic hemagglutinin (HA) polypeptide. In other embodiments, a host cell is stably transfected with an expression vector containing a nucleic acid sequence encoding a mosaic hemagglutinin (HA) polypeptide.

For stable transfection of mammalian cells, it is known that, depending upon the expression vector and transfection technique used, only a small fraction of cells may integrate the foreign DNA into their genome. In order to identify and select these integrants, a nucleic acid that encodes a selectable marker (e.g., for resistance to antibiotics) is generally introduced into the host cells along with the nucleic acid of interest. Examples of selectable markers include those which confer resistance to drugs, such as G418, hygromycin and methotrexate. Cells stably transfected with the introduced nucleic acid sequence can be identified by drug selection (e.g., cells that have incorporated the selectable marker gene will survive, while the other cells die).

As an alternative to recombinant expression of a mosaic hemagglutinin (HA) polypeptide using a host cell, an expression vector containing a nucleic acid sequence encoding a mosaic hemagglutinin (HA) polypeptide can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce a mosaic hemagglutinin (HA) polypeptide.

Once a mosaic hemagglutinin (HA) polypeptide has been produced, it may be isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins.

Accordingly, provided herein are methods for producing a mosaic influenza virus hemagglutinin (HA) polypeptide. In one embodiment, the method comprises culturing a host cell containing a nucleic acid sequence comprising a nucleotide sequence encoding the polypeptide in a suitable medium such that the polypeptide is produced. In some embodiments, the method further comprises isolating the polypeptide from the medium or the host cell.

Also provided herein are methods for producing a virus (e.g., an influenza virus (see Section 5.4, infra) or a non-influenza virus vector (e.g., a baculovirus) comprising a mosaic influenza virus HA polypeptide described herein, comprising propagating the virus in any substrate that allows the virus to grow to titers that permit their use in accordance with the methods described herein. In one embodiment, the substrate allows the viruses to grow to titers comparable to those determined for the corresponding wild-type viruses. In a specific embodiment, the virus is propagated in embryonated eggs (e.g., chicken eggs). In a specific embodiment, the virus is propagated in 8 day old, 9-day old, 8-10 day old, 10 day old, 11-day old, 10-12 day old, or 12-day old embryonated eggs (e.g., chicken eggs). In some embodiments, the virus is propagated in embryonated eggs (e.g., chicken eggs) that are IFN-deficient. In certain embodiments, the virus is propagated in MDCK cells, Vero cells, 293T cells, or other cell lines known in the art. See, e.g., Section 5.3, supra, for examples of cell lines. In certain embodiments, the virus is propagated in cells derived from embryonated eggs. In certain embodiments, the virus is propagated in an embryonated egg (e.g., chicken eggs) and then in MDCK cells, Vero cells, 293T cells, or other cell lines known in the art.

### 5.4 Influenza Virus Vectors

In one aspect, provided herein are influenza viruses containing a mosaic influenza virus hemagglutinin polypeptide described herein. In a specific embodiment, the mosaic influenza virus hemagglutinin (HA) polypeptide is incorporated into the virions of the influenza virus. The influenza viruses may be conjugated to moieties that target the viruses to particular cell types, such as immune cells. In some embodiments, the virions of the influenza virus have incorporated into them or express a heterologous polypeptide in addition to a mosaic hemagglutinin (HA) polypeptide. The heterologous polypeptide may be a polypeptide that has immunopotentiating activity, or that targets the influenza virus to a particular cell type, such as an antibody that binds to an antigen on a specific cell type or a ligand that binds a specific receptor on a specific cell type.

Influenza viruses containing a mosaic influenza virus hemagglutinin (HA) polypeptide may be produced by supplying in trans the mosaic influenza virus hemagglutinin (HA) polypeptide during production of virions using techniques known to one skilled in the art, such as reverse genetics and helper-free plasmid rescue. Alternatively, the replication of a parental influenza virus comprising a genome engineered to express a mosaic influenza virus hemagglutinin (HA) polypeptide in cells susceptible to infection with the virus, wherein hemagglutinin function is provided in trans will produce progeny influenza viruses containing the mosaic influenza virus hemagglutinin (HA) polypeptide.

In another aspect, provided herein are influenza viruses comprising a genome engineered to express a mosaic influenza virus hemagglutinin (HA) polypeptide. In a specific embodiment, the genome of a parental influenza virus is engineered to encode a mosaic influenza virus hemagglutinin (HA) polypeptide, which is expressed by progeny influenza virus. In another specific embodiment, the genome of a parental influenza virus is engineered to encode a mosaic influenza virus hemagglutinin (HA) polypeptide, which is expressed and incorporated into the virions of progeny influenza virus. Thus, the progeny influenza virus resulting from the replication of the parental influenza virus contain a mosaic influenza virus hemagglutinin (HA) polypeptide. In specific embodiments, the parental influenza virus is an influenza A virus.

In some embodiments, the virions of the parental influenza virus have incorporated into them a heterologous polypeptide. In certain embodiments, the genome of a parental influenza virus is engineered to encode a heterologous polypeptide and a mosaic influenza virus hemagglutinin (HA) polypeptide, which are expressed by progeny influenza virus. In specific embodiments, the mosaic influenza virus hemagglutinin (HA) polypeptide, the heterologous polypeptide or both are incorporated into virions of the progeny influenza virus.

In some embodiments, the virions of the parental influenza virus have incorporated into them an influenza virus neuraminidase, wherein the strain of the influenza virus neuraminidase is not the same strain as the strain of the globular head domain or stem domain of the mosaic influenza virus HA polypeptide. In some embodiments, the influenza virus neuraminidase corresponds to the influenza virus neuraminidase of the HA stem domain of the mosaic influenza virus HA polypeptide.

Since the genome of influenza A and B viruses consist of eight (8) single-stranded, negative sense segments (influenza C viruses consist of seven (7) single-stranded, negative sense segments), the genome of a parental influenza virus may be engineered to express a mosaic hemagglutinin (HA) polypeptide (and any other polypeptide, such as a heterologous polypeptide) using a recombinant segment and techniques known to one skilled in the art, such a reverse genetics and helper-free plasmid rescue. In one embodiment, the recombinant segment comprises a nucleic acid encoding the mosaic influenza virus hemagglutinin (HA) polypeptide as well as the 3′ and 5′ incorporation signals which are required for proper replication, transcription and packaging of the vRNAs (Fujii et al., 2003, Proc. Natl. Acad. Sci. USA 100:2002-2007; Zheng, et al., 1996, Virology 217:242-251, International Publication No. WO 2011/014645, all of which are incorporated by reference herein in their entireties). In a specific embodiment, the recombinant segment uses the 3′ and 5′ noncoding and/or nontranslated sequences of segments of influenza viruses that are from a different or the same type, subtype/lineage or strain as the parental influenza virus. In some embodiments, the recombinant segment comprises the 3′ noncoding region of an influenza virus hemagglutinin polypeptide, the untranslated regions of an influenza virus hemagglutinin polypeptide, and the 5′ non-coding region of an influenza virus hemagglutinin polypeptide. In specific embodiments, the recombinant segment comprises the 3′ and 5′ noncoding and/or nontranslated sequences of the HA segment of an influenza virus that is the same type, subtype/lineage or strain as the influenza virus type, subtype/lineage or strain as the HA2 stem domain of a mosaic hemagglutinin (HA) polypeptide. In specific embodiments, the recombinant segment comprises packaging signals, such as the 5′ and 3′ non-coding regions and signal peptide of the HA segment of an influenza virus, from the same type, lineage, or strain as the influenza virus backbone. For example, if the mosaic influenza virus HA is engineered to be expressed from an influenza A virus, then the nucleotide sequence encoding mosaic HA comprises the 5′ and 3′ non-coding regions and the nucleotide sequence encoding the signal peptide of the HA segment of the influenza A virus. In certain embodiments, the recombinant segment encoding the mosaic influenza virus hemagglutinin (HA) polypeptide may replace the HA segment of a parental influenza virus.

In some embodiments, a hemagglutinin gene segment (e.g., mosaic hemagglutinin gene segment) encodes a mosaic influenza virus hemagglutinin (HA) polypeptide. In specific embodiments, the influenza virus hemagglutinin (HA) gene segment (e.g., mosaic hemagglutinin gene segment) and at least one other influenza virus gene segment comprise packaging signals that enable the mosaic influenza virus hemagglutinin (HA) gene segment and the at least one other gene segment to segregate together during replication of a recombinant influenza virus (see, Gao & Palese 2009, PNAS 106:15891-15896; U.S. Pat. No. 8,828,406; and International Application Publication No. WO11/014645, each of which is incorporated herein by reference in its entirety).

In some embodiments, the genome of a parental influenza virus may be engineered to express a mosaic influenza virus hemagglutinin (HA) polypeptide using a recombinant segment that is bicistronic. Bicistronic techniques allow the engineering of coding sequences of multiple proteins into a single mRNA through the use of internal ribosome entry site (IRES) sequences. IRES sequences direct the internal recruitment of ribosomes to the RNA molecule and allow downstream translation in a cap independent manner. Briefly, a coding region of one protein is inserted into the open reading frame (ORF) of a second protein. The insertion is flanked by an IRES and any untranslated signal sequences necessary for proper expression and/or function. The insertion must not disrupt the ORF, polyadenylation or transcriptional promoters of the second protein (see, e.g., García-Sastre et al., 1994, J. Virol. 68:6254-6261 and García-Sastre et al., 1994 Dev. Biol. Stand. 82:237-246, each of which is hereby incorporated by reference in its entirety). See also, e.g., U.S. Pat. Nos. 6,887,699, 6,001,634, 5,854,037 and 5,820,871, each of which is incorporated herein by reference in its entirety. Any IRES known in the art or described herein may be used in accordance with the invention (e.g., the IRES of BiP gene, nucleotides 372 to 592 of GenBank database entry HUMGRP78; or the IRES of encephalomyocarditis virus (EMCV), nucleotides 1430-2115 of GenBank database entry CQ867238.). Thus, in certain embodiments, a parental influenza virus is engineered to contain a bicistronic RNA segment that expresses the mosaic influenza virus hemagglutinin (HA) polypeptide and another polypeptide, such as a gene expressed by the parental influenza virus. In some embodiments, the parental influenza virus gene is the HA gene.

Techniques known to one skilled in the art may be used to produce an influenza virus containing a mosaic influenza virus hemagglutinin (HA) polypeptide and an influenza virus comprising a genome engineered to express a mosaic influenza virus hemagglutinin (HA) polypeptide. For example, reverse genetics techniques may be used to generate such an influenza virus. Briefly, reverse genetics techniques generally involve the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. patent application Ser. No. 09/152,845; in International Patent Publications PCT WO 97/12032 published Apr. 3, 1997; WO 96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

Alternatively, helper-free plasmid technology may be used to produce an influenza virus containing a mosaic hemagglutinin (HA) polypeptide and an influenza virus comprising a genome engineered to express a mosaic hemagglutinin (HA) polypeptide. Briefly, full length cDNAs of viral segments are amplified using PCR with primers that include unique restriction sites, which allow the insertion of the PCR product into the plasmid vector (Flandorfer et al., 2003, J. Virol. 77:9116-9123; Nakaya et al., 2001, J. Virol. 75:11868-11873; both of which are incorporated herein by reference in their entireties). The plasmid vector is designed so that an exact negative (vRNA sense) transcript is expressed. For example, the plasmid vector may be designed to position the PCR product between a truncated human RNA polymerase I promoter and a hepatitis delta virus ribozyme sequence such that an exact negative (vRNA sense) transcript is produced from the polymerase I promoter. Separate plasmid vectors comprising each viral segment as well as expression vectors comprising necessary viral proteins may be transfected into cells leading to production of recombinant viral particles. In another example, plasmid vectors from which both the viral genomic RNA and mRNA encoding the necessary viral proteins are expressed may be used. For a detailed description of helper-free plasmid technology see, e.g., International Publication No. WO 01/04333; U.S. Pat. Nos. 6,951,754, 7,384,774, 6,649,372, and 7,312,064; Fodor et al., 1999, J. Virol. 73:9679-9682; Quinlivan et al., 2005, J. Virol. 79:8431-8439; Hoffmann et al., 2000, Proc. Natl. Acad. Sci. USA 97:6108-6113; and Neumann et al., 1999, Proc. Natl. Acad. Sci. USA 96:9345-9350, each of which is incorporated herein by reference in its entirety. In a specific embodiment, a method analogous to that described in Section 6 is used to construct a mosaic influenza virus HA polypeptide. In a specific embodiment, a method analogous to that described in Section 6 is used to construct and propagate a mosaic influenza virus HA polypeptide.

The influenza viruses described herein may be propagated in any substrate that allows the virus to grow to titers that permit their use in accordance with the methods described herein. Thus, in certain embodiments, provided herein is a method for producing a virus described herein comprising propagating the virus in a substrate. In one embodiment, the substrate allows the viruses to grow to titers comparable to those determined for the corresponding wild-type viruses. In certain embodiments, the substrate is one which is biologically relevant to the influenza virus or to the virus from which the HA function is derived. In a specific embodiment, an attenuated influenza virus by virtue of, e.g., a mutation in the NS1 gene, may be propagated in an IFN-deficient substrate. For example, a suitable IFN-deficient substrate may be one that is defective in its ability to produce or respond to interferon, or is one which an IFN-deficient substrate may be used for the growth of any number of viruses which may require interferon-deficient growth environment. See, for example, U.S. Pat. No. 6,573,079, issued Jun. 3, 2003, U.S. Pat. No. 6,852,522, issued Feb. 8, 2005, and U.S. Pat. No. 7,494,808, issued Feb. 24, 2009, the entire contents of each of which is incorporated herein by reference in its entirety. In a specific embodiment, the virus is propagated in embryonated eggs (e.g., chicken eggs). In a specific embodiment, the virus is propagated in 8 day old, 9-day old, 8-10 day old, 10 day old, 11-day old, 10-12 day old, or 12-day old embryonated eggs (e.g., chicken eggs). In some embodiments, the virus is propagated in embryonated eggs (e.g., chicken eggs) that are IFN-deficient. In certain embodiments, the virus is propagated in MDCK cells, Vero cells, 293T cells, or other cell lines known in the art. See, e.g., Section 5.3, supra, for examples of cell lines. In certain embodiments, the virus is propagated in cells derived from embryonated eggs.

The influenza viruses described herein may be isolated and purified by any method known to those of skill in the art. In one embodiment, the virus is removed from cell culture and separated from cellular components, typically by well known clarification procedures, e.g., such as gradient centrifugation and column chromatography, and may be further purified as desired using procedures well known to those skilled in the art, e.g., plaque assays.

In certain embodiments, the influenza viruses, or influenza virus polypeptides, genes or genome segments for use as described herein are obtained or derived from an influenza A virus. In certain embodiments, the influenza viruses, or influenza virus polypeptides, genes or genome segments for use as described herein are obtained or derived from a single influenza A virus subtype/lineage or strain. In other embodiments, the influenza viruses, or influenza virus polypeptides, genes or genome segments for use as described herein are obtained or derived from two or more influenza A virus subtypes or strains. In a specific embodiment, the influenza A virus is an influenza virus of the H1, H2, H3, H4, H5, H6, H7, H8, H9, H10, H11, H12, H13, H14, H15, H16, H17, or H18 subtype. In a specific embodiment, the influenza A virus is an influenza virus of the H2, H4, H5, H6, H7, H8, H9, H10, H11, H12, H13, H14, H15, H16, H17, or H18 subtype. In a specific embodiment, the influenza A virus is an influenza virus of the H5, H8, H11, H12, or H13 subtype. In a specific embodiment, the influenza A virus is an influenza virus of the H5 subtype. In a specific embodiment, the influenza A virus is an influenza virus of the H8 subtype. In a specific embodiment, the influenza A virus is an influenza virus of the H11 subtype. In a specific embodiment, the influenza A virus is an influenza virus of the H12 subtype. In a specific embodiment, the influenza A virus is an influenza virus of the H13 subtype. In a specific embodiment, the influenza A virus is an influenza virus of the H10 or H14 subtype. In a specific embodiment, the influenza A virus is an avian influenza virus.

Non-limiting examples of influenza A viruses include subtype H10N4, subtype H10N5, subtype H10N8, subtype, H14N5, subtype H10N7, subtype H10N8, subtype H10N9, subtype H11N1, subtype H11N13, subtype H11N2, subtype H11N4, subtype H11N6, subtype H11N8, subtype H11N9, subtype H12N1, subtype H12N4, subtype H12N5, subtype H12N8, subtype H13N2, subtype H13N3, subtype H13N6, subtype H13N7, subtype H14N5, subtype H14N6, subtype H15N8, subtype H15N9, subtype H16N3, subtype H1N1, subtype H1N2, subtype H1N3, subtype H1N6, subtype H1N9, subtype H2N1, subtype H2N2, subtype H2N3, subtype H2N5, subtype H2N7, subtype H2N8, subtype H2N9, subtype H3N1, subtype H3N2, subtype H3N3, subtype H3N4, subtype H3N5, subtype H3N6, subtype H3N8, subtype H3N9, subtype H4N1, subtype H4N2, subtype H4N3, subtype H4N4, subtype H4N5, subtype H4N6, subtype H4N8, subtype H4N9, subtype H5N1, subtype H5N2, subtype H5N3, subtype H5N4, subtype H5N6, subtype H5N7, subtype H5N8, subtype H5N9, subtype H6N1, subtype H6N2, subtype H6N3, subtype H6N4, subtype H6N5, subtype H6N6, subtype H6N7, subtype H6N8, subtype H6N9, subtype H7N1, subtype H7N2, subtype H7N3, subtype H7N4, subtype H7N5, subtype H7N7, subtype H7N8, subtype H7N9, subtype H8N4, subtype H8N5, subtype H9N1, subtype H9N2, subtype H9N3, subtype H9N5, subtype H9N6, subtype H9N7, subtype H9N8, and subtype H9N9.

Specific examples of strains of influenza A virus include, but are not limited to: A/Victoria/361/2011 (H3N2); A/California/4/2009 (H1N1); A/California/7/2009 (H1N1); A/Perth/16/2009 (H3N2); A/Brisbane/59/2007 (H1N1); A/Brisbane/10/2007 (H3N2); A/sw/Iowa/15/30 (H1N1); A/WSN/33 (H1N1); A/eq/Prague/1/56 (H7N7); A/PR/8/34; A/mallard/Potsdam/178-4/83 (H2N2); A/herring gull/DE/712/88 (H16N3); A/sw/Hong Kong/168/1993 (H1N1); A/mallard/Alberta/211/98 (H1N1); A/shorebird/Delaware/168/06 (H16N3); A/sw/Netherlands/25/80 (H1N1); A/sw/Germany/2/81 (H1N1); A/sw/Hannover/1/81 (H1N1); A/sw/Potsdam/1/81 (H1N1); A/sw/Potsdam/15/81 (H1N1); A/sw/Potsdam/268/81 (H1N1); A/sw/Finistere/2899/82 (H1N1); A/sw/Potsdam/35/82 (H3N2); A/sw/Cote d'Armor/3633/84 (H3N2); A/sw/Gent/1/84 (H3N2); A/sw/Netherlands/12/85 (H1N1); A/sw/Karrenzien/2/87 (H3N2); A/sw/Schwerin/103/89 (H1N1); A/turkey/Germany/3/91 (H1N1); A/sw/Germany/8533/91 (H1N1); A/sw/Belgium/220/92 (H3N2); A/sw/Gent/V230/92 (H1N1); A/sw/Leipzig/145/92 (H3N2); A/sw/Re220/92 hp (H3N2); A/sw/Bakum/909/93 (H3N2); A/sw/Schleswig-Holstein/1/93 (H1N1); A/sw/Scotland/419440/94 (H1N2); A/sw/Bakum/5/95 (H1N1); A/sw/Best/5C/96 (H1N1); A/sw/England/17394/96 (H1N2); A/sw/Jena/5/96 (H3N2); A/sw/Oedenrode/7C/96 (H3N2); A/sw/Lohne/1/97 (H3N2); A/sw/Cote d'Armor/790/97 (H1N2); A/sw/Bakum/1362/98 (H3N2); A/sw/Italy/1521/98 (H1N2); A/sw/Italy/1553-2/98 (H3N2); A/sw/Italy/1566/98 (H1N1); A/sw/Italy/1589/98 (H1N1); A/sw/Bakum/8602/99 (H3N2); A/sw/Cotes d'Armor/604/99 (H1N2); A/sw/Cote d'Armor/1482/99 (H1N1); A/sw/Gent/7625/99 (H1N2); A/Hong Kong/1774/99 (H3N2); A/sw/Hong Kong/5190/99 (H3N2); A/sw/Hong Kong/5200/99 (H3N2); A/sw/Hong Kong/5212/99 (H3N2); A/sw/Ille et Villaine/1455/99 (H1N1); A/sw/Italy/1654-1/99 (H1N2); A/sw/Italy/2034/99 (H1N1); A/sw/Italy/2064/99 (H1N2); A/sw/Berlin/1578/00 (H3N2); A/sw/Bakum/1832/00 (H1N2); A/sw/Bakum/1833/00 (H1N2); A/sw/Cote d'Armor/800/00 (H1N2); A/sw/Hong Kong/7982/00 (H3N2); A/sw/Italy/1081/00 (H1N2); A/sw/Belzig/2/01 (H1N1); A/sw/Belzig/54/01 (H3N2); A/sw/Hong Kong/9296/01 (H3N2); A/sw/Hong Kong/9745/01 (H3N2); A/sw/Spain/33601/01 (H3N2); A/sw/Hong Kong/1144/02 (H3N2); A/sw/Hong Kong/1197/02 (H3N2); A/sw/Spain/39139/02 (H3N2); A/sw/Spain/42386/02 (H3N2); A/Switzerland/8808/2002 (H1N1); A/sw/Bakum/1769/03 (H3N2); A/sw/Bissendorf/IDT1864/03 (H3N2); A/sw/Ehren/IDT2570/03 (H1N2); A/sw/Gescher/IDT2702/03 (H1N2); A/sw/Haselünne/2617/03 hp (H1N1); A/sw/Löningen/IDT2530/03 (H1N2); A/sw/IVD/IDT2674/03 (H1N2); A/sw/Nordkirchen/IDT1993/03 (H3N2); A/sw/Nordwalde/IDT2197/03 (H1N2); A/sw/Norden/IDT2308/03 (H1N2); A/sw/Spain/50047/03 (H1N1); A/sw/Spain/51915/03 (H1N1); A/sw/Vechta/2623/03 (H1N1); A/sw/Visbek/IDT2869/03 (H1N2); A/sw/Waltersdorf/IDT2527/03 (H1N2); A/sw/Damme/IDT2890/04 (H3N2); A/sw/Geldern/IDT2888/04 (H1N1); A/sw/Granstedt/IDT3475/04 (H1N2); A/sw/Greven/IDT2889/04 (H1N1); A/sw/Gudensberg/IDT2930/04 (H1N2); A/sw/Gudensberg/IDT2931/04 (H1N2); A/sw/Lohne/IDT3357/04 (H3N2); A/sw/Nortrup/IDT3685/04 (H1N2); A/sw/Seesen/IDT3055/04 (H3N2); A/sw/Spain/53207/04 (H1N1); A/sw/Spain/54008/04 (H3N2); A/sw/Stolzenau/IDT3296/04 (H1N2); A/sw/Wedel/IDT2965/04 (H1N1); A/sw/Bad Griesbach/IDT4191/05 (H3N2); A/sw/Cloppenburg/IDT4777/05 (H1N2); A/sw/Dötlingen/IDT3780/05 (H1N2); A/sw/Dötlingen/IDT4735/05 (H1N2); A/sw/Egglham/IDT5250/05 (H3N2); A/sw/Harkenblek/IDT4097/05 (H3N2); A/sw/Hertzen/IDT4317/05 (H3N2); A/sw/Krogel/IDT4192/05 (H1N1); A/sw/Laer/IDT3893/05 (H1N1); A/sw/Laer/IDT4126/05 (H3N2); A/sw/Merzen/IDT4114/05 (H3N2); A/sw/Muesleringen-S./IDT4263/05 (H3N2); A/sw/Osterhofen/IDT4004/05 (H3N2); A/sw/Sprenge/IDT3805/05 (H1N2); A/sw/Stadtlohn/IDT3853/05 (H1N2); A/sw/Voglarn/IDT4096/05 (H1N1); A/sw/Wohlerst/IDT4093/05 (H1N1); A/sw/Bad Griesbach/IDT5604/06 (H1N1); A/sw/Herzlake/IDT5335/06 (H3N2); A/sw/Herzlake/IDT5336/06 (H3N2); A/sw/Herzlake/IDT5337/06 (H3N2); and A/wild boar/Germany/R169/2006 (H3N2).

Other specific examples of strains of influenza A virus include, but are not limited to: A/Toronto/3141/2009 (H1N1); A/Regensburg/D6/2009 (H1N1); A/Bayern/62/2009 (H1N1); A/Bayern/62/2009 (H1N1); A/Bradenburg/19/2009 (H1N1); A/Bradenburg/20/2009 (H1N1); A/Distrito Federal/2611/2009 (H1N1); A/Mato Grosso/2329/2009 (H1N1); A/Sao Paulo/1454/2009 (H1N1); A/Sao Paulo/2233/2009 (H1N1); A/Stockholm/37/2009 (H1N1); A/Stockholm/41/2009 (H1N1); A/Stockholm/45/2009 (H1N1); A/swine/Alberta/OTH-33-1/2009 (H1N1); A/swine/Alberta/OTH-33-14/2009 (H1N1); A/swine/Alberta/OTH-33-2/2009 (H1N1); A/swine/Alberta/OTH-33-21/2009 (H1N1); A/swine/Alberta/OTH-33-22/2009 (H1N1); A/swine/Alberta/OTH-33-23/2009 (H1N1); A/swine/Alberta/OTH-33-24/2009 (H1N1); A/swine/Alberta/OTH-33-25/2009 (H1N1); A/swine/Alberta/OTH-33-3/2009 (H1N1); A/swine/Alberta/OTH-33-7/2009 (H1N1); A/Beijing/502/2009 (H1N1); A/Firenze/10/2009 (H1N1); A/Hong Kong/2369/2009 (H1N1); A/Italy/85/2009 (H1N1); A/Santo Domingo/572N/2009 (H1N1); A/Catalonia/385/2009 (H1N1); A/Catalonia/386/2009 (H1N1); A/Catalonia/387/2009 (H1N1); A/Catalonia/390/2009 (H1N1); A/Catalonia/394/2009 (H1N1); A/Catalonia/397/2009 (H1N1); A/Catalonia/398/2009 (H1N1); A/Catalonia/399/2009 (H1N1); A/Sao Paulo/2303/2009 (H1N1); A/Akita/1/2009 (H1N1); A/Castro/JXP/2009 (H1N1); A/Fukushima/1/2009 (H1N1); A/Israel/276/2009 (H1N1); A/Israel/277/2009 (H1N1); A/Israel/70/2009 (H1N1); A/Iwate/1/2009 (H1N1); A/Iwate/2/2009 (H1N1); A/Kagoshima/1/2009 (H1N1); A/Osaka/180/2009 (H1N1); A/Puerto Montt/Bio87/2009 (H1 N1); A/Sao Paulo/2303/2009 (H1N1); A/Sapporo/1/2009 (H1N1); A/Stockholm/30/2009 (H1N1); A/Stockholm/31/2009 (H1N1); A/Stockholm/32/2009 (H1N1); A/Stockholm/33/2009 (H1N1); A/Stockholm/34/2009 (H1N1); A/Stockholm/35/2009 (H1N1); A/Stockholm/36/2009 (H1N1); A/Stockholm/38/2009 (H1N1); A/Stockholm/39/2009 (H1N1); A/Stockholm/40/2009 (H1N1;) A/Stockholm/42/2009 (H1N1); A/Stockholm/43/2009 (H1N1); A/Stockholm/44/2009 (H1N1); A/Utsunomiya/2/2009 (H1N1); A/WRAIR/0573N/2009 (H1N1); and A/Zhejiang/DTID-ZJU01/2009 (H1N1).

Other examples of influenza viruses may be found elsewhere in the application, such as in, e.g., Section 5.1 above and Section 6 below.

In certain embodiments, the influenza viruses provided herein have an attenuated phenotype. In specific embodiments, the attenuated influenza virus is based on influenza A virus. In specific embodiments, the attenuated influenza virus comprises, encodes, or both, a mosaic influenza virus HA polypeptide and has a backbone of an influenza A virus.

In specific embodiments, attenuation of influenza virus is desired such that the virus remains, at least partially, infectious and can replicate in vivo, but only generate low titers resulting in subclinical levels of infection that are non-pathogenic. Such attenuated viruses are especially suited for embodiments described herein wherein the virus or an immunogenic composition thereof is administered to a subject to induce an immune response. Attenuation of the influenza virus can be accomplished according to any method known in the art, such as, e.g., selecting viral mutants generated by chemical mutagenesis, mutation of the genome by genetic engineering, selecting reassortant viruses that contain segments with attenuated function (e.g., truncated NS1 protein (see, e.g., Hai et al., 2008, Journal of Virology 82(21):10580-10590, which is incorporated by reference herein in its entirety) or NS1 deletion (see, e.g., Wressnigg et al., 20009, Vaccine 27:2851-2857, which is incorporated by reference herein in its entirety)), or selecting for conditional virus mutants (e.g., cold-adapted viruses, see, e.g., Alexandrova et al., 1990, Vaccine, 8:61-64, which is incorporated by reference herein in its entirety). Alternatively, naturally occurring attenuated influenza viruses may be used as influenza virus backbones for the influenza virus vectors.

In a specific embodiment, the influenza A virus A/Puerto Rico/8/34 strain is used as the backbone to express a mosaic influenza virus HA polypeptide described herein. In another specific embodiment, the virion of the influenza A virus A/Puerto Rico/8/34 strain contains a mosaic influenza virus HA polypeptide described herein. In another specific embodiment, the influenza A virus A/Puerto Rico/8/34 strain is used to express a mosaic influenza virus HA polypeptide described herein and the virion of the A/Puerto Rico/8/34 strain contains the mosaic influenza virus HA polypeptide.

In a specific embodiment, an influenza A virus lacking the NS1 protein (e.g., a delNS1 virus, such as described, e.g., in U.S. Pat. No. 6,468,544; Garcia-Sastre et al., 1998, Virology 252:324; or Mossler et al., 2013, Vaccine 31:6194) is used as the backbone to express a mosaic influenza virus HA polypeptide described herein. In another specific embodiment, the virion of an influenza virus lacking the NS1 protein (e.g., a delNS1 virus, such as described, e.g., in U.S. Pat. No. 6,468,544; Garcia-Sastre et al., 1998, Virology 252:324; or Mössler et al., 2013, Vaccine 31:6194) contains a mosaic influenza virus HA polypeptide described herein. In another specific embodiment, an influenza virus lacking the NS1 protein (e.g., a delNS1 virus, such as described, e.g., in U.S. Pat. No. 6,468,544; Garcia-Sastre et al., 1998, Virology 252:324; or Mössler et al., 2013, Vaccine 31:6194) is used to express a mosaic influenza virus HA polypeptide described herein and the virion of such a virus contains the mosaic influenza virus HA polypeptide.

In a specific embodiment, a cold-adapted influenza A virus strain is used as the backbone to express a mosaic influenza virus HA polypeptide described herein. In another specific embodiment, the virion of the cold-adapted strain contains a mosaic influenza virus HA polypeptide described herein. In another specific embodiment, the cold-adapted influenza A virus is used to express a mosaic influenza virus HA polypeptide described herein and the virion of the cold-adapted influenza virus contains the mosaic influenza virus HA polypeptide. In one embodiment, the cold-adapted influenza A virus is A/Ann Arbor/6/60. In another embodiment, the cold-adapted influenza A virus is A/Leningrad/134/17/57.

In certain embodiments, an influenza virus comprising a mosaic influenza virus HA described herein has one, two, or more of the functions of an influenza virus comprising a wild-type influenza virus HA. Nonlimiting examples of functions of a wild-type influenza virus HA include fusogenic activity, receptor binding activity, budding, and particle formation. In a specific embodiment, an influenza virus comprising a mosaic influenza virus HA polypeptide described herein has fusogenic activity. Assays known to one skilled in the art can be utilized to assess the fusogenic activity of an influenza virus comprising a mosaic influenza virus HA polypeptide described herein, such as, for example, immunofluorescence assays and pseudotyped virus-like-particle assays. In a specific embodiment, an influenza virus comprising a mosaic influenza virus HA polypeptide described herein has replication activity. Assays known to one skilled in the art can be utilized the assess the replication activity of an influenza virus comprising a mosaic influenza virus HA polypeptide described herein, such as, for example, plaque assay and western blot analyses.

### 5.5 Virus-Like Particles and Virosomes

The mosaic influenza virus hemagglutinin polypeptides described herein can be incorporated into virus-like particle (VLP) vectors, e.g., purified/isolated VLPs. VLPs generally comprise a viral polypeptide(s) typically derived from a structural protein(s) of a virus. In some embodiments, the VLPs are not capable of replicating. In certain embodiments, the VLPs may lack the complete genome of a virus or comprise a portion of the genome of a virus. In some embodiments, the VLPs are not capable of infecting a cell. In some embodiments, the VLPs express on their surface one or more of viral (e.g., virus surface glycoprotein) or non-viral (e.g., antibody or protein) targeting moieties known to one skilled in the art or described herein. In some embodiments, the VLPs comprise a mosaic influenza virus hemagglutinin (HA) polypeptide and a viral structural protein, such as HIV gag. In a specific embodiment, the VLPs comprise a mosaic influenza virus hemagglutinin (HA) polypeptide and an HIV gag polypeptide. In another specific embodiment, the VLPs comprise a mosaic influenza virus hemagglutinin (HA) polypeptide and influenza virus neuraminidase polypeptide. In another specific embodiment, the VLPs comprise a mosaic influenza virus hemagglutinin (HA) polypeptide, influenza virus neuraminidase polypeptide, and influenza virus M1 polypeptide.

In some embodiments, a VLP comprises a mosaic influenza virus HA polypeptide that has been activated by trypsin (or a similar protease). In a specific embodiment, a VLP comprises a mosaic influenza virus HA polypeptide that has been activated by trypsin (or a similar protease) and exhibits fusogenic activity. In other embodiments, a VLP comprises a mosaic influenza virus HA polypeptide in the HA0 configuration and does not exhibit fusogenic activity.

Also provided herein are methods for producing and characterizing recombinantly produced VLPs comprising a mosaic HA described herein. Methods for producing and characterizing recombinantly produced VLPs have been described based on several viruses, including influenza virus (Bright et al. (2007) Vaccine. 25:3871), human papilloma virus type 1 (Hagnesee et al. (1991) J. Virol. 67:315), human papilloma virus type 16 (Kirnbauer et al. Proc. Natl. Acad. Sci. (1992) 89:12180), HIV-1 (Haffer et al., (1990) J. Virol. 64:2653), and hepatitis A (Winokur (1991) 65:5029), each of which is incorporated herein in its entirety. Methods for expressing VLPs that contain NDV proteins are provided by Pantua et al. (2006) J. Virol. 80:11062-11073, and in United States patent application Publication No. 20090068221, published Mar. 12, 2009, each of which is incorporated in its entirety herein. In a specific embodiment, the VLPs comprising mosaic influenza virus hemagglutinin (HA) polypeptide described herein are generated using baculovirus. In other embodiments, the VLPs comprising mosaic influenza virus hemagglutinin (HA) polypeptides described herein are generated using 293T cells.

In specific embodiments, VLPs, e.g., VLPs comprising a mosaic influenza virus hemagglutinin (HA) polypeptide are expressed in cells (such as, e.g., mammalian cells (e.g., 293T cells) and insect cells (e.g., High Five cells and Sf9 cells). In certain embodiments, the VLPs are expressed in cells that express surface glycoproteins that comprise sialic acid. In certain embodiments, VLPs, e.g., VLPs comprising a mosaic influenza virus hemagglutinin (HA) polypeptide, are expressed in cells that do not express surface glycoproteins that comprise sialic acid.

In a specific embodiment, a mosaic influenza virus hemagglutinin (HA) polypeptide may be incorporated into a virosome. A virosome containing a mosaic influenza virus hemagglutinin (HA) polypeptide may be produced using techniques known to those skilled in the art. For example, a virosome may be produced by disrupting a purified virus, extracting the genome, and reassembling particles with the viral proteins (e.g., a mosaic hemagglutinin (HA) polypeptide) and lipids to form lipid particles containing viral proteins.

### 5.6 Compositions

The nucleic acids, vectors, and polypeptides described herein (sometimes referred to herein as “active compounds”) may be incorporated into compositions. In specific embodiments, an active compound described herein is a mosaic influenza virus hemagglutinin (HA) polypeptide described herein, a nucleic acid encoding such a polypeptide(s), or a vector (e.g., a viral vector) either containing, expressing, or both such a polypeptide(s). In a specific embodiment, the compositions are pharmaceutical compositions, such as immunogenic compositions (e.g., vaccine formulations). The pharmaceutical compositions provided herein can be in any form that allows for the composition to be administered to a subject. In a specific embodiment, the pharmaceutical compositions are suitable for veterinary and/or human administration. The compositions may be used in methods of preventing an influenza virus disease. The compositions may be used in methods to induce an immune response against influenza virus.

In a specific embodiment, a pharmaceutical composition (e.g., immunogenic composition) comprises a mosaic influenza virus hemagglutinin polypeptide, and optionally an adjuvant. In another specific embodiment, a pharmaceutical composition (e.g., immunogenic composition) comprises a mosaic influenza virus hemagglutinin polypeptide in an admixture with a pharmaceutically acceptable carrier. In a specific embodiment, a pharmaceutical composition (e.g., immunogenic composition) comprises a mosaic influenza virus hemagglutinin polypeptide and an adjuvant (e.g., an adjuvant described in Section 5.6.5 below), in an admixture with a pharmaceutically acceptable carrier.

In another embodiment, a pharmaceutical composition (e.g., an immunogenic composition) comprises a nucleic acid sequence comprising a nucleotide sequence encoding a mosaic hemagglutinin (HA) polypeptide described herein and optionally, an adjuvant (e.g., an adjuvant described in Section 5.6.5 below). In another embodiment, a pharmaceutical composition (e.g., an immunogenic composition) comprises (1) a first nucleic acid sequence comprising a first nucleotide sequence encoding a mosaic hemagglutinin (HA) polypeptide described herein, (2) a second nucleic acid sequence comprising a second nucleotide sequence encoding an influenza A virus neuraminidase, and optionally, (3) an adjuvant (e.g., an adjuvant described in Section 5.6.5 below). In another embodiment, a pharmaceutical composition (e.g., an immunogenic composition) comprises (1) a first nucleic acid sequence comprising a first nucleotide sequence encoding a mosaic hemagglutinin (HA) polypeptide described herein, (2) a second nucleic acid sequence comprising a second nucleotide sequence encoding an influenza A virus neuraminidase, (3) a third nucleic acid sequence comprising a nucleotide sequence encoding an influenza A virus nucleoprotein, and optionally, (4) an adjuvant (e.g., an adjuvant described in Section 5.6.5 below). In a specific embodiment, one, two or all of the following are RNA sequences: first nucleic acid sequence, second nucleic acid sequence, and third nucleic acid sequence.

In another embodiment, a pharmaceutical composition (e.g., an immunogenic composition) comprises a nucleic acid sequence comprising a nucleotide sequence encoding a mosaic hemagglutinin (HA) polypeptide described herein and optionally, an adjuvant (e.g., an adjuvant described in Section 5.6.5 below), in an admixture with a pharmaceutically acceptable carrier. In another embodiment, a pharmaceutical composition (e.g., an immunogenic composition) comprises (1) a first nucleic acid sequence comprising a first nucleotide sequence encoding a mosaic hemagglutinin (HA) polypeptide described herein, (2) a second nucleic acid sequence comprising a second nucleotide sequence encoding an influenza A virus neuraminidase, and optionally, (3) an adjuvant (e.g., an adjuvant described in Section 5.6.5 below), in an admixture with a pharmaceutically acceptable carrier. In another embodiment, a pharmaceutical composition (e.g., an immunogenic composition) comprises (1) a first nucleic acid sequence comprising a first nucleotide sequence encoding a mosaic hemagglutinin (HA) polypeptide described herein, (2) a second nucleic acid sequence comprising a second nucleotide sequence encoding an influenza A virus neuraminidase, (3) a third nucleic acid sequence comprising a nucleotide sequence encoding an influenza A virus nucleoprotein, and optionally, (4) an adjuvant (e.g., an adjuvant described in Section 5.6.5 below), in an admixture with a pharmaceutically acceptable carrier. In a specific embodiment, one, two or all of the following are RNA sequences: first nucleic acid sequence, second nucleic acid sequence, and third nucleic acid sequence.

In another embodiment, a pharmaceutical composition (e.g., an immunogenic composition) comprises an expression vector comprising a nucleic acid sequence encoding a mosaic influenza virus hemagglutinin (HA) polypeptide and optionally, an adjuvant (e.g., an adjuvant described in Section 5.6.5 below), in an admixture with a pharmaceutically acceptable carrier. In another embodiment, a pharmaceutical composition (e.g., an immunogenic composition) comprises (1) a first expression vector comprising a first nucleic acid sequence encoding a mosaic influenza virus hemagglutinin (HA) polypeptide, (2) a second expression vector comprising a second nucleic acid sequence comprising a second nucleotide sequence encoding influenza A virus neuraminidase, and optionally, (3) an adjuvant (e.g., an adjuvant described in Section 5.6.5 below), in an admixture with a pharmaceutically acceptable carrier. In another embodiment, a pharmaceutical composition (e.g., an immunogenic composition) comprises (1) a first expression vector comprising a first nucleic acid sequence encoding a mosaic influenza virus hemagglutinin (HA) polypeptide, (2) a second expression vector comprising a second nucleic acid sequence comprising a second nucleotide sequence encoding influenza A virus neuraminidase, (3) a third expression vector comprising a third nucleic acid sequence comprising a third nucleotide sequence encoding influenza A virus nucleoprotein, and optionally, (4) an adjuvant (e.g., an adjuvant described in Section 5.6.5 below), in an admixture with a pharmaceutically acceptable carrier.

In another embodiment, a pharmaceutical composition (e.g., an immunogenic composition) comprises an influenza virus or non-influenza virus containing a mosaic influenza virus hemagglutinin (HA) polypeptide. In another embodiment, a pharmaceutical composition (e.g., an immunogenic composition) comprises an influenza virus or non-influenza virus containing a mosaic influenza virus hemagglutinin (HA) polypeptide, in an admixture with a pharmaceutically acceptable carrier. In another embodiment, a pharmaceutical composition comprises an influenza virus or non-influenza virus having a genome engineered to express a mosaic influenza virus hemagglutinin (HA) polypeptide, in admixture with a pharmaceutically acceptable carrier. In certain embodiments, the pharmaceutical composition (e.g., an immunogenic composition) further comprises an adjuvant (e.g., an adjuvant described in Section 5.6.5 below).

In another embodiment, a pharmaceutical composition comprises a virus-like particle or virosome containing a mosaic influenza virus hemagglutinin (HA) polypeptide. In another embodiment, a pharmaceutical composition comprises a virus-like particle or virosome containing a mosaic influenza virus hemagglutinin (HA) polypeptide, in an admixture with a pharmaceutically acceptable carrier. In another embodiment, a pharmaceutical composition comprises bacteria expressing or engineered to express a mosaic influenza virus hemagglutinin (HA) polypeptide, in an admixture with a pharmaceutically acceptable carrier. In certain embodiments, the pharmaceutical composition (e.g., an immunogenic composition) further comprises an adjuvant (e.g., an adjuvant described in Section 5.6.5 below).

In some embodiments, a pharmaceutical composition (e.g., an immunogenic composition) may comprise one or more other therapies in addition to a therapy that utilizes a mosaic influenza virus hemagglutinin (HA) polypeptide described herein.

As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, pharmaceutical compositions are formulated to be suitable for the intended route of administration to a subject. For example, the pharmaceutical composition may be formulated to be suitable for parenteral, oral, intradermal, transdermal, colorectal, intraperitoneal, and rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration. In a specific embodiment, the pharmaceutical composition may be formulated for intramuscular administration. In a specific embodiment, the pharmaceutical composition may be formulated for subcutaneous administration.

In specific embodiments, immunogenic compositions described herein are monovalent formulations. In other embodiments, immunogenic compositions described herein are multivalent formulations. In one example, a multivalent formulation comprises more than one mosaic influenza virus hemagglutinin (HA) polypeptide. In another example, a multivalent formulation comprises more than one vector expressing a mosaic influenza virus hemagglutinin (HA) polypeptide. In another example, a multivalent formulation comprises more than one virus containing a mosaic hemagglutinin (HA) polypeptide. In certain embodiments, a multivalent formulation may comprise one or more different mosaic hemagglutinin (HA) polypeptides expressed using a single vector. In certain embodiments, immunogenic compositions described herein are trivalent vaccines which comprise at least one mosaic influenza virus hemagglutinin (HA) polypeptide. In some embodiments, immunogenic compositions described herein are trivalent vaccines which comprise three different influenza viruses, each influenza virus comprising a different mosaic influenza virus HA polypeptide. In some embodiments, immunogenic compositions described herein are quadrivalent vaccines which comprise at least four different mosaic influenza virus hemagglutinin (HA) polypeptides described herein. In some embodiments, immunogenic compositions described herein are quadrivalent vaccines which comprise four different influenza viruses, each influenza virus comprising a different mosaic influenza virus HA.

In specific embodiments, an immunogenic composition described herein comprises: (1) a first mosaic influenza A virus HA in which the HA ectodomain is derived from a group 1 influenza A virus strain HA ectodomain, such as described in Section 5.1.1, and (2) a second mosaic influenza A virus HA in which the HA ectodomain is derived from a group 2 influenza A virus strain HA ectodomain, such as described in Section 5.1.2. In certain embodiments, a composition described herein comprises 2, 3, or more of the mosaic HA polypeptides described herein. The immunogenic composition may be an inactivated vaccine, such as subunit vaccine, split vaccine or whole inactivated virus vaccine.

In some embodiments, an immunogenic composition described herein comprises: (1) a first mosaic influenza A virus HA in which the HA ectodomain is derived from a group 1 influenza A virus strain HA ectodomain, such as described in Section 5.1.1, and (2) one or more of the chimeric influenza virus hemagglutinin (HA) polypeptides in which the HA ectodomain is derived from an influenza B virus HA ectodomain, such as described in International Publication No. WO 2017/218624, which is incorporated by reference in its entirety. In certain embodiments, an immunogenic composition described herein comprises: (1) a first mosaic influenza A virus HA in which the HA ectodomain is derived from a group 2 influenza A virus strain HA ectodomain, such as described in Section 5.1.1, and (2) one or more of the chimeric influenza virus hemagglutinin (HA) polypeptides in which the HA ectodomain is derived from an influenza B virus HA ectodomain, such as described in International Publication No. WO 2017/218624, which is incorporated by reference in its entirety. In some embodiments, an immunogenic composition described herein comprises: (1) a first mosaic influenza A virus HA in which the HA ectodomain is derived from a group 1 influenza A virus strain HA ectodomain, such as described in Section 5.1.1, (2) a second mosaic influenza A virus HA in which the HA ectodomain is derived from a group 2 influenza A virus strain HA ectodomain, such as described in Section 5.1.2, and (3) one or more of the chimeric influenza virus hemagglutinin (HA) polypeptides in which the HA ectodomain is derived from an influenza B virus HA ectodomain, such as described in International Publication No. WO 2017/218624, which is incorporated by reference in its entirety. The immunogenic composition may be an inactivated vaccine, such as subunit vaccine, split vaccine or whole inactivated virus vaccine.

In some embodiments, an immunogenic composition described herein comprises: (1) a first mosaic influenza A virus HA in which the HA ectodomain is derived from a group 1 influenza A virus strain HA ectodomain, such as described in Section 5.1.1 or Section 6, and (2) a second mosaic HA polypeptide comprising an HA ectodomain of an influenza B virus HA, wherein the HA ectodomain comprises the HA globular head of the influenza B virus HA and the HA stem domain of the influenza B virus HA, and wherein the HA globular head domain of the influenza B virus HA comprises one, two, three, four, five, six, seven, eight, nine, ten or more amino acid substitutions in the one, two, three or all of the 120 loop, 150 loop, 160 loop and 190 helix, such as described in International Publication No. WO 2017/218624 (which is referred to in the publication as a chimeric HA polypeptide), which is incorporated by reference in its entirety. The immunogenic composition may be an inactivated vaccine, such as subunit vaccine, split vaccine or whole inactivated virus vaccine.

In certain embodiments, an immunogenic composition described herein comprises: (1) a first mosaic influenza A virus HA in which the HA ectodomain is derived from a group 2 influenza A virus strain HA ectodomain, such as described in Section 5.1.2 or Section 6, and (2) a second mosaic HA polypeptide, which comprises an HA ectodomain of an influenza B virus HA, wherein the HA ectodomain comprises the HA globular head of the influenza B virus HA and the HA stem domain of the influenza B virus HA, and wherein the HA globular head domain of the influenza B virus HA comprises one, two, three, four, five, six, seven, eight, nine, ten or more amino acid substitutions in the one, two, three or all of the 120 loop, 150 loop, 160 loop and 190 helix, such as described in International Publication No. WO 2017/218624 (which is referred to in the publication as a chimeric HA polypeptide), which is incorporated by reference in its entirety. The immunogenic composition may be an inactivated vaccine, such as subunit vaccine, split vaccine or whole inactivated virus vaccine.

In some embodiments, an immunogenic composition described herein comprises: (1) a first mosaic influenza A virus HA in which the HA ectodomain is derived from a group 1 influenza A virus strain HA ectodomain, such as described in Section 5.1.1, (2) a second mosaic influenza A virus HA in which the HA ectodomain is derived from a group 2 influenza A virus strain HA ectodomain, such as described in Section 5.1.2, and (3) a third mosaic HA polypeptide, which comprises an HA ectodomain of an influenza B virus HA, wherein the HA ectodomain comprises the HA globular head of the influenza B virus HA and the HA stem domain of the influenza B virus HA, and wherein the HA globular head domain of the influenza B virus HA comprises one, two, three, four, five, six, seven, eight, nine, ten or more amino acid substitutions in the one, two, three or all of the 120 loop, 150 loop, 160 loop and 190 helix, such as described in International Publication No. WO 2017/218624 (which is referred to in the publication as a chimeric HA polypeptide), which is incorporated by reference in its entirety. The immunogenic composition may be an inactivated vaccine, such as subunit vaccine, split vaccine or whole inactivated virus vaccine.

An immunogenic composition described herein may be used to immunize a subject against influenza virus. An immunogenic composition described herein may also be used to prevent an influenza virus disease in a subject. In a specific embodiment, an immunogenic composition described herein may be used in a method described in Section 5.7, infra.

In certain embodiments, the pharmaceutical compositions (e.g., immunogenic compositions) described herein additionally comprise one or more components used to inactivate a virus, e.g., formalin or formaldehyde or a detergent such as sodium deoxycholate, octoxynol 9 (Triton X-100), and octoxynol 10. In other embodiments, the pharmaceutical compositions described herein do not comprise any components used to inactivate a virus.

In certain embodiments, the pharmaceutical compositions (e.g., immunogenic compositions) described herein additionally comprise one or more buffers, e.g., phosphate buffer and sucrose phosphate glutamate buffer. In other embodiments, the pharmaceutical compositions described herein do not comprise buffers.

The pharmaceutical compositions (e.g., immunogenic compositions) described herein can be included in a container, pack, or dispenser together with instructions for administration.

The pharmaceutical compositions (e.g., immunogenic compositions) described herein can be stored before use, e.g., the pharmaceutical compositions can be stored frozen (e.g., at about −20° C. or at about −70° C.); stored in refrigerated conditions (e.g., at about 4° C.); or stored at room temperature (see International Application No. PCT/IB2007/001149 published as International Publication No. WO 07/110776, which is herein incorporated by reference in its entirety, for methods of storing compositions comprising influenza vaccines without refrigeration).

In a specific embodiment, an immunogenic composition is an inactivated vaccine comprising an adjuvant (e.g., an adjuvant described in Section 5.6.5 below) and a mosaic HA polypeptide. The inactivated vaccine may be a whole virus inactivated vaccine, split virion vaccine, or subunit vaccine. Techniques for producing such vaccines are known to one of skill in the art.

### 5.6.1 SUBUNIT VACCINES

In a specific embodiment, provided herein are subunit vaccines comprising a mosaic influenza virus hemagglutinin polypeptide described herein. In a specific embodiment, provided herein are subunit vaccines comprising a mosaic influenza virus hemagglutinin polypeptide described herein and an adjuvant (e.g., an adjuvant described in Section 5.6.5 below). In some embodiments, a subunit vaccine comprises a mosaic hemagglutinin (HA) polypeptide and one or more surface glycoproteins (e.g., influenza virus neuraminidase), and optionally, other targeting moieties and an adjuvant (e.g., an adjuvant described in Section 5.6.5 below).

In specific embodiments, a subunit vaccine comprises a single mosaic hemagglutinin (HA) polypeptide. In other embodiments, a subunit vaccine comprises two, three, four or more mosaic hemagglutinin (HA) polypeptides. In specific embodiments, the mosaic hemagglutinin (HA) polypeptide(s) used in a subunit vaccine are not membrane-bound, i.e., are soluble. In specific embodiments, the polypeptide components of the subunit vaccine are generated in a baculovirus expression system. In a particular embodiment, a subunit vaccine comprises a purified mosaic HA polypeptide described herein which is produced in a continuous insect cell line, such as one derived from the fall armyworm Spodoptera frugiperda using a baculovirus vector (e.g., Autographa californica nuclear polyhedrosis virus). The mosaic HA polypeptide may be extracted from the cells and further purified by column chromatography. In some embodiments, a subunit vaccine comprises more than one mosaic HA polypeptide described herein.

In a specific embodiment, the subunit vaccine is prepared using influenza virus that was propagated in embryonated chicken eggs (i.e., the components of the subunit vaccine (e.g., a mosaic hemagglutinin (HA) polypeptide) are isolated from virus that was propagated in embryonated chicken eggs). In another specific embodiment, the subunit vaccine is prepared using influenza virus that was not propagated in embryonated chicken eggs (i.e., the components of the subunit vaccine (e.g., a mosaic hemagglutinin (HA) polypeptide) are isolated from virus that was not propagated in embryonated chicken eggs). In another specific embodiment, the subunit vaccine is prepared using influenza virus that was propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) (i.e., the components of the subunit vaccine (e.g., a mosaic hemagglutinin (HA) polypeptide) are isolated from virus that was propagated in mammalian cells). In another specific embodiment, the mosaic hemagglutinin (HA) polypeptide(s) in a subunit vaccine are prepared using an expression vector, e.g., a viral vector, plant vector, or baculovirus vector (i.e., the mosaic hemagglutinin (HA) polypeptide(s) in the subunit vaccine are obtained/isolated from an expression vector).

### 5.6.2 LIVE VIRUS VACCINES

In one embodiment, provided herein are immunogenic compositions (e.g., vaccines) comprising a live influenza virus containing a mosaic influenza virus hemagglutinin polypeptide. In another embodiment, provided herein are immunogenic compositions (e.g., vaccines) comprising a live virus that is engineered to encode a mosaic hemagglutinin (HA) polypeptide, which is expressed by progeny virus produced in the subjects administered the compositions. In certain embodiments, such immunogenic compositions may further comprise an adjuvant (e.g., an adjuvant described in Section 5.6.5 below). In specific embodiments, the mosaic hemagglutinin (HA) polypeptide is membrane-bound. In other specific embodiments, the mosaic hemagglutinin (HA) polypeptide is not membrane-bound, i.e., it is soluble. In particular embodiments, the live virus is an influenza virus, such as described in Section 5.4 above. In some embodiments, the live virus is attenuated. In some embodiments, an immunogenic composition comprises two, three, four or more live viruses containing or engineered to express two, three, four or more different mosaic hemagglutinin (HA) polypeptides.

An immunogenic composition comprising a live influenza virus for administration to a subject may be preferred because multiplication of the virus in the subject may lead to a prolonged stimulus of similar kind and magnitude to that occurring in natural infections, and therefore, confer substantial, long lasting immunity.

In a specific embodiment, the live virus that contains a mosaic hemagglutinin (HA) polypeptide is propagated in embryonated chicken eggs before its use in an immunogenic composition described herein. In another specific embodiment, the live virus that contains a mosaic hemagglutinin (HA) polypeptide is not propagated in embryonated chicken eggs before its use in an immunogenic composition described herein. In another specific embodiment, the live virus that contains a mosaic hemagglutinin (HA) polypeptide is propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) before its use in an immunogenic composition described herein.

### 5.6.3 INACTIVATED VIRUS VACCINES

In one embodiment, provided herein are immunogenic compositions (e.g., vaccines) comprising an inactivated virus containing a mosaic influenza virus hemagglutinin polypeptide. In one embodiment, provided herein are immunogenic compositions (e.g., vaccines) comprising an adjuvant (e.g., an adjuvant described in Section 5.6.5 below) and an inactivated virus containing a mosaic influenza virus hemagglutinin polypeptide. In specific embodiments, the mosaic hemagglutinin (HA) polypeptide is membrane-bound. In particular embodiments, the inactivated virus is an influenza virus, such as described in Section 5.4 above. In some embodiments, an immunogenic composition comprises two, three, four or more inactivated viruses containing two, three, four or more different mosaic hemagglutinin (HA) polypeptides. In certain embodiments, the inactivated virus immunogenic compositions comprise one or more adjuvants.

Techniques known to one of skill in the art may be used to inactivate viruses containing a mosaic hemagglutinin (HA) polypeptide. Common methods use formalin, heat, or detergent for inactivation. See, e.g., U.S. Pat. No. 6,635,246, which is herein incorporated by reference in its entirety. Other methods include those described in U.S. Pat. Nos. 5,891,705; 5,106,619, 4,693,981, 7,238,349, and 7,316,813, U.S. Patent Application Publication Nos. 2008/0181911 and 2009/0263422, and International Patent Application Publication Nos. WO 2001/022992, WO 2006/100109, WO 2002/097072, and WO 2008/009309, each which are incorporated herein by reference in their entireties.

In a specific embodiment, the inactivated virus that contains a mosaic hemagglutinin (HA) polypeptide was propagated in embryonated chicken eggs before its inactivation and subsequent use in an immunogenic composition described herein. In another specific embodiment, the inactivated virus that contains a mosaic hemagglutinin (HA) polypeptide was not propagated in embryonated chicken eggs before its inactivation and subsequent use in an immunogenic composition described herein. In another specific embodiment, the inactivated virus that contains a mosaic hemagglutinin (HA) polypeptide was propagated in mammalian cells, e.g., immortalized human cells (see, e.g., International Application No. PCT/EP2006/067566 published as International Publication No. WO 07/045674 which is herein incorporated by reference in its entirety) or canine kidney cells such as MDCK cells (see, e.g., International Application No. PCT/IB2007/003536 published as International Publication No. WO 08/032219 which is herein incorporated by reference in its entirety) before its inactivation and subsequent use in an immunogenic composition described herein.

### 5.6.4 SPLIT VIRUS VACCINES

In one embodiment, an immunogenic composition comprising a mosaic influenza virus hemagglutinin polypeptide is a split virus vaccine. In one embodiment, an immunogenic composition comprising an adjuvant (e.g., an adjuvant described in Section 5.6.5 below) and a mosaic influenza virus hemagglutinin polypeptide is a split virus vaccine. In some embodiments, split virus vaccine contains two, three, four or more different mosaic hemagglutinin (HA) polypeptides. In certain embodiments, the mosaic hemagglutinin (HA) polypeptide and/or the influenza virus neuraminidase polypeptide is/was membrane-bound.

Techniques for producing split virus vaccines are known to those skilled in the art. By way of non-limiting example, an influenza virus split vaccine may be prepared using inactivated particles disrupted with detergents. One example of a split virus vaccine that can be adapted for use in accordance with the methods described herein is the Fluzone®, Influenza Virus Vaccine (Zonal Purified, Subvirion) for intramuscular use, which is formulated as a sterile suspension prepared from influenza viruses propagated in embryonated chicken eggs. The virus-containing fluids are harvested and inactivated with formaldehyde. Influenza virus is concentrated and purified in a linear sucrose density gradient solution using a continuous flow centrifuge. The virus is then chemically disrupted using a nonionic surfactant, octoxinol-9, (Triton® X-100-A registered trademark of Union Carbide, Co.) producing a “split virus.” The split virus is then further purified by chemical means and suspended in sodium phosphate-buffered isotonic sodium chloride solution.

In a specific embodiment, the split virus vaccine is prepared using influenza virus that was propagated in embryonated chicken eggs. In another specific embodiment, the split virus vaccine is prepared using influenza virus that was not propagated in embryonated chicken eggs. In another specific embodiment, the split virus vaccine is prepared using influenza virus that was propagated in mammalian cells, e.g., immortalized human cells (see, e.g., PCT/EP2006/067566 published as WO 07/045674 which is herein incorporated by reference in its entirety) or canine kidney cells such as MDCK cells (see, e.g., PCT/IB2007/003536 published as WO 08/032219 which is herein incorporated by reference in its entirety). Other methods for preparing the split virus vaccine are known in the art, such as, e.g., those described in U.S. Pat. Nos. 7,238,349 and 7,316,813, U.S. Patent Application Publication Nos. 2008/0181911 and 2009/0263422, and International Patent Application Publication Nos. WO 2001/022992, WO 2006/100109, WO 2002/097072, and WO 2008/009309, each which are incorporated herein by reference in their entireties.

### 5.6.5 ADJUVANTS

In certain embodiments, the compositions described herein comprise, or are administered in combination with, an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to a mosaic hemagglutinin (HA) polypeptide, but when the compound is administered alone does not generate an immune response to the polypeptide. In some embodiments, the adjuvant generates an immune response to the polypeptide and does not produce an allergy or other adverse reaction. Adjuvants can enhance an immune response by several mechanisms including, e.g., lymphocyte recruitment, stimulation of B and/or T cells, and stimulation of macrophages. In a specific embodiment, an adjuvant increases the IgG levels against influenza A virus HA when used with a composition comprising a mosaic HA polypeptide. See, e.g., Section 6.3, infra regarding an increase in IgG levels.

In certain embodiments, an adjuvant augments the intrinsic response to the chimeric hemagglutinin (HA) polypeptide without causing conformational changes in the polypeptide that affect the qualitative form of the response. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In specific embodiments, the adjuvant is AS03 (GlaxoSmithKline). In specific embodiments, the adjuvant is MF59 (Novartis). In some embodiments, the adjuvant is Freund's adjuvant (complete or incomplete). In some embodiments, the adjuvant is an oil-in-water adjuvant (e.g., AddaVax such as noted in Section 6.3, infra). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as polyglutamic acid or polylysine. It should be understood that different formulations of mosaic HA polypeptide or chimeric HA polypeptides may comprise different adjuvants or may comprise the same adjuvant.

### 5.7 Prophylactic and Therapeutic Uses

In one aspect, provided herein are methods for inducing an immune response in a subject utilizing an active compound (e.g., a mosaic hemagglutinin (HA) polypeptide described herein, a nucleic acid sequence encoding such a polypeptide(s), a vector (e.g., a viral vector) containing or expressing such a polypeptide(s), or a composition described herein). In a specific embodiment, a method for inducing an immune response to an influenza virus hemagglutinin polypeptide in a subject comprises administering to a subject in need thereof an effective amount of an immunogenic composition described herein. In another embodiment, a method for inducing an immune response to an influenza virus hemagglutinin polypeptide in a subject comprises administering to a subject in need thereof an effective amount of a mosaic hemagglutinin (HA) polypeptide described herein, or an immunogenic composition thereof. In certain embodiments, a mosaic hemagglutinin (HA) polypeptide described herein used in the method is a purified mosaic hemagglutinin (HA) polypeptide described herein derived from a mammalian cell, a plant cell, or an insect cell. In another embodiment, a method for inducing an immune response to an influenza virus hemagglutinin polypeptide in a subject comprises administering to a subject in need thereof an effective amount of a nucleic acid sequence comprising a nucleotide sequence encoding a mosaic hemagglutinin (HA) polypeptide described herein, or an immunogenic composition thereof. In another embodiment, a method for inducing an immune response to an influenza virus hemagglutinin polypeptide in a subject comprises administering to a subject in need thereof an effective amount of a viral vector (e.g., an influenza virus vector) containing, expressing or both containing and expressing a mosaic hemagglutinin (HA) polypeptide described herein, or an immunogenic composition thereof. In another embodiment, a method for inducing an immune response to an influenza virus hemagglutinin polypeptide in a subject comprises administering to a subject in need thereof a virus-like particle vaccine described herein, or an immunogenic composition thereof. In another embodiment, a method for inducing an immune response to an influenza hemagglutinin polypeptide comprises administering to a subject in need thereof a virosome described herein, or an immunogenic composition thereof.

In a specific embodiment, a method for inducing an immune response to an influenza virus hemagglutinin polypeptide in a subject comprises administering to a subject in need thereof a subunit vaccine described herein. In another embodiment, a method for inducing an immune response to an influenza virus hemagglutinin polypeptide in a subject comprises administering to a subject in need thereof a live virus vaccine described herein. In particular embodiments, the live virus vaccine comprises an attenuated virus. In another embodiment, a method for inducing an immune response to an influenza virus hemagglutinin polypeptide in a subject comprises administering to a subject in need thereof an inactivated virus vaccine described herein. In another embodiment, a method for inducing an immune response to an influenza virus hemagglutinin polypeptide in a subject comprises administering to a subject in need thereof a split virus vaccine described herein.

In one embodiment, provided herein is a method for immunizing against influenza virus in a subject, comprising administering to the subject an immunogenic composition described herein (e.g., in Section 5.6 above). In some embodiments, the immunogenic composition comprises a mosaic HA polypeptide described herein (e.g., in Section 5.1 above or Section 6) and optionally an adjuvant described herein (e.g., in Section 5.6.5 above). In another embodiment, provided herein is a method for immunizing against influenza virus in a subject, comprising administering to the subject an effective amount of a nucleic acid sequence comprising a nucleotide sequence encoding a mosaic hemagglutinin (HA) polypeptide described herein, or an immunogenic composition thereof. In another embodiment, provided herein is a method for immunizing against influenza virus in a subject, comprising administering to the subject an effective amount of a viral vector (e.g., an influenza virus vector) containing, expressing or both containing and expressing a mosaic hemagglutinin (HA) polypeptide described herein, or an immunogenic composition thereof. In another embodiment, provided herein is a method for immunizing against influenza virus in a subject, comprising administering to the subject a virus-like particle vaccine described herein, or an immunogenic composition thereof. In another embodiment, provided herein is a method for immunizing against influenza virus in a subject, comprising administering to the subject a virosome described herein, or an immunogenic composition thereof.

In another aspect, provided herein is a method for immunizing against influenza virus in a subject, comprising administering to the subject an immunogenic composition described herein (e.g., in Section 5.6 above) and administering to the subject an adjuvant described herein (e.g., in Section 5.6.5 above). In one embodiment, provided herein is a method for immunizing against influenza virus in a subject, comprising administering to the subject an immunogenic composition described herein (e.g., in Section 5.6 above) in combination with an adjuvant described herein (e.g., in Section 5.6.5 above). The immunogenic composition may be administered to the subject concurrently with, prior to (e.g., less than 5 minutes, less than 10 minutes, less than 15 minutes, less than 30 minutes, less than 45 minutes, less than 60 minutes, less than 1.5 hours, or less than 2 hours prior to), or subsequent to (e.g., less than 5 minutes, less than 10 minutes, less than 15 minutes, less than 30 minutes, less than 45 minutes, less than 60 minutes, less than 1.5 hours, or less than 2 hours after) the administration of the liposomal adjuvant described herein. In a specific embodiment, the immunogenic composition and the adjuvant described herein are administered via the same route of administration. In other embodiments, the immunogenic composition and the adjuvant are administered via different routes of administration. In a specific embodiment, the immunogenic composition comprises an inactivated influenza virus containing a mosaic HA polypeptide described herein. In another specific embodiment, the immunogenic composition comprises a split influenza virus, wherein the split influenza virus comprises a mosaic HA polypeptide described herein. In some embodiments, the immunogenic composition does not comprise an adjuvant.

In another embodiment, provided herein are immunization regimens involving a first immunization (e.g., priming) with an immunogenic composition (e.g., a vaccine) described herein followed by one, two, or more additional immunizations (e.g., boostings) with an immunogenic composition (e.g., a vaccine). In a specific embodiment, the immunogenic composition (e.g., a vaccine) used in the first immunization is the same type of an immunogenic composition (e.g., a vaccine) used in one, two or more additional immunizations. For example, if the immunogenic composition (e.g., vaccine) used in the first immunization is an inactivated influenza virus vaccine formulation, the immunogenic composition (e.g., vaccine) used for the one, two or more additional immunizations may be the same type of vaccine formulation, i.e., an inactivated influenza virus vaccine formulation. In other specific embodiments, the immunogenic composition (e.g., vaccine) used in the first immunization is different from the type of immunogenic composition (e.g., vaccine) used in one, two or more additional immunizations. For example, if the immunogenic composition (e.g., vaccine) used in the first immunization is a live influenza virus vaccine formulation, the immunogenic composition (e.g., vaccine) used in the one, two or more additional immunizations is another type of vaccine formulation, such as an inactivated influenza virus. In another example, if the immunogenic composition (e.g., vaccine) used in the first immunization is a live attenuated influenza virus vaccine formulation, the immunogenic composition (e.g., vaccine) used in the one, two or more additional immunizations is another type of vaccine formulation, such as an inactivated influenza virus. In certain embodiments, the vaccine formulation used in the additional immunizations changes. For example, if a live attenuated influenza virus vaccine formulation is used for one additional immunization, then one or more additional immunizations may use a different vaccine formulation, such as an inactivated vaccine formulation. In a particular embodiment, a live influenza virus vaccine formulation is administered to a subject followed by an inactivated vaccine formulation (e.g., split virus vaccine or subunit vaccine).

In some embodiments, the immune response induced by an immunogenic composition described herein is effective to prevent an influenza virus disease caused by one, two, or more subtypes of influenza A virus. In some embodiments, the immune response induced by an immunogenic composition described herein is effective to prevent an influenza virus disease caused by one, two, three or more strains of influenza virus. In certain embodiments, the immune response induced by an immunogenic composition described herein is effective to prevent an influenza virus disease caused by a subtype of influenza virus that belongs to one HA group and not the other HA group. For example, the immune response induced by an immunogenic composition described herein may be effective to prevent an influenza virus disease caused by an influenza A virus that belongs to the a group 1 influenza A virus (e.g, H1, H2, H5, H6, H8, H9, H11, H12, H13, H16 or H17) but not a group 2 virus (e.g., H3, H4, H7, H10, H14 or H15). Alternatively, the immune response induced by an immunogenic composition described herein may be effective to prevent an influenza virus disease caused by an influenza virus that belongs to the a group 2 virus (e.g., H3, H4, H7, H10, H14 or H15) but not a group 1 influenza A virus (e.g, H1, H2, H5, H6, H8, H9, H11, H12, H13, H16 or H17). In some embodiments, the immune response induced by an immunogenic composition described herein is effective to prevent an influenza virus disease caused by one or more variants within the same subtype of influenza A virus. In certain embodiments, the immune response induced by an immunogenic composition described herein is effective to prevent an influenza virus disease caused by one, two, three or more strains within the same subtype of influenza A virus.

In some embodiments, the immune response induced by an immunogenic composition described herein is effective to reduce the number of symptoms resulting from an influenza virus disease/infection. In certain embodiments, the immune response induced by an immunogenic composition described herein is effective to reduce the duration of one or more symptoms resulting from an influenza virus disease/infection. Symptoms of influenza virus disease/infection include, but are not limited to, body aches (especially joints and throat), fever, nausea, headaches, irritated eyes, fatigue, sore throat, reddened eyes or skin, and abdominal pain.

In some embodiments, the immune response induced by an immunogenic composition described herein is effective to reduce the hospitalization of a subject suffering from an influenza virus disease/infection. In some embodiments, the immune response induced by an immunogenic composition described herein is effective to reduce the duration of hospitalization of a subject suffering from an influenza virus disease/infection.

In certain embodiments, the methods for immunizing against influenza virus in a subject (e.g., a human or non-human animal) provided herein result in a hemagglutination inhibition (“HI”) antibody titer at least 1:40. In certain embodiments, the methods for immunizing against influenza virus in a subject (e.g., a human or non-human animal) provided herein result in seroconversion. In certain embodiments, seroconversion is when a subject has (a) an HI titer of less than 1:10 prior to being administered an immunogenic composition described herein, and (b) an HI titer of at least 1:40 and a minimum four-fold rise in HI antibody titer after being administered the immunogenic composition described herein. In certain embodiments, the methods for immunizing against influenza virus in a subject (e.g., a human or non-human animal) provided herein result in the lower bound of a two-sided 95% confidence interval (“CI”) for the percent of subjects receiving the immunogenic composition(s) of the methods achieving seroconversion for HI antibody meeting or exceeding 40%, wherein the subjects are less than 65 years of age. In certain embodiments, the methods for immunizing against influenza virus in a subject (e.g., a human or non-human animal) provided herein result in the lower bound of a two-sided 95% CI for the percent of subjects receiving the immunogenic composition(s) of the methods achieving an HI antibody titer of at least 1:40 meeting or exceeding 70%, wherein the subjects are less than 65 years of age. In certain embodiments, the methods for immunizing against influenza virus in a subject (e.g., a human or non-human animal) provided herein result in the lower bound of a two-sided 95% CI for the percent of subjects receiving the immunogenic composition(s) of the methods achieving seroconversion for HI antibody meeting or exceeding 30%, wherein the subjects are at least 65 years of age. In certain embodiments, the methods for immunizing against influenza virus in a subject (e.g., a human or non-human animal) provided herein result in the lower bound of a two-sided 95% CI for the percent of subjects receiving the immunogenic composition(s) of the method achieving an HI antibody titer of at least 1:40 meeting or exceeding 60%, wherein the subjects are at least 65 years of age. Techniques for determining HI antibody titer are known to one skilled in the art. Methods for evaluating the lower bound of the two-sided 95% CI are known to one skilled in the art. See, e.g., the U.S. Food & Drug Administration Guidance for Industry: Clinical Data Needed to Support the Licensure of Pandemic Influenza Vaccines and Guidance for Industry: Clinical Data Needed to Support the Licensure of Seasonal Inactivated Influenza Vaccines. In specific embodiments, a method of immunization described herein meet one, two, three, or more criteria for demonstrating effectiveness of an influenza vaccine as determined according to the U.S. Food & Drug Administration Guidance for Industry: Clinical Data Needed to Support the Licensure of Pandemic Influenza Vaccines or Guidance for Industry: Clinical Data Needed to Support the Licensure of Seasonal Inactivated Influenza Vaccines.

In a specific embodiment, the immune response induced by an immunogenic composition described herein induces HA stem-specific and HA globular head domain-specific antibodies (e.g., IgG). In another specific embodiment, the immune response induced by an immunogenic composition described herein induces antibodies with hemagglutination inhibition activity as assessed by a technique known to one of skill in the art or described herein (see, e.g., Section 5.9, infra and Section 6, infra). In another specific embodiment, the immune response induced by an immunogenic composition described herein induces antibodies with microneutralization activity as assessed by a technique known to one of skill in the art or described herein (see, e.g., Section 5.9, infra and Section 6, infra), such as an in vitro microneutralization assay or other assay. In another specific embodiment, the immune response induced by an immunogenic composition described herein induces antibodies with ADCC activity as assessed by a technique known to one of skill in the art or described herein (see, e.g., Section 5.9, infra and Section 6, infra). In another specific embodiment, the immune response induced by an immunogenic composition described herein induces antibodies with one, two or all of the following properties: (1) microneutralization activity as assessed by a technique known to one of skill in the art or described herein (see, e.g., Section 5.9, infra and Section 6, infra), (2) hemagglutination inhibition activity as assessed by a technique known to one of skill in the art or described herein (see, e.g., Section 5.9, infra and Section 6, infra), and (3) ADCC activity as assessed by a technique known to one of skill in the art or described herein (see, e.g., Section 5.9, infra and Section 6, infra). In another specific embodiment, the immune response induced by an immunogenic composition described herein induces antibodies with one, two or all of the properties of the antibodies described in Section 6, infra (e.g., Section 6.2 or Section 6.3, infra).

In another aspect, provided herein are methods for preventing an influenza virus disease in a subject utilizing an immunogenic composition described herein. In a specific embodiment, a method for preventing an influenza virus disease in a subject comprises administering to a subject in need thereof a subunit vaccine, a live virus vaccine, an inactivated virus vaccine, a split virus vaccine or a virus-like particle vaccine described herein. In a specific embodiment, a method for preventing an influenza virus disease in a subject comprises administering to a subject in need thereof a subunit vaccine described herein. In another embodiment, a method for preventing an influenza virus disease in a subject comprises administering to a subject in need thereof a live virus vaccine described herein. In particular embodiments, the live virus vaccine comprises an attenuated virus. In another embodiment, a method for preventing an influenza virus disease in a subject comprises administering to a subject in need thereof an inactivated virus vaccine described herein. In another embodiment, a method for preventing or an influenza virus disease in a subject comprises administering to a subject in need thereof a split virus vaccine described herein. In another embodiment, a method for preventing an influenza virus disease comprises administering to a subject in need thereof a virus-like particle vaccine described herein. In another embodiment, a method for preventing an influenza virus disease in a subject, comprising administering to a subject in need thereof a virosome described herein.

In another aspect, provided herein are methods for preventing an influenza virus disease, or treating an influenza virus infection or an influenza virus disease in a subject comprising administering to a subject an anti-influenza A virus HA antibody(ies), wherein the anti-influenza A virus HA antibody(ies) was generated utilizing a mosaic influenza virus HA polypeptide described herein. For example, a mosaic influenza virus HA polypeptide described herein may be administered to a non-human subject (e.g., a non-human subject that expresses or is capable of expression human antibody) to generate anti-influenza A virus HA antibody(ies). In a specific embodiment, provided herein is a method for preventing an influenza virus disease in a human subject comprising administering the subject a human or humanized anti-influenza A virus HA antibody(ies), wherein the anti-influenza A virus HA antibody(ies) was generated utilizing a mosaic influenza virus HA polypeptide described herein. In another specific embodiment, provided herein is a method for preventing an influenza virus disease in a human subject comprising administering the subject a human or humanized anti-influenza A virus HA antibody(ies), wherein the anti-influenza A virus HA antibody(ies) was isolated/identified utilizing a mosaic influenza virus HA polypeptide described herein. In a specific embodiment, provided herein is a method for preventing an influenza virus disease in a human subject comprising administering the subject a human or humanized anti-influenza A virus HA antibody(ies), wherein the anti-influenza A virus HA antibody(ies) was generated and isolated/identified utilizing a mosaic influenza virus HA polypeptide described herein. A hemagglutination inhibition assay, such as described herein (see, e.g., Section 6), which utilizes a mosaic influenza virus HA polypeptide may be useful in identifying an anti-influenza A virus HA antibody(ies). For example, a library of antibodies may be tested in a hemagglutination inhibition assay, which utilizes a mosaic influenza virus HA polypeptide to identify an anti-influenza A virus HA antibody(ies). An ELISA which measures anti-HA antibodies induced by a mosaic HA polypeptide may be useful.

In certain embodiments, the methods for preventing an influenza virus disease, or treating an influenza virus infection or an influenza virus disease in a subject (e.g., a human or non-human animal) provided herein result in a reduction in the replication of the influenza virus in the subject as measured by in vivo and in vitro assays known to those of skill in the art and described herein. In some embodiments, the replication of the influenza virus is reduced by approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. In specific embodiments, the methods for preventing an influenza virus disease, or treating an influenza virus infection or an influenza virus disease in a subject (e.g., a human or non-human animal) provided herein result in a reduction of the titer of an influenza virus detected in the subject. In specific embodiments, the methods for preventing an influenza virus disease, or treating an influenza virus infection or an influenza virus disease in a subject results in one, two, or more of the following: (1) reduces the number of symptoms of the infection/disease, (2) reduces the severity of the symptoms of the infection/disease, (3) reduces the length of the infection/disease, (4) reduces hospitalization or complications resulting from the infection/disease, (5) reduces the length of hospitalization of the subject, (6) reduces organ failure associated with the influenza virus infection/disease, and (7) increases survival of the subject. In a specific embodiment, the methods for preventing an influenza virus disease, or treating an influenza virus infection or an influenza virus disease in a subject inhibits the development or onset of an influenza virus disease or one or more symptoms thereof.

In certain embodiments, provided herein are methods for generating antibodies comprising administering an active compound (e.g., a mosaic influenza virus hemagglutinin polypeptide described herein, a nucleic acid sequence encoding such a polypeptide(s), or a vector (e.g., a viral vector) either containing, expressing, or both such polypeptide(s)) or composition described herein may be administered to a subject (e.g., a non-human subject). In some embodiments, an active compound (e.g., a mosaic influenza virus hemagglutinin polypeptide described herein, a nucleic acid sequence encoding such a polypeptide(s), or a vector (e.g., a viral vector) either containing, expressing, or both such polypeptide(s)) or composition described herein may be administered to a subject (e.g., a non-human subject) and the antibodies may be isolated. The isolated antibodies may be cloned. The antibodies may be humanized and/or optimized. In some embodiments, hybridomas are produced which produce a particular antibody of interest. Techniques for isolating, cloning, humanizing, optimizing and for generating hybridomas are known to one of skill in the art. In a specific embodiment, antibodies generated by a method described herein may be utilized in assays (e.g., assays described herein) as well as in passive immunization of a subject (e.g., a human subject). Thus, provided herein, in certain embodiments, are methods for treating influenza virus infection or preventing influenza virus disease, comprising administering antibodies generated by a method described herein.

### 5.7.1 COMBINATION THERAPIES

In various embodiments, a mosaic influenza virus hemagglutinin polypeptide described herein, a nucleic acid sequence encoding such a polypeptide(s), or a vector (e.g., a viral vector) either containing, expressing, or both such polypeptide(s), may be administered to a subject in combination with one or more other therapies (e.g., an antiviral, antibacterial, or immunomodulatory therapies). In some embodiments, a pharmaceutical composition (e.g., an immunogenic composition) described herein may be administered to a subject in combination with one or more therapies. The one or more other therapies may be beneficial in the prevention of an influenza virus disease or may ameliorate a symptom or condition associated with an influenza virus disease. In some embodiments, the one or more other therapies are pain relievers, anti-fever medications, or therapies that alleviate or assist with breathing. In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patient visit.

### 5.7.2 PATIENT POPULATIONS

In certain embodiments, an active compound (e.g., a mosaic hemagglutinin (HA) polypeptide, a nucleic acid sequence encoding such a polypeptide(s), or a vector (e.g., a viral vector) either containing, expressing, or both such polypeptide(s)), or composition described herein may be administered to a naïve subject, i.e., a subject that does not have a disease caused by influenza virus infection or has not been and is not currently infected with an influenza virus infection. In one embodiment, an active compound or composition described herein is administered to a naïve subject that is at risk of acquiring an influenza virus infection. In another embodiment, an active compound or composition described herein is administered to a subject that does not have a disease caused by the specific influenza virus, or has not been and is not infected with the specific influenza virus to which the mosaic hemagglutinin (HA) polypeptide induces an immune response. An active compound or composition described herein may also be administered to a subject that is and/or has been infected with the influenza virus or another type, subtype/lineage or strain of the influenza virus to which the mosaic hemagglutinin (HA) polypeptide induces an immune response.

In certain embodiments, an active compound (e.g., a mosaic hemagglutinin (HA) polypeptide described herein, a nucleic acid sequence encoding such a polypeptide(s), or a vector (e.g., a viral vector) either containing, expressing, or both such a polypeptide(s)), or composition described herein is administered to a patient who has been diagnosed with an influenza virus infection. In some embodiments, an active compound or composition described herein is administered to a patient infected with an influenza virus before symptoms manifest or symptoms become severe (e.g., before the patient requires hospitalization).

In some embodiments, a subject to be administered an active compound (e.g., a mosaic hemagglutinin (HA) described herein, a nucleic acid sequence encoding such a polypeptide(s), or a vector (e.g., a viral vector) either containing, expressing, or both such a polypeptide(s)) or composition described herein is an animal. In certain embodiments, the animal is a bird. In certain embodiments, the animal is a canine. In certain embodiments, the animal is a feline. In certain embodiments, the animal is a horse. In certain embodiments, the animal is a cow. In certain embodiments, the animal is a mammal, e.g., a horse, swine, mouse, or primate, preferably a human.

In specific embodiments, a subject administered an active compound (e.g., a mosaic hemagglutinin (HA) polypeptide described herein, a nucleic acid sequence encoding such a polypeptide(s), or a vector (e.g., a viral vector) either containing, expressing, or both such a polypeptide(s)) or composition described herein is a human infant. As used herein, the term “human infant” refers to a newborn to 1 year old human. In specific embodiments, a subject administered an active compound (e.g., a mosaic hemagglutinin (HA) polypeptide described herein, a nucleic acid sequence encoding such a polypeptide(s), or a vector (e.g., a viral vector) either containing, expressing, or both such a polypeptide(s)) or composition described herein is a human child. As used herein, the term “human child” refers to a human that is 1 year to 18 years old. In specific embodiments, a subject administered an active compound (e.g., a mosaic hemagglutinin (HA) polypeptide described herein, a nucleic acid sequence encoding such a polypeptide(s), or a vector (e.g., a viral vector) either containing, expressing, or both such a polypeptide(s)) or composition described herein is a human adult. As used herein, the term “human adult” refers to a human that is 18 years or older. In specific embodiments, a subject administered an active compound (e.g., a mosaic hemagglutinin (HA) polypeptide described herein, a nucleic acid sequence encoding such a polypeptide(s), or a vector (e.g., a viral vector) either containing, expressing, or both such a polypeptide(s)) or composition described herein is an elderly human. As used herein, the term “elderly human” refers to a human 65 years or older.

In some embodiments, the human subject to be administered an active compound (e.g., a mosaic hemagglutinin (HA) polypeptide described herein, a nucleic acid sequence encoding such a polypeptide(s), or a vector (e.g., a viral vector) containing or expressing such a polypeptide(s)), or composition described herein is any individual at increased risk of influenza virus infection or disease resulting from influenza virus infection (e.g., an immunocompromised or immunodeficient individual). In some embodiments, the human subject to be administered an active compound or composition described herein is any individual in close contact with an individual with increased risk of influenza virus infection or disease resulting from influenza virus infection (e.g., immunocompromised or immunosuppressed individuals).

In some embodiments, the human subject to be administered an active compound (e.g., a mosaic hemagglutinin (HA) polypeptide described herein, a nucleic acid sequence encoding such a polypeptide(s), or a vector (e.g., a viral vector) containing or expressing such a polypeptide(s)), or composition described herein is an individual affected by any condition that increases susceptibility to influenza virus infection or complications or disease resulting from influenza virus infection. In other embodiments, an active compound or composition described herein is administered to a subject in whom an influenza virus infection has the potential to increase complications of another condition that the individual is affected by, or for which they are at risk.

In certain embodiments, an immunogenic formulation comprising a live virus vector is not given concurrently with other live-virus vaccines.

### 5.8 Modes of Administration

### 5.8.1 ROUTES OF DELIVERY

An active compound (e.g., a mosaic hemagglutinin (HA) polypeptide, a nucleic acid sequence encoding such a polypeptide(s), or a vector (e.g., a viral vector) containing, expressing, or both such a polypeptide(s)), or composition described herein may be delivered to a subject by a variety of routes. These include, but are not limited to, intranasal, intratracheal, oral, intradermal, intramuscular, intraperitoneal, transdermal, intravenous, conjunctival and subcutaneous routes. In some embodiments, a composition is formulated for topical administration, for example, for application to the skin. In specific embodiments, the route of administration is nasal, e.g., as part of a nasal spray. In certain embodiments, a composition is formulated for intramuscular administration. In some embodiments, a composition is formulated for subcutaneous administration. In certain embodiments, a composition is not formulated for administration by injection. In specific embodiments for live virus vaccines, the vaccine is formulated for administration by a route other than injection.

In a specific embodiment, a subunit vaccine is administered intramuscularly. In another embodiment, a live influenza virus vaccine is administered intranasally. In another embodiment, an inactivated influenza virus vaccine (e.g., an inactivated whole virus vaccine, a split influenza virus vaccine, or subunit vaccine) is administered intramuscularly. In another embodiment, a split virus vaccine is administered intramuscularly. In another embodiment, a virus-like particle or composition thereof is administered intramuscularly.

### 5.8.2 DOSAGE AND FREQUENCY OF ADMINISTRATION

The amount of an active compound (e.g., a mosaic hemagglutinin (HA) polypeptide described herein, a nucleic acid sequence encoding such a polypeptide(s), or a vector (e.g., a viral vector) either containing, expressing, or both such a polypeptide(s)) or composition described herein which will be effective in the prevention of an influenza virus disease will depend on the nature of the disease, and can be determined by standard clinical techniques.

The precise dose to be employed in the formulation will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, health), whether the patient is human or an animal, other medications administered, and whether treatment is prophylactic or therapeutic. Usually, the patient is a human but nonhuman mammals including transgenic mammals can also be treated. Treatment dosages are optimally titrated to optimize safety and efficacy.

As used herein, the term “effective amount” in the context of administering a therapy to a subject refers to the amount of a therapy which may have a prophylactic effect(s), therapeutic effect(s), or both a prophylactic and therapeutic effect(s). In certain embodiments, an “effective amount” in the context of administration of a therapy to a subject refers to the amount of a therapy which is sufficient to achieve one, two, three, four, or more of the following effects: (i) reduce or ameliorate the severity of an influenza virus infection, disease or symptom associated therewith; (ii) reduce the duration of an influenza virus infection, disease or symptom associated therewith; (iii) prevent the progression of an influenza virus infection, disease or symptom associated therewith; (iv) cause regression of an influenza virus infection, disease or symptom associated therewith; (v) prevent the development or onset of an influenza virus infection, disease or symptom associated therewith; (vi) prevent the recurrence of an influenza virus infection, disease or symptom associated therewith; (vii) reduce or prevent the spread of an influenza virus from one cell to another cell, one tissue to another tissue, or one organ to another organ; (viii) prevent or reduce the spread of an influenza virus from one subject to another subject; (ix) reduce organ failure associated with an influenza virus infection; (x) reduce hospitalization of a subject; (xi) reduce hospitalization length; (xii) increase the survival of a subject with an influenza virus infection or disease associated therewith; (xiii) eliminate an influenza virus infection or disease associated therewith; (xiv) inhibit or reduce influenza virus replication; (xv) inhibit or reduce the entry of an influenza virus into a host cell(s); (xvi) inhibit or reduce replication of the influenza virus genome; (xvii) inhibit or reduce synthesis of influenza virus proteins; (xviii) inhibit or reduce assembly of influenza virus particles; (xix) inhibit or reduce release of influenza virus particles from a host cell(s); (xx) reduce influenza virus titer; and/or (xxi) enhance or improve the prophylactic or therapeutic effect(s) of another therapy.

In certain embodiments, the effective amount does not result in complete protection from an influenza virus disease, but results in a lower titer or reduced number of influenza viruses compared to an untreated subject with an influenza virus infection. In certain embodiments, the effective amount results in a 0.5 fold, 1 fold, 1.5 fold, 2 fold, 3 fold, 4 fold, 5 fold, 6 fold, 7 fold, 8 fold, 9 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer of influenza virus relative to an untreated subject with an influenza virus infection. In some embodiments, the effective amount results in a reduction in titer of influenza virus relative to an untreated subject with an influenza virus infection of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. Benefits of a reduction in the titer, number or total burden of influenza virus include, but are not limited to, less severe symptoms of the infection, fewer symptoms of the infection and a reduction in the length of the disease associated with the infection.

In certain embodiments, an effective amount of a therapy (e.g., a mosaic HA polypeptide described herein or a composition thereof) results in an anti-influenza virus HA titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-influenza virus HA titer in a blood sample from the subject prior to immunization. In certain embodiments, an effective amount of a therapy (e.g., a mosaic HA polypeptide described herein or a composition thereof) results in an anti-influenza virus HA stalk titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-influenza virus HA stalk titer in a blood sample from the subject prior to immunization.

In certain embodiments, the dose of a viral vector (e.g., an influenza virus) described herein may be 104 plaque forming units (PFU) to 108 PFU. In certain embodiments, the dose of a mosaic hemagglutinin (HA) polypeptide described herein (e.g., as provided in split virus vaccines and subunit vaccines) may range from about 1 μg to 150 μg. In certain embodiments, the dose of a mosaic hemagglutinin (HA) polypeptide described herein (e.g., as provided in split virus vaccines and subunit vaccines) is 15 μg. In certain embodiments, the dose for VLPs may range from about 1 μg to about 150 μg of a mosaic HA polypeptide. In certain embodiments, the dose for VLPs is 15 μg of a mosaic HA polypeptide. In some embodiments, an inactivated vaccine is formulated such that it may contain about 1 μg to about 150 μg of a mosaic hemagglutinin (HA) polypeptide described herein. In some embodiments, an inactivated vaccine is formulated such that it contains 15 μg of a mosaic hemagglutinin (HA) polypeptide described herein.

In certain embodiments, the dose of anti-influenza A virus HA antibody described herein may be 1 to 50 mg/kg. In some embodiments, the dose of anti-influenza A virus HA antibody described herein may be 10 to 15 mg/kg.

Exemplary doses for nucleic acids encoding a mosaic hemagglutinin (HA) polypeptide described herein range from about 10 ng to 1 g nucleic acid, e.g., DNA, cDNA, or RNA per patient.

In certain embodiments, a subject is administered a first active compound (e.g., a mosaic hemagglutinin (HA) polypeptide described herein, a nucleic acid sequence encoding such a polypeptide(s), or a vector (e.g., a viral vector) either containing, expressing, or both such a polypeptide(s)) or composition described herein and subsequently administered one, two or more boosters of the same or a different active compound or composition.

### 5.9 Biological Assays

Also provided herein are biological assays that may be used to characterize a mosaic HA, nucleic acid sequence encoding such mosaic HA, and viruses containing, expressing, or both such mosaic HA. See, also, Section 6. In a specific embodiment, an assay described in Section 6 is used to characterize a mosaic HA, a nucleic acid sequence encoding such mosaic HA, or virus containing, expressing, or both such mosaic HA. In another specific embodiment, the immunogenicity or effectiveness of an immunogenic composition described herein is assessed using one, two, or more assays described in Section 6.

### 5.9.1 ASSAYS FOR TESTING ACTIVITY OF MOSAIC INFLUENZA VIRUS HEMAGGLUTININ POLYPEPTIDES

Assays for testing the expression of a mosaic hemagglutinin (HA) polypeptide in a vector disclosed herein may be conducted using any assay known in the art. For example, an assay for incorporation into a viral vector comprises growing the virus as described in this Section, Section 5.2 above or Section 5.3 above, purifying the viral particles by centrifugation through a sucrose cushion, and subsequent analysis for mosaic hemagglutinin (HA) polypeptide expression by an immunoassay, such as western blotting, using methods well known in the art. Methods for determining whether a hemagglutinin polypeptide is mosaic are known to those of skill in the art. For example, the ability of a particular anti-influenza A virus HA antibody(ies) to bind and neutralize hemagglutination by a mosaic influenza virus HA polypeptide may be used to determine whether the HA globular head domain of the mosaic influenza virus HA polypeptide has been altered. See, e.g., the methods described in Section 6, infra. In another example, the nucleic acid sequence of a mosaic influenza virus HA polypeptide may be determined using a sequencing technique known to one of skill in the art.

In one embodiment, a mosaic hemagglutinin (HA) polypeptide disclosed herein is assayed for proper folding and functionality by testing its ability to bind specifically to a neutralizing antibody directed to an influenza virus hemagglutinin polypeptide, such as the stalk region of the mosaic HA polypeptide, using any assay for antibody-antigen interaction known in the art. Neutralizing antibodies for use in such assays include, for example, the neutralizing antibodies described in Ekiert et al., 2009, Science Express, 26 Feb. 2009; Kashyap et al., 2008, Proc Natl Acad Sci USA 105:5986-5991; Sui et al. 2009, Nature Structural and Molecular Biology, 16:265-273; Wang et al., 2010, PLOS Pathogens 6(2): 1-9; U.S. Pat. Nos. 5,589,174, 5,631,350, 6,337,070, and 6,720,409; International Application No. PCT/US2007/068983 published as International Publication No. WO 2007/134237; International Application No. PCT/US2008/075998 published as International Publication No. WO 2009/036157; International Application No. PCT/EP2007/059356 published as International Publication No. WO 2008/028946; and International Application No. PCT/US2008/085876 published as International Publication No. WO 2009/079259. These antibodies include CR6261, CR6325, CR6329, CR6307, CR6323, 2A, D7, D8, F10, G17, H40, A66, D80, E88, E90, H98, C179 (FERM BP-4517), AI3C (FERM BP-4516), among others.

In another embodiment, a mosaic hemagglutinin (HA) polypeptide disclosed herein is assayed for proper folding by determination of the structure or conformation of the mosaic hemagglutinin (HA) polypeptide using any method known in the art such as, e.g., NMR, X-ray crystallographic methods, or secondary structure prediction methods, e.g., circular dichroism.

In another embodiment, a mosaic HA disclosed herein is assayed for retention of one, two, or more, or all of the functions of a wild-type influenza HA. Nonlimiting examples of functions of a wild-type influenza HA include fusogenic activity, receptor binding activity, budding, and particle formation. In a specific embodiment, a mosaic HA disclosed herein is assayed for fusogenic activity. Assays known to one skilled in the art can be utilized the assess the fusogenic activity of a mosaic influenza hemagglutinin (HA) polypeptide described herein, such as, for example, immunofluorescence assays and pseudotyped virus-like-particle assays. In certain embodiments, the activity of a mosaic HA polypeptide described herein is assessed in one or more of the following assays: hemagglutination assay(s), fusion assay(s) or budding assay(s).

### 5.9.2 ASSAYS FOR TESTING ACTIVITY OF ANTIBODIES GENERATED USING MOSAIC INFLUENZA VIRUS HEMAGGLUTININ POLYPEPTIDES

Antibodies generated using a mosaic HA polypeptide may be characterized in a variety of ways known to one of skill in the art (e.g. ELISA, Surface Plasmon resonance display (BIAcore), western blot, immunofluorescence, immunostaining, biolayer interferometry, and/or microneutralization assays). In some embodiments, antibodies are assayed for the ability to specifically bind to a mosaic hemagglutinin (HA) polypeptide, or a vector comprising said polypeptide. Such an assay may be performed in solution (e.g., Houghten, 1992, Bio/Techniques 13:412 421), on beads (Lam, 1991, Nature 354:82 84), on chips (Fodor, 1993, Nature 364:555 556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865 1869) or on phage (Scott and Smith, 1990, Science 249:386 390; Cwirla et al., 1990, Proc. Natl. Acad. Sci. USA 87:6378 6382; and Felici, 1991, J. Mol. Biol. 222:301 310) (each of these references is incorporated herein in its entirety by reference).

Specific binding of an antibody to a mosaic hemagglutinin (HA) polypeptide or a domain thereof and cross-reactivity with other antigens can be assessed by any method known in the art. Immunoassays which can be used to analyze specific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety).

The binding affinity of an antibody to a mosaic hemagglutinin (HA) polypeptide or a domain thereof and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody for a mosaic hemagglutinin (HA) polypeptide and the binding off-rates can be determined from the data by Scatchard plot analysis. Competition with a second antibody can also be determined using radioimmunoassays. In this case, a mosaic hemagglutinin (HA) polypeptide is incubated with the test antibody conjugated to a labeled compound (e.g., ³H or ¹²⁵I) in the presence of increasing amounts of an unlabeled second antibody.

In a specific embodiment, the binding affinity of an antibody to a mosaic HA polypeptide or a domain thereof is determined using an assay described in Nachbagauer et al., mBio. 2016 January-February; 7(1): e01996-15.

In certain embodiments, antibody binding affinity and rate constants are measured using the KinExA 3000 System (Sapidyne Instruments, Boise, ID). In some embodiments, surface plasmon resonance (e.g., BIAcore kinetic) analysis is used to determine the binding on and off rates of the antibodies to an influenza virus hemagglutinin polypeptide. In specific embodiments, an assay described in Tan et al., PLoS Pathog. 2016 April; 12(4): e1005578 is used to determine the binding on and off rates of antibodies to a mosaic HA polypeptide.

The neutralizing activity of an antibody can be determined utilizing any assay known to one skilled in the art. Antibodies described herein can be assayed for their ability to inhibit the binding of an influenza virus, or any other composition comprising a mosaic hemagglutinin (HA) polypeptide, to its host cell receptor (i.e., sialic acid) using techniques known to those of skill in the art. In a specific embodiment, an assay described in one of the following articles is used to determine the neutralizing activity of an antibody Tan et al., PLoS Pathog. 2016 April; 12(4): e1005578; Pica et al., Proc Natl Acad Sci USA. 2012 Feb. 14; 109(7): 2573-2578; and Nachbagauer et al., mBio. 2016 January-February; 7(1): e01996-15.

In other embodiments, an antibody suitable for use in the methods described herein does not inhibit influenza virus receptor binding, yet is still found to be neutralizing in an assay described herein. In some embodiments, an antibody suitable for use in accordance with the methods described herein reduces or inhibits virus-host membrane fusion in an assay known in the art or described herein.

In one embodiment, virus-host membrane fusion is assayed in an in vitro assay using an influenza virus containing a reporter and a host cell capable of being infected with the virus. An antibody inhibits fusion if reporter activity is inhibited or reduced compared to a negative control (e.g., reporter activity in the presence of a control antibody or in the absence of antibody). In a specific embodiment, a reporter assay described in Heaton et al., J Virol. 2013 August; 87(15):8272-81 is used.

In certain embodiments, the microneutralization activity of antibodies induced by a mosaic HA polypeptide or composition described herein is assessed using a technique known to one of skill in the art or described herein (see, e.g., Section 6, infra for an exemplary in vitro microneutralization assay). In some embodiments, the hemagglutination inhibition activity of antibodies induced by a mosaic HA polypeptide or composition described herein is assessed using a technique known to one of skill in the art or described herein (see, e.g., Section 6, infra for an exemplary assay). In certain embodiments, the ADCC activity of antibodies induced by a mosaic HA polypeptide or composition described herein is assessed using a technique known to one of skill in the art or described herein (see, e.g., Section 6, infra for an exemplary in vitro assay).

### 5.9.3 CYTOTOXICITY ASSAYS

Many assays well-known in the art can be used to assess viability of cells (infected or uninfected) or cell lines following exposure to an active compound or a composition thereof and, thus, determine the cytotoxicity of the compound or composition. For example, cell proliferation can be assayed by measuring Bromodeoxyuridine (BrdU) incorporation (See, e.g., Hoshino et al., 1986, Int. J. Cancer 38, 369; Campana et al., 1988, J. Immunol. Meth. 107:79), (3H) thymidine incorporation (See, e.g., Chen, J., 1996, Oncogene 13:1395-403; Jeoung, J., 1995, J. Biol. Chem. 270:18367 73), by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability.

In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes. These changes are given a designation of T (100% toxic), PVH (partially toxic—very heavy—80%), PH (partially toxic—heavy—60%), P (partially toxic—40%), Ps (partially toxic—slight—20%), or 0 (no toxicity—0%), conforming to the degree of cytotoxicity seen. A 50% cell inhibitory (cytotoxic) concentration (IC₅₀) is determined by regression analysis of these data.

In a specific embodiment, the cells used in the cytotoxicity assay are animal cells, including primary cells and cell lines. In some embodiments, the cells are human cells. In certain embodiments, cytotoxicity is assessed in one or more of the following cell lines: U937, a human monocyte cell line; primary peripheral blood mononuclear cells (PBMC); Huh7, a human hepatoblastoma cell line; 293T, a human embryonic kidney cell line; and THP-1, monocytic cells. In certain embodiments, cytotoxicity is assessed in one or more of the following cell lines: MDCK, MEF, Huh 7.5, Detroit, or human tracheobronchial epithelial (HTBE) cells.

Active compounds or compositions thereof can be tested for in vivo toxicity in animal models. For example, animal models, described herein and/or others known in the art, used to test the activities of active compounds can also be used to determine the in vivo toxicity of these compounds. For example, animals are administered a range of concentrations of active compounds. Subsequently, the animals are monitored over time for lethality, weight loss or failure to gain weight, and/or levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and/or regimen in addition to dosages.

Further, any assays known to those skilled in the art can be used to evaluate the prophylactic utility of the active compounds and compositions described herein, for example, by measuring viral infection or a condition or symptoms associated therewith.

The cytotoxicity assays described herein and known to those skilled in the art are particularly useful for live attenuated influenza viruses.

### 5.9.4 IN VIVO ANTIVIRAL ACTIVITY

Active compounds and compositions thereof are preferably assayed in vivo for the desired prophylactic activity prior to use in humans. For example, in vivo assays can be used to determine whether it is preferable to administer an active compound or composition thereof and/or another therapy. For example, to assess the use of an active compound or composition thereof to prevent an influenza virus disease, the composition can be administered before the animal is infected with influenza virus. Alternatively, or in addition, an active compound or composition thereof can be administered to the animal at the same time that the animal is infected with influenza virus. In a specific embodiment, an active compound or composition thereof is administered to the animal more than one time.

Active compounds and compositions thereof can be tested for antiviral activity in animal model systems including, but are not limited to, rats, mice, chicken, cows, monkeys, pigs, ferrets, goats, sheep, dogs, rabbits, guinea pigs, etc. In a specific embodiment, active compounds and compositions thereof are tested in a mouse model system. Such model systems are widely used and well-known to the skilled artisan. In a specific embodiment, active compounds and compositions thereof are tested in a mouse model system. Non-limiting examples of animal models for influenza virus are provided in this Section.

In general, animals are infected with influenza virus and concurrently treated with an active compound or composition thereof, or placebo. Alternatively, animals are treated with an active compound or composition thereof or placebo and subsequently infected with influenza virus. Samples obtained from these animals (e.g., serum, urine, sputum, semen, saliva, plasma, or tissue sample) can be tested for viral replication via well known methods in the art, e.g., those that measure altered viral titers (as determined, e.g., by plaque formation), the production of viral proteins (as determined, e.g., by Western blot, ELISA, or flow cytometry analysis) or the production of viral nucleic acids (as determined, e.g., by RT-PCR or northern blot analysis). For quantitation of virus in tissue samples, tissue samples are homogenized in phosphate-buffered saline (PBS), and dilutions of clarified homogenates are adsorbed for 1 hour at 37° C. onto monolayers of cells (e.g., Vero, CEF or MDCK cells). In other assays, histopathologic evaluations are performed after infection, preferably evaluations of the organ(s) the virus is known to target for infection. Virus immunohistochemistry can be performed using a viral-specific monoclonal antibody.

The effect of an active compound or composition thereof on the virulence of a virus can also be determined using in vivo assays in which the titer of the virus in an infected subject administered an active compound or composition thereof, the length of survival of an infected subject administered an active compound or composition thereof, the immune response in an infected subject administered an active compound or composition thereof, the number, duration and/or severity of the symptoms in an infected subject administered an active compound or composition thereof, and/or the time period before onset of one or more symptoms in an infected subject administered an active compound or composition thereof, is assessed. Techniques known to one of skill in the art can be used to measure such effects. In certain embodiments, an active compound or composition thereof results in a 0.5 fold, 1 fold, 2 fold, 4 fold, 6 fold, 8 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer of influenza virus relative to an untreated subject. In some embodiments, an active compound or composition thereof results in a reduction in titer of influenza virus relative to an untreated subject of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs.

Influenza virus animal models, such as ferret, mouse, guinea pig, squirrel monkey, macaque, and chicken, developed for use to test antiviral agents against influenza virus have been described. See, e.g., Sidwell et al., Antiviral Res., 2000, 48:1-16; Lowen A. C. et al. PNAS., 2006, 103:9988-92; and McCauley et al., Antiviral Res., 1995, 27:179-186 and Rimmelzwann et al., Avian Diseases, 2003, 47:931-933. For mouse models of influenza, non-limiting examples of parameters that can be used to assay antiviral activity of active compounds administered to the influenza-infected mice include pneumonia-associated death, serum al-acid glycoprotein increase, animal weight, lung virus assayed by hemagglutinin, lung virus titers assayed by plaque assays, and histopathological change in the lung. Statistical analysis is carried out to calculate significance (e.g., a P value of 0.05 or less).

In other assays, histopathologic evaluations are performed after infection of an animal model subject. Nasal turbinates and trachea may be examined for epithelial changes and subepithelial inflammation. The lungs may be examined for bronchiolar epithelial changes and peribronchiolar inflammation in large, medium, and small or terminal bronchioles. The alveoli are also evaluated for inflammatory changes. The medium bronchioles are graded on a scale of 0 to 3+ as follows: 0 (normal: lined by medium to tall columnar epithelial cells with ciliated apical borders and basal pseudostratified nuclei; minimal inflammation); 1+ (epithelial layer columnar and even in outline with only slightly increased proliferation; cilia still visible on many cells); 2+ (prominent changes in the epithelial layer ranging from attenuation to marked proliferation; cells disorganized and layer outline irregular at the luminal border); 3+ (epithelial layer markedly disrupted and disorganized with necrotic cells visible in the lumen; some bronchioles attenuated and others in marked reactive proliferation).

The trachea is graded on a scale of 0 to 2.5+ as follows: 0 (normal: Lined by medium to tall columnar epithelial cells with ciliated apical border, nuclei basal and pseudostratified. Cytoplasm evident between apical border and nucleus. Occasional small focus with squamous cells); 1+ (focal squamous metaplasia of the epithelial layer); 2+ (diffuse squamous metaplasia of much of the epithelial layer, cilia may be evident focally); 2.5+ (diffuse squamous metaplasia with very few cilia evident).

Virus immunohistochemistry is performed using a viral-specific monoclonal antibody (e.g. NP-, N- or HN-specific monoclonal antibodies). Staining is graded 0 to 3+ as follows: 0 (no infected cells); 0.5+ (few infected cells); 1+ (few infected cells, as widely separated individual cells); 1.5+ (few infected cells, as widely separated singles and in small clusters); 2+ (moderate numbers of infected cells, usually affecting clusters of adjacent cells in portions of the epithelial layer lining bronchioles, or in small sublobular foci in alveoli); 3+ (numerous infected cells, affecting most of the epithelial layer in bronchioles, or widespread in large sublobular foci in alveoli).

In one example, the ability to induce lung lesions and cause infection in an animal model of virus infection is compared using wild-type virus and mock virus. Lung lesions can be assessed as a percentage of lung lobes that are healthy by visual inspection. Animals are euthanized 5 days p.i. by intravenous administration of pentobarbital, and their lungs are removed in toto. The percentage of the surface of each pulmonary lobe that is affected by macroscopic lesions is estimated visually. The percentages are averaged to obtain a mean value for the 7 pulmonary lobes of each animal. In other assays, nasal swabs can be tested to determine virus burden or titer. Nasal swabs can be taken during necropsy to determine viral burden post-infection.

In one embodiment, virus is quantified in tissue samples. For example, tissue samples are homogenized in phosphate-buffered saline (PBS), and dilutions of clarified homogenates adsorbed for 1 h at 37° C. onto monolayers of cells (e.g., MDCK cells). Infected monolayers are then overlaid with a solution of minimal essential medium containing 0.1% bovine serum albumin (BSA), 0.01% DEAE-dextran, 0.1% NaHCO₃, and 1% agar. Plates are incubated 2 to 3 days until plaques could be visualized. Tissue culture infectious dose (TCID) assays to titrate virus from PR8-infected samples are carried out as follows. Confluent monolayers of cells (e.g., MDCK cells) in 96-well plates are incubated with log dilutions of clarified tissue homogenates in media. Two to three days after inoculation, 0.05-ml aliquots from each well are assessed for viral growth by hemagglutination assay (HA assay).

### 5.10 Assessment of Antibodies in a Subject

In another aspect, a mosaic influenza virus hemagglutinin polypeptide described herein, or virus either expressing, containing, or both a mosaic influenza virus hemagglutinin polypeptide described herein, can be used to assess the antibody response of a subject (e.g., a naive subject or an immunized/vaccinated subject) or a population of subjects to an influenza virus hemagglutinin polypeptide (e.g., a mosaic influenza virus hemagglutinin polypeptide (see, e.g., Example 8 of International Publication No. WO 2013/043729, which is incorporated herein by reference in its entirety). In specific embodiments, a mosaic influenza virus hemagglutinin polypeptide or a virus either, containing, expressing, or both, a mosaic influenza virus hemagglutinin polypeptide can be used to assess the presence of stem-specific antibodies in the subject or population of subjects.

In a specific embodiment, the antibody response of a subject or a population of subjects that has been an immunized/vaccinated with a mosaic influenza virus hemagglutinin polypeptide, or a virus expressing and/or containing a mosaic influenza virus hemagglutinin polypeptide, is assessed to identify the types of stalk-specific antibodies in the subject or population of subjects (see, e.g., Section 6). Such an assessment may allow for the identification surrogate markers/endpoints important in determining the clinical response to administration of a, mosaic influenza virus hemagglutinin polypeptide) described herein, or a virus expressing and/or a mosaic influenza virus hemagglutinin polypeptide described herein. In such an approach, a biological sample, e.g., blood, from the subject or population of subjects may be isolated and tested directly for the presence of antibodies, or may be processed (e.g., to obtain sera) and subsequently tested for the presence of antibodies.

In another specific embodiment, the antibody profile of a naive subject (i.e., a subject that has not been immunized/vaccinated with a mosaic influenza virus hemagglutinin polypeptide described herein, or a virus containing and/or expressing a mosaic influenza virus hemagglutinin polypeptide or a population of naive subjects is assessed to determine whether said subject or population of subjects possesses globular head-specific and/or stem specific antibodies against various influenza virus strains or subtypes. Such an assessment may allow for the generation of a mosaic influenza virus hemagglutinin polypeptide), or viruses containing and/or expressing a mosaic influenza virus hemagglutinin polypeptide, that are suitable for administration to said subject or population of subjects, e.g., a mosaic influenza virus hemagglutinin polypeptide, comprising an HA globular head domain to which said subject or population of subjects is naive (does not have antibodies against). Such an assessment may determine an immunization strategy for the patient.

In another specific embodiment, provided herein is a method of assessing/detecting the presence of antibodies in a subject that are specific for a stem domain of a particular influenza virus strain or subtype comprising contacting in vitro a biological sample (e.g., blood, sera) from said subject with a mosaic influenza virus hemagglutinin polypeptide described herein. In another specific embodiment, provided herein is a method of assessing/detecting the presence of antibodies in a subject that are specific for a stem domain of a particular influenza virus strain or subtype comprising contacting in vitro a biological sample (e.g., blood, sera) from said subject with a virus expressing and/or containing a mosaic influenza virus hemagglutinin polypeptide described herein.

In specific embodiment, the antibody response of a subject administered an active compound or composition described herein, or another influenza virus vaccine (e.g., a seasonal influenza virus vaccine) may be assessed in a hemagglutination inhibition assay, such as described in Section 6 below.

In a specific embodiment, provided herein is a method of determining a change in a subject's (e.g., a human subject's) immune response to a first influenza A virus, comprising: a. measuring hemagglutination inhibition in a series of wells containing red blood cells and either inactivated plasma or sera from the subject from a first time point or inactivated plasma or sera from the subject from a second time point, wherein each of the series of wells contains a different influenza A virus, wherein each of the different influenza A viruses comprises a different mosaic influenza virus HA polypeptide, wherein each mosaic influenza virus HA polypeptide comprises an HA ectodomain of the first influenza A virus HA, wherein the HA ectodomain comprises an HA stem domain of the first influenza A virus HA and an HA globular head domain of the first influenza A virus HA, and wherein the HA globular head domain of the first influenza A virus HA has been engineered to comprise amino acid substitutions in one, two, three, four or more of the antigenic sites; and b. comparing the hemagglutination inhibition in each of the wells, wherein a difference in the inhibition of the hemagglutination in wells containing the plasma or sera from the first time point relative to the inhibition of hemagglutination in wells containing the plasma or sera from the second time point indicates a change in the subject's immune response to the first influenza A virus. Changes in a subject's hemagglutination inhibition profile may indicate active influenza infection, aging, or subsequent immunizations. In certain embodiments, the difference is an increase in inhibition of hemagglutination using inactivated plasma or sera from the second time point relative to the inhibition of hemagglutinin using inactivated plasma or sera from the first time point. In a specific embodiment, the change in the subject's immune response to the first influenza A virus is an improvement. In a specific embodiment, the hemagglutination inhibition assay may be conducted as described in Section 6, infra. In certain embodiments, the first time point is prior to vaccination with an influenza virus vaccine and the second time point is post-vaccination. In some embodiments, the first time point is 6 months, 1 year, 2 years, 3 years, 4 years, 5 years, 6 years, 7 years, 8 years, 9 years, 10 years or more before the second time point. In certain embodiments, the first time point is 1 to 5 years, 1 to 10 years, 5 to 10 years, 5 to 15 years, 10 to 15 years, 10 to 20 years, 15 to 25 years, 25 years to 40 years before the second time point.

### 5.11 Kits

Provided herein is a pharmaceutical pack or kit for immunizing against an influenza virus in a subject comprising one or more containers filled with one or more of the ingredients of the pharmaceutical/immunogenic compositions described herein, such as one or more active compounds provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in accordance with the methods described herein. In one embodiment, a kit comprises an active compound described herein, (e.g., one or more mosaic influenza virus hemagglutinin polypeptides described herein (such as described in Section 5.1 above or Section 6), in one or more containers. In another embodiment, a kit comprises one or more immunogenic compositions described herein in one or more containers. In certain embodiments, a kit comprises a vaccine described herein, e.g., a split virus vaccine, a subunit vaccine, an inactivated influenza virus vaccine, or a live influenza virus vaccine, wherein said vaccine comprises one or more one or more mosaic influenza virus hemagglutinin polypeptides described herein and optionally, an adjuvant described herein (e.g., in Section 5.6.5 above or Section 6). In certain embodiments, a kit comprises a vaccine described herein, e.g., a split virus vaccine, a subunit vaccine, an inactivated influenza virus vaccine, or a live influenza virus vaccine, wherein said vaccine comprises one or more mosaic influenza virus hemagglutinin polypeptides. In a specific embodiment, provided herein are kits comprising a mosaic influenza virus hemagglutinin polypeptide described herein and instructions for using the mosaic influenza virus hemagglutinin polypeptide described herein to assess the antibodies present in a subject.

In certain embodiments, a kit described herein comprises: (a) a first container comprising an immunogenic composition described herein, wherein the immunogenic composition comprises a mosaic influenza virus hemagglutinin (HA) polypeptide described herein (e.g., described in Section 5.1 above or Section 6); and (b) a second container comprising an adjuvant described herein (e.g., in Section 5.6.5 above or Section 6). In specific embodiments, the immunogenic composition is an inactivated whole virus vaccine. In specific embodiments, the immunogenic composition is a split virus vaccine. In specific embodiments, the immunogenic composition is a subunit vaccine.

### 5.12 Some of the Sequences in Specification

| | SEQ |
|---|---|
| | ID |
| Sequence | NO: |
| KKGNS | 1 |
| PKLNQS | 2 |
| KKNST | 3 |
| PTIKRS | 4 |
| TTADQQSLYQNA | 5 |
| DAAEQTKLYQNP | 6 |
| INDKG | 7 |
| NNTTG | 8 |
| PHAGAK | 9 |
| PYQGKS | 10 |
| LSTASS | 11 |
| LNVPE | 12 |
| NNESFNWT-GVTQNGTSSACIRRSSSS | 13 |
| NNESFNWT-GVTQNGTSSACMRNGGNS | 14 |
| THL-NYK | 15 |
| GTDKDQIFLYAQ | 16 |
| THL-NQK | 17 |
| GTNQDQIFLYAQ | 18 |
| QNSSIGEICDS | 19 |
| PIG-KCKSE | 20 |
| ESTGINRLCMK | 21 |
| PIDNNCESK | 22 |
| RITVSTKRSQQAVIPNIGS | 23 |
| LRIGRS | 24 |
| RITVSTSTYQQAVIPNIGS | 25 |
| GFQNKKWDLFVERSKAY | 27 |
| IRSGKS | 28 |
| GFQNKMWDLFVERSKAY | 29 |
| Mosaic H1 | 30 |
| DTLCIGYHANNSTDTVDTVLEKNVTVTHSVNLLEDKHNGKLCKLRGVAPLH | |
| LGKCNIAGWILGNPECESLNVPEWSYIVETSNSDNGTCYPGDFINYEELREQLS | |
| SVSSFERFEIFPKTSSWPSHDSNKGVTAACPYQGKSSFYKNLIWLVKKNSTYPT | |
| IKRSYNNTTGKEVLVLWGIHHPSDAAEQTKLYQNPDAYVFVGTSSYSKKFKP | |
| EIATRPKVNDQEGRMNYYWTLVHPGDKITFEATGNLVVPRYAFTMERNAGS | |
| GIIISDTPVHDCNTTCQTPEGAINTSLPFQNIHPITIGKCPKYVKSTKLRLATGLR | |
| NVPSIQSRGLFGAIAGFIEGGWTGMVDGWYGYHHQNEQGSGYAADLKSTQN | |
| AIDKITNKVNSVIEKMNTQFTAVGKEFNHLEKRIENLNKKVDDGFLDIWTYN | |
| AELLVLLENERTLDYHDSNVKNLYEKVRNQLKNNAKEIGNGCFEFYHKCDN | |
| TCMESVKNGTYDYPKYSEEAKLNREKIDGVKLESTRIYQILAIYSTVASSLVL | |
| VVSLGAISFWMCSNGSLQCRICI* | |
| Mosaic H3 | 31 |
| QKIPGNDNSTATLCLGHHAVPNGTIVKTITNDRIEVTNATELVESTGINRLCMK | |
| PHQILDGGNCHLIDALLGDPQCDGFQNKMWDLFVERSKAYSNCYPYDVPDY | |
| ASLRSLVASSGTLEFNNESFNWTGVTQNGTSSACMRNGGNSFFSRLNWLTHL | |
| NQKYPALNVTMPNNEQFDKLYIWGVHHPGTNQDQIFLYAQSSGRITVSTSTY | |
| QQAVIPNIGSRPRIRDIPSRISIYWTIVKPGDILLINSTGNLIAPRGYFKLRIGRSSI | |
| MRSDAPIDNNCESKCITPNGSIPNDKPFQNVNRITYGACPRYVKHSTLKLATG | |
| MRNVPEKQTRGIFGAIAGFIENGWEGMVDGWYGFRHQNSEGRGQAADLKST | |
| QAAIDQINGKLNRLIGKTNEKFHQIEKEFSEVEGRIQDLEKYVEDTKIDLWSYN | |
| AELLVALENQHTIDLTDSEMNKLFEKTKKQLRENAEDMGNGCFKIYHKCDN | |
| ACIGSIRNGTYDHNVYRDEALNNRFQIKGVELKSGYKDWILWISFAISCFLLC | |
| VALLGFIMWACQKGNIRCNICI* | |
| Signal Sequence of A/Michigan/45/2015 HA | 32 |
| MKAILVVLLYTFTTANA | |
| Ectodomain of A/Michigan/45/2015 HA | 33 |
| DTLCIGYHANNSTDTVDTVLEKNVTVTHSVNLLEDKHNGKLCKLRGVAPLH | |
| LGKCNIAGWILGNPECESLSTASSWSYIVETSNSDNGTCYPGDFINYEELREQL | |
| SSVSSFERFEIFPKTSSWPNHDSNKGVTAACPHAGAKSFYKNLIWLVKKGNSY | |
| PKLNQSYINDKGKEVLVLWGIHHPSTTADQQSLYQNADAYVFVGTSRYSKKF | |
| KPEIATRPKVRDQEGRMNYYWTLVEPGDKITFEATGNLVVPRYAFTMERNA | |
| GSGIIISDTPVHDCNTTCQTPEGAINTSLPFQNIHPITIGKCPKYVKSTKLRLATG | |
| LRNVPSIQSRGLFGAIAGFIEGGWTGMVDGWYGYHHQNEQGSGYAADLKST | |
| QNAIDKITNKVNSVIEKMNTQFTAVGKEFNHLEKRIENLNKKVDDGFLDIWT | |
| YNAELLVLLENERTLDYHDSNVKNLYEKVRNQLKNNAKEIGNGCFEFYHKC | |
| DNTCMESVKNGTYDYPKYSEEAKLNREKIDGVKLESTRIYQIL | |
| HA1 Domain of A/Michigan/45/2015 HA | 34 |
| DTLCIGYHANNSTDTVDTVLEKNVTVTHSVNLLEDKHNGKLCKLRGVAPLH | |
| LGKCNIAGWILGNPECESLSTASSWSYIVETSNSDNGTCYPGDFINYEELREQL | |
| SSVSSFERFEIFPKTSSWPNHDSNKGVTAACPHAGAKSFYKNLIWLVKKGNSY | |
| PKLNQSYINDKGKEVLVLWGIHHPSTTADQQSLYQNADAYVFVGTSRYSKKF | |
| KPEIATRPKVRDQEGRMNYYWTLVEPGDKITFEATGNLVVPRYAFTMERNA | |
| GSGIIISDTPVHDCNTTCQTPEGAINTSLPFQNIHPITIGKCPKYVKSTKLRLATG | |
| LRNVPSIQSR | |
| HA2 Domain of A/Michigan/45/2015 HA | 35 |
| GLFGAIAGFIEGGWTGMVDGWYGYHHQNEQGSGYAADLKSTQNAIDKITNK | |
| VNSVIEKMNTQFTAVGKEFNHLEKRIENLNKKVDDGFLDIWTYNAELLVLLE | |
| NERTLDYHDSNVKNLYEKVRNQLKNNAKEIGNGCFEFYHKCDNTCMESVKN | |
| GTYDYPKYSEEAKLNREKIDGVKLESTRIYQILAIYSTVASSLVLVVSLGAISF | |
| WMCSNGSLQCRICI | |
| Stem Domain of A/Michigan/45/2015 HA | 36 |
| DTLCIGYHANNSTDTVDTVLEKNVTVTHSVNLLEDKHNGKLC; | |
| CNTTCQTPEGAINTSLPFQNIHPITIGKCPKYVKSTKLRLATGLRNVPSIQSRGL | |
| FGAIAGFIEGGWTGMVDGWYGYHHQNEQGSGYAADLKSTQNAIDKITNKVN | |
| SVIEKMNTQFTAVGKEFNHLEKRIENLNKKVDDGFLDIWTYNAELLVLLENE | |
| RTLDYHDSNVKNLYEKVRNQLKNNAKEIGNGCFEFYHKCDNTCMESVKNGT | |
| YDYPKYSEEAKLNREKIDGVKLESTRIYQIL | |
| Globular Head Domain of A/Michigan/45/2015 HA | 37 |
| KLRGVAPLHLGKCNIAGWILGNPECESLSTASSWSYIVETSNSDNGTCYPGDF | |
| INYEELREQLSSVSSFERFEIFPKTSSWPNHDSNKGVTAACPHAGAKSFYKNLI | |
| WLVKKGNSYPKLNQSYINDKGKEVLVLWGIHHPSTTADQQSLYQNADAYVF | |
| VGTSRYSKKFKPEIATRPKVRDQEGRMNYYWTLVEPGDKITFEATGNLVVPR | |
| YAFTMERNAGSGIIISDTPVHD | |
| Transmembrane Domain of A/Michigan/45/2015 HA | 38 |
| AIYSTVASSLVLVVSLGAISF | |
| Cytoplasmic Domain of A/Michigan/45/2015 HA | 39 |
| WMCSNGSLQCRICI | |
| Signal Sequence of A/Hong Kong/4801/2014 | 40 |
| MKTIIALSYILCLVFA | |
| Ectodomain of A/Hong Kong/4801/2014 | 41 |
| QKIPGNDNSTATLCLGHHAVPNGTIVKTITNDRIEVTNATELVQNSSIGEICDS | |
| PHQILDGENCTLIDALLGDPQCDGFQNKKWDLFVERSKAYSNCYPYDVPDYA | |
| SLRSLVASSGTLEFNNESFNWTGVTQNGTSSACIRRSSSSFFSRLNWLTHLNYT | |
| YPALNVTMPNNEQFDKLYIWGVHHPGTDKDQIFLYAQSSGRITVSTKRSQQA | |
| VIPNIGSRPRIRDIPSRISIYWTIVKPGDILLINSTGNLIAPRGYFKIRSGKSSIMRS | |
| DAPIGKCKSECITPNGSIPNDKPFQNVNRITYGACPRYVKHSTLKLATGMRNV | |
| PEKQTRGIFGAIAGFIENGWEGMVDGWYGFRHQNSEGRGQAADLKSTQAAID | |
| QINGKLNRLIGKTNEKFHQIEKEFSEVEGRIQDLEKYVEDTKIDLWSYNAELL | |
| VALENQHTIDLTDSEMNKLFEKTKKQLRENAEDMGNGCFKIYHKCDNACIGS | |
| IRNGTYDHNVYRDEALNNRFQIKGVELKSGYKDWI | |
| HA1 Domain of A/Hong Kong/4801/2014 | 42 |
| QKIPGNDNSTATLCLGHHAVPNGTIVKTITNDRIEVTNATELVQNSSIGEICDS | |
| PHQILDGENCTLIDALLGDPQCDGFQNKKWDLFVERSKAYSNCYPYDVPDYA | |
| SLRSLVASSGTLEFNNESFNWTGVTQNGTSSACIRRSSSSFFSRLNWLTHLNYT | |
| YPALNVTMPNNEQFDKLYIWGVHHPGTDKDQIFLYAQSSGRITVSTKRSQQA | |
| VIPNIGSRPRIRDIPSRISIYWTIVKPGDILLINSTGNLIAPRGYFKIRSGKSSIMRS | |
| DAPIGKCKSECITPNGSIPNDKPFQNVNRITYGACPRYVKHSTLKLATGMRNV | |
| PEKQTR | |
| HA2 Domain of A/Hong Kong/4801/2014 | 43 |
| GIFGAIAGFIENGWEGMVDGWYGFRHQNSEGRGQAADLKSTQAAIDQINGK | |
| LNRLIGKTNEKFHQIEKEFSEVEGRIQDLEKYVEDTKIDLWSYNAELLVALEN | |
| QHTIDLTDSEMNKLFEKTKKQLRENAEDMGNGCFKIYHKCDNACIGSIRNGT | |
| YDHNVYRDEALNNRFQIKGVELKSGYKDWILWISFAISCFLLCVALLGFIM | |
| WACQKGNIRCNICI | |
| Stem Domain of A/Hong Kong/4801/2014 | 44 |
| QKIPGNDNSTATLCLGHHAVPNGTIVKTITNDRIEVTNATELVQNSSIGEIC; | |
| CKSECITPNGSIPNDKPFQNVNRITYGACPRYVKHSTLKLATGMRNVPEKQTR | |
| GIFGAIAGFIENGWEGMVDGWYGFRHQNSEGRGQAADLKSTQAAIDQINGKL | |
| NRLIGKTNEKFHQIEKEFSEVEGRIQDLEKYVEDTKIDLWSYNAELLVALENQ | |
| HTIDLTDSEMNKLFEKTKKQLRENAEDMGNGCFKIYHKCDNACIGSIRNGTY | |
| DHNVYRDEALNNRFQIKGVELKSGYKDWI | |
| Globular Head Domain of A/Hong Kong/4801/2014 | 45 |
| DSPHQILDGENCTLIDALLGDPQCDGFQNKKWDLFVERSKAYSNCYPYDVPD | |
| YASLRSLVASSGTLEFNNESFNWTGVTQNGTSSACIRRSSSSFFSRLNWLTHLN | |
| YTYPALNVTMPNNEQFDKLYIWGVHHPGTDKDQIFLYAQSSGRITVSTKRSQ | |
| QAVIPNIGSRPRIRDIPSRISIYWTIVKPGDILLINSTGNLIAPRGYFKIRSGKSSIM | |
| RSDAPIGK | |
| Transmembrane Domain of A/Hong Kong/4801/2014 | 46 |
| LWISFAISCFLLCVALLGFIM | |
| Cytoplasmic Domain of A/Hong Kong/4801/2014 | 47 |
| WACQKGNIRCNICI | |
| Signal Sequence of A/Jiangxi-Donghu/346-1/2013 HA | 48 |
| MYKIVVIIALLGAVKG | |
| Ectodomain of A/Jiangxi-Donghu/346-1/2013 HA | 49 |
| LDKICLGHHAVANGTIVKTLTNEQEEVTNATETVESTGINRLCMKGRKHKDL | |
| GNCHPIGMLIGTPACDLHLTGMWDTLIERENAIAYCYPGATVNVEALRQKIM | |
| ESGGINKISTGFTYGSSINSAGTTRACMRNGGNSFYAELKWLVSKSKGQNFPQ | |
| TTNTYRNTDTAEHLIMWGIHHPSSTQEKNDLYGTQSLSISVGSSTYRNNFVPV | |
| VGARPQVNGQSGRIDFHWTLVQPGDNITFSHNGGLIAPSRVSKLIGRGLGIQS | |
| DAPIDNNCESKCFWRGGSINTRLPFQNLSPRTVGQCPKYVNRRSLMLATGMR | |
| NVPELIQGRGLFGAIAGFLENGWEGMVDGWYGFRHQNAQGTGQAADYKST | |
| QAAIDQITGKLNRLVEKTNTEFESIESEFSEIEHQIGNVINWTKDSITDIWTYQA | |
| ELLVAMENQHTIDMADSEMLNLYERVRKQLRQNAEEDGKGCFEIYHACDDS | |
| CMESIRNNTYDHSQYREEALLNRLNINPVTLSSGYKDII | |
| HA1 Domain of A/Jiangxi-Donghu/346-1/2013 HA | 50 |
| LDKICLGHHAVANGTIVKTLTNEQEEVTNATETVESTGINRLCMKGRKHKDL | |
| GNCHPIGMLIGTPACDLHLTGMWDTLIERENAIAYCYPGATVNVEALRQKIM | |
| ESGGINKISTGFTYGSSINSAGTTRACMRNGGNSFYAELKWLVSKSKGQNFPQ | |
| TTNTYRNTDTAEHLIMWGIHHPSSTQEKNDLYGTQSLSISVGSSTYRNNFVPV | |
| VGARPQVNGQSGRIDFHWTLVQPGDNITFSHNGGLIAPSRVSKLIGRGLGIQS | |
| DAPIDNNCESKCFWRGGSINTRLPFQNLSPRTVGQCPKYVNRRSLMLATGMR | |
| NVPELIQGR | |
| HA2 Domain of A/Jiangxi-Donghu/346-1/2013 HA | 51 |
| GLFGAIAGFLENGWEGMVDGWYGFRHQNAQGTGQAADYKSTQAAIDQITG | |
| KLNRLVEKTNTEFESIESEFSEIEHQIGNVINWTKDSITDIWTYQAELLVAMEN | |
| QHTIDMADSEMLNLYERVRKQLRQNAEEDGKGCFEIYHACDDSCMESIRNNT | |
| YDHSQYREEALLNRLNINPVTLSSGYKDIILWFSFGASCFVLLAVVMGLFF | |
| FCLKNGNMRCTICI | |
| Stem Domain of A/Jiangxi-Donghu/346-1/2013 HA | 52 |
| LDKICLGHHAVANGTIVKTLTNEQEEVTNATETVESTGINRLC; | |
| CESKCFWRGGSINTRLPFQNLSPRTVGQCPKYVNRRSLMLATGMRNVPELIQ | |
| GRGLFGAIAGFLENGWEGMVDGWYGFRHQNAQGTGQAADYKSTQAAIDQI | |
| TGKLNRLVEKTNTEFESIESEFSEIEHQIGNVINWTKDSITDIWTYQAELLVAM | |
| ENQHTIDMADSEMLNLYERVRKQLRQNAEEDGKGCFEIYHACDDSCMESIRN | |
| NTYDHSQYREEALLNRLNINPVTLSSGYKDII | |
| Globular Head Domain of A/Jiangxi-Donghu/346-1/2013 HA | 53 |
| MKGRKHKDLGNCHPIGMLIGTPACDLHLTGMWDTLIERENAIAYCYPGATV | |
| NVEALRQKIMESGGINKISTGFTYGSSINSAGTTRACMRNGGNSFYAELKWLV | |
| SKSKGQNFPQTTNTYRNTDTAEHLIMWGIHHPSSTQEKNDLYGTQSLSISVGS | |
| STYRNNFVPVVGARPQVNGQSGRIDFHWTLVQPGDNITFSHNGGLIAPSRVSK | |
| LIGRGLGIQSDAPIDNN | |
| Transmembrane Domain of A/Jiangxi-Donghu/346-1/2013 HA | 54 |
| LWFSFGASCFVLLAVVMGLFF | |
| Cytoplasmic Domain of A/Jiangxi-Donghu/346-1/2013 HA | 126 |
| FCLKNGNMRCTICI | |
| Signal Sequence of A/Vietnam/1203/04 HA | 55 |
| MEKIVLLFAIVSLVKS | |
| Ectodomain of A/Vietnam/1203/04 HA | 56 |
| DQICIGYHANNSTEQVDTIMEKNVTVTHAQDILEKKHNGKLCDLDGVKPLILR | |
| DCSVAGWLLGNPMCDEFINVPEWSYIVEKANPVNDLCYPGDFNDYEELKHLL | |
| SRINHFEKIQIIPKSSWSSHEASLGVSSACPYQGKSSFFRNVVWLIKKNSTYPTI | |
| KRSYNNTNQEDLLVLWGIHHPNDAAEQTKLYQNPTTYISVGTSTLNQRLVPRI | |
| ATRSKVNGQSGRMEFFWTILKPNDAINFESNGNFIAPEYAYKIVKKGDSTIMK | |
| SELEYGNCNTKCQTPMGAINSSMPFHNIHPLTIGECPKYVKSNRLVLATGLRN | |
| SPQRERRRKKRGLFGAIAGFIEGGWQGMVDGWYGYHHSNEQGSGYAADKES | |
| TQKAIDGVTNKVNSIIDKMNTQFEAVGREFNNLERRIENLNKKMEDGFLDVW | |
| TYNAELLVLMENERTLDFHDSNVKNLYDKVRLQLRDNAKELGNGCFEFYHK | |
| CDNECMESVRNGTYDYPQYSEEARLKREEISGVKLESIGIYQIL | |
| HA1 Domain of A/Vietnam/1203/04 HA | 57 |
| DQICIGYHANNSTEQVDTIMEKNVTVTHAQDILEKKHNGKLCDLDGVKPLILR | |
| DCSVAGWLLGNPMCDEFINVPEWSYIVEKANPVNDLCYPGDENDYEELKHLL | |
| SRINHFEKIQIIPKSSWSSHEASLGVSSACPYQGKSSFFRNVVWLIKKNSTYPTI | |
| KRSYNNTNQEDLLVLWGIHHPNDAAEQTKLYQNPTTYISVGTSTLNQRLVPRI | |
| ATRSKVNGQSGRMEFFWTILKPNDAINFESNGNFIAPEYAYKIVKKGDSTIMK | |
| SELEYGNCNTKCQTPMGAINSSMPFHNIHPLTIGECPKYVKSNRLVLATGLRN | |
| SPQRERRRKKR | |
| HA2 Domain of A/Vietnam/1203/04 HA | 58 |
| GLFGAIAGFIEGGWQGMVDGWYGYHHSNEQGSGYAADKESTQKAIDGVTN | |
| KVNSIIDKMNTQFEAVGREFNNLERRIENLNKKMEDGFLDVWTYNAELLVL | |
| MENERTLDFHDSNVKNLYDKVRLQLRDNAKELGNGCFEFYHKCDNECMESV | |
| RNGTYDYPQYSEEARLKREEISGVKLESIGIYQILSIYSTVASSLALAIMVAGLS | |
| WMCSNGSLQCRICI | |
| Stem Domain of A/Vietnam/1203/04 HA | 59 |
| DQICIGYHANNSTEQVDTIMEKNVTVTHAQDILEKKHNGKLC; | |
| CNTKCQTPMGAINSSMPFHNIHPLTIGECPKYVKSNRLVLATGLRNSPQRERR | |
| RKKRGLFGAIAGFIEGGWQGMVDGWYGYHHSNEQGSGYAADKESTQKAID | |
| GVTNKVNSIIDKMNTQFEAVGREFNNLERRIENLNKKMEDGELDVWTYNAEL | |
| LVLMENERTLDFHDSNVKNLYDKVRLQLRDNAKELGNGCFEFYHKCDNEC | |
| MESVRNGTYDYPQYSEEARLKREEISGVKLESIGIYQIL | |
| Globular Head Domain of A/Vietnam/1203/04 HA | 60 |
| DLDGVKPLILRDCSVAGWLLGNPMCDEFINVPEWSYIVEKANPVNDLCYPGD | |
| FNDYEELKHLLSRINHFEKIQIIPKSSWSSHEASLGVSSACPYQGKSSFFRNVV | |
| WLIKKNSTYPTIKRSYNNTNQEDLLVLWGIHHPNDAAEQTKLYQNPTTYISVG | |
| TSTLNQRLVPRIATRSKVNGQSGRMEFFWTILKPNDAINFESNGNFIAPEYAYK | |
| IVKKGDSTIMKSELEYGN | |
| Transmembrane Domain of A/Vietnam/1203/04 HA | 61 |
| SIYSTVASSLALAIMVAGLSL | |
| Cytoplasmic Domain of A/Vietnam/1203/04 HA | 62 |
| WMCSNGSLQCRICI | |
| HA1 C-Terminal Stem Segment of A/Hong Kong/4801/2014 HA | 63 |
| CKSECITPNGSIPNDKPFQNVNRITYGACPRYVKHSTLKLATGMRNVPEKQTR | |
| HA1 N-Terminal Stem Segment of A/Hong Kong/4801/2014 HA | 64 |
| QKIPGNDNSTATLCLGHHAVPNGTIVKTITNDRIEVTNATELVQNSSIGEIC | |
| HA1 C-Terminal Stem Segment of A/Jiangxi-Donghu/346-1/2013 HA | 65 |
| CESKCFWRGGSINTRLPFQNLSPRTVGQCPKYVNRRSLMLATGMRNVPELIQ | |
| GR | |
| HA1 N-Terminal Stem Segment of A/Jiangxi-Donghu/346-1/2013 HA | 66 |
| LDKICLGHHAVANGTIVKTLTNEQEEVTNATETVESTGINRLC | |
| HA1 C-Terminal Stem Segment of A/Michigan/45/2015 HA | 67 |
| CNTTCQTPEGAINTSLPFQNIHPITIGKCPKYVKSTKLRLATGLRNVPSIQSR | |
| HA1 N-Terminal Stem Segment of A/Michigan/45/2015 HA | 68 |
| DTLCIGYHANNSTDTVDTVLEKNVTVTHSVNLLEDKHNGKLC | |
| HA1 C-Terminal Stem Segment of A/Vietnam/1203/04 HA | 69 |
| CNTKCQTPMGAINSSMPFHNIHPLTIGECPKYVKSNRLVLATGLRNSPQRERR | |
| RKKR | |
| HA1 N-Terminal Stem Segment of A/Vietnam/1203/04 HA | 70 |
| DQICIGYHANNSTEQVDTIMEKNVTVTHAQDILEKKHNGKLC | |
| HA2 Stem Domain of A/Hong Kong/4801/2014 HA | 71 |
| GIFGAIAGFIENGWEGMVDGWYGFRHQNSEGRGQAADLKSTQAAIDQINGKL | |
| NRLIGKTNEKFHQIEKEFSEVEGRIQDLEKYVEDTKIDLWSYNAELLVALENQ | |
| HTIDLTDSEMNKLFEKTKKQLRENAEDMGNGCFKIYHKCDNACIGSIRNGTY | |
| DHNVYRDEALNNRFQIKGVELKSGYKDWI | |
| HA2 Stem Domain of A/Jiangxi-Donghu/346-1/2013 HA | 72 |
| GLFGAIAGFLENGWEGMVDGWYGFRHQNAQGTGQAADYKSTQAAIDQITG | |
| KLNRLVEKTNTEFESIESEFSEIEHQIGNVINWTKDSITDIWTYQAELLVAMEN | |
| QHTIDMADSEMLNLYERVRKQLRQNAEEDGKGCFEIYHACDDSCMESIRNNT | |
| YDHSQYREEALLNRLNINPVTLSSGYKDII | |
| HA2 Stem Domain of A/Michigan/45/2015 HA | 73 |
| GLFGAIAGFIEGGWTGMVDGWYGYHHQNEQGSGYAADLKSTQNAIDKITNK | |
| VNSVIEKMNTQFTAVGKEFNHLEKRIENLNKKVDDGFLDIWTYNAELLVLLE | |
| NERTLDYHDSNVKNLYEKVRNQLKNNAKEIGNGCFEFYHKCDNTCMESVKN | |
| GTYDYPKYSEEAKLNREKIDGVKLESTRIYQIL | |
| HA2 Stem Domain of A/Vietnam/1203/2004 HA | 74 |
| GLFGAIAGFIEGGWQGMVDGWYGYHHSNEQGSGYAADKESTQKAIDGVTN | |
| KVNSIIDKMNTQFEAVGREFNNLERRIENLNKKMEDGFLDVWTYNAELLVL | |
| MENERTLDFHDSNVKNLYDKVRLQLRDNAKELGNGCFEFYHKCDNECMESV | |
| RNGTYDYPQYSEEARLKREEISGVKLESIGIYQIL | |
| Signal Sequence of A/black headed gull/Sweden/1/1999 HA | 75 |
| MDIPVVAFLILTSTCVQA | |
| Ectodomain of A/black headed gull/Sweden/1/1999 HA | 76 |
| DRICVGYLSTNSSEKVDTLLENDVPVTSSVDLVETNHTGTYCSLGGISPVHLG | |
| DCSFEGWIVGNPACASNLGIREWSYLIEDPSAPHGLCYPGELDNNGELRHLFS | |
| GIRSFSRTELIAPTSWGAVNDGVSSACPDKGASSFYRNLVWFVKRGNQYPVIR | |
| GTYNNTTGRDVLVIWGIHHPVSTDEAKQLYVNNNPYTLVSTSSWSRKYNLET | |
| GTRPGYNGQKSWMKIYWYLMHPGESISFESNGGLLAPRYGYIIEEYGKGRIFQ | |
| SRIRIAKCNTKCQTSVGGINTNKTFQNIERNALGDCPKYIKSGQLKLATGLRN | |
| VPAISNRGLFGAIAGFIEGGWPGLINGWYGFQHQNEQGVGMAADKESTQKAI | |
| DQITTKINNIIEKMNGNYDSIRGEFNQVEQRINMLADRIDDAVIDVWSYNAKL | |
| LVLLENDKTLDMHDANVRNLHDQVRRALKTNAIDEGNGCFELLHKCNDSCM | |
| ETIRNGTYNHTEYEEESKLKRQEIEGIKLKSDDSVYKAL | |
| HA1 Domain of A/black headed gull/Sweden/1/1999 HA | 77 |
| DRICVGYLSTNSSEKVDTLLENDVPVTSSVDLVETNHTGTYCSLGGISPVHLG | |
| DCSFEGWIVGNPACASNLGIREWSYLIEDPSAPHGLCYPGELDNNGELRHLFS | |
| GIRSFSRTELIAPTSWGAVNDGVSSACPDKGASSFYRNLVWFVKRGNQYPVIR | |
| GTYNNTTGRDVLVIWGIHHPVSTDEAKQLYVNNNPYTLVSTSSWSRKYNLET | |
| GTRPGYNGQKSWMKIYWYLMHPGESISFESNGGLLAPRYGYIIEEYGKGRIFQ | |
| SRIRIAKCNTKCQTSVGGINTNKTFQNIERNALGDCPKYIKSGQLKLATGLRN | |
| VPAISNR | |
| HA2 Domain of A/black headed gull/Sweden/1/1999 HA | 78 |
| GLFGAIAGFIEGGWPGLINGWYGFQHQNEQGVGMAADKESTQKAIDQITTKI | |
| NNIIEKMNGNYDSIRGEFNQVEQRINMLADRIDDAVIDVWSYNAKLLVLLEN | |
| DKTLDMHDANVRNLHDQVRRALKTNAIDEGNGCFELLHKCNDSCMETIRNG | |
| TYNHTEYEEESKLKRQEIEGIKLKSDDSVYKALSIYSCIASSIVLVGLILTFIMW | |
| ACSSGNCRFNICI | |
| Stem Domain of A/black headed gull/Sweden/1/1999 HA | 79 |
| DRICVGYLSTNSSEKVDTLLENDVPVTSSVDLVETNHTGTYC; | |
| CNTKCQTSVGGINTNKTFQNIERNALGDCPKYIKSGQLKLATGLRNVPAISNR | |
| GLFGAIAGFIEGGWPGLINGWYGFQHQNEQGVGMAADKESTQKAIDQITTKI | |
| NNIIEKMNGNYDSIRGEFNQVEQRINMLADRIDDAVIDVWSYNAKLLVLLEN | |
| DKTLDMHDANVRNLHDQVRRALKTNAIDEGNGCFELLHKCNDSCMETIRNG | |
| TYNHTEYEEESKLKRQEIEGIKLKSDDSVYKAL | |
| HA1 C-Terminal Stem Segment of A/black headed gull/Sweden/1/1999 HA | 80 |
| CNTKCQTSVGGINTNKTFQNIERNALGDCPKYIKSGQLKLATGLRNVPAISNR | |
| HA1 N-Terminal Stem Segment of A/black headed gull/Sweden/1/1999 HA | 81 |
| DRICVGYLSTNSSEKVDTLLENDVPVTSSVDLVETNHTGTYC | |
| Globular Head Domain of A/black headed gull/Sweden/1/1999 HA | 82 |
| SLGGISPVHLGDCSFEGWIVGNPACASNLGIREWSYLIEDPSAPHGLCYPGELD | |
| NNGELRHLFSGIRSFSRTELIAPTSWGAVNDGVSSACPDKGASSFYRNLVWFV | |
| KRGNQYPVIRGTYNNTTGRDVLVIWGIHHPVSTDEAKQLYVNNNPYTLVSTS | |
| SWSRKYNLETGTRPGYNGQKSWMKIYWYLMHPGESISFESNGGLLAPRYGYI | |
| IEEYGKGRIFQSRIRIAK | |
| Transmembrane Domain of A/black headed gull/Sweden/1/1999 HA | 83 |
| SIYSCIASSIVLVGLILTFIM | |
| Cytoplasmic Domain of A/black headed gull/Sweden/1/1999 HA | 84 |
| WACSSGNCRFNICI | |
| Exemplary influenza A HA subtype H1 signal peptide | 85 |
| MKANLLVLLCALAAADA | |
| Exemplary influenza A HA subtype H2 signal peptide | 86 |
| MAIIYLILLFTAVRG | |
| Exemplary influenza A HA subtype H3 signal peptide | 87 |
| MKTIIALSYIFCLALG | |
| HA2 Stem Domain of A/black headed gull/Sweden/1/1999 HA | 88 |
| GLFGAIAGFIEGGWPGLINGWYGFQHQNEQGVGMAADKESTQKAIDQITTKI | |
| NNIIEKMNGNYDSIRGEFNQVEQRINMLADRIDDAVIDVWSYNAKLLVLLEN | |
| DKTLDMHDANVRNLHDQVRRALKTNAIDEGNGCFELLHKCNDSCMETIRNG | |
| TYNHTEYEEESKLKRQEIEGIKLKSDDSVYKAL | |
| Exemplary influenza A HA subtype H4 and H5 signal peptide | 89 |
| MLSIVILFLLIAENSS | |
| Exemplary influenza A HA subtype H6 signal peptide | 90 |
| MIAIIVVAILATAGRS | |
| Exemplary influenza A HA subtype H7 signal peptide | 91 |
| MNTQILVFALVAVIPTNA | |
| Exemplary influenza A HA subtype H8 signal peptide | 92 |
| MEKFIAIATLASTNAY | |
| Exemplary influenza A HA subtype H9 signal peptide | 93 |
| METKAIIAALLMVTAA | |
| Exemplary influenza A HA subtype H10 signal peptide | 94 |
| MYKVVVIIALLGAVKG | |
| Exemplary influenza A HA subtype H11 signal peptide | 95 |
| MEKTLLFAAIFLCVKA | |
| Exemplary influenza A HA subtype H12 signal peptide | 96 |
| MEKFIILSTVLAASFAY | |
| Exemplary influenza A HA subtype H13 signal peptide | 97 |
| MALNVIATLTLISVCVHA | |
| Exemplary influenza A HA subtype H14 signal peptide | 98 |
| MIALILVALALSHTAYS | |
| Exemplary influenza A HA subtype H15 signal peptide | 99 |
| MNTQIIVILVLGLSMVKS | |
| Exemplary influenza A HA subtype H16 signal peptide | 100 |
| MMIKVLYFLIIVLGRYSKA | |
| Primer H3 | 101 |
| CCGAAGTTGGGGGGGAGCAAAAGCAGGGGATAATTC | |
| Primer Arev | 102 |
| GGCCGCCGGGTTATTAGTAGAAACAAGGGTGTTTTTAATTAATG | |
| Exemplary H1 HA | 103 |
| MKANLLVLLCALAAADADTICIGYHANNSTDTVDTVLEKNVTVTHSVNLLE | |
| DSHNGKLCRLKGIAPLQLGKCNIAGWLLGNPECDPLLPVRSWSYIVETPNSEN | |
| GICYPGDFIDYEELREQLSSVSSFERFEIFPKESSWPNHNTNGVTAACSHEGKSS | |
| FYRNLLWLTEKEGSYPKLKNSYVNKKGKEVLVLWGIHHPPNSKEQQNLYQN | |
| ENAYVSVVTSNYNRRFTPEIAERPKVRDQAGRMNYYWTLLKPGDTIIFEANG | |
| NLIAPMYAFALSRGFGSGIITSNASMHECNTKCQTPLG | |
| AINSSLPYQNIHPVTIGECPKYVRSAKLRMVTGLRNNPSIQSRGLFGAIAGFIEG | |
| GWTGMIDGWYGYHHQNEQGSGYAADQKSTQNAINGITNKVNTVIEKMNIQF | |
| TAVGKEFNKLEKRMENLNKKVDDGFLDIWTYNAELLVLLENERTLDFHDSN | |
| VKNLYEKVKSQLKNNAKEIGNGCFEFYHKCDNECMESVRNGTYDYPKYSEE | |
| SKLNREKVDGVKLESMGIYQILAIYSTVASSLVLLVSLGAISFWMCSNGSLQC | |
| RICI | |
| Exemplary H2 HA | 104 |
| MAIIYLILLFTAVRGDQICIGYHSNNSTEKVDTILERNVTVTHAQNILEKTHNG | |
| KLCKLNGIPPLELGDCSIAGWLLGNPECDRLLTVPEWSYIMEKENPRNGLCYP | |
| GSFNDYEELKHLLSSVTHFEKVKILPKDRWTQHTTTGGSRACAVSGNPSFFRN | |
| MVWLTKKGSNYPIAKGSYNNTSGEQMLIIWGVHHPSNDETEQRTLYQNVGT | |
| YVSIGTSTLNKRSIPVIATRPKVNGQGGRMEFSWTILDIWDTINFESTGNLIAPE | |
| YGFRISKRGSSGIMKTEGTLENCETKCQTPLGAINTTLPFHNVHPLTIGECPKY | |
| VKSERLVLATGLRNVPQIESRGLFGAIAGFIEGGWQGMIDGWYGYHHSNDQG | |
| SGYAADKESTQKAIDGITNRVNSVIEKMNTQFEAVGKEFSNLEKRLENLNKK | |
| MEDGFLDVWTYNAELLVLMENERTLDFHDSNVKNLYDRVRMQLRDNAKEL | |
| GNGCFEFYHKCDDECMNSVKNGTYDYPKYEEESKLNRNEIKGVKLSNMGVY | |
| QILAIYATVAGSLSLAIMIAGISLWMCSNGSLQCRICI | |
| Exemplary H3 HA | 105 |
| MKTIIALSYIFCLALGQDLPGNDNSTATLCLGHHAVPNGTLVKTITDDQIEVTN | |
| ATELVQSSSTGKICNNPHRILDGIDCTLIDALLGDPHCDVFQNETWDLFVERSK | |
| AFSNCYPYDVPDYASLRSLVASSGTLEFITEGFTWTGVTQNGGSNACKRGPG | |
| NGFFSRLNWLTKSGSTYPVLNVTMPNNDNFDKLYIWGVHHPSTNQEQTSLYV | |
| QESGRVTVSTRRSQQSIIPNIGSRPWVRGQSSRISIYWTIVKPGDVLVINSNGNL | |
| IAPRGYFKMRTGKSSIMSSDAPIDTCISECITPNGSIPNDKPFQNVNKITYGACP | |
| KYVKQNTLKLATGMRNVPEKQTRGLFGAIAGFIENGWEGMIDGWYGFRHQN | |
| SEGTGQAADLKSTQAAIDQINGKLNRVIEKTNEKFHQIEKEFSEVEGRIQDLEK | |
| YVEDTKIDLWSYNAELLVALENQHTIDLTDSEMNKLFEKTRRQLRENAEDMG | |
| NGCFKIYHKCDNACIESIRNGTYDHDVYRDEALNNRFQIKGVELKSGYKDWI | |
| LWISFAISCFLLCVVLLGFIMWACQRGNIRCNICI | |
| Exemplary H4 HA | 106 |
| MLSIVILFLLIAENSSQNYTGNPVICMGHHAVANGTMVKTLADDQVEVVTAQ | |
| ELVESQNLPELCPSPLRLVDGQTCDIINGALGSPGCDHLNGAEWDVFIERPNA | |
| VDTCYPFDVPEYQSLRSILANNGKFEFIAEEFQWNTVKQNGKSGACKRANVD | |
| DFFNRLNWLVKSDGNAYPLQNLTKINNGDYARLYIWGVHHPSTSTEQTNLY | |
| KNNPGRVTVSTKTSQTSVVPDIGSRPLVRGQSGRVSFYWTIVEPGDLIVENTIG | |
| NLIAPRGHYKLNNQKKSTILNTAIPIGSCVSKCHTDKGSLSTTKPFQNISRIAVG | |
| DCPRYVKQGSLKLATGMRNIPEKASRGLFGAIAGFIENGWQGLIDGWYGFRH | |
| QNAEGTGTAADLKSTQAAIDQINGKLNRLIEKTNDKYHQIEKEFEQVEGRIQD | |
| LENYVEDTKIDLWSYNAELLVALENQHTIDVTDSEMNKLFERVRRQLRENAE | |
| DKGNGCFEIFHKCDNNCIESIRNGTYDHDIYRDEAINNRFQIQGVKLTQGYKDI | |
| ILWISFSISCELLVALLLAFILWACQNGNIRCQICI | |
| Exemplary H5 HA | 107 |
| MERIVLLLAIVSLVKSDQICIGYHANKSTKQVDTIMEKNVTVTHAQDILERTH | |
| NGKLCSLNGVKPLILRDCSVAGWLLGNPMCDEFLNLPEWLYIVEKDNPINSLC | |
| YPGDFNDYEELKYLLSSTNHFEKIRIIPRSSWSNHDASSGVSSACPYIGRSSFLR | |
| NVVWLIKKNNTYPTIKRSYNNTNQEDLLILWGIHHPNDAAEQTKLYQNPTTY | |
| VSVGTSTLNQRSIPEIATRPKVNGQSGRMEFFWTILKPNDAINFESNGNFIAPR | |
| YAYKIVKKGDSAIMKSGLAYGNCDTKCQTPVGEINSSMPFHNIHPHTIGECPK | |
| YVKSDRLVLATGLRNVPQRKKRGLFGAIAGFIEGGWQGMVDGWYGYHHSN | |
| EQGSGYAADKESTQKAIDGITNKVNSIIDKMNTRFEAVGKEFNNLERRVENLN | |
| KKMEDGFLDVWTYNVELLVLMENERTLDFHDSNVNNLYDKVRLQLKDNAR | |
| ELGNGCFEFYHKCDNECMESVRNGTYDYPQYSEEARLNREEISGVKLESMGV | |
| YQILSIYSTVASSLALAIMIAGLSFWMCSNGSLQCRICI | |
| Exemplary H6 HA | 108 |
| MIAIIVVAILATAGRSDKICIGYHANNSTTQIDTILEKNVTVTHSVELLENQKEE | |
| RFCKILKKAPLDLKGCTIEGWILGNPQCDLLLGDQSWSYIVERPTAQNGICYP | |
| GVLNEVEELKALIGSGERVERFEMFPKSTWTGVDTSSGVTRACPYNSGSSFYR | |
| NLLWIIKTKSAAYSVIKGAYNNTGNQPILYFWGVHHPPDTNEQNTLYGSGDR | |
| YVRMGTESMNFAKSPEIAARPAVNGQRGRIDYYWSILKPGETLNVESNGNLI | |
| APWYAFRFVSTSNKGAVFKSNLPIENCDATCQTVAGVLRTNKTFQNVSPLWI | |
| GECPKYVKSESLRLATGLRNVPQIETRGLFGAIAGFIEGGWTGMIDGWYGYH | |
| HENSQGSGYAADRESTQKAVDGITNKVNSIIDKMNTQFEAVDHEFSNLERRID | |
| NLNKRMEDGFLDVWTYNAELLVLLENERTLDLHDANVKNLYERVKSQLRD | |
| NAMILGNGCFEFWHKCDDECMESVKNGTYDYPKYQDESKLNRQEIESVKLE | |
| SLGVYQILAIYSTVSSSLVLVGLIIAVGLWMCSNGSMQCRICI | |
| Exemplary H7 HA | 109 |
| MNTQILVFALVAVIPTNADKICLGHHAVSNGTKVNTLTERGVEVVNATETVE | |
| RTNIPKICSKGKRTTDLGQCGLLGTITGPPQCDQFLEFSADLIIERREGNDVCYP | |
| GKFVNEEALRQILRGSGGIDKETMGFTYSGIRTNGTTSACRRSGSSFYAEMEW | |
| LLSNTDNASFPQMTKSYKNTRRESALIVWGIHHSGSTTEQTKLYGSGNKLITV | |
| GSSKYHQSFVPSPGTRPQINGQSGRIDFHWLILDPNDTVTFSFNGAFIAPNRASF | |
| LRGKSMGIQSDVQVDANCEGECYHSGGTITSRLPFQNINSRAVGKCPRYVKQ | |
| ESLLLATGMKNVPEPSKKRKKRGLFGAIAGFIENGWEGLVDGWYGFRHQNA | |
| QGEGTAADYKSTQSAIDQITGKLNRLIEKTNQQFELIDNEFTEVEKQIGNLINW | |
| TKDSITEVWSYNAELIVAMENQHTIDLADSEMNRLYERVRKQLRENAEEDGT | |
| GCFEIFHKCDDDCMASIRNNTYDHSKYREEAMQNRIQIDPVKLSSGYKDVIL | |
| WFSFGASCFLLLAIAMGLVFICVKNGNMRCTICI | |
| Exemplary H8 HA | 110 |
| MEKFIAIATLASTNAYDRICIGYQSNNSTDTVNTLIEQNVPVTQTMELVETEKH | |
| PAYCNTDLGAPLELRDCKIEAVIYGNPKCDIHLKDQGWSYIVERPSAPEGMCY | |
| PGSVENLEELRFVFSSAASYKRIRLFDYSRWNVTRSGTSKACNASTGGQSFYR | |
| SINWLTKKEPDTYDFNEGAYVNNEDGDIIFLWGIHHPPDTKEQTTLYKNANTL | |
| SSVTTNTINRSFQPNIGPRPLVRGQQGRMDYYWGILKRGETLKIRTNGNLIAPE | |
| FGYLLKGESYGRIIQNEDIPIGNCNTKCQTYAGAINSSKPFQNASRHYMGECPK | |
| YVKKASLRLAVGLRNTPSVEPRGLFGAIAGFIEGGWSGMIDGWYGFHHSNSE | |
| GTGMAADQKSTQEAIDKITNKVNNIVDKMNREFEVVNHEFSEVEKRINMIND | |
| KIDDQIEDLWAYNAELLVLLENQKTLDEHDSNVKNLFDEVKRRLSANAIDAG | |
| NGCFDILHKCDNECMETIKNGTYDHKEYEEEAKLERSKINGVKLEENTTYKIL | |
| SIYSTVAASLCLAILIAGGLILGMQNGSCRCMFCI | |
| Exemplary H9 HA | 111 |
| METKAIIAALLMVTAANADKICIGYQSTNSTETVDTLTESNVPVTHTKELLHT | |
| EHNGMLCATDLGHPLILDTCTIEGLIYGNPSCDILLGGKEWSYIVERSSAVNG | |
| MCYPGNVENLEELRSLFSSAKSYKRIQIFPDKTWNVTYSGTSRACSNSFYRSM | |
| RWLTHKSNSYPFQNAHYTNNERENILFMWGIHHPPTDTEQTDLYKNADTTTS | |
| VTTEDINRTFKPVIGPRPLVNGQQGRIDYYWSVLKPGQTLRIRSNGNLIAPWY | |
| GHVLTGESHGRILKTDLNNGNCVVQCQTEKGGLNTTLPFHNISKYAFGNCPK | |
| YVGVKSLKLPVGLRNVPAVSSRGLFGAIAGFIEGGWPGLVAGWYGFQHSND | |
| QGVGMAADKGSTQKAIDKITSKVNNIIDKMNKQYEVIDHEFNELEARLNMIN | |
| NKIDDQIQDIWAYNAELLVLLENQKTLDEHDANVNNLYNKVKRALGSNAVE | |
| DGNGCFELYHKCDDQCMETIRNGTYDRQKYQEESRLERQKIEGVKLESEGTY | |
| KILTIYSTVASSLVLAMGFAAFLFWAMSNGSCRCNICI | |
| Exemplary H10 HA | 112 |
| MYKVVVIIALLGAVKGLDRICLGHHAVANGTIVKTLTNEQEEVTNATETVES | |
| TNLNKLCMKGRSYKDLGNCHPVGMLIGTPVCDPHLTGTWDTLIERENAIAHC | |
| YPGATINEEALRQKIMESGGISKMSTGFTYGSSITSAGTTKACMRNGGDSFYA | |
| ELKWLVSKTKGQNFPQTTNTYRNTDTAEHLIIWGIHHPSSTQEKNDLYGTQSL | |
| SISVESSTYQNNFVPVVGARPQVNGQSGRIDFHWTLVQPGDNITFSDNGGLIA | |
| PSRVSKLTGRDLGIQSEALIDNSCESKCFWRGGSINTKLPFQNLSPRTVGQCPK | |
| YVNQRSLLLATGMRNVPEVVQGRGLFGAIAGFIENGWEGMVDGWYGFRHQ | |
| NAQGTGQAADYKSTQAAIDQITGKLNRLIEKTNTEFESIESEFSETEHQIGNVIN | |
| WTKDSITDIWTYNAELLVAMENQHTIDMADSEMLNLYERVRKQLRQNAEED | |
| GKGCFEIYHTCDDSCMESIRNNTYDHSQYREEALLNRLNINPVKLSSGYKDIIL | |
| WFSFGESCFVLLAVVMGLVFFCLKNGNMRCTICI | |
| Exemplary H11 HA | 113 |
| MEKTLLFAAIFLCVKADEICIGYLSNNSTDKVDTIIENNVTVTSSVELVETEHT | |
| GSFCSINGKQPISLGDCSFAGWILGNPMCDELIGKTSWSYIVEKPNPTNGICYP | |
| GTLESEEELRLKFSGVLEFNKFEVFTSNGWGAVNSGVGVTAACKFGGSNSFF | |
| RNMVWLIHQSGTYPVIKRTFNNTKGRDVLIVWGIHHPATLTEHQDLYKKDSS | |
| YVAVGSETYNRRFTPEINTRPRVNGQAGRMTFYWKIVKPGESITFESNGAFLA | |
| PRYAFEIVSVGNGKLFRSELNIESCSTKCQTEIGGINTNKSFHNVHRNTIGDCPK | |
| YVNVKSLKLATGPRNVPAIASRGLFGAIAGFIEGGWPGLINGWYGFQHRDEE | |
| GTGIAADKESTQKAIDQITSKVNNIVDRMNTNFESVQHEFSEIEERINQLSKHV | |
| DDSVVDIWSYNAQLLVLLENEKTLDLHDSNVRNLHEKVRRMLKDNAKDEG | |
| NGCFTFYHKCDNKCIERVRNGTYDHKEFEEESKINRQEIEGVKLDSSGNVYKI | |
| LSIYSCIASSLVLAALIMGFMFWACSNGSCRCTICI | |
| Exemplary H12 HA | 114 |
| MEKFIILSTVLAASFAYDKICIGYQTNNSTETVNTLSEQNVPVTQVEELVHRGI | |
| DPILCGTELGSPLVLDDCSLEGLILGNPKCDLYLNGREWSYIVERPKEMEGVC | |
| YPGSIENQEELRSLFSSIKKYERVKMFDFTKWNVTYTGTSKACNNTSNQGSFY | |
| RSMRWLTLKSGQFPVQTDEYKNTRDSDIVFTWAIHHPPTSDEQVKLYKNPDT | |
| LSSVTTVEINRSFKPNIGPRPLVRGQQGRMDYYWAVLKPGQTVKIQTNGNLIA | |
| PEYGHLITGKSHGRILKNNLPMGQCVTECQLNEGVMNTSKPFQNTSKHYIGK | |
| CPKYIPSGSLKLAIGLRNVPQVQDRGLFGAIAGFIEGGWPGLVAGWYGFQHQ | |
| NAEGTGIAADRDSTQRAIDNMQNKLNNVIDKMNKQFEVVNHEFSEVESRINM | |
| INSKIDDQITDIWAYNAELLVLLENQKTLDEHDANVRNLHDRVRRVLRENAID | |
| TGDGCFEILHKCDNNCMDTIRNGTYNHKEYEEESKIERQKVNGVKLEENSTY | |
| KILSIYSSVASSLVLLLMIIGGFIFGCQNGNVRCTFCI | |
| Exemplary H13 HA | 115 |
| MALNVIATLTLISVCVHADRICVGYLSTNSSERVDTLLENGVPVTSSIDLIETN | |
| HTGTYCSLNGVSPVHLGDCSFEGWIVGNPACTSNFGIREWSYLIEDPAAPHGL | |
| CYPGELNNNGELRHLFSGIRSFSRTELIPPTSWGEVLDGTTSACRDNTGTNSFY | |
| RNLVWFIKKNTRYPVISKTYNNTTGRDVLVLWGIHHPVSVDETKTLYVNSDP | |
| YTLVSTKSWSEKYKLETGVRPGYNGQRSWMKIYWSLIHPGEMITFESNGGFL | |
| APRYGYIIEEYGKGRIFQSRIRMSRCNTKCQTSVGGINTNRTFQNIDKNALGDC | |
| PKYIKSGQLKLATGLRNVPAISNRGLFGAIAGFIEGGWPGLINGWYGFQHQNE | |
| QGTGIAADKESTQKAIDQITTKINNIIDKMNGNYDSIRGEFNQVEKRINMLADR | |
| IDDAVTDIWSYNAKLLVLLENDKTLDMHDANVKNLHEQVRRELKDNAIDEG | |
| NGCFELLHKCNDSCMETIRNGTYDHTEYAEESKLKRQEIDGIKLKSEDNVYK | |
| ALSIYSCIASSVVLVGLILSFIMWACSSGNCRENVCI | |
| Exemplary H14 HA | 116 |
| MIALILVALALSHTAYSQITNGTTGNPIICLGHHAVENGTSVKTLTDNHVEVVS | |
| AKELVETNHTDELCPSPLKLVDGQDCHLINGALGSPGCDRLQDTTWDVFIERP | |
| TAVDTCYPFDVPDYQSLRSILASSGSLEFIAEQFTWNGVKVDGSSSACLRGGR | |
| NSFFSRLNWLTKATNGNYGPINVTKENTGSYVRLYLWGVHHPSSDNEQTDLY | |
| KVATGRVTVSTRSDQISIVPNIGSRPRVRNQSGRISIYWTLVNPGDSIIFNSIGNL | |
| IAPRGHYKISKSTKSTVLKSDKRIGSCTSPCLTDKGSIQSDKPFQNVSRIAIGNC | |
| PKYVKQGSLMLATGMRNIPGKQAKGLFGAIAGFIENGWQGLIDGWYGFRHQ | |
| NAEGTGTAADLKSTQAAIDQINGKLNRLIEKTNEKYHQIEKEFEQVEGRIQDL | |
| EKYVEDTKIDLWSYNAELLVALENQHTIDVTDSEMNKLFERVRRQLRENAED | |
| QGNGCFEIFHQCDNNCIESIRNGTYDHNIYRDEAINNRIKINPVTLTMGYKDIIL | |
| WISFSMSCFVFVALILGFVLWACQNGNIRCQICI | |
| Exemplary H15 HA | 117 |
| MNTQIIVILVLGLSMVKSDKICLGHHAVANGTKVNTLTERGVEVVNATETVEI | |
| TGIDKVCTKGKKAVDLGSCGILGTIIGPPQCDLHLEFKADLIIERRNSSDICYPG | |
| RFTNEEALRQIIRESGGIDKESMGFRYSGIRTDGATSACKRTVSSFYSEMKWLS | |
| SSMNNQVFPQLNQTYRNTRKEPALIVWGVHHSSSLDEQNKLYGTGNKLITVG | |
| SSKYQQSFSPSPGARPKVNGQAGRIDFHWMLLDPGDTVTFTFNGAFIAPDRAT | |
| FLRSNAPSGIEYNGKSLGIQSDAQIDESCEGECFYSGGTINSPLPFQNIDSRAVG | |
| KCPRYVKQSSLPLALGMKNVPEKIRTRGLFGAIAGFIENGWEGLIDGWYGFR | |
| HQNAQGQGTAADYKSTQAAIDQITGKLNRLIEKTNKQFELIDNEFTEVEQQIG | |
| NVINWTRDSLTEIWSYNAELLVAMENQHTIDLADSEMNKLYERVRRQLREN | |
| AEEDGTGCFEIFHRCDDQCMESIRNNTYNHTEYRQEALQNRIMINPVKLSSGY | |
| KDVILWFSFGASCVMLLAIAMGLIFMCVKNGNLRCTICI | |
| Exemplary H16 HA | 118 |
| MMIKVLYFLIIVLGRYSKADKICIGYLSNNSSDTVDTLTENGVPVTSSVDLVET | |
| NHTGTYCSLNGISPIHLGDCSFEGWIVGNPSCATNINIREWSYLIEDPNAPNKF | |
| CYPGELDNNGELRHLFSGVNSFSRTELINPSKWGNVLDGVTASCLDRGASSFY | |
| RNLVWIVKKDEKYPVIKGDYNNTTGRDVLVLWGIHHPDTETTATNLYVNKN | |
| PYTLVSTKEWSKRYELEIGTRIGDGQRSWMKLYWHLMHPGERIMFESNGGLI | |
| APRYGYIIEKYGTGRIFQSGVRMARCNTKCQTSLGGINTNKTFQNIERNALGD | |
| CPKYIKSGQLKLATGLRNVPSIGERGLFGAIAGFIEGGWPGLINGWYGFQHQN | |
| EQGTGIAADKASTQKAINEITTKINNIIEKMNGNYDSIRGEFNQVEKRINMLAD | |
| RVDDAVTDIWSYNAKLLVLLENDRTLDLHDANVRNLHDQVKRALKSNAIDE | |
| GDGCFNLLHKCNDSCMETIRNGTYNHEDYREESQLKRQEIEGIKLKTEDNVY | |
| KVLSIYSCIASSIVLVGLILAFIMWACSNGSCRFNVCI | |
| Exemplary H17 HA | 119 |
| MELIVLLILLNPYTFVLGDRICIGYQANQNNQTVNTLLEQNVPVTGAQEILETN | |
| HNGKLCSLNGVPPLDLQSCTLAGWLLGNPNCDSLLEAEEWSYIKINESAPDDL | |
| CFPGNFENLQDLLLEMSGVQNFTKVKLFNPQSMTGVTTNNVDQTCPFEGKPS | |
| FYRNLNWIQGNSGLPFNIEIKNPTSNPLLLLWGIHNTKDAAQQRNLYGNDYSY | |
| TIFNFGEKSEEFRPEIGQRDEVKAHQDRIDYYWGSLPAQSTLRIESTGNLIAPEY | |
| GFYYKRKEGKGGLMKSKLPISDCSTKCQTPLGALNSTLPFQNVHQQTIGNCPK | |
| YVKATSLMLATGLRNNPQMEGRGLFGAIAGFIEGGWQGMIDGWYGYHHEN | |
| QEGSGYAADKEATQKAVDAITNKVNSIIDKMNSQFESNIKEFNRLELRIQHLS | |
| DRVDDALLDIWSYNTELLVLLENERTLDFHDANVKNLFEKVKAQLKDNAIDE | |
| GNGCFLLLHKCNNSCMDDIKNGTYKYMDYREESHIEKQKIDGVKLTDYSRY | |
| YIMTLYSTIASSVVLGSLIIAAFLWGCQKGSIQCKICI | |
| His-His-His-His-His-His | 120 |
| GSGYIPEAPRDGQAYVRKDGEWVLLSTFL | 121 |
| LVPRGSP | 122 |
| Glu-Asn-Leu-Tyr-Phe-Gln-(Gly/Ser) | 123 |
| ENCT | 124 |
| GNCH | 125 |

### 6. EXAMPLES

### 6.1 Example 1: Species-Specific Immunodominance of Antigenic Sites in Influenza H1 Hemagglutinin

This example indicates the usefulness of a panel of influenza viruses expressing a mosaic influenza virus HA polypeptide, such as described in this Example, in which amino acid residues in an antigenic site in the globular head have been substituted with other residues, to assess changes in a subject's hemagglutination inhibition profile. For example, such a panel of influenza viruses may be useful to indicate a change in a subject's immune response, e.g., after influenza vaccination. In addition, the data for an influenza virus comprising a mosaic influenza virus HA polypeptide when amino acid residues in 5 antigenic sites are substituted reduces or eliminates hemagglutination inhibition detected in plasma samples from human subjects receiving the seasonal vaccine in Table 9. This data indicates that a mosaic influenza virus HA polypeptide in which amino acid residues in the 5 antigenic sites of an H1 virus are substituted with other amino acid residues may be used as an immunogen to boost the immune response to the conserved antigenic sites in the HA stem domain of an H1 virus and the conserved antigenic sites in the HA globular domain of an H1 virus.

### 6.1.1 INTRODUCTION

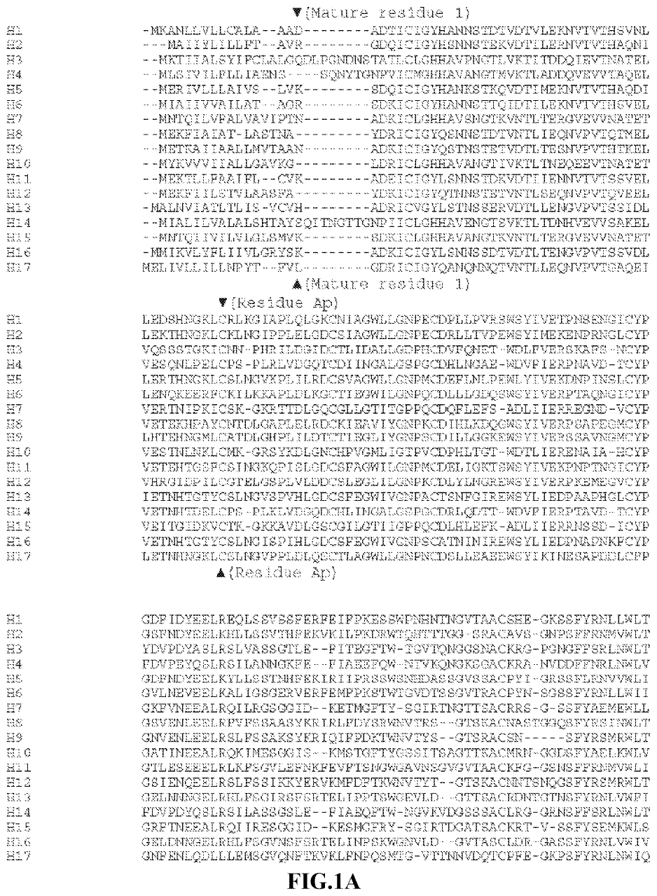

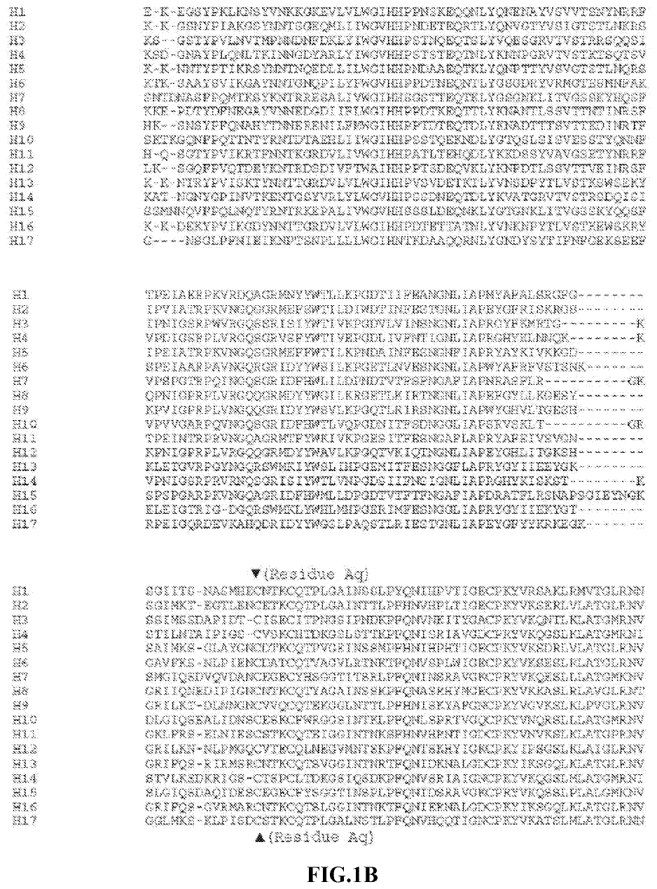

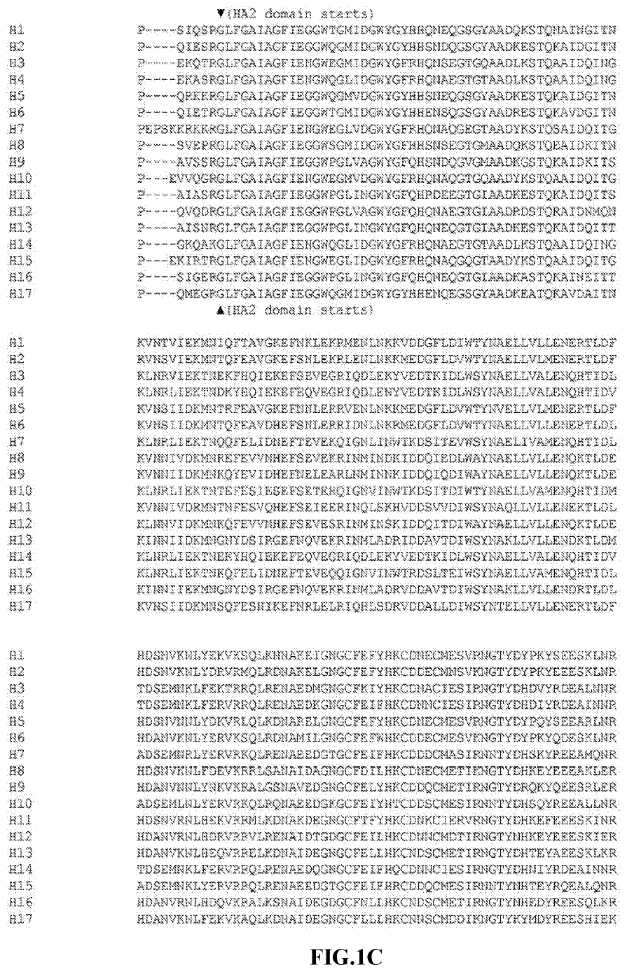

Influenza virus is an eight-segmented, single-stranded, negative-sense RNA virus of the family Orthomyxoviridae [7]. The immunodominant surface protein, HA, that coats the viral lipid membrane is composed of a head domain and a stalk domain. Classically, five antigenic sites were identified in the head domain of the laboratory adapted H1N1 strain, A/Puerto Rico/8/1934 (PR8) [8]. These antigenic sites, defined as Sa, Sb, Ca1, Ca2, and Cb (FIG. 1A), were characterized using virus escape mutants and a panel of 58 monoclonal antibodies [9]. Sa and Sb are located on the distal tip of each HA monomer, while Ca1, Ca2, and Cb are located proximally, near the stalk domain. Virus-host attachment occurs at the sialic acid receptor binding domain (RBD) located between Sb, Ca2, and Sa [10].

The pandemic-like H1N1 influenza virus strain, A/Michigan/45/2015, was recently included as a component of the seasonal influenza vaccine [6], and has been recommended as a vaccine component for the 2018-2019 northern hemisphere influenza season. Hemagglutination inhibition (HI) titers are a major correlate of protection for influenza-related illness. The influenza virus hemagglutinin possesses antigenic sites that are the target of HI active antibodies. This Example characterizes the antigenicity of five classically defined antigenic sites within the hemagglutinin (HA) head domain of this H1 strain in animals and humans.

Hemagglutination inhibition (HI) hierarchies in humans including all antigenic sites of pH1N1 have never been established. The present Example used a reverse genetics system to create a panel of mutant viruses encoding mutant HAs that lack one of the five HI active antigenic sites. In particular, a panel of mutant viruses each lacking a classically defined antigenic site was created to compare the species-specific immunodominance of the antigenic sites in a clinically relevant hemagglutinin. When antisera to A/Michigan/45/2015 were tested against this panel of mutant viruses, relative reductions in HI titers defined the HI dominance of specific antigenic sites.

HI active antibodies of guinea pigs were not directed against any specific antigenic site, although trends were observed towards Sb, Ca2, and Sa. HI titers of antisera from infected ferrets were significantly affected by site Sa. HI active antibodies of adult humans followed yet another immunodominance pattern, Sb and Sa were immunodominant. When comparing the HI profiles between different species by antigenic cartography, animals and humans grouped separately. Thus, this Example provides novel characterizations of the antibody-mediated immune responses against the head domain of a recent H1 hemagglutinin in animals and humans.

### 6.1.2 MATERIALS AND METHODS

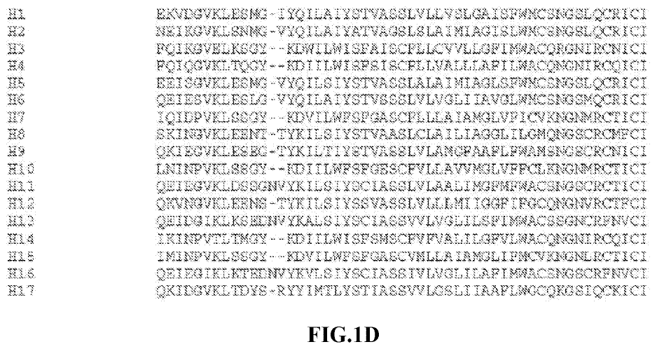

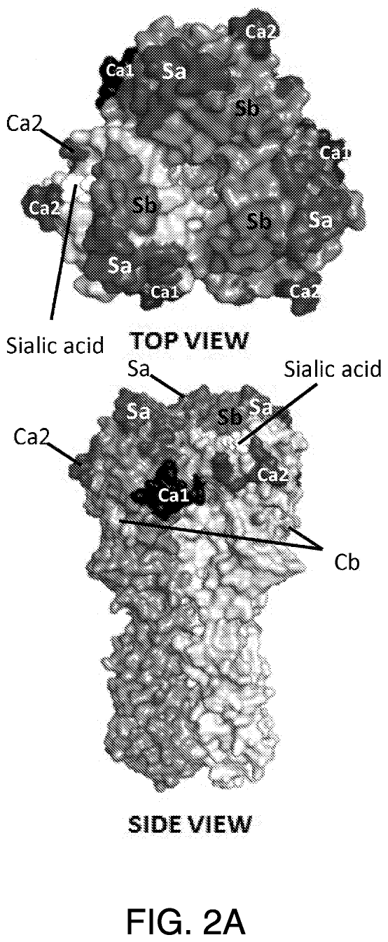

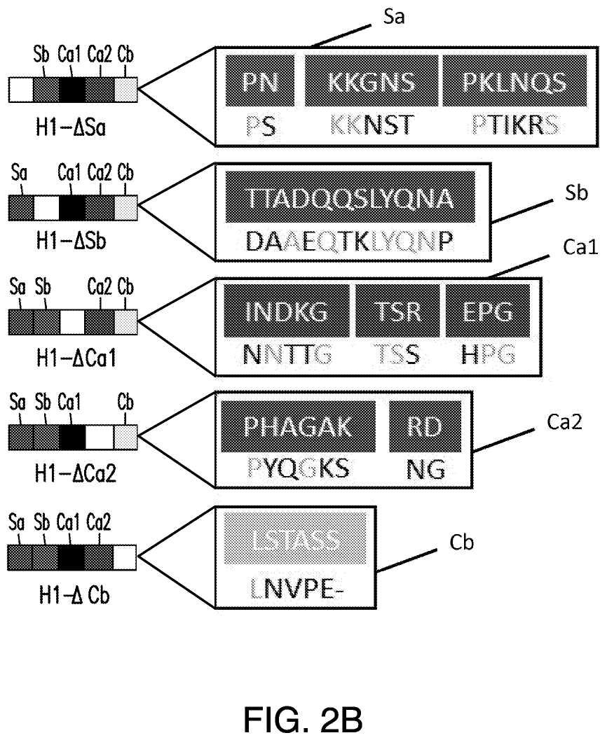

Schematic representation of classically defined epitopes on the hemagglutinin (HA) 3D protein structure: The major antigenic sites (Sa, Sb, Ca1, Ca2, and Cb, see FIG. 2A) of A/Michigan/45/2015 virus were colored on the pandemic H1 HA protein structure (PDB:3UBE) using PyMOL (The PyMOL Molecular Graphics System, Version 2.0.1, Schrödinger, LLC).

Recombinant virus construction and purification: Mutant viruses were constructed using a reverse genetics system in a similar manner as previously described [16]. Briefly, classically described epitopes of pdm09-like H1 (A/Michigan/45/2015), were substituted with the corresponding potential epitopes of H5 (A/Vietnam/1203/2004), and/or H13 (A/black headed gull/Sweden/1/1999). Constructed ambisense DNA plasmids were cloned and transfected into 293T cells with a 7-segment plasmid encoding essential viral proteins of PR8. Scraped cells and supernatants were injected into 8-10 day old embryonated chicken eggs (Charles River Laboratories) for viral rescue at 37 C for 48 hours. Viruses were plaque purified on Madin-Darby Canine Kidney (MDCK) cells grown in Dulbecco's Modified Eagles Medium (DMEM, Gibco) containing 10% fetal bovine serum (FBS, Hyclone) and penicillin-streptomycin mix (100 units/ml of penicillin and 100 μg/ml of streptomycin, Gibco). Individual plaques were injected into embryonated eggs, and viral RNAs were extracted from the allantoic fluids and HA segments were Sanger sequenced.

Study approval. The Icahn School of Medicine at Mount Sinai's Institutional Review Board approved the human studies. Informed consent was received from participants prior to inclusion in the study. Animal experiments were performed in accordance with protocols approved by the Institutional Animal Care and Use Committee at the Icahn School of Medicine at Mount Sinai.

Animal sample collection and seroconversion: Six- to eight-week old female BALB/c mice (n=10, Jackson Laboratories), five- to six-week old female Hartley guinea pigs (n=4, Charles River Laboratories), and circulating H1N1 influenza virus seronegatice-confirmed four- to five-month-old male Fitch ferrets (n=3, Triple F Farms, Sayre, PA) were anesthetized and intranasally infected with 1×10⁵ PFU of A/Michigan/45/2015 per animal. Four weeks post infection samples were collected and animals were euthanized. Antisera from two ferrets immunized independently were harvested three weeks post infection. Samples were then Vibrio cholera Receptor-destroying enzyme (RDE) (Denka Seiken, Chuo-Ku, Tokyo, Japan) treated for use in HI assays as described previously [25].

Human sample collection and treatment: Eighteen individuals provided informed consented and donated blood before or on the day of seasonal influenza vaccination as well as four-eight weeks later. Plasma samples were stored at −80° C. until use. Each 100 μl human plasma sample was heat treated at 56° C. for 30 minutes. Samples were then Vibrio cholerae RDE treated for use in HI assays as described previously [25].

Hemagglutination inhibition assay: Chicken red blood cells (Lampire) were washed in PBS and resuspended at a concentration of 0.5% hematocrit. Receptor-destroying enzyme (RDE) treated human samples that resulted in a 10-fold dilution were further serially diluted 1:2 in 25 μl volumes across a 96-well V-bottom plate. Allantoic fluid containing wild-type (wt) or mutant H1 viruses was diluted to eight HA-units and then incubated in equal volumes to antisera (25 μl each) for 30 minutes at 25° C. Chicken red blood cells were then added and HI titers were visually determined. All samples were tested in duplicates.

Antigenic cartography: Antigenic map construction was performed as described previously [26]. Briefly, modified multi-dimensional scaling methods arrange point distances between antisera and viruses based on HI titers. The map displayed in this study was generated from Acmacs Web Cherry (https://acmacs-web.antigenic-cartography.org).

Statistics. Statistical data were generated with the GraphPad Prism program version 7.02 (GraphPad Software). Statistical significance between groups was determined by performing One-way ANOVA analysis with Dunn's-corrected Kruskal-Wallis tests

### 6.1.3 COMBINED RESULTS AND DISCUSSION

### Creation of a Mutant Virus Panel for A/Michigan/45/2015

Using a reverse genetics system [16], a panel of five mutant viruses (H1-ΔSa, H1-ΔSb, H1-ΔCa1, H1-ΔCa2, H1-ΔCb) was created in which classically defined H1 antigenic sites (Sa, Sb, Ca1, Ca2 and Cb, respectively) were substituted with heterologous antigenic sites from either H5 or H13 HAs (FIG. 2B). Mutant viruses were designed with an HA encoded by A/Michigan/45/2015 and the 7 remaining segments encoded by A/Puerto Rico/8/1934 H1N1 (PR8). Previous observations suggested that antigenically drifted influenza virus strains generally had four or more amino acid substitutions in two or more antigenic sites [17]. To ensure the loss of antigenicity for an individual epitope, each mutant virus in the panel contained five or more amino acid substitutions within one antigenic site. Additionally, several amino acid substitutions that were included in the panel were consistent with previously described escape mutations [18]. While this Example is focused on the classically defined epitopes of pandemic H1 hemagglutinin, several non-classical epitopes have been described as being HI sensitive [11, 19, 20]. To measure the HI activities of these non-classical epitopes, two additional mutant viruses were constructed: a mosaic H5/1 virus (mH5/1), wherein all five classically defined H1 epitopes were replaced with H5 epitopes, leaving the non-classical epitopes intact (SEQ ID NO: 30), and a chimeric H5/1 (cH5/1), where the entire H1 head domain was replaced with an H5 head domain. All mutant viruses were plaque purified and sequenced. Egg-adaptive mutations were rare and only observed outside of the targeted epitopes. In summary, a robust panel of mutant viruses lacking antigenic epitopes in the head domain was rescued.

### Hemagglutination Inhibition (HI) Profiles of Animal Antisera

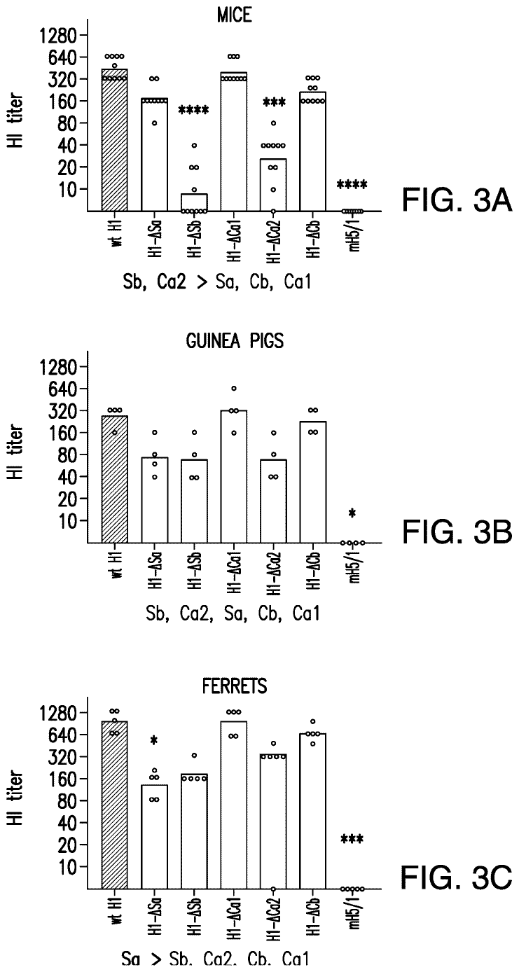

Naïve mice, guinea pigs, and ferrets were intranasally infected with 105 plaque forming units (PFU) of A/Michigan/45/2015 virus (H1N1). Sera were collected 4 weeks post-infection (except for two of the five ferret antisera, which were collected at 3 weeks post-infection). Animal antisera showed high HI titers (>1:160) against wild-type H1 virus. Ferret antisera had the highest HI titers against wild-type H1 followed by mouse antisera then guinea pig antisera. HI assays with the panel of mutant viruses (see FIG. 2B) revealed the greatest reductions in HI titers to mutant viruses containing substitutions surrounding the Receptor Binding Domain (RBD). Mouse antisera had significant reductions in HI titers against H1-ΔSb and H1-ΔCa2. Minimal reductions were observed against H1-ΔSa, H1-ΔCb, and H1-ΔCa1 (FIG. 3A). Guinea pig antisera showed no significant reductions for any specific site, but minimal reductions in HI titers trended towards H1-ΔSb, H1-ΔCa2, and H1-ΔSa viruses (FIG. 3B). Ferret antisera had significant reduction in the HI titer against H1-ΔSa (FIG. 3C). Using a novel panel of mutant viruses, HI hierarchies have been established for the antibody response of mice, guinea pigs, and ferrets.

Mice, guinea pigs and ferrets are used as model animals to examine immune responses to influenza virus [21]. The results of the present Example provide the first description of the HI profiles of infected guinea pigs. The HI antibody responses of guinea pig antisera showed no significant preferences toward any specific sites; however, trends were observed for antigenic sites proximal to the RBD, specifically Sa, Sb, and Ca2. These results support previous animal studies that demonstrated the importance of the HI active antigenic sites surrounding the RBD [12, 15].

### Seasonal Vaccination Maintains the Hemagglutination Inhibition (HI) Profile of Humans

Human plasma samples were collected from eighteen adult volunteers prior to vaccination with 2017-2018 seasonal vaccines and grater than four weeks after vaccination. Study participants varied in age, sex, sample collection times, and specific type of vaccination (Table 9).

TABLE 9
Demographics of human donors and respective seasonal
vaccinations. Donors varied in sex, age, sample collection
times, and the type of seasonal vaccines that they received.

| | Samples | Sample | | | |
|---|---|---|---|---|---|
| | collection | collection | | | Seasonal |
| | pre- | post- | | | vaccine |
| Donor | vaccination | vaccination | Sex | Age | received |
| ID | (Days) | (Days) | (F/M) | group | (2017-2018) |
| A | 0 | 49 | F | 50-59 | Flucelvax |
| B | 5 | 43 | M | 30-39 | Flucelvax |
| C | 0 | 41 | M | >60 | Fluzone |
| | | | | | High-Dose |
| D | 1 | 36 | F | >60 | Fluzone |
| | | | | | High-Dose |
| E | 0 | 33 | F | 40-49 | Flucelvax |
| F | 260 | 42 | F | 50-59 | Flucelvax |
| G | 275 | 57 | M | 40-49 | Flucelvax |
| H | 270 | 52 | M | 50-59 | Flucelvax |
| I | 12 | 28 | M | 30-39 | Fluarix |
| | | | | | Quadrivalent |
| J | 0 | 29 | M | >60 | Fluvirin |
| K | 0 | 29 | M | 50-59 | Fluarix |
| | | | | | Quadrivalent |
| L | 3 | 28 | M | 40-49 | Fluarix |
| | | | | | Quadrivalent |
| M | 3 | 28 | F | 30-39 | Fluzone |
| | | | | | Quadrivalent |
| N | 0 | 28 | M | 20-29 | Fluarix |
| | | | | | Quadrivalent |
| O | 9 | 28 | F | 40-49 | Fluarix |
| | | | | | Quadrivalent |
| P | 2 | 27 | M | 30-39 | Flublok |
| | | | | | Quadrivalent |
| Q | 0 | 28 | F | 30-39 | Fluarix |
| | | | | | Quadrivalent |
| R | 0 | 28 | M | 30-39 | Fluzone |
| | | | | | Quadrivalent |

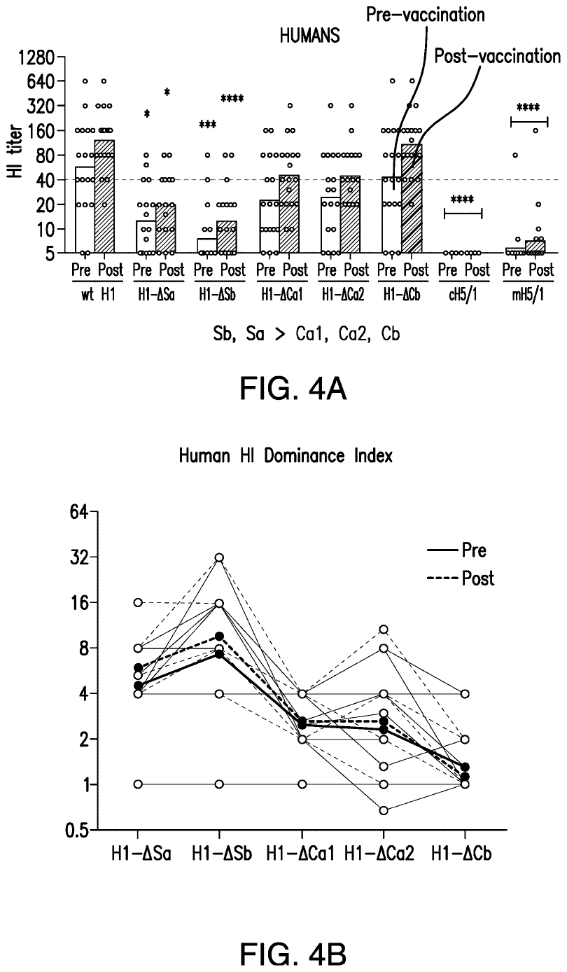

Human plasma samples were tested for HI activity against the wild-type H1 virus and against the panel of mutant viruses (FIG. 2B). There was a broad range of HI titers observed against wild-type H1 virus (FIG. 4A). Generally, HI titers against all viruses increased upon seasonal vaccination. Geometric mean analysis of the pre-vaccination antisera showed the significant reductions in HI titers against H1-ΔSb and H1-ΔSa. H1-ΔCa1 and H1-ΔCa2 showed minimal but insignificant reductions. H1-ΔCb showed similar HI titers to wild-type H1 virus. The HI profile was preserved in the post-vaccination antisera. These results confirm that the human HI activity of human plasma mostly depends on antigenic sites surrounding the RBD.

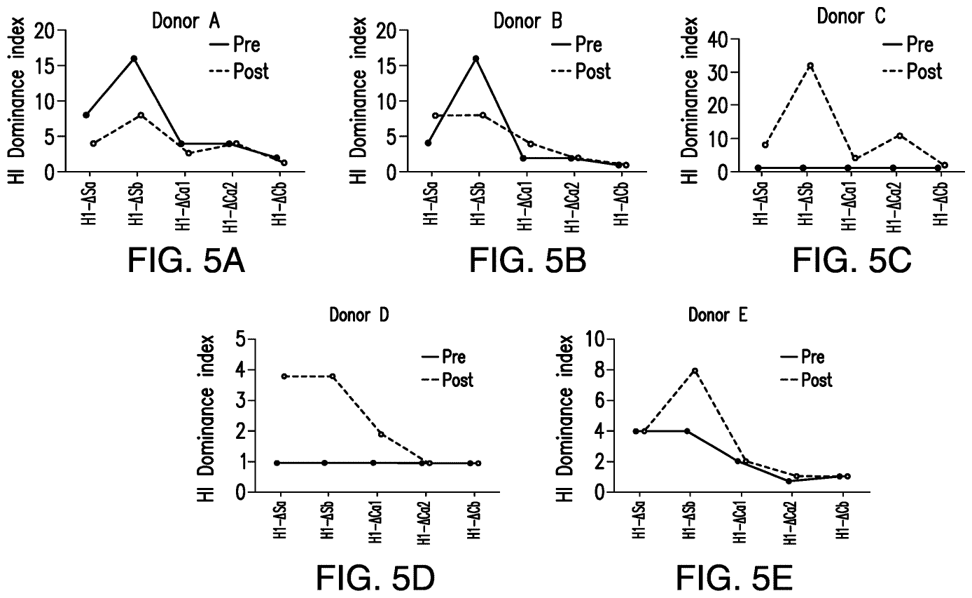

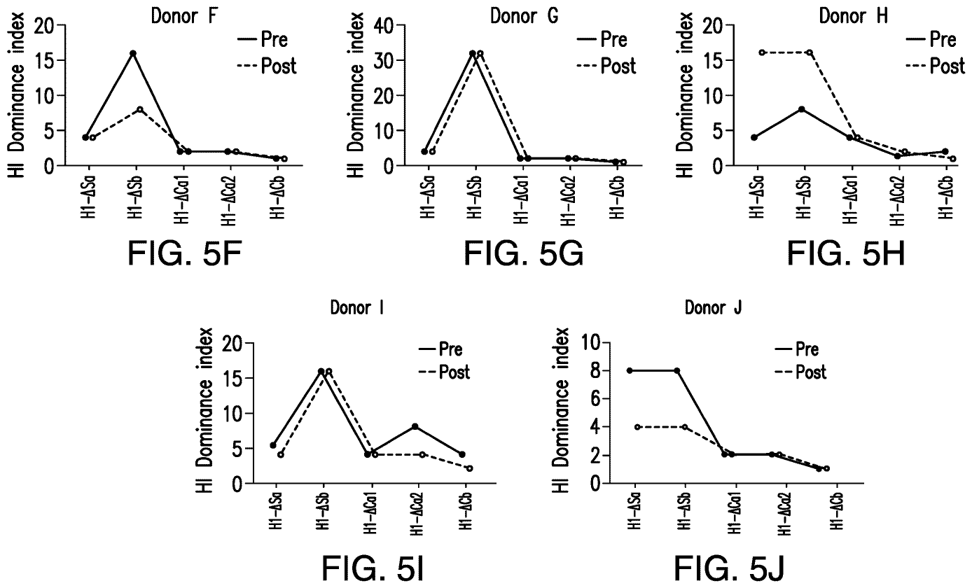

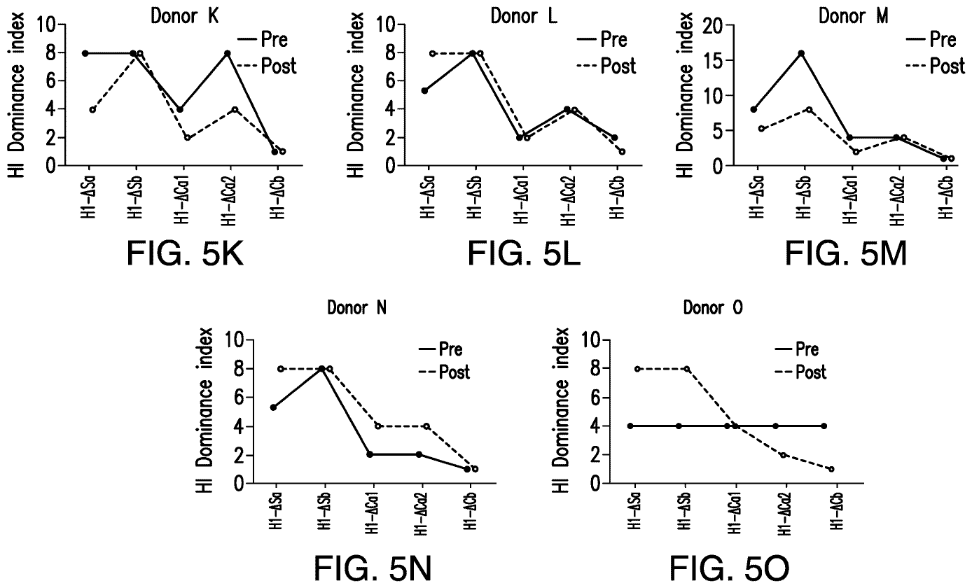

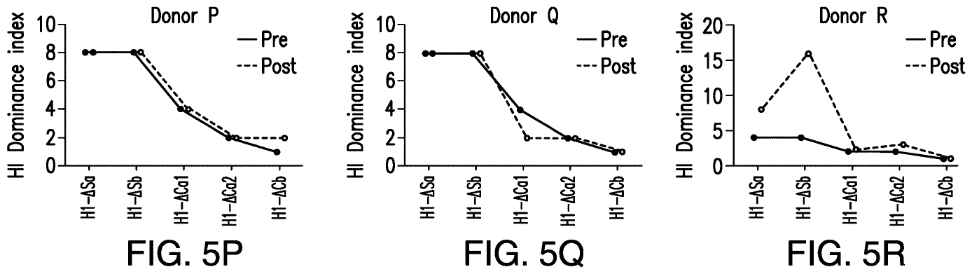

An HI dominance index was created to help analyze the datasets (FIG. 4B). The HI dominance index equals the fold reduction in HI titer of a specific mutant virus compared to the respective wild-type H1 virus. By plotting the HI dominance indices of all five mutant viruses, subtle shifts in averaged HI dominance due to seasonal vaccination were revealed. A closer analysis of the full HI profile of each individual donor showed wide variations in HI profiles for both pre- and post-vaccination antisera (FIGS. 5A-5R). The clinical significance of these subtle changes has yet to be determined. Unfortunately, due to the relatively small number of study participants, the analysis in this Example was not powered to detect any potential correlations between HI profiles and patient demographics. However, no obvious unusual distributions were discovered for the various types of seasonal vaccines that were administered.

HI titers were not detected in donor samples using the cH5/1 virus which displays a head domain to which humans are typically naïve. Interestingly, HI activity was detected with the mH5/1 virus, a virus lacking all five classically defined antigenic sites. Two of 18 donors had HI titers against mH5/1 in pre-vaccination samples, and 5 of 18 donors developed HI titers post-vaccination. The detection of these HI titers suggest that naturally occurring antibody responses against non-classical HA-head epitopes is uncommon. Importantly, they can be detected in a fraction of the population, and HI activity against these non-classical epitopes can be boosted by immunization. It is possible that the mH5/1 virus may contain incompletely substituted epitopes that are targeted upon vaccination. However, this is unlikely given that antisera of all animals infected with wild-type H1 did not show any HI titers to mH5/1 virus (FIGS. 3A-3C). Another explanation could be antibodies that target the receptor binding pocket directly using a long CDR3 region. Such monoclonal antibodies have been isolated from humans and typically exhibit binding that is independent from mutations in the major antigenic sites [20, 22-24].

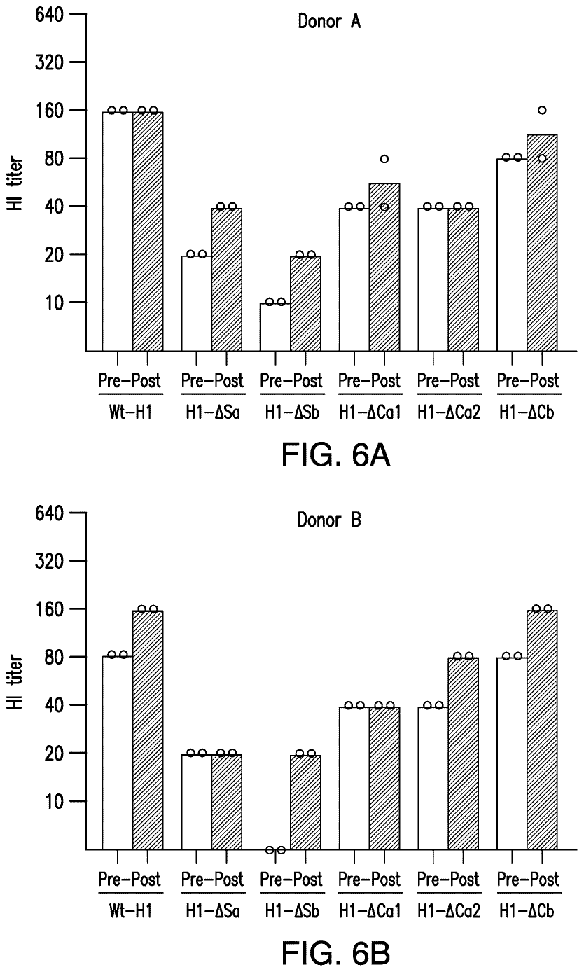

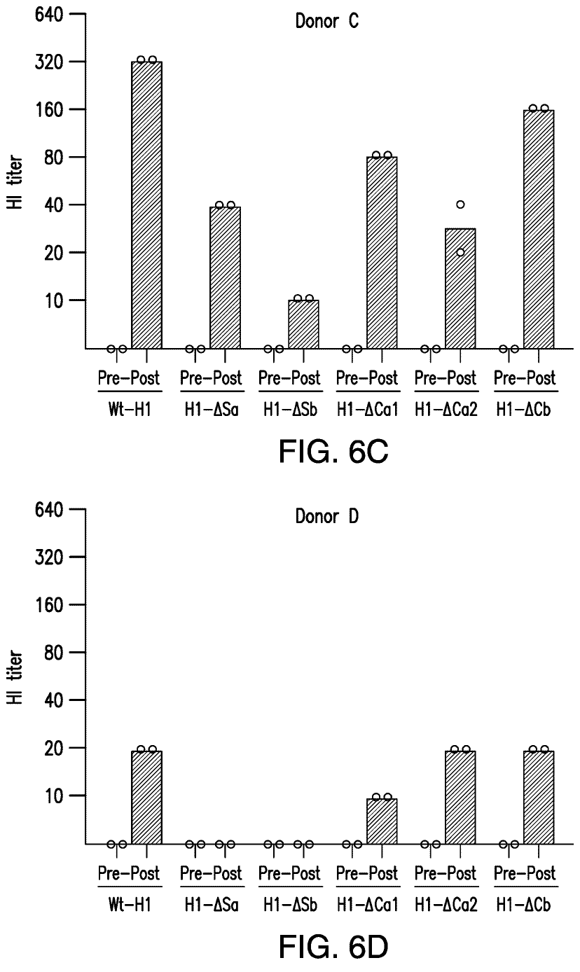

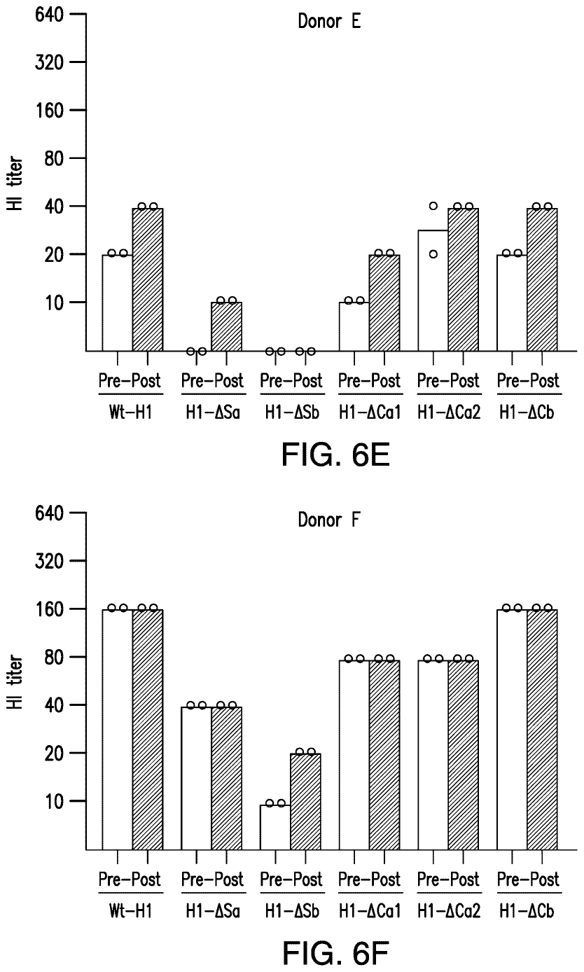

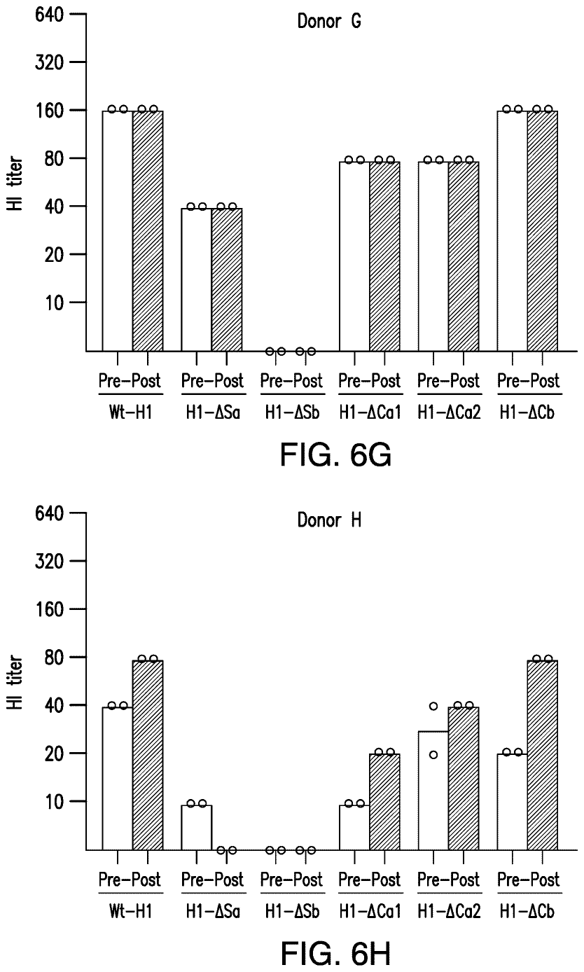

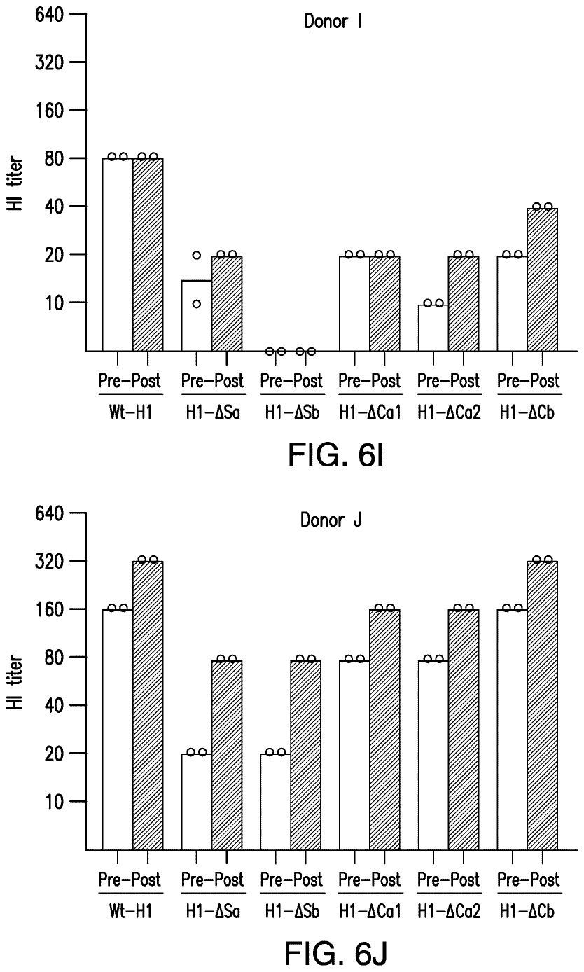

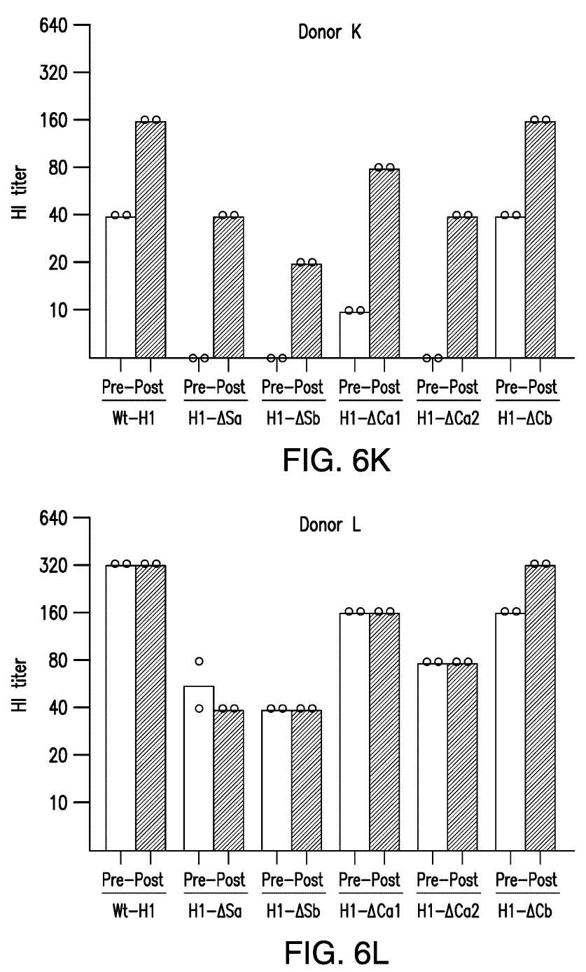

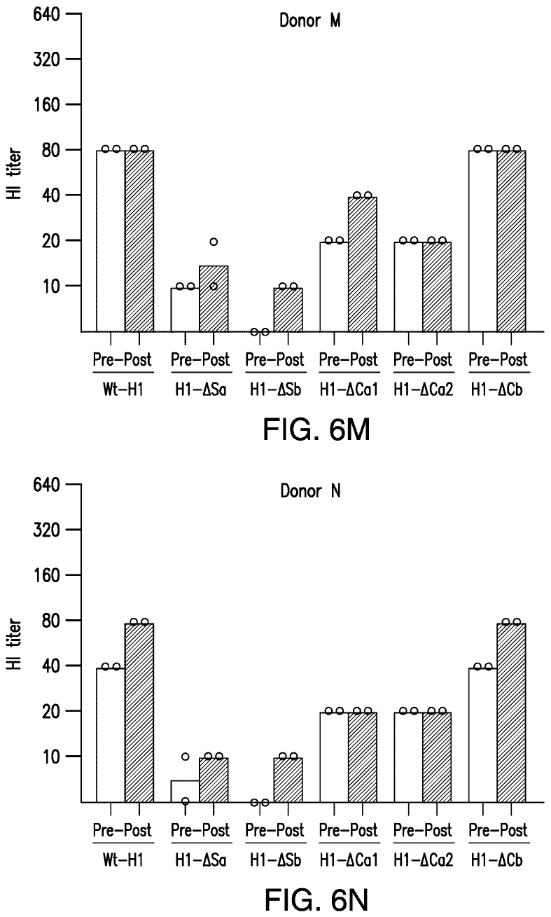

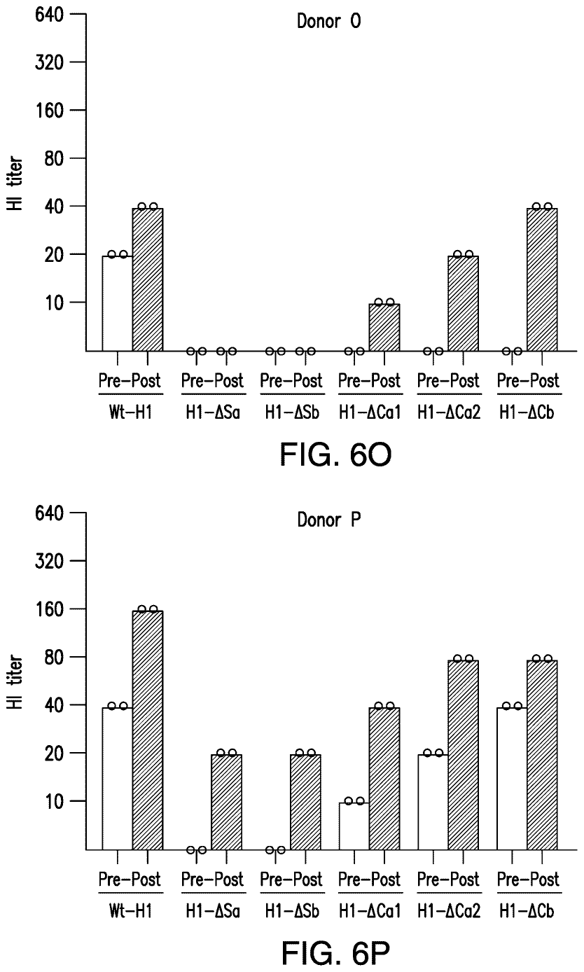

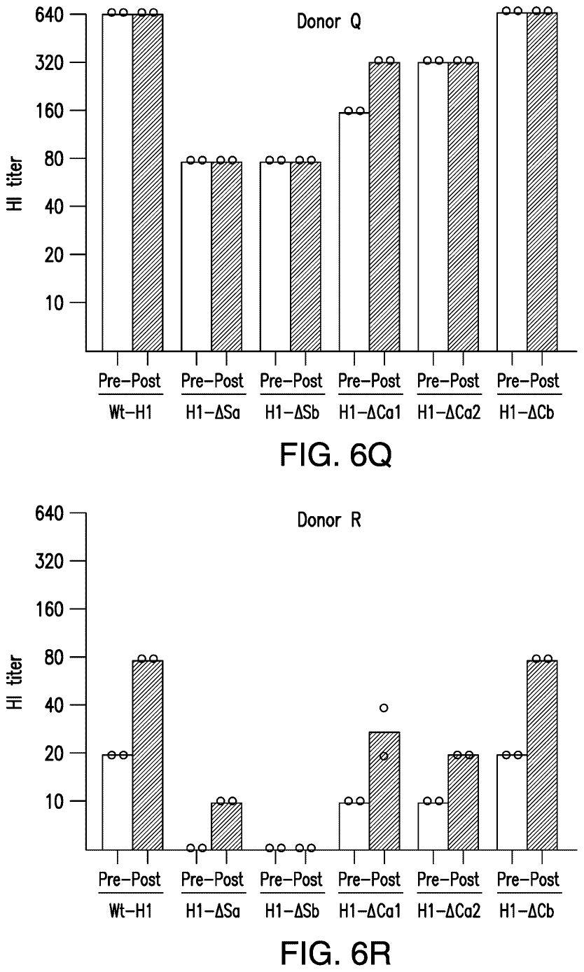

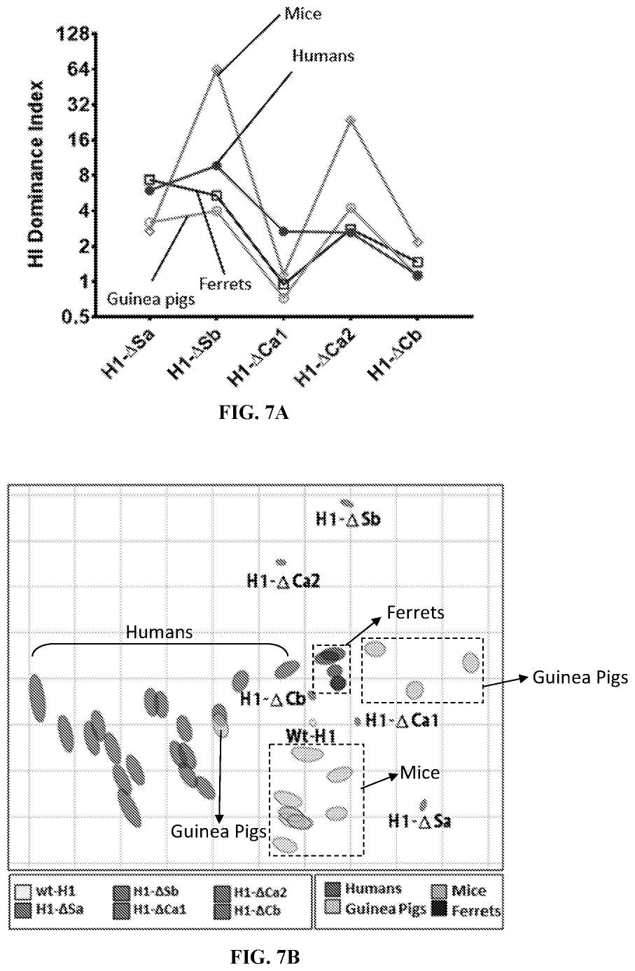

HI profiling offers a new method to measure immune responses in individuals whose HI titers do not change against wild-type H1 virus after vaccination. This is important since antigenic site immunodominance could change even in the absence of an increase in total HI activity. Seven of the 18 donors (39%) did not change their HI titers against wild-type H1 virus (donors A, F, G, I, L, M, and Q of FIGS. 6A-6R). Of these seven donors, three donors (G, L, Q) showed no changes in their HI profiles, pre- and post-vaccination. One donor (M) changed from Sb dominance to Sa and Sb co-dominance upon vaccination. Three donors maintained their Sb dominance but had reductions in the intensity of Sb dominance. Further studies may demonstrate that certain isolated changes in HI profiles are sufficient to confer protection towards different strains.

### Comparison of Species-Specific Hemagglutination Inhibition (HI) Profiles

This Example provides a comparison of HI profiles amongst different species. Despite similar tendencies to target the RBD, animals and humans have distinct HI profiles. These differences can be seen when plotting the averaged HI dominance indices of all of the species together (FIG. 7A). Ferrets and humans have higher Sa HI dominance indices than mice and guinea pigs. Mice have particularly high HI dominance indices for Sb and Ca2. The Ca1 HI dominance index of humans is higher than the Ca1 HI dominance indices of all three tested animal species.

Differences in HI profiles can also be visualized with antigenic cartography (FIG. 7B). Mapping the HI titers of different species is possible because the antisera of animals and humans were all exposed to wild-type A/Michigan/45/2015 virus. Clear divisions appear between human, ferret, and mouse antisera. Ferret antisera are tightly grouped, while mice, guinea pig, and human antisera are broadly distributed and less superimposed. A single guinea pig antiserum interestingly overlaps with human antisera. This individual antiserum may be an outlier due to the limited number of animals studied during this investigation, or it may be a consequence of the genetic variations of outbred guinea pigs. These observed interspecies differences are intriguing and warrant future species to species direct comparisons.

This panel of mutant reagents can make it possible to monitor the HI profiles of individuals in a longitudinal manner. Changes in an individual's HI profile may indicate active influenza infection, aging, or subsequent immunizations. Larger cohorts can assess whether HI titers against specific antigenic sites may correlate strongly with protection against influenza related illness.

### 6.1.4 REFERENCES

1. Budd, A. P., et al., Update: Influenza Activity—United States, Oct. 1, 2017-Feb. 3, 2018. MMWR Morb Mortal Wkly Rep, 2018. 67(6): p. 169-179.
2. Nichol, K. L., et al., The effectiveness of vaccination against influenza in healthy, working adults. N Engl J Med, 1995. 333(14): p. 889-93.
3. Flannery, B., et al., Interim Estimates of 2017-18 Seasonal Influenza Vaccine Effectiveness—United States, February 2018. MMWR Morb Mortal Wkly Rep, 2018. 67(6): p. 180-185.
4. Grohskopf, L. A., et al., Prevention and Control of Seasonal Influenza with Vaccines: Recommendations of the Advisory Committee on Immunization Practices—United States, 2017-18 Influenza Season. MMWR Recomm Rep, 2017. 66(2): p. 1-20.
7. Knipe, D. M. and P. M. Howley, Fields virology. 6th ed. 2013, Philadelphia, PA: Wolters Kluwer/Lippincott Williams & Wilkins Health. 2 volumes.
8. Caton, A. J., et al., The antigenic structure of the influenza virus A/PR/8/34 hemagglutinin (H1 subtype). Cell, 1982. 31 (2 Pt 1): p. 417-27.
9 Gerhard, W., et al., Antigenic structure of influenza virus haemagglutinin defined by hybridoma antibodies. Nature, 1981. 290(5808): p. 713-7.
10. Xu, R., et al., Structural characterization of the hemagglutinin receptor specificity from the 2009 H1N1 influenza pandemic. J Virol, 2012. 86(2): p. 982-90.
11. Matsuzaki, Y., et al., Epitope mapping of the hemagglutinin molecule of A/(H1N1)pdm09 influenza virus by using monoclonal antibody escape mutants. J Virol, 2014. 88(21): p. 12364-73.
12. Angeletti, D., et al., Defining B cell immunodominance to viruses. Nat Immunol, 2017. 18(4): p. 456-463.
13. Ohmit, S. E., et al., Influenza hemagglutination-inhibition antibody titer as a correlate of vaccine-induced protection. J Infect Dis, 2011. 204(12): p. 1879-85.
14. Coudeville, L., et al., Relationship between hemagglutination-inhibiting antibody titres and clinical protection against influenza: development and application of a bayesian random-effects model. BMC Med Res Methodol, 2010. 10: p. 18.
15. Koel, B. F., et al., Substitutions near the receptor binding site determine major antigenic change during influenza virus evolution. Science, 2013. 342(6161): p. 976-9.
16. Chen, C. J., et al., Influenza A Viruses Expressing Intra- or Intergroup Chimeric Hemagglutinins. J Virol, 2016. 90(7): p. 3789-93.
17. Wilson, I. A. and N. J. Cox, Structural basis of immune recognition of influenza virus hemagglutinin. Annu Rev Immunol, 1990. 8: p. 737-71.
18. Retamal, M., et al., Epitope mapping of the 2009 pandemic and the A/Brisbane/59/2007 seasonal (H1N1) influenza virus haemagglutinins using mAbs and escape mutants. J Gen Virol, 2014. 95 (Pt 11): p. 2377-89.
19. Zhao, R., et al., Identification of a highly conserved H1 subtype-specific epitope with diagnostic potential in the hemagglutinin protein of influenza A virus. PLoS One, 2011. 6(8): p. e23374.
20. Krause, J. C., et al., A broadly neutralizing human monoclonal antibody that recognizes a conserved, novel epitope on the globular head of the influenza H1N1 virus hemagglutinin. J Virol, 2011. 85(20): p. 10905-8.
21. Bouvier, N. M. and A. C. Lowen, Animal Models for Influenza Virus Pathogenesis and Transmission. Viruses, 2010. 2(8): p. 1530-1563.
22. Ekiert, D. C., et al., Cross-neutralization of influenza A viruses mediated by a single antibody loop. Nature, 2012. 489(7417): p. 526-32.
23. Tsibane, T., et al., Influenza human monoclonal antibody 1F1 interacts with three major antigenic sites and residues mediating human receptor specificity in H1N1 viruses. PLoS Pathog, 2012. 8(12): p. e1003067.
24. Whittle, J. R., et al., Broadly neutralizing human antibody that recognizes the receptor-binding pocket of influenza virus hemagglutinin. Proc Natl Acad Sci USA, 2011. 108(34): p. 14216-21.
25. Robinson, R. Q. and W. R. Dowdle., Influenza viruses., in Diagnostic procedures for viral and rickettsial infections., E. H. Lennette and N. J. Schmidt, Editors. 1969, American Public Health Association, Inc.: New York. p. 414-433.
26. Smith, D. J., et al., Mapping the antigenic and genetic evolution of influenza virus. Science, 2004. 305(5682): p. 371-6.

### 6.2 Example 2: Immunodominance of Antigenic Site B in the Hemagglutinin of the Current H3N2 Influenza Virus in Humans and Mice

This example indicates the usefulness of a panel of influenza viruses expressing a mosaic influenza virus HA, such as described in this Example, in which amino acid residues in an antigenic site in the globular head have been substituted with other residues, to assess changes in a subject's hemagglutination inhibition profile. For example, such a panel of influenza viruses may be useful to indicate a charge in a subject's immune response, e.g., after influenza vaccination. In addition, the data for an influenza virus comprising a mosaic influenza virus HA polypeptide when amino acid residues in antigenic sites are substituted reduces or eliminates hemagglutinin inhibition titer detected in plasma samples from human sunjects receiving the seasonal vaccine in Table 10. This data indicates that a mosaic influenza virus HA polypeptide with mutations in the antigenic sites of an H3 virus may be used as an immunogen to boost the immune response to the conserved antigenic sites in the HA stem domain of an H3 virus and the conserved antigenic sites in the HA globular domain of an H3 virus.

The hemagglutinin protein of H3N2 influenza viruses is the major target of neutralizing antibodies induced by infection and vaccination. However, the virus frequently escapes antibody-mediated neutralization due to mutations in the globular head domain. Five topologically distinct antigenic sites in the head domain of H3 hemagglutinin, A-E, have been previously described by mapping the binding sites of monoclonal antibodies. Yet, little is known about the contribution of each site to the immunogenicity of modern H3 hemagglutinins, as measured by hemagglutination inhibition activity that is known to correlate with protection. To investigate the hierarchy of antibody immunodominance, five ‘Δ1’ recombinant influenza viruses expressing hemagglutinin of the A/Hong Kong/4801/2014 (H3N2) strain with mutations in single antigenic sites were generated. Next, the Δ1 viruses were used to determine the hierarchy of immunodominance by measuring the hemagglutination inhibition reactivity of mouse antisera and plasma from 18 human subjects before and after seasonal influenza vaccination in 2017/2018. In both mice and humans, mutations in antigenic site B caused the most significant decrease in hemagglutination inhibition titers compared to wildtype hemagglutinin. This Example revealed that antigenic site B is immunodominant in the H3N2 influenza virus strain included in the current vaccine preparations.

### 6.2.1 INTRODUCTION

Infections with influenza viruses cause the death of 12,000-56,000 people in the United States annually (1-3). Two influenza A virus subtypes, H1N1 and H3N2, as well as influenza B viruses are currently circulating in humans. Protection provided by seasonal vaccination is only modest, especially against H3N2 viruses, with an average vaccine effectiveness of 33% reported between 2004 and 2015 (4), and an estimated 25% in the 2017-18 season (5). One of the reasons for the poor effectiveness is the rapid accumulation of mutations in the hemagglutinin (HA) surface glycoprotein, the major target of neutralizing antibodies (6-8). This antigenic drift makes it necessary to reformulate and readminister current vaccines almost yearly (9). The HA protein consists of two structurally distinct domains: the globular head composed of the majority of the HA1 polypeptide, and the stalk domain made up of portions of HA1 and the entire ectodomain of the HA2 polypeptide (10). From 1968 to 2010, 108 amino acid changes in the major epidemic strains occurred within HA1 at 63 residue positions, whereby 85.5% are clustered into regions called antigenic sites (11). Four antigenic sites, A-D, were identified in 1980 by Webster and layer by determining the reactivity patterns of monoclonal antibodies (mAbs) using hemagglutination inhibition (HI) assays (12). A fifth site, E, was later described by Skehel and colleagues in 1984 by identifying escape mutations from a panel of mAbs (13, 14). mAbs to each of the five antigenic sites have shown HI activity (12, 15).

Early studies on the HA of the A/Memphis/1/1971 H3N2 virus suggested that site A was immunodominant, as HI reactivity of ferret antiserum was completely ablated by mutations in this site (16). Moreover, human plasma samples collected in 1976 showed decreased binding to mutants in antigenic site A of the A/Aichi/2/1968 virus (17). Further research suggested that the immunodominance hierarchies may vary over time. Computational analyses provided evidence that site A was immunodominant between 1968-1971 and 1989-1995, while site B was dominant in 1972-1987 and 1996-2003 (18). Studies on plasma samples collected after seasonal vaccination in the 2006-07 and 2008-09 seasons suggested that site B was immunodominant over site A in these years, however, the other antigenic sites were not investigated (11).

To systematically study the hierarchy of antibody immunodominance of all antigenic sites, five ‘Δ1’ influenza viruses expressing the HA of the A/Hong Kong/4801/2014 virus (the H3N2 component of the 2016-17 and 2017-18 seasonal vaccines; abbreviated HK2014 in the following) were generated in the A/Puerto Rico/8/34 (PR8) backbone, each with one antigenic site mutated. These viruses served as probes to interrogate the immunodominance, as measured by HI reactivity, in mouse antisera and human plasma samples obtained before and after vaccination in the 2017-18 season. The data provided herein demonstrates that site B is immunodominant in both mice and humans.

### 6.2.2 MATERIALS AND METHODS

Recombinant HA genes and cloning: To obtain the HA gene segment of the A/Hong Kong/4801/2014 virus, RNA was isolated with the High Pure Viral RNA kit (Roche) from the New York Medical College (NYMC) X-263 strain, a 6:2 reassortant virus expressing the HK2014 surface glycoproteins on a PR8 backbone obtained from NIBSC. The viral RNA was used as a template for reverse transcription PCR amplification using the SuperScript III One-Step RT-PCR System with Platinum Taq High Fidelity DNA Polymerase (Thermo Fisher), with primers H3-for (CCGAAGTTGGGGGGGAGCAAAAGCAGGGGATAATTC) (SEQ ID NO: 101) and H3-rev (GGCCGCCGGGTTATTAGTAGAAACAAGGGTGTTTTTAATTAATG) (SEQ ID NO: 102). Cycling conditions were 15 min 60° C., 2 min 94° C., (15 s 94° C., 30 s 60° C., 2 min 68° C.)×40, 5 min 68° C., then hold at 4° C. The PCR product was purified from a preparative agarose gel with the NucleoSpin Gel and PCR Clean-up kit (Macherey-Nagel). The HA gene segment was cloned into an ambisense pDZ vector that was digested with the SapI restriction enzyme (New England Biolabs), using the In-Fusion HD Cloning Kit (Clontech). HA gene segments with mutations in the antigenic sites were designed by aligning the HA gene sequences of HK2014 and A/Jiangxi-Donghu/346-1/2013 (H10N8; sequence obtained from the Global Initiative on Sharing Avian Influenza Data [GISAID, http://gisaid.org], accession no. EPI530526) with Clustal X 2.0 (54) and replacing the respective coding sequences with the H10 virus sequences. The mutant HA gene fragments were either ordered as synthetic genes from Integrated DNA Technologies or generated by overlap extension PCR using the CloneAmp HiFi PCR Premix (Clontech) and cycling conditions that were adapted to the amplicon lengths. All HA gene segments included 15 bp overhangs at both ends that allowed for cloning into the SapI digested pDZ vector, as described above. Sequences were confirmed by Sanger sequencing (Macrogen for plasmids and GeneWiz for PCR fragments). Primers were obtained from Integrated DNA Technologies.

Viral rescue: To generate reassortant viruses, human embryonic kidney 293T cells were transfected with 0.7 μg of HA-encoding pDZ plasmid, 2.8 μg of a pRS-7 segment plasmid that drives ambisense expression of the seven gene segments of the A/Puerto Rico/8/34 (PR8) virus except HA described elsewhere (Fulton et al., submitted), and 0.5 μg of a pCAGGS plasmid expressing the HA protein of PR8 virus, using TransIT-LT1 transfection reagent (Mirus Bio) according to the manufacturer's recommendations. A 6:2 reassortant virus designated as PR8-H3N2 (HK2014) with the surface glycoproteins of HK2014, HA and NA, and the remaining six gene segments of PR8 (Fulton et al., submitted) was generated analogously. After 48 h, cells were treated for 30 min with 1 μg per mL tosyl phenylalanyl chloromethyl ketone (TPCK)-treated trypsin. Supernatants were collected, clarified by low speed centrifugation, and injected into 8 to 10-day old specific pathogen-free embryonated chicken eggs (Charles River Laboratories) that were incubated at 37° C. (28, 29). Forty-eight hours post-injection, the eggs were cooled to 4° C. for at least 4 h, then allantoic fluids were harvested and clarified by low speed centrifugation. The presence of influenza virus in the allantoic fluid was determined by performing HA assays as described below. Positive virus cultures were plaque purified on confluent MDCK cell layers in the presence of TPCK-treated trypsin and expanded in embryonated chicken eggs. Virus titers were determined by plaque assays on MDCK cells. Plaques were stained using a commercial anti-NP antibody (cat. no. PA5-32242, Invitrogen) or mAb 9H10 (32). Sequences of the HA and NA genes were confirmed by Sanger sequencing, as described in the previous paragraph.

Generation of mouse antiserum and whole virus ELISA: To generate antisera to the surface glycoproteins of HK2014, 6-8 weeks old female BALB/c or C57BL/6 mice obtained from Jackson Laboratories were primed with the 6:2 reassortant PR8-H3N2 (HK2014) virus described above either intranasally with 107 PFU or intraperitoneally with 4×10⁶ PFU and boosted four weeks later either intranasally or intraperitoneally with the same virus. Four weeks after the booster immunization, mice were euthanized and blood was drawn. Sera were separated from red blood cells by centrifugation. Animal experiments were performed in accordance with protocols approved by the Icahn School of Medicine at Mount Sinai Institutional Animal Care and Use Committee (IACUC). The presence of antibodies to the H3 HA protein was determined by whole virus ELISA as described in the following. 96-well plates were coated with H3-wt virus (allantoic fluid) diluted 1:100 in coating buffer (100 mM NaHCO₃, pH9.4) for 16 h at 4° C., washed once with phosphate-buffered saline (PBS) and blocked with 5% (w/v) skim milk powder in PBS for 1 h. Then, plates were washed once with PBS and serial two-fold dilutions of mouse sera in PBS added. After incubation for 1 h, the plates were washed three times with PBS and wells were incubated with horseradish peroxidase-conjugated anti-mouse IgG antibody (GE Healthcare) diluted 1:5,000 in 5% (w/v) skim milk powder in PBS for 1 h. Plates were washed three times with PBS and developed with SigmaFast OPD (Sigma-Aldrich) for 10 min. The reaction was stopped using 3 M hydrochloric acid and absorbance at 490 nm was measured on a Synergy 4 plate reader (BioTek).

Immunofluorescence and whole cell ELISA: MDCK monolayers in 96-well tissue culture plates were infected with a multiplicity of infection (MOI) of five with the different viruses and incubated for 16 h at 37° C. Medium was aspirated, cells were washed twice with PBS, and fixed with a methanol-free 4% paraformaldehyde solution for 15 min. After washing three times with PBS, wells were blocked for 30 min with a 5% (w/v) bovine serum albumin (BSA) solution in PBS. Cells were washed once with PBS and incubated for 2 h with mAb 9H10 (32) at 5 μg per mL or pooled mouse serum (five mice) diluted 1:400 in 1% (w/v) BSA in PBS and then washed three times with PBS. For immunofluorescence studies, cells were incubated with fluorescence-labeled anti-mouse IgG Alexa-488 antibody (Life Technologies) diluted 1:2,000 in 1% (w/v) BSA in PBS for 1 h, then washed three times with PBS before pictures were taken on a Zeiss LSM 880 Airyscan laser scanning confocal fluorescence microscope at the Microscopy Core of the Icahn School of Medicine at Mount Sinai. For whole cell ELISAs, cells were incubated with the same primary antibodies used for immunofluorescence. Cells were then incubated with horseradish peroxidase (HRP)-conjugated anti-mouse IgG antibody (GE Healthcare) diluted 1:5,000 in 1% (w/v) BSA in PBS for 1 h, then washed three times with PBS and developed with SigmaFast OPD (Sigma-Aldrich) for 10 min. The reaction was stopped using 3 M hydrochloric acid (HCl) and absorbance at 490 nm was measured on a Synergy 4 plate reader (BioTek).

ELISA with recombinant HA proteins: Recombinant HA proteins (33) were coated at a concentration of 2 μg per mL in PBS (50 μL per well) for 16 h at 4° C. After washing once with PBS containing 0.1% (v/v) Tween-20 (PBS-T), wells were blocked with 5% (w/v) skim milk powder in PBS for 1 h. Wells were washed once with PBS-T, and pooled mouse antiserum diluted in PBS (50 μL per well) was added. After incubation for 1 h, wells were incubated with HRP-conjugated anti-mouse IgG antibody (GE Healthcare) diluted 1:5,000 in 5% (w/v) skim milk powder in PBS for 1 h, then washed three times with PBS-T and developed with SigmaFast OPD (Sigma-Aldrich) for 15 min. The reaction was stopped using 3 M HCl and absorbance at 490 nm was measured on a Synergy 4 plate reader (BioTek).

Hemagglutination assays: Serial two-fold dilutions of influenza virus samples (allantoic fluids) were prepared in 96 V-well microtiter plates, using PBS as diluent to a final volume of 50 μL per well. Then, 50 μL of a 0.5% suspension of turkey red blood cells (Lampire) in PBS were added to each well. The plates were incubated at 4° C. until the red blood cells in PBS control samples settled to the bottom. The HA titer (HA units) was defined as the reciprocal of the highest dilution of virus that caused red blood cell hemagglutination.

Receptor-destroying enzyme (RDE) treatment of plasma and serum: Human plasma samples were pre-treated at 56° C. for 30 min. One volume of pre-treated human plasma or mouse serum was treated with three volumes of Vibrio cholerae RDE (Denka Seiken, Chuo-ku, Tokyo, Japan) solution in PBS at 37° C. for 16 h according to manufacturer's recommendations. To the RDE-treated samples were added three volumes of a 2.5% sodium citrate solution. After incubation at 56° C. for 30 min, three volumes of PBS were added to each sample for a final dilution of 1:10.

Hemagglutination inhibition (HI) assays: HI assays were performed as described previously (30, 55). Allantoic fluid samples of each influenza virus strain were diluted with PBS to a final HA titer of 8 HA units per 50 μL. Two-fold dilutions (25 μL) of RDE-treated plasma/serum in PBS prepared in 96 V-well microtiter plates were then combined with 25 μL of the diluted influenza viruses. The plates were then incubated for 30 min at room temperature to allow HA-specific antibodies in the plasma/serum to bind to the virus. Then, 50 μL of a 0.5% suspension of turkey red blood cells (Lampire) that was washed once with PBS was added to each well, and the plates were incubated at 4° C. until the red blood cells in PBS control samples settled to the bottom. Human plasma samples were tested in duplicates and pooled mouse sera in triplicates. Because of the limited volume, individual mouse sera were tested once. The HI titer was defined as the reciprocal of the highest dilution of plasma (serum) that inhibited red blood cell hemagglutination.

Human subjects: Eighteen individuals provided informed consented and donated blood before/on the day of seasonal influenza vaccination as well as four to eight weeks later. Plasma samples were stored at −80° C. until use. The Institutional Review Board (IRB) of the Icahn School of Medicine at Mount Sinai approved the study.

Statistics: Statistical data were generated using GraphPad Prism version 5.03 (GraphPad Software). Statistical significance between groups was determined by transforming HI titers into logarithmic values and performing One-way analysis of variance (ANOVA) with Newman-Keuls post test, as described previously (56). Normalized HI titers were compared by Dunn-corrected Kruskal-Wallis tests.

### 6.2.3 RESULTS

### Rescue and Characterization of Δ1 Viruses

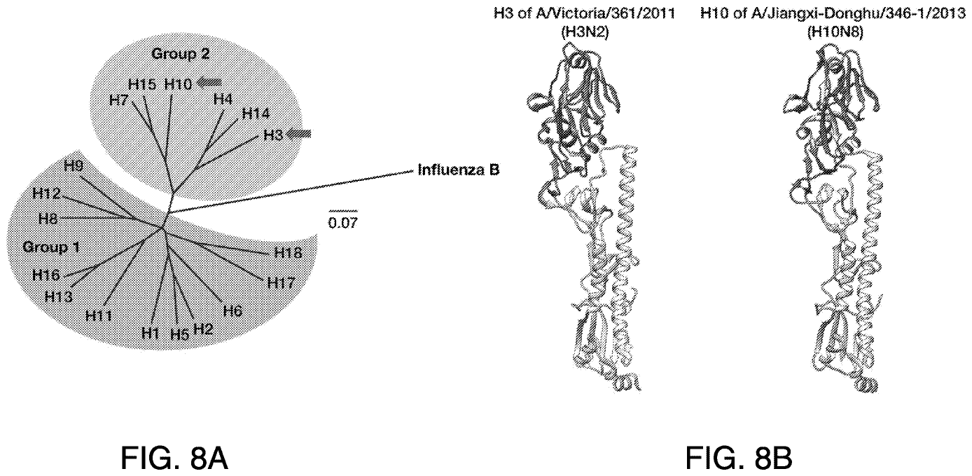

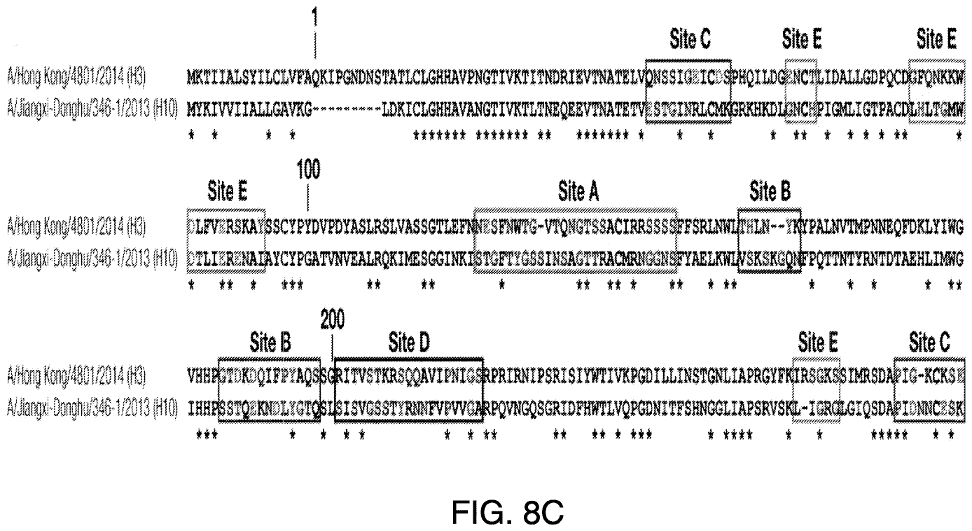

A 7:1 reassortant virus expressing the H3 protein of the HK2014 virus (“H3-wt”) was successfully rescued in the PR8 backbone. Using the same backbone, the next step was to rescue Δ1 viruses in which single antigenic sites of the HK2014 HA were replaced with the corresponding sequences of the HA of the avian A/Jiangxi-Donghu/346-1/2013 (H10N8) virus (19). Both H10 and H3 are group 2 HAs (FIG. 8A) with highly similar crystal structures (FIG. 8B) but little amino acid sequence similarity in their head domains (FIG. 8C). Various mutant HA proteins were tested for their ability to generate viable Δ1 viruses, as determined by hemagglutination assays. In the following, the rescued viruses are designated as H3-ΔA through H3-ΔE, depending on which antigenic site was mutated in the HA protein. A previous analysis of H3N2 strains from 1968-2003 demonstrated that one to three amino acid substitutions within an antigenic site were sufficient to cause major antigenic change, as measured by HI reactivity (24). Similarly, two to three mutations within an antigenic site of the PR8 H1 hemagglutinin were previously shown to be sufficient to ablate binding of antigenic site-specific mAbs (24). Thus, Δ1 viruses were rescued with at least three amino acid substitutions in the respective antigenic site that should thereby be antigenically altered. Additionally, several amino acids that were mutated are at positions of previously described escape mutations from mAbs or polyclonal sera (11, 14-16, 24, 26, 27).

The amino acid residues that were successfully exchanged in the HA protein of the five Δ1 viruses are depicted in FIG. 9A. In the H3-ΔA virus, HA residues 140 and 142-145 of site A that forms an exposed loop were replaced (FIG. 9B). Site B, located at the tip of the head domain, consists of a loop (residues 155-160) and an α-helix (residues 187-196). The H3-AB virus contains mutations in both regions, at positions 159 and 189, as well as an additional compensatory mutation at position 188 in the helix. The H3-AC virus was rescued with a total of 15 amino acid substitutions in site C, which is located in the interface between the head and stalk domains. Site D, located at the trimer interface, tolerated three substitutions at positions 207-209 that according to the crystal structure are exposed on the surface of the HA trimer (FIG. 9A). An additional compensatory mutation was observed in site D at position 212. Mutations in site E, which is located at the center of the head domain, were successfully introduced in three different regions in the primary sequence. This virus, termed H3-ΔE, contained a total of six amino acid substitutions. A virus containing all the mutations of the five Δ1 viruses, termed H3-45, was also successfully rescued. The H3-45 virus, in which all these amino acid changes were combined, did not contain the compensatory mutation in site B at position 188, but one at residue 246 (asparagine to histidine) outside of the antigenic sites. The mosaic influenza virus HA of H3-45 comprises the amino acid sequence in SEQ ID NO: 31. In addition, a virus with a chimeric HA (9, 28-31) designated as cH10/3, with the entire head domain of the HK2014 HA replaced with the H10 head, was successfully rescued.

After growing for 48 h in embryonated chicken eggs, the various plaque-purified reassortant viruses reached titers between 4.9×10⁷ (H3-AC) and 3.5×10⁹ (cH10/3) plaque-forming units per milliliter (PFU/mL) (FIG. 9C). Hemagglutination titers ranged between 64 (H3-AC and H3-45) and 512 (H3-ΔD) HA units per 50 μL (FIG. 9D). Immunofluorescence microscopy experiments of virus-infected Madin-Darby canine kidney (MDCK) cells, using mAb 9H10 that recognizes a conformational epitope in the stalk of group 2 HAs (32), verified surface expression of the various HA proteins (FIG. 9E). A similar staining pattern was observed with polyclonal mouse antiserum generated against a 6:2 reassortant virus expressing HA and neuraminidase of HK2014 in the PR8 backbone, designated as PR8-H3N2 (HK2014) in the following. The results obtained by immunofluorescence microscopy were confirmed by whole cell ELISA experiments that indicated comparable HA expression levels for the various recombinant viruses (FIG. 9F). Binding of the antiserum to the surface of cells infected with H3-45 and cH10/3 viruses indicated the presence of stalk-specific antibodies. To assess their presence, ELISA experiments was performed using plates coated with recombinant HA proteins (33). The mouse antiserum bound to the recombinant HK2014 H3 protein (FIG. 9G). Weaker binding was observed for the cH14/3 recombinant protein that consists of the HK2014 stalk domain and the HA head domain of the avian A/mallard/Gurjev/263/1982 (H14N5) virus. In contrast, no binding was observed when the full-length H14 protein was coated. Although stalk-reactive antibodies raised with H3 are expected to partially cross-react with the H14 stalk domain (both are group 2 HAs), their abundance was likely too low to give a detectable signal for full-length H14 protein. Overall, the ELISA measurements confirmed the presence of H3 stalk-reactive antibodies in the murine antiserum.

### Hierarchy of Immunodominance in Immunized Mice

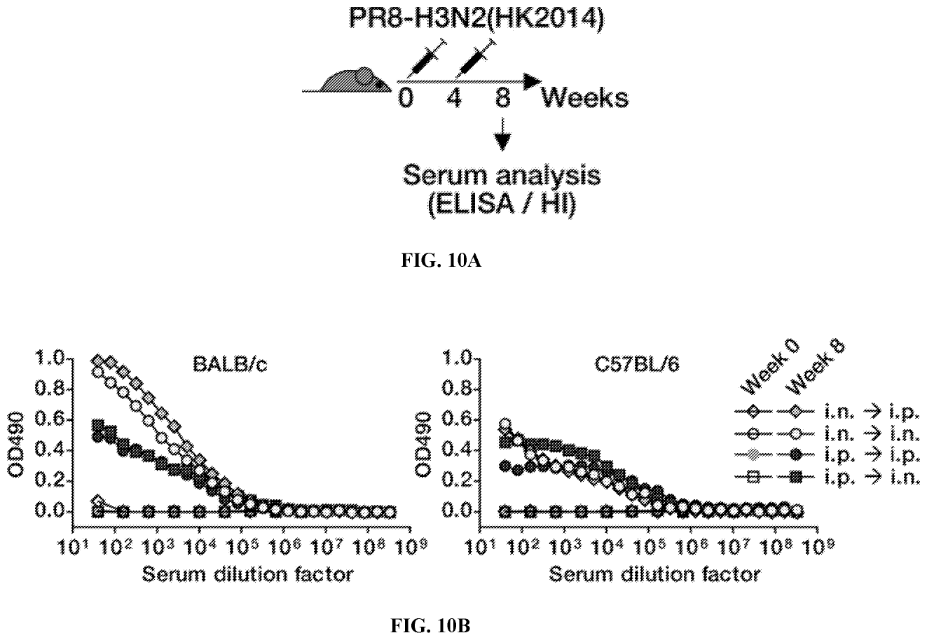

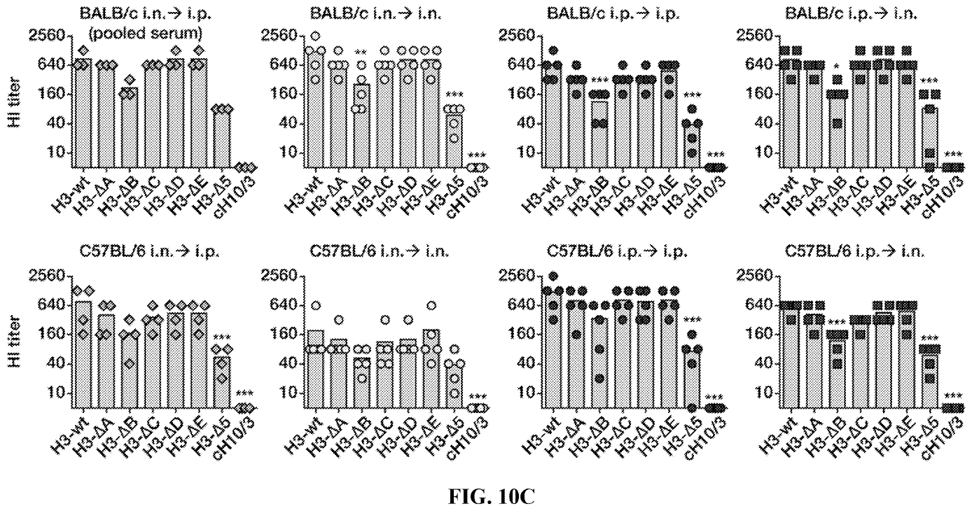

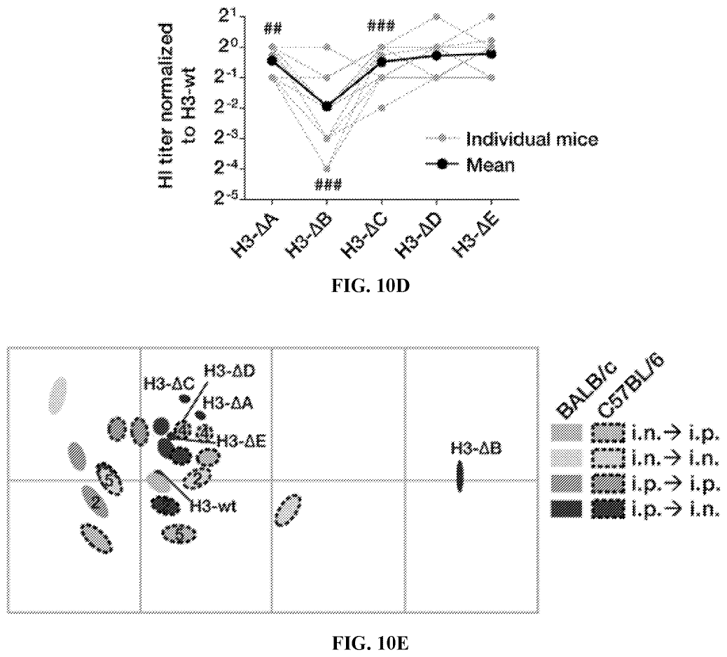

A recent study in mice showed varying antibody responses to the antigenic sites of the PR8 (H1N1) HA protein, depending on the genetic background of the mice (BALB/c versus C57BL/6 strains) and whether animals were infected with virus intranasally or immunized intraperitoneally or intramuscularly (34). To investigate if recognition of HK2014 HA epitopes also depended on mouse strain and route of virus administration, groups of four to five animals were primed with the live PR8-H3N2 (HK2014) virus either intranasally or intraperitoneally and boosted four weeks later either intranasally or intraperitoneally (four combinations per mouse strain). Serum drawn four weeks after the second immunization was further analyzed (FIG. 10A). To test whether mice developed antibodies to the HK2014 HA protein, pooled sera were subjected to ELISA measurements, whereby the H3-wt virus was coated onto the ELISA plates (FIG. 10B). All immunization regimes elicited detectable levels of IgG in both strains. The strongest ELISA signals were observed for BALB/c mice that were primed intranasally.

To determine the contribution of each antigenic site to the immunogenicity of H3 HA, HI assays were performed with the panel of eight recombinant viruses described above (FIG. 10C). HI titers have been shown to correlate with neutralizing activity (35) and with influenza immunity (36-38). All animals mounted HI titers of 80 or higher against H3-wt virus. HI titers against cH10/3 virus were below the level of detection in all mice, suggesting that antibodies against the HK2014 head domain do not cross-react with the H10 head domain. On average, HI titers against the H3-45 virus were about 8-fold lower than those to H3-wt, and below the limit of detection in some animals, indicating that the antigenic sites were successfully antigenically altered. Irrespective of the mouse strain or route of immunization, HI titers against the H3-AB virus were consistently lower than those to the H3-wt virus, indicating that site B was immunodominant by HI reactivity. To compare the immunodominance profiles, HI titers against the 41 mutant viruses were normalized to those observed for the H3-wt virus for each mouse (FIG. 10D). The normalized data revealed a significant contribution of site B and, to a lesser extent, sites A and C to the immunodominance hierarchy. Mutating the other two antigenic sites, D and E, had no significant effect on HI reactivity. Plotting the HI data of all mice on one map by using antigenic cartography (24) revealed that there were no measurable differences in the immunodominance hierarchies between the two mouse strains or the route of administration (FIG. 10E).

Hierarchy of Immunodominance in Humans Before and After Seasonal Vaccination

Next, hierarchies of immunodominance in plasma samples obtained from 18 healthy human subjects before and four to eight weeks (average: 35 days; range: 27-57 days) following vaccination in the 2017-18 season were investigated (Table 10). Eleven of the individuals received tri- or quadrivalent vaccines manufactured in eggs, all of which contained an HK2014-like virus as H3N2 component. Six additional subjects received Flucelvax, a vaccine propagated in MDCK suspension cells that contained an A/Singapore/GP2050/2015-like H3N2 component (39). One individual received the quadrivalent Flublok vaccine that is produced in insect cells and contains recombinant proteins instead of inactivated viruses (40). In the 2017-18 season, Flublok contained an HK2014-like HA protein as H3N2 component.

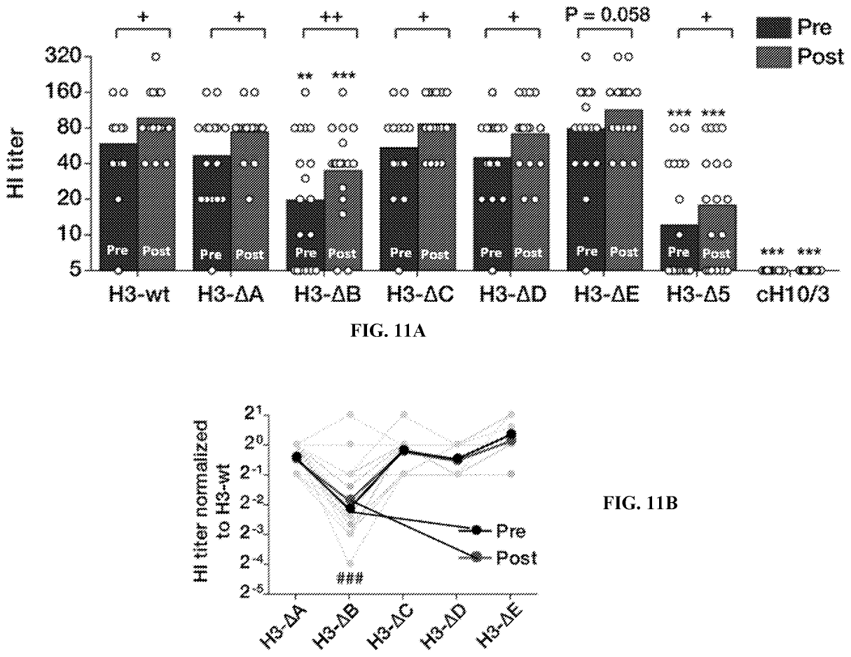

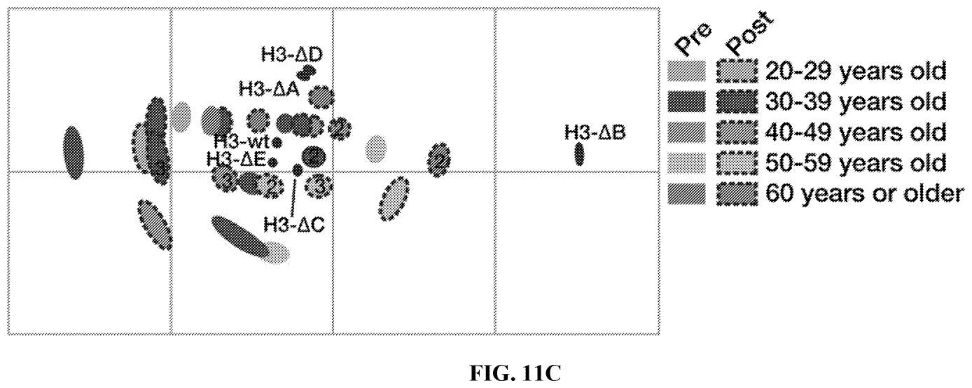

To determine the relative contributions of the antigenic sites to the antibody repertoire in the 18 individuals, HI assays with the eight recombinant viruses were performed. Vaccination boosted HI titers against all viruses with statistical significance, except for H3-ΔE (P=0.058) and cH10/3 (no measurable titer) (FIG. 11A). The geometric mean HI titers against H3-wt virus pre- and post-vaccination were 59 and 97, respectively. Ten of eighteen individuals had higher HI titers post-vaccination than pre-vaccination against H3-wt virus with six showing a two-fold and three a four-fold increase; one individual had an undetectable titer before vaccination and a titer of 40 post-vaccination. Six subjects had equal titers pre- and post-vaccination, and two showed a two-fold decrease in HI titers post-vaccination. None of the plasma samples had a measurable HI titer against the cH10/3 virus, confirming the absence of HI reactive H10 head specific antibodies in these subjects. Both pre- and post-vaccination HI titers were significantly lower against the H3-AB virus compared to H3-wt virus.

TABLE 10
Plasma samples analyzed in the present Example.

| | | | Vaccine | No. of | | |
|---|---|---|---|---|---|---|
| Subject | | | trade | vaccine | | |
| no. | Sex | Age | name | components | Produced in | Manufacturer |
| 1 | F | 50-59 | Flucelvax | 4 | Cell culture | Seqirus, Inc. |
| 2 | M | 30-39 | Flucelvax | 4 | Cell culture | Seqirus, Inc. |
| 3 | M | >60 | Fluzone | 3 | Eggs | Sanofi Pasteur |
| | | | High-Dose | | | |
| 4 | F | >60 | Fluzone | 3 | Eggs | Sanofi Pasteur |
| | | | High-Dose | | | |
| 5 | F | 40-49 | Flucelvax | 4 | Cell culture | Seqirus, Inc. |
| 6 | F | 50-59 | Flucelvax | 4 | Cell culture | Seqirus, Inc. |
| 7 | M | 40-49 | Flucelvax | 4 | Cell culture | Seqirus, Inc. |
| 8 | M | 50-59 | Flucelvax | 4 | Cell culture | Seqirus, Inc. |
| 9 | M | 30-39 | Fluarix | 4 | Eggs | GSK |
| | | | Quadrivalent | | | |
| 10 | M | >60 | Fluvirin | 3 | Eggs | Seqirus, Inc. |
| 11 | M | 50-59 | Fluarix | 4 | Eggs | GSK |
| | | | Quadrivalent | | | |
| 12 | M | 40-49 | Fluarix | 4 | Eggs | GSK |
| | | | Quadrivalent | | | |
| 13 | F | 30-39 | Fluzone | 4 | Eggs | Sanofi Pasteur |
| | | | Quadrivalent | | | |
| 14 | M | 20-29 | Fluarix | 4 | Eggs | GSK |
| | | | Quadrivalent | | | |
| 15 | F | 40-49 | Fluarix | 4 | Eggs | GSK |
| | | | Quadrivalent | | | |
| 16 | M | 30-39 | Flublok | 4 | Insect cells | Protein Sciences |
| | | | Quadrivalent | | | Corp. |
| 17 | F | 30-39 | Fluarix | 4 | Eggs | GSK |
| | | | Quadrivalent | | | |
| 18 | M | 30-39 | Fluzone | 4 | Eggs | Sanofi Pasteur |
| | | | Quadrivalent | | | |

Vaccine components are as follows.
Fluarix Quadrivalent, A/Singapore/GP1908/2015 (H1N1) IVR-180, (an A/Michigan/45/2015 (H1N1) pdm09-like virus), A/Hong Kong/4801/2014 (H3N2)NYMC X-263B, B/Brisbane/60/2008, B/Phuket/3073/2013;
Flucelvax, A/Brisbane/10/2010(wildtype) (an A/California/7/2009 (H1N1)pdm09-like virus), A/Singapore/GP2050/2015 (wildtype) (an A/Hong Kong/4801/2014 (H3N2)-like virus), B/Utah/9/2014 (a B/Phuket/3073/2013-like virus), B/Hong Kong/259/2010 (a B/Brisbane/60/08-like virus);
Fluzone High-Dose, A/Michigan/45/2015 X-275 (H1N1), A/Hong Kong/4801/2014 (H3N2) NYMC X-263B, B/Brisbane/60/2008;
Fluvirin, A/Singapore/GP1908/2015, IVR-180 (an A/Michigan/45/2015 (H1N1)pdm09-like virus), A/Hong Kong/4801/2014 (H3N2) NYMC X-263B, B/Brisbane/60/2008;
Flublok Quadrivalent, A/Michigan/45/2015 (H1N1), A/Hong Kong/4801/2014 (H3N2), B/Brisbane/60/2008, B/Phuket/3073/2013;
Fluzone Quadrivalent, A/Michigan/45/2015 X-275 (H1N1), A/Hong Kong/4801/2014 (H3N2) NYMC X-263B, B/Brisbane/60/2008, B/Phuket/3073/2013 and B/Brisbane/60/2008.

To better compare the immunodominance profiles, HI titers against the Δ1 mutant viruses were normalized to those observed for the H3-wt virus for each individual (FIG. 11B). The normalized data confirmed a significant contribution of site B to the HI reactive antibody response. Exchanging the other four antigenic sites had no significant effect on the HI reactivity. Furthermore, immunodominance hierarchies were similar in plasma taken pre- and post-vaccination. Plotting the HI data of all subjects on one antigenic map confirmed that there were no significant differences in the immunodominance hierarchies before and after vaccination (FIG. 11C). In addition, the age of the subjects did not have a significant impact on the immunodominance profiles.

### Discussion

In this Example, a panel of Δ1 influenza viruses expressing HA of the current H3N2 vaccine component, each with mutations in one of the five antigenic sites, were generated. In addition, a 45 virus with mutations in all five sites was rescued. These viruses were used to interrogate the hierarchy of immunodominance in mice and humans, as measured by HI reactivity. ELISA studies with recombinant HA proteins revealed that the majority of serum antibodies in immunized mice are directed against the head domain, antibodies that are usually HI reactive. Stalk-specific antibodies, which are generally HI inactive, are also present, but to a lower extent, which confirms the known immunodominance of the head over the stalk (41). A substantially lower HI reactivity to the H3-45 virus compared to H3-wt virus indicated that the introduced mutations successfully altered the antigenicity of the antigenic sites. Residual HI reactivity to this virus may have been due to antibodies binding to head domain epitopes outside of the major antigenic sites, or antibodies targeting the receptor binding pocket directly using a long CDR3 region. Such mAbs have been isolated from humans and typically exhibit binding that is unaffected by mutations in the major antigenic sites (42-47). In addition, the fact that the H3-45 virus is missing the compensatory mutation at position 188 in site B, which is present in the H3-AB virus, may have caused residual HI reactivity of that antigenic site.

The results in humans are in good agreement with a previous study that reported the immunodominance of site B in human subjects vaccinated in the 2006-07 and 2008-09 seasons, as measured by ELISA using recombinant HA proteins (11). The latter study, however, only investigated the effect of mutations in antigenic sites A and B on serum reactivity. This Example shows that mutations in sites A, C, D and E of the HK2014 HA did not significantly affect HI reactivity in the human plasma samples tested.

Ten out of eighteen individuals showed increased HI reactivity against the H3-wt virus after vaccination, whereas eight subjects had similar or lower HI reactivity post-vaccination. Despite the varying responses to vaccination with respect to HI reactivity, antigenic site B was dominant both pre- and post-vaccination, suggesting that vaccination did not alter the immunodominance hierarchy of HI reactive antibodies. There were no apparent differences in the post-vaccine HI reactivities between vaccines based on egg- or cell culture-propagated viruses, or the vaccine using recombinant proteins. However, the sample sizes were too small to allow for statistically meaningful analyses. Irrespective of the age of the subjects, antigenic site B was immunodominant, suggesting that the immune history to influenza (48) did not majorly affect the immunodominance hierarchy of HI reactive antibodies in the tested individuals.

In contrast to humans, HI reactivity of mouse antisera was also significantly decreased by mutations in sites A and C, albeit to a lower extent than through mutations in site B. The immunodominance hierarchy in mice was independent of whether antiserum was raised in the BALB/c or C57BL/6 strains and was also not affected by the route of immunization, intranasal vs. intraperitoneal. In contrast, the immunodominance hierarchy to antigenic sites of the PR8 (H1N1) virus, as measured by ELISA, was previously reported to vary depending on mouse strain and antigen delivery (34). In accordance with the findings reported herein, however, the latter study found that irrespective of the mouse strain and route of immunization, most of the antibodies four weeks post-immunization bound to antigenic sites Sa and Sb of the PR8 HA that correspond to site B in the H3 HA (34). The fact that site B is immunodominant with respect to HI reactivity in both mice and humans suggests that the rules governing the immunodominance to the current H3 protein are largely conserved between the two species.

A better understanding of the immunodominance hierarchy can assist in the efforts towards vaccines that provide better and longer lasting protection (41). For instance, vaccines eliciting a more balanced immunity against the head domain may be superior to current vaccines, as thereby susceptibility to drifted strains will be lower (49). Using the panel of mosaic viruses described herein, no change was observed in the immunodominance hierarchy pre- and post-vaccination, indicating that the seasonal vaccines were like boosting preexisting antibody responses to the antigenic sites, which are prone to antigenic drift (11). Knowledge on the immunodominance may help to design antigens that are able to focus antibody responses to more conserved but immunosubdominant epitopes in the H3 head (27, 43, 50, 51) or stalk domain (32, 52, 53), which could limit immune evasion by H3N2 influenza viruses.

### 6.2.4 REFERENCES

1. Fiore A E, Uyeki T M, Broder K, Finelli L, Euler G L, Singleton J A, Iskander J K, Wortley P M, Shay D K, Bresee J S, Cox N J; Centers for Disease Control and Prevention (DCD). 2010. Prevention and control of influenza with vaccines: recommendations of the Advisory Committee on Immunization Practices (ACIP), 2010. MMWR Recomm Rep 59:1-62.
2. Rolfes M A, Foppa I M, Garg S, Flannery B, Brammer L, Singleton J A, Burns E, Jernigan D, Olsen S J, Bresee J, Reed C. 2018. Annual estimates of the burden of seasonal influenza in the United States: A tool for strengthening influenza surveillance and preparedness. Influenza Other Respir Viruses 12:132-137. https://doi.org/10.1111/irv.12486.
3. Matias G, Haguinet F, Lustig R L, Edelman L, Chowell G, Taylor R J. 2016. Model estimates of the burden of outpatient visits attributable to influenza in the United States. BMC Infect Dis 16:641.
4. Belongia E A, Simpson M D, King J P, Sundaram M E, Kelley N S, Osterholm M T, McLean H Q. 2016. Variable influenza vaccine effectiveness by subtype: a systematic review and meta-analysis of test-negative design studies. Lancet Infect Dis 16:942-951. https://doi.org/10.1016/S1473-3099(16)00129-8.
5. Flannery B, Chung J R, Belongia E A, McLean H Q, Gaglani M, Murthy K, Zimmerman R K, Nowalk M P, Jackson M L, Jackson L A, Monto A S, Martin E T, Foust A, Sessions W, Berman L, Barnes J R, Spencer S, Fry A M. 2018. Interim Estimates of 2017-18 Seasonal Influenza Vaccine Effectiveness—United States, February 2018. MMWR Morb Mortal Wkly Rep 67:180-185. https:/doi.org/10.15585/mmwr.mm6706a2.
6. Altman M O, Angeletti D, Yewdell J W. 2018. Antibody Immunodominance: The Key to Understanding Influenza Virus Antigenic Drift. Viral Immunol 31:142-149. https://doi.org/10.1089/vim.2017.0129.
7. Fitch W M, Bush R M, Bender C A, Cox N J. 1997. Long term trends in the evolution of H (3) HA1 human influenza type A. Proc Natl Acad Sci USA 94:7712-7718.
8. Bhatt S, Holmes E C, Pybus O G. 2011. The genomic rate of molecular adaptation of the human influenza A virus. Mol Biol Evol 28:2443-2451. https://doi.org/10.1093/molbev/msr044.
9. Krammer F. 2017. Strategies to induce broadly protective antibody responses to viral glycoproteins. Expert Rev Vaccines 16:503-513. https://doi.org/10.1080/14760584.2017.1299576.
10. Wilson I A, Skehel J J, Wiley D C. 1981. Structure of the haemagglutinin membrane glycoprotein of influenza virus at 3 A resolution. Nature 289:366-373.
11. Popova L, Smith K, West A H, Wilson P C, James J A, Thompson L F, Air G M. 2012. Immunodominance of antigenic site B over site A of hemagglutinin of recent H3N2 influenza viruses. PLoS One 7: e41895. https://doi.org/10.1371/journal.pone.0041895.
12. Webster R G, Laver W G. 1980. Determination of the number of nonoverlapping antigenic areas on Hong Kong (H3N2) influenza virus hemagglutinin with monoclonal antibodies and the selection of variants with potential epidemiological significance. Virology 104:139-148.
13. Skehel J J, Stevens D J, Daniels R S, Douglas A R, Knossow M, Wilson I A, Wiley D C. 1984. A carbohydrate side chain on hemagglutinins of Hong Kong influenza viruses inhibits recognition by a monoclonal antibody. Proc Natl Acad Sci USA 81: 1779-1783.
14. Wiley D C, Skehel J J. 1987. The structure and function of the hemagglutinin membrane glycoprotein of influenza virus. Annu Rev Biochem 56:365-394.
15. Nakajima S, Nobusawa E, Nakajima K. 2000. Variation in response among individuals to antigenic sites on the HA protein of human influenza virus may be responsible for the emergence of drift strains in the human population. Virology 274:220-231.
16. Laver W G, Air G M, Webster R G. 1981. Mechanism of antigenic drift in influenza virus. Amino acid sequence changes in an antigenically active region of Hong Kong (H3N2) influenza virus hemagglutinin. J Mol Biol 145:339-361.
17. Abe Y, Takashita E, Sugawara K, Matsuzaki Y, Muraki Y, Hongo S. 2004. Effect of the addition of oligosaccharides on the biological activities and antigenicity of influenza A/H3N2 virus hemagglutinin. J Virol 78:9605-9611. https://doi.org/10.1128/JVI.78.18.9605-9611.2004.
18. Pan K, Long J, Sun H, Tobin G J, Nara P L, Deem M W. 2011. Selective pressure to increase charge in immunodominant epitopes of the H3 hemagglutinin influenza protein. J Mol Evol 72:90-103. https://doi.org/10.1007/s00239-010-9405-4.
19. Chen H, Yuan H, Gao R, Zhang J, Wang D, Xiong Y, Fan G, Yang F, Li X, Zhou J, Zou S, Yang L, Chen T, Dong L, Bo H, Zhao X, Zhang Y, Lan Y, Bai T, Dong J, Li Q, Wang S, Zhang Y, Li H, Gong T, Shi Y, Ni X, Li J, Zhou J, Fan J, Wu J, Zhou X, Hu M, Wan J, Yang W, Li D, Wu G, Feng Z, Gao G F, Wang Y, Jin Q, Liu M, Shu Y. 2014. Clinical and epidemiological characteristics of a fatal case of avian influenza A H10N8 virus infection: a descriptive study. Lancet 383:714-721. https://doi.org/10.1016/S0140-6736(14)60111-2.
20. Krammer F. 2015. Emerging influenza viruses and the prospect of a universal influenza virus vaccine. Biotechnol J 10:690-701. https://doi.org/10.1002/biot.201400393.
21. Lee P S, Ohshima N, Stanfield R L, Yu W, Iba Y, Okuno Y, Kurosawa Y, Wilson I A. 2014. Receptor mimicry by antibody F045-092 facilitates universal binding to the H3 subtype of influenza virus. Nat Commun 5:3614. https://doi.org/10.1038/ncomms4614.
22. Zhang H, de Vries R P, Tzarum N, Zhu X, Yu W, McBride R, Paulson J C, Wilson I A. 2015. A human-infecting H10N8 influenza virus retains a strong preference for avian-type receptors. Cell Host Microbe 17:377-384. https://doi.org/10.1016/j.chom.2015.02.006.
23. Pettersen E F, Goddard T D, Huang C C, Couch G S, Greenblatt D M, Meng E C, Ferrin T E. 2004. UCSF Chimera—a visualization system for exploratory research and analysis. J Comput Chem 25:1605-1612.
24. Koel B F, Burke D F, Bestebroer T M, van der Vliet S, Zondag G C, Vervaet G, Skepner E, Lewis N S, Spronken M I, Russell C A, Eropkin M Y, Hurt A C, Barr I G, de Jong J C, Rimmelzwaan G F, Osterhaus A D, Fouchier R A, Smith D J. 2013. Substitutions near the receptor binding site determine major antigenic change during influenza virus evolution. Science 342:976-979. https://doi.org/10.1126/science. 1244730.
25. Das S R, Hensley S E, Ince W L, Brooke C B, Subba A, Delboy M G, Russ G, Gibbs J S, Bennink J R, Yewdell J W. 2013. Defining influenza A virus hemagglutinin antigenic drift by sequential monoclonal antibody selection. Cell Host Microbe 13:314-323. https://doi.org/10.1016/j.chom.2013.02.008.
26. Wrammert J, Smith K, Miller J, Langley W A, Kokko K, Larsen C, Zheng N Y, Mays I, Garman L, Helms C, James J, Air G M, Capra J D, Ahmed R, Wilson P C. 2008. Rapid cloning of high-affinity human monoclonal antibodies against influenza virus. Nature 453, 667-671. https://doi.org/10.1038/nature06890.
27. Benjamin E, Wang W, Mcauliffe J M, Palmer-Hill F J, Kallewaard N L, Chen Z, Suzich J A, Blair W S, Jin H, Zhu Q. 2014. A broadly neutralizing human monoclonal antibody directed against a novel conserved epitope on the influenza virus H3 hemagglutinin globular head. J Virol 88:6743-6750. https://doi.org/10.1128/JVI.03562-13.
28. Hai R, Krammer F, Tan G S, Pica N, Eggink D, Maamary J, Margine I, Albrecht R A, Palese P. 2012. Influenza viruses expressing chimeric hemagglutinins: globular head and stalk domains derived from different J subtypes. Virol 86:5774-5781. https://doi.org/10.1128/JVI.00137-12.
29. Chen C J, Ermler M E, Tan G S, Krammer F, Palese P, Hai R. 2016. Influenza A Viruses Expressing Intra- or Intergroup Chimeric Hemagglutinins. J Virol 90:3789-3793. https://doi.org/10.1128/JVI.03060-15.
30. Nachbagauer R, Liu W C, Choi A, Wohlbold T J, Atlas T, Rajendran M, Solórzano A, Berlanda-Scorza F, García-Sastre A, Palese P, Albrecht R A, Krammer F. 2017. A universal influenza virus vaccine candidate confers protection against pandemic H1N1 infection in preclinical ferret studies. NPJ Vaccines 2:26. https://doi.org/10.1038/s41541-017-0026-4.
31. Ermler M E, Kirkpatrick E, Sun W, Hai R, Amanat F, Chromikova V, Palese P, Krammer F. 2017. Chimeric Hemagglutinin Constructs Induce Broad Protection against Influenza B Virus Challenge in the Mouse Model. J Virol 91, pii=e00286-17. https://doi.org/10.1128/JVI.00286-17.
32. Tan G S, Lee P S, Hoffman R M, Mazel-Sanchez B, Krammer F, Leon P E, Ward A B, Wilson I A, Palese P. 2014. Characterization of a broadly neutralizing monoclonal antibody that targets the fusion domain of group 2 influenza A virus hemagglutinin. J Virol 88:13580-13592. https://doi.org/10.1128/JVI.02289-14.
33. Margine I, Hai R, Albrecht R A, Obermoser G, Harrod A C, Banchereau J, Palucka K, García-Sastre A, Palese P, Treanor J J, Krammer F. 2013. H3N2 influenza virus infection induces broadly reactive hemagglutinin stalk antibodies in humans and mice. J Virol 87:4728-4737. https://doi.org/10.1128/JVI.03509-12.
34. Angeletti D, Gibbs J S, Angel M, Kosik I, Hickman H D, Frank G M, Das S R, Wheatley A K, Prabhakaran M, Leggat D J, McDermott A B, Yewdell J W. 2017. Defining B cell immunodominance to viruses. Nat Immunol 18:456-463. https://doi.org/10.1038/ni.3680.
35. Truelove S, Zhu H, Lessler J, Riley S, Read J M, Wang S, Kwok K O, Guan Y, Jiang C Q, Cummings D A. 2016. A comparison of hemagglutination inhibition and neutralization assays for characterizing immunity to seasonal influenza A. Influenza Other Respir Viruses 10:518-524. https://doi.org/10.1111/irv.12408.
36. Memoli M J, Shaw P A, Han A, Czajkowski L, Reed S, Athota R, Bristol T, Fargis S, Risos K, Powers J H, Davey R T Jr, Taubenberger J K. 2016. Evaluation of Antihemagglutinin and Antineuraminidase Antibodies as Correlates of Protection in an Influenza A/H1N1 Virus Healthy Human Challenge Model. MBio 7: e00417-16. https://doi.org/10.1128/mBio.00417-16.
37. Potter C W, Oxford JS. 1979. Determinants of immunity to influenza infection in man. Br Med Bull 35:69-75.
38. Benoit A, Beran J, Devaster J M, Esen M, Launay O, Leroux-Roels G, McElhaney J E, Oostvogels L, van Essen G A, Gaglani M, Jackson L A, Vesikari T, Legrand C, Tibaldi F, Innis B L, Dewe W. 2015. Hemagglutination Inhibition Antibody Titers as a Correlate of Protection Against Seasonal A/H3N2 Influenza Disease. Open Forum Infect Dis 2: ofv067. https://doi.org/10.1093/ofid/ofv067.
39. Manini I, Domnich A, Amicizia D, Rossi S, Pozzi T, Gasparini R, Panatto D, Montomoli E. 2015. Flucelvax (Optaflu) for seasonal influenza. Expert Rev Vaccines 14:789-804.
40. Yang L P. 2013. Recombinant trivalent influenza vaccine (flublok (®)): a review of its use in the prevention of seasonal influenza in adults. Drugs 73:1357-1366. https://doi.org/10.1007/s40265-013-0103-6.
41. Angeletti D, Yewdell J W. Is It Possible to Develop a “Universal” Influenza Virus Vaccine? Outflanking Antibody Immunodominance on the Road to Universal Influenza Vaccination. 2017. Cold Spring Harb Perspect Biol pii: a028852 [Epub ahead of print]. https://doi.org/10.1101/cshperspect.a028852.
42. Ekiert D C, Kashyap A K, Steel J, Rubrum A, Bhabha G, Khayat R, Lee J H, Dillon M A, O'Neil R E, Faynboym A M, Horowitz M, Horowitz L, Ward A B, Palese P, Webby R, Lerner R A, Bhatt R R, Wilson I A. 2012. Cross-neutralization of influenza A viruses mediated by a single antibody loop. Nature. 2012 Sep. 27; 489(7417):526-32. https://doi.org/10.1038/nature11414.
43. Ohshima N, Iba Y, Kubota-Koketsu R, Asano Y, Okuno Y, Kurosawa Y. 2011. Naturally occurring antibodies in humans can neutralize a variety of influenza virus strains, including H3, H1, H2, and H5. J Virol 85:11048-11057. https://doi.org/10.1128/JVI.05397-11.
44. Lee P S, Yoshida R, Ekiert D C, Sakai N, Suzuki Y, Takada A, Wilson I A. 2012. Heterosubtypic antibody recognition of the influenza virus hemagglutinin receptor binding site enhanced by avidity. Proc Natl Acad Sci USA 109:17040-17045. https://doi.org/10.1073/pnas.1212371109.
45. Krause J C, Tsibane T, Tumpey T M, Huffman C J, Basler C F, Crowe J E Jr. 2011. A broadly neutralizing human monoclonal antibody that recognizes a conserved, novel epitope on the globular head of the influenza H1N1 virus hemagglutinin. J Virol 85:10905-10908. https://doi.org/10.1128/JVI.00700-11.
46. Tsibane T, Ekiert D C, Krause J C, Martinez O, Crowe J E Jr, Wilson I A, Basler C F. 2012. Influenza human monoclonal antibody 1F1 interacts with three major antigenic sites and residues mediating human receptor specificity in H1N1 viruses. PLoS Pathog 8: e1003067. https://doi.org/10.1371/journal.ppat.1003067.
47. Whittle J R, Zhang R, Khurana S, King L R, Manischewitz J, Golding H, Dormitzer P R, Haynes B F, Walter E B, Moody M A, Kepler T B, Liao H X, Harrison S C. 2011. Broadly neutralizing human antibody that recognizes the receptor-binding pocket of influenza virus hemagglutinin. Proc Natl Acad Sci USA 108:14216-14221. https://doi.org/10.1073/pnas.1111497108.
48. Cobey S, Hensley S E. 2017. Immune history and influenza virus susceptibility. Curr Opin Virol 22:105-111. https://doi.org/10.1016/j.coviro.2016.12.004.
49. Khurana S, Verma N, Yewdell J W, Hilbert A K, Castellino F, Lattanzi M, Del Giudice G, Rappuoli R, Golding H. 2011. MF59 adjuvant enhances diversity and affinity of antibody-mediated immune response to pandemic influenza vaccines. Sci Transl Med 2011 3: 85ra48. https://doi.org/10.1126/scitranslmed.3002336.
50. Iba Y, Fujii Y, Ohshima N, Sumida T, Kubota-Koketsu R, Ikeda M, Wakiyama M, Shirouzu M, Okada J, Okuno Y, Kurosawa Y, Yokoyama S. 2014. Conserved neutralizing epitope at globular head of hemagglutinin in H3N2 influenza viruses. J Virol 88:7130-7144. https://doi.org/10.1128/JVI.00420-14.
51. He W, Chen C J, Mullarkey C E, Hamilton J R, Wong C K, Leon P E, Uccellini M B, Chromikova V, Henry C, Hoffman K W, Lim J K, Wilson P C, Miller M S, Krammer F, Palese P, Tan G S. 2017. Alveolar macrophages are critical for broadly-reactive antibody-mediated protection against influenza A virus in mice. Nat Commun 8:846. https://doi.org/10.1038/s41467-017-00928-3.
52. Ekiert D C, Friesen R H, Bhabha G, Kwaks T, Jongeneelen M, Yu W, Ophorst C, Cox F, Korse H J, Brandenburg B, Vogels R, Brakenhoff J P, Kompier R, Koldijk M H, Cornelissen L A, Poon L L, Peiris M, Koudstaal W, Wilson I A, Goudsmit J. 2011. A highly conserved neutralizing epitope on group 2 influenza A viruses. Science 333:843-850. https://doi.org/10.1126/science.1204839.
53. Friesen R H, Lee P S, Stoop E J, Hoffman R M, Ekiert D C, Bhabha G, Yu W, Juraszek J, Koudstaal W, Jongeneelen M, Korse H J, Ophorst C, Brinkman-van der Linden E C, Throsby M, Kwakkenbos M J, Bakker A Q, Beaumont T, Spits H, Kwaks T, Vogels R, Ward A B, Goudsmit J, Wilson I A. 2014. A common solution to group 2 influenza virus neutralization. Proc Natl Acad Sci USA 111:445-450. https://doi.org/10.1073/pnas.1319058110.
54. Larkin M A, Blackshields G, Brown N P, Chenna R, McGettigan P A, McWilliam H, Valentin F, Wallace I M, Wilm A, Lopez R, Thompson J D, Gibson T J, Higgins D G. 2007. Clustal W and Clustal X version 2.0. Bioinformatics 23:2947-2948.
55. Wang S, Taaffe J, Parker C, Solórzano A, Cao H, García-Sastre A, Lu S. 2006. Hemagglutinin (HA) proteins from H1 and H3 serotypes of influenza A viruses require different antigen designs for the induction of optimal protective antibody responses as studied by codon-optimized HA DNA vaccines. J Virol 80:11628-11637.

### 6.3 Example 3: a Mosaic Hemagglutinin-Based Influenza Virus Vaccine Candidate Protects Mice from Challenge with Divergent H3N2 Strains

This example describes the production of mosaic influenza virus hemagglutinins (mHAs) in which the major antigenic sites of an influenza A virus hemagglutinin are exchanged with exotic HA sequences. The mHAs were designed to elicit antibodies not only against the conserved stalk domain but also against epitopes in the head domain outside of the major antigenic sites. This example describes two 7:1 reassortant viruses expressing mHAs based on the recent H3N2 vaccine strain A/Hong Kong/4801/2014 (HK2014) with major antigenic sites mutated using sequences of two different avian HAs were rescued in the A/Puerto Rico/8/1934 (PR8) backbone. The example demonstrates that intramuscularly administered inactivated mHA viruses elicited anti-stalk antibodies at levels comparable to those elicited by the corresponding chimeric influenza virus hemagglutinins (cHAs) and at higher levels than a seasonal vaccine control. The antibodies raised by both the mHA and cHA vaccines induced significant levels of antibodies with in vitro Fc-mediated effector functions measured in a reporter assay. Moreover, the mHA vaccine, but not the corresponding cHA vaccine, induced antibodies with in vitro neutralization and hemagglutination inhibition (HI) activity against HK2014 virus, indicating the ability to induce head-specific antibodies. Serum transfer studies showed that antibodies raised with the mHA vaccines significantly protected against challenge with historical drifted H3N2 strains. Thus, the data in this a mHA vaccine provides a viable option for a universal influenza virus vaccine.

### 6.3.1 MATERIALS AND METHODS

Recombinant hemagglutinin genes and cloning. The chimeric and mosaic HA gene segments were based on the H3 gene of A/Hong Kong/4801/2014 virus as present in the New York Medical College (NYMC) X-263 strain obtained from NIBSC. The mosaic HA gene segments were designed by aligning the H3 gene sequence with the HA sequence of A/Jiangxi-Donghu/346-1/2013 (H10N8; sequence obtained from the Global Initiative on Sharing Avian Influenza Data [http://gisaid.org], accession no. EPI530526), as described before²⁵ or with the HA sequence of A/mallard/Gurjev/263/1982 (H14N5; sequence obtained from the Influenza Research Database [www.fludb.org], accession no. GQ247868), using the Clustal X 2.0 program³⁷, and exchanging key amino residues of H3 with corresponding sequences of H10 or H14. The gene segments were obtained as synthetic double-stranded DNA fragments from Integrated DNA Technologies, using the gBlocks® Gene Fragments service, with 15 bp cloning sites specific for the pDZ vector at the 5′ and 3′ ends. All sequences are shown in FIGS. 18A-18C. The HA gene segments were cloned into an ambisense pDZ vector that was digested with the SapI restriction enzyme (New England Biolabs), using the In-Fusion HD Cloning Kit (Clontech) according to the manufacturer's protocol. Sequences were confirmed by Sanger sequencing (Macrogen for plasmids and GeneWiz for PCR fragments). Primer sequences are shown in Table 11. Primers were purchased from Life Technologies (pDZ_forward and pDZ_reverse) or Integrated DNA Technologies (all other primers).

TABLE 11
Primers used in this study.

| Primerd | Purpose | Sequence (5′ to 3′) |
|---|---|---|
| pDZ_forward | Sequencing of | TACAGCTCCTGGGCAACGTGCTGG |
| | plasmids | (SEQ ID NO: 154) |
| pDZ_reverse | Sequencing of | AGGTGTCCGTGTCGCGCGTCGCC |
| | plasmids | (SEQ ID NO: 155) |
| H3_forward | Sequencing of | GGGAGCAAAAGCAGGGGATAATTC |
| | PCR fragments | (SEQ ID NO: 156) |
| H3_internal | Sequencing of | TACCCAGCATTGAACGTGAC |
| | plasmids and | (SEQ ID NO: 157) |
| | PCR fragments | |
| H3_reverse | Sequencing of | GGGTTATTAGTAGAAACAAGGGTG |
| | PCR fragments | TTTTTAATTAATG |
| | | (SEQ ID NO: 158) |

Cell culture. Human embryonic kidney 293T cells were maintained in Dulbecco's Modified Eagle Medium (DMEM; Gibco) supplemented with 10% fetal bovine serum (FBS) (Hyclone) and antibiotics (100 units/mL penicillin-100 μg/mL streptomycin [Pen-Strep]; Gibco). Madin-Darby Canine Kidney (MDCK) cells were maintained in Minimum Essential Medium (MEM; Gibco) supplemented with 10% FBS, Pen-Strep, L-glutamine (Gibco), sodium bicarbonate (Corning) and 4-(2-hydroxyethyl)-1-piperazineethanesulfonic acid (HEPES, Gibco). Cell lines were maintained at 37° C. with 5% CO₂.

Rescue of influenza viruses. Reassortant viruses were rescued by transfecting human embryonic kidney 293T cells with 0.7 μg of HA-encoding pDZ plasmid, 2.8 μg of a pRS-7 segment plasmid that drives ambisense expression of the seven gene segments of PR8 virus except HA that is described elsewhere³⁸, and 0.5 μg of a pCAGGS plasmid expressing the PR8 HA protein, using the TransIT-LT1 transfection reagent (Mirus Bio) according to the manufacturer's protocol. After 48 hours of incubation, cells were treated with 1 μg per mL tosyl phenylalanyl chloromethyl ketone (TPCK)-treated trypsin for 30 min. Then, supernatants were collected, clarified by low speed centrifugation, and injected into 8 to 10-day old specific pathogen-free embryonated chicken eggs (Charles River Laboratories) that were incubated at 37° C., as described.^{17,39} Forty-eight hours after injection, eggs were cooled to 4° C. overnight, allantoic fluids were harvested and clarified by low speed centrifugation. The presence of influenza virus in allantoic fluids was determined by hemagglutination assays as described below. Positive virus cultures were plaque purified on confluent MDCK cell layers in the presence of TPCK-treated trypsin and expanded in embryonated chicken eggs. Sequences of the HA and NA genes were confirmed by Sanger sequencing, as described above.

Generation and inactivation of viruses for vaccination. Plaque-purified and sequenced influenza viruses were expanded in 8 to 10 days old embryonated chicken eggs. Pooled allantoic fluids of approximately 20 eggs were added on top of 3 mL of a 20% sucrose solution in 0.1M NaCl, 1 mM ethylenediaminetetraacetic acid (EDTA) and 10 mM Tris-HCl, pH 7.4, in 38.5 mL ultracentrifuge tubes (Denville). After ultracentrifugation at 25,000 rpm in an L7-65 ultracentrifuge (Beckman) equipped with an SW28 rotor for 2 hours at 4° C. the pellets were recovered in 1 mL of PBS. After addition of 0.03% (v/v) formaldehyde, virus suspensions were incubated at 4° C. while shaking. After 48 hours, virus suspensions were diluted with PBS and subjected to purification by ultracentrifugation as described above to remove the formaldehyde. Pellets were resuspended in sterile PBS and the total protein concentration was determined with the Pierce BCA Protein Assay Kit (Thermo Fisher) according to the manufacturer's protocol.

Immunization studies. All animal experiments were performed with 6-8 weeks old female BALB/c mice (Charles River) in accordance with protocols approved by the Institutional Animal Care and Use Committee (IACUC) of the Icahn School of Medicine at Mount Sinai. Plasmid DNA immunizations were performed with 80 μg of pCAGGS plasmid expressing H4 of the A/duck/Czechoslovakia/1956 (H4N6) virus diluted in 100 μL of sterile PBS via the intramuscular route, using a TriGrid electroporation device (Ichor Medical Systems). Formaldehyde-inactivated viruses were administered intramuscularly at a dose of 10 μg total protein per mouse diluted in a total volume of 100 μL sterile PBS or 50 μL sterile PBS combined with 50 μL of AddaVax adjuvant (Invivogen). QIV was administered intramuscularly at a dose of 1 μg HA protein in a total volume of 100 μL of sterile PBS. The QIV was the Fluarix Quadrivalent vaccine produced by GaxoSmithKline (2016/2017 formulation) that contained the following influenza virus strains: A/Christchurch/16/2010 (H1N1), A/Hong Kong/4801/2014 (H3N2), B/Brisbane/60/2008 and B/Phuket/3073/2013. Four weeks after the final immunization, mice were euthanized and blood was obtained by cardiac puncture. Sera were prepared by removing red blood cells by centrifugation and were stored at −20° C. until use.

Challenge studies. Mice received 200 μL of pooled serum or sterile PBS via the intraperitoneal route. After 2 hours, mice were infected intranasally with five 50% mouse lethal doses (mLD₅₀) of either X-31 virus, a mouse-adapted reassortant virus with the HA and NA of A/Hong Kong/1/1968 and the internal proteins of PR8 or X-79 virus, a reassortant virus expressing NA and HA of A/Philippines/2/1982 and the internal proteins of PR8 in 50 μL of sterile PBS after sedation with a ketamine/xylazine cocktail administered intraperitoneally. Mice were monitored for weight loss and survival for 14 days post-challenge, whereby mice that lost more than 25% of weight were sacrificed, consistent with previous challenge studies with H3N2 viruses.^{28,40}

Enzyme-linked immunosorbent assays (ELISA). Recombinant HA proteins were produced as described.⁴¹ Proteins were coated onto Immulon® 4 HBX 96-well microtiter plates (Thermo Scientific) at 2 μg per mL in PBS (50 μL per well) for 16 hours at 4° C. After washing once using PBS with 0.1% (v/v) Tween-20 (PBS-T), wells were blocked for 1 hour with 5% (w/v) skim milk powder in PBS. Wells were washed once with PBS-T. Mouse antisera diluted in PBS (50 μL per well) were added and incubated for 1 hour. Then, wells were incubated with HRP-conjugated anti-mouse IgG antibody (GE Healthcare) diluted 1:5,000 in 5% (w/v) skim milk powder in PBS for 1 hour, washed three times with PBS-T and developed using SigmaFast OPD (Sigma-Aldrich) for 20 min. Reactions were stopped by adding 3 M hydrochloric acid (HCl) and absorbance at 490 nm was determined on a Synergy 4 plate reader (BioTek). Hemagglutinins of HK2014 (H3), HK1968 (H3), H15 (from A/shearwater/West Australia/2576/1979 [H15N9]) and cH5/3 (a chimeric HA with an H5 head domain and the stalk domain of H3 of HK2014) were produced as trimeric proteins in the Krammer laboratory using published methods.⁴¹ The HA1 proteins of A/Aichi/2/1968 (H3N2) and A/Hong Kong/4801/2014 (H3N2) were purchased from Immune Technology Corp. For each ELISA plate, the average plus three standard deviations of absorbance values of blank wells was used as a cutoff to calculate AUC values using GraphPad Prism 5.03.

Hemagglutination assays. Using PBS as diluent, serial two-fold dilutions of allantoic fluid samples were prepared in 96 V-bottom well microtiter plates to a final volume of 50 μL per well. 50 μL of a 0.5% suspension of turkey red blood cells (Lampire) in PBS were then added to each well and samples were mixed by pipetting. Plates were incubated at 4° C. until red blood cells in PBS control samples settled to the bottom of the wells. The HA titer (HA units) was defined as the reciprocal of the highest dilution of virus that caused hemagglutination of red blood cells.

Treatment of serum samples with receptor-destroying enzyme (RDE). One volume of mouse or ferret serum was treated with three volumes of RDE from Vibrio cholerae (Denka Seiken, Chuo-ku, Tokyo, Japan) at 37° C. for 16 hours according to manufacturer's protocol. To the RDE-treated samples three volumes of a 2.5% sodium citrate solution were added. After incubation at 56° C. for 30 min, three volumes of PBS were added to each sample for a final dilution of 1:10.

Hemagglutination inhibition (HI) assays. HI assays were performed as previously described.²² Allantoic fluid samples were diluted in PBS to a final HA titer of 8 HA units per 50 μL. Two-fold dilutions (25 μL) of RDE-treated serum in PBS prepared in 96 V-well microtiter plates were then combined with 25 μL of the diluted influenza viruses. The plates were incubated for 30 min at room temperature to allow HA-specific antibodies to bind to the virus. Then, 50 μL of a 0.5% suspension of turkey red blood cells (Lampire) that was washed once with PBS were added to each well, and the plates were incubated at 4° C. until the red blood cells in PBS control samples settled to the bottom of the wells. HI titers were defined as the reciprocal of the highest dilution of serum that inhibited hemagglutination of red blood cells. Antisera obtained from two ferrets immunized intranasally with egg-adapted A/Hong Kong/4801/2014 virus was kindly provided by Dr. Randy Albrecht (Icahn School of Medicine at Mount Sinai).

Microneutralization (MNT) assays. Microneutralization assays were performed as described previously⁴⁰ with modifications. Briefly, pooled, heat-inactivated and RDE-treated sera (starting concentration 1:50 and serially diluted 2-fold) and viruses (1,000 plaque-forming units) were pre-incubated at room temperature for 1 hour to allow for antibodies binding to virions. After incubation, the mixture was added to monolayers of MDCK cells in 96-well tissue culture plates and incubated at 37° C. for 1 hour to allow for attachment of virions to the cells. After washing with PBS three times to remove non-attached virions, the plates were re-incubated at 37° C. with infection medium containing the appropriate serum dilution. Eighteen hours later, the cells were fixed with 80% acetone in PBS and then stained for the NP protein using a primary biotinylated antibody (EMD Millipore) (1:2,000) and a secondary streptavidin conjugated to HRP antibody (EMD Millipore) (1:5,000). Wells were developed by incubating with SigmaFast OPD (Sigma-Aldrich) for 20 min. Reactions were stopped by adding 3 M HCl and absorbance at 490 nm was determined on a Synergy 4 plate reader (BioTek). Endpoint titers were defined as the reciprocal of the highest serum dilution that neutralized virus.

Antibody-dependent cellular cytotoxicity (ADCC) reporter assays. ADCC reporter assays were performed as previously described.^{30,33} Briefly, 96-well white flat-bottom plates (Costar Corning) were seeded with 2× 104 MDCK cells per well. After 18 hours of incubation at 37° C., the MDCK cells were washed once with PBS and then infected with a 6:2 reassortant virus expressing HA and NA of A/Hong Kong/4801/2014 virus and the internal proteins of PR8 virus²⁵ or a 6:2 reassortant virus expressing HA and NA of A/Hong Kong/1/1968 virus and the internal proteins of PR8 virus (X-31 virus) at a multiplicity of infection (MOI) of 5 for single cycle replication. The infected cells were incubated at 37° C. for 24 hours. The next day, the culture medium was removed and 25 μL of assay buffer (RPMI 1640 supplemented with 4% low-IgG FBS) was added to each well. Then, sera were added in a volume of 25 μL at a starting dilution of 1:60 and serially diluted 2-fold in assay buffer in triplicates. The sera were then incubated with the infected MDCK cells for 30 min at 37° C. Genetically modified Jurkat cells expressing the mouse FcγRIV with a luciferase reporter gene under transcriptional control of the nuclear factor-activated T cells (NFAT) promoter were added at 7.5×10⁴ cells in 25 μL per well (Promega). Cells were then incubated for another 6 hours at 37° C. A volume of 75 μL of Bio-Glo Luciferase assay reagent (Promega) was added to each well and luminescence was quantified using a microplate reader. Fold induction was measured in relative light units and calculated by subtracting the background signal from wells without effector cells, then dividing signals of wells with antibody by those with no antibody added. Fold induction was calculated as follows: (RLU_{induced}−RLU_{background})/(RLU_{uninduced}−RLU_{background}).

Immunofluorescence microscopy. Infected cells: In 96-well culture plates, MDCK cell monolayers were infected with influenza viruses at an MOI of 5 and incubated for 16 hours at 37° C. Transfected cells: HEK 293T cells were plated in 96-well tissue culture plates at a density of 2×10⁴ cells per well. After incubation for 4 hours, cells were transfected with 100 ng of either a pCAGGS plasmid expressing H4 of A/duck/Czechoslovakia/1956 (H4N6) virus or a pDZ plasmid expressing H7 of A/Hunan/02285/2017 (H7N9) virus using the TransIT-LT1 transfection reagent (Mirus Bio) according to the manufacturer's protocol and incubated for 16 hours at 37° C. The culture medium was aspirated, the cells were washed twice with PBS and then fixed with a methanol-free 4% (v/v) paraformaldehyde in PBS solution for 15 min. After washing twice with PBS, the wells were blocked with 5% (w/v) skim milk powder in PBS for 30 min. The cells were washed once with PBS and then incubated with mAbs 9H10²⁸ (anti-H3 stalk), KL-H4-1E8⁴² (anti-H4), 1A8⁴³ (anti-H7) or CR9114^{44,45} (pan anti-HA stalk) at 10 μg per mL, or pooled mouse sera at 1:50, diluted in 5% (w/v) skim milk powder in PBS for 2 hours. After washing three times with PBS, the cells were incubated with fluorescence-labeled anti-human (for CR9114) or anti-mouse (for all other mAbs and sera) IgG Alexa Fluor 488 antibody (Life Technologies) diluted 1:2,000 in 5% (w/v) skim milk powder in PBS for 1 hour and then washed three times with PBS before pictures were taken on an EVOS fl inverted fluorescence microscope (AMG).

Statistics. Statistical data was generated with GraphPad Prism version 5.03 (GraphPad Software). For ELISA data, statistical significance between groups was determined by performing One-way analysis of variance (ANOVA) tests with Bonferroni correction for multiple comparisons. For HI data, statistical significance between groups was determined by transforming reciprocal HI titers into logarithmic values and performing ANOVA with the Newman-Keuls posttest, as described previously.⁴⁶ Survival curves were compared using log rank Mantel-Cox tests against the Mock groups (DNA prime only or untreated). Levels of significance are indicated as follows: *P≤0.05, **P≤0.01, ***P≤0.001. For all statistical evaluations, the groups with H4 DNA prime and the groups without H4 DNA prime were analyzed separately.

### 6.3.2 RESULTS

Rescue and characterization of recombinant influenza viruses expressing mosaic hemagglutinin proteins. A 7:1 reassortant influenza virus in the PR8 backbone expressing a mosaic HA protein based on the egg-adapted H3 of HK2014 with key residues of the major antigenic sites exchanged with H10 sequences of A/Jiangxi-Donghu/346-1/2013 (H10N8) has been described previously²⁵ (FIG. 14A). This virus is designated as mH10/3. A second mosaic virus, mH14/3, was generated by replacing the same amino acid residues with the corresponding residues of H14 of A/mallard/Gurjev/263/1982 (H14N5) (FIG. 14B). In addition, the corresponding chimeric viruses, termed cH10/3 and cH14/3, were rescued in which the entire head domain was exchanged with the H10 and H14 sequences, respectively. A virus with unchanged wildtype egg-adapted H3 served as control. After growing for 48 hours in embryonated chicken eggs, the plaque-purified viruses reached hemagglutination titers of 1:64 (mH10/3), 1:128 (mH14/3) and 1:256 (wildtype, cH10/3, and cH14/3) HA units per 50 μL (FIG. 14C). Immunofluorescence microscopy experiments of virus-infected Madin-Darby canine kidney (MDCK) cells, using a monoclonal antibody (mAb) 9H10 that recognizes a conformational epitope in the stalk domain of group 2 HAs28, verified that the various HA proteins were expressed on the cellular surface and that the H3 stalk domain of each virus retained the native conformation (FIG. 14D). Ferret antiserum raised against the egg-adapted HK2014 wildtype virus reacted strongly against the virus expressing the sequence-identical HA (H3-wt) in hemagglutination inhibition (HI) assays (FIG. 14E). In contrast, no detectable HI reactivities were measured against the mH10/3 and mH14/3 viruses, supporting that the major antigenic sites had been successfully antigenically altered by the introduced mutations. There were no measurable HI reactivities against the cH10/3 and cH14/3 viruses, as HI active antibodies typically target the head domain. All HA sequences are shown in FIGS. 18A-18C.

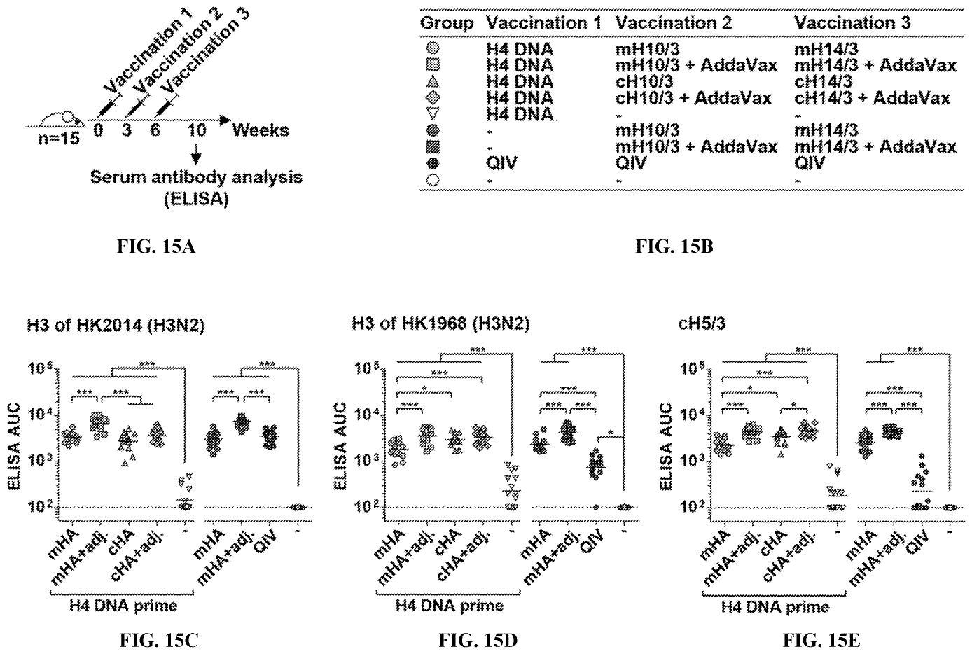

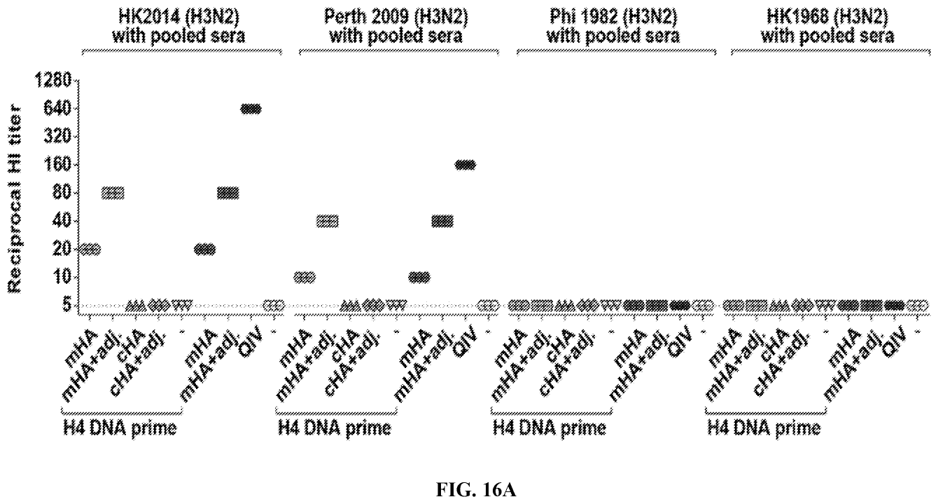

Immunization with inactivated mHA viruses elicits antibodies with broad reactivities against H3 proteins. To investigate their potential as vaccines, the mosaic viruses were expanded in embryonated chicken eggs, purified by sucrose cushion ultracentrifugation and inactivated with formaldehyde. Five groups of 15 mice each received a priming immunization with an expression plasmid for H4 hemagglutinin of A/duck/Czechoslovakia/1956 (H4N6) virus, which aimed to mimic the effect of preexisting immunity to a group 2 HA (FIGS. 15A, 15B). These mice subsequently either received two doses of inactivated mHA viruses (mH10/3 followed by mH14/3) or two doses of inactivated cHA viruses (cH10/3 followed by cH14/3) intramuscularly in three-week intervals. The vaccine candidates were administered either with or without the presence of the oil-in-water adjuvant AddaVax. A control group of mice received the H4 plasmid priming only. Three additional groups that did not receive a plasmid DNA priming immunization received two doses of inactivated mHA viruses with or without Adda Vax, or three doses of commercial inactivated quadrivalent influenza vaccine (QIV) containing H3N2 (HK2014) components. In addition, a naïve control group was included. Sera were obtained from all immunized mice four weeks after the last immunization to assess the antibody responses.

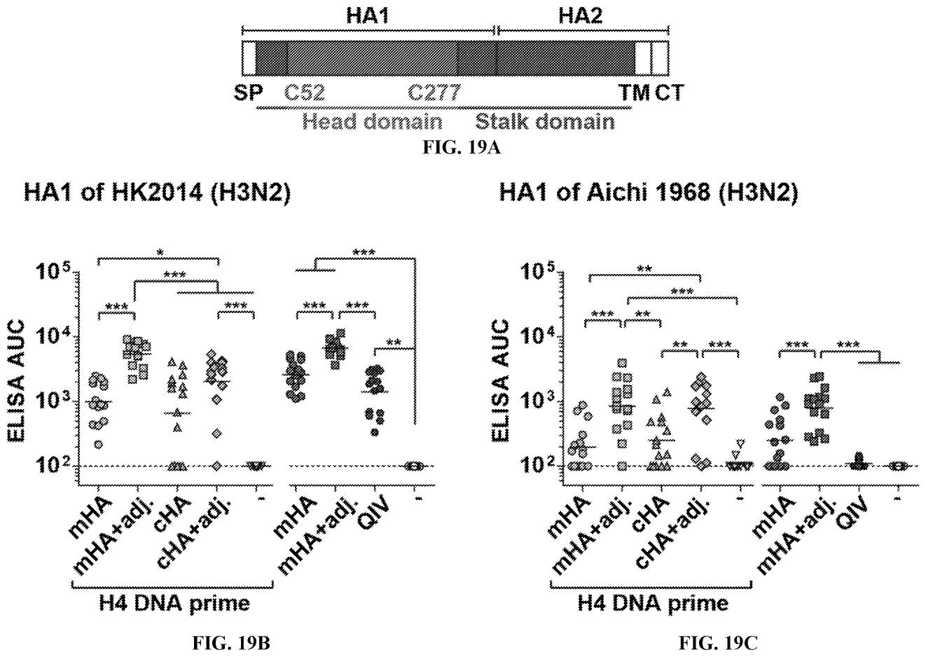

First, total serum IgG responses to a panel of recombinant trimeric HA proteins as well as HA1 polypeptides were determined using enzyme-linked immunosorbent assays (ELISAs). All groups of mice except for the control groups (prime only and naïve) mounted significant IgG responses to the H3 of HK2014 (FIG. 15C). The unadjuvanted mHA and cHA vaccines induced comparable antibody levels, while the addition of adjuvant further boosted IgG responses significantly. Levels of IgG against HK2014 H3 in mice receiving the adjuvanted mHA vaccine were significantly higher than those in mice receiving adjuvanted cHA vaccine. This suggested that additional head-specific antibodies may have been induced by the adjuvanted mHA vaccine. QIV induced IgG to HK2014 H3 at levels comparable to the unadjuvanted mHA vaccine. IgG raised by both the mHA and cHA vaccines cross-reacted with H3 of A/Hong Kong/1/1968 (HK1968) virus at comparable levels (FIG. 15D). Again, Adda Vax significantly increased the IgG levels against HK 1968 H3. In contrast, QIV raised sera had significantly lower levels of IgG binding to HK1968 HA than those induced by the mHA and cHA vaccines. Levels of IgG against the HA1 polypeptide of HK2014 (which includes the entire head domain and a portion of the stalk domain, see FIG. 19A) were higher for the adjuvanted mHA vaccine than for the adjuvanted cHA vaccine, providing further evidence that the adjuvanted mHA vaccine was able to elicit head-specific antibodies (FIG. 19B). By contrast, there were no significant differences in the IgG titers between adjuvanted mHA and cHA vaccines against HA1 of A/Aichi/2/1968 (Aichi 1968) (FIG. 19C). The reactivity of serum IgG to the HA1 proteins raised with the cHA vaccine may be explained by stalk epitopes present in HA1. QIV induced measurable IgG titers against HA1 of HK2014 but not against HA1 of Aichi 1968, supporting the notion that the seasonal vaccine mainly induced strain-specific antibodies. Prior immunization with the H4 DNA plasmid had no significant effect on the IgG titers against any of the tested proteins.

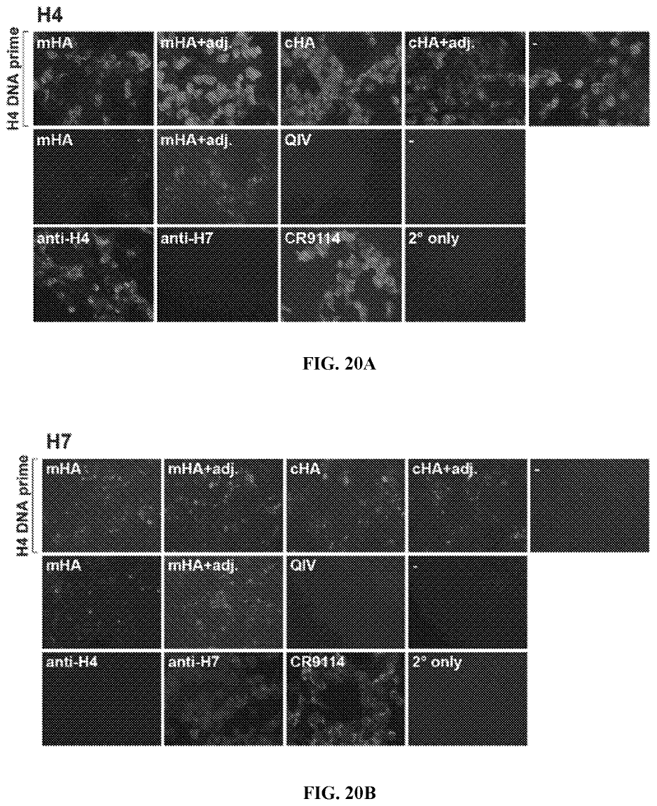

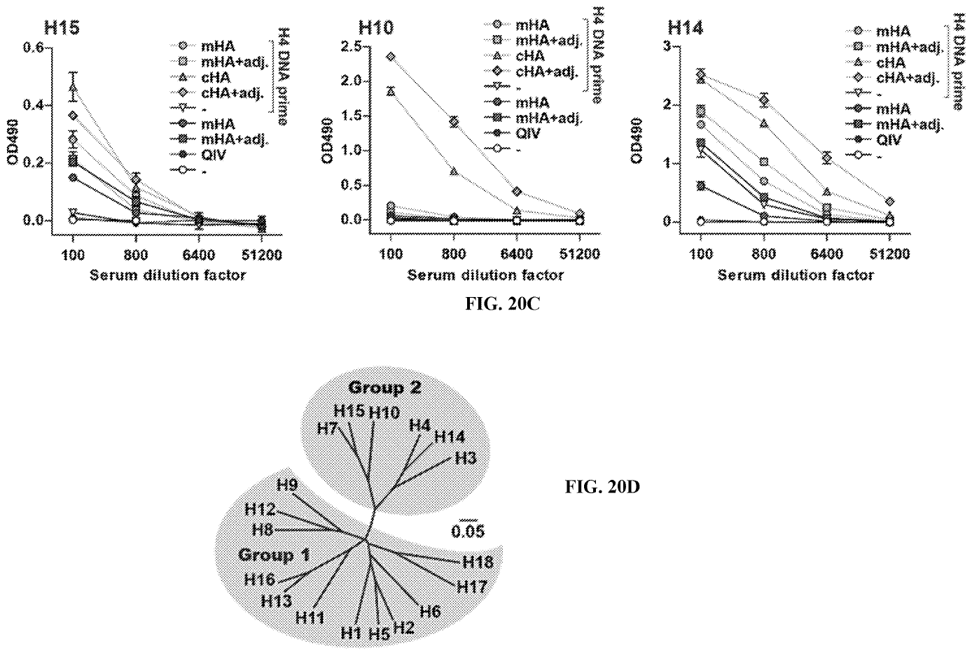

Next, the presence of stalk-reactive antibodies was assessed by performing ELISAs with a chimeric cH5/3 hemagglutinin protein that has a group 1 head domain (H5) on top of the stalk domain of HK2014 (FIG. 15E). The mHA and cHA vaccines induced comparable levels of stalk-reactive IgG and again a significant increase of these antibody titers was observed when the vaccines were adjuvanted. Of note, QIV did not induce significant amounts of IgG to the stalk domain when compared to naïve mice. Pooled sera were used to assess the cross-reactivity of antisera to other group 2 HAs, namely H4, H7 and H15. Immunofluorescence microscopy experiments revealed that the mHA vaccine induced antibodies binding to cell surface-expressed H4 (FIG. 20A), and both the mHA and cHA vaccines induced antibodies binding to cell surface-expressed H7 (FIG. 20B). Furthermore, all mouse groups primed with the H4 DNA plasmid showed reactivity to H4, indicating that DNA vaccination successfully induced anti-H4 immunity. In contrast to the mHA and cHA vaccines, QIV did not induce detectable levels of anti-H4 or anti-H7 IgG binding to surface-expressed proteins. In addition, cross-reactive IgGs to H15, as determined by ELISA, were induced by mHA and cHA vaccines, but not by QIV (FIG. 20C).

In conclusion, the cHA and mHA vaccines induced comparable levels of IgG cross-reacting with H3 proteins from 2014 and 1968 and to other group 2 HAs. Both mHA and cHA vaccines induced high levels of stalk-reactive antibodies. By contrast, the specificity of QIV-induced IgG was narrower and mainly focused to H3 of the matched HK2014 virus. Adda Vax significantly increased the total IgG levels elicited by the mHA and cHA vaccines. Antibody levels against trimeric H3 as well as the HA1 polypeptide of HK2014 were significantly higher with adjuvanted mHA vaccine compared to adjuvanted cHA vaccine, suggesting that the mHA vaccine elicited additional head-specific antibodies compared to the cHA vaccine.

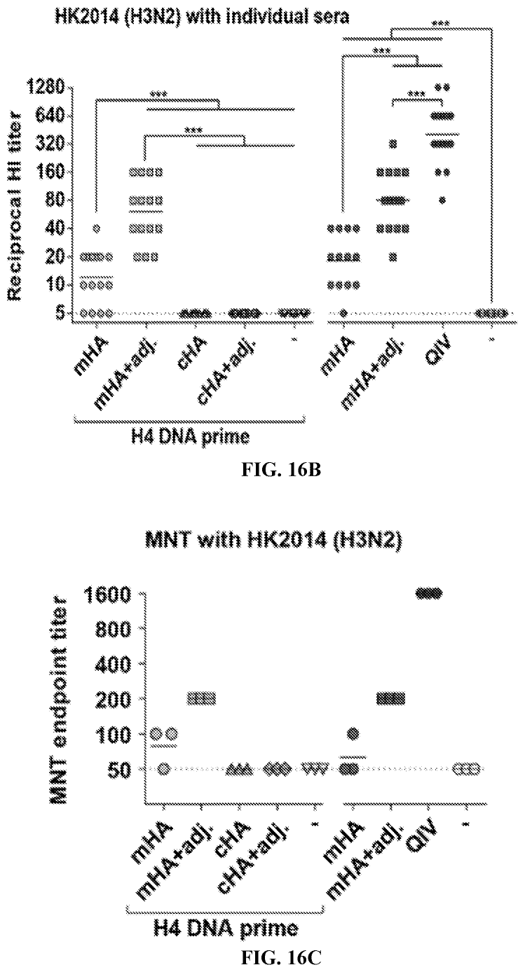

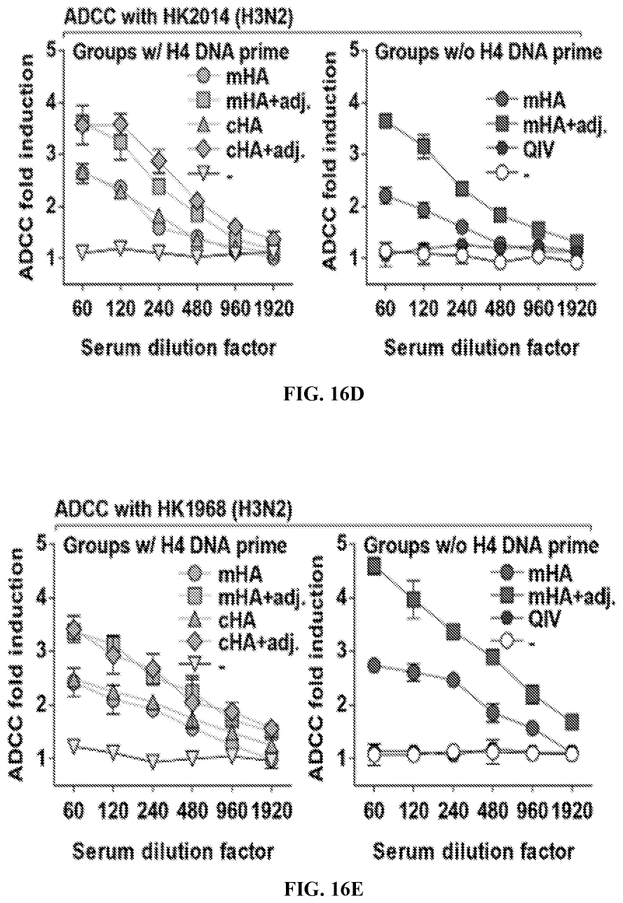

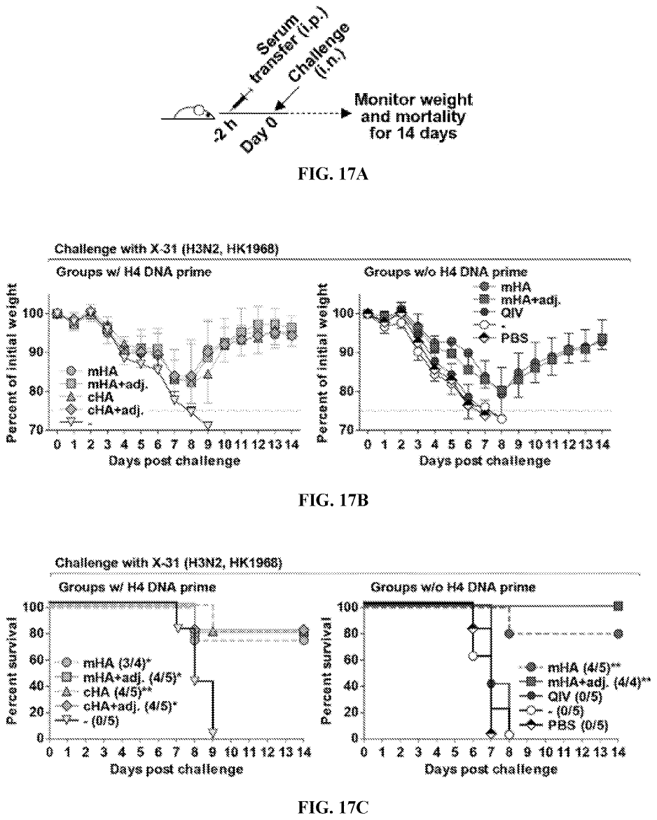

The mosaic, but not the chimeric, vaccine induces antibodies with hemagglutination inhibition and in vitro neutralization activity. Next, the functionality of the antibodies induced by the various vaccine candidates was determined. HI reactivity is a known correlate of protection, with a titer of ≥1:40 considered to confer 50% protection against seasonal influenza in human adults.²⁹ First, the HI reactivity of pooled sera of all groups of mice against a panel of H3N2 viruses from 1968-2014 was assessed (FIG. 16A). The mHA vaccine elicited detectable HI titers against HK2014 virus when administered without (1:20) or with adjuvant (1:80). As expected, QIV induced the highest HI titers (1:640) and the cHA vaccine induced no detectable HI titers, as only head-specific antibodies are HI active. HI reactivity was also detected against A/Perth/16/2009 virus (Perth 2009), with non-adjuvanted and adjuvanted mHA vaccines eliciting titers of 1:10 and 1:40, respectively, and QIV inducing an HI titer of 1:160. In contrast, no HI reactivity was observed against the more drifted viruses, A/Philippines/2/1982 (Phi 1982) and HK1968, for any of the antisera. A statistically significant induction of HI reactive antibodies by the mHA vaccine and QIV was confirmed, but not by the cHA vaccine, against HK2014 (FIG. 16B). Priming with the H4 DNA plasmid did not have a detectable impact on the HI titers.

To assess the neutralizing activity of the antibodies elicited by the various vaccines, in vitro microneutralization (MNT) assays were performed using pooled sera (FIG. 16C). The assay setup primarily detects strongly neutralizing antibodies targeting the HA head. Both the adjuvanted mHA vaccine and QIV elicited antisera with detectable MNT activity with 1:200 and 1:1,600 endpoint titers, respectively. In contrast, the cHA vaccine did not elicit detectable MNT activity. In summary, the mHA vaccine elicited detectable levels of HI active and neutralizing antibodies, whereas the cHA vaccine did not.

Mosaic and chimeric HA vaccines induce comparable levels of antibodies with in vitro ADCC reporter assay activity. The ability to engage Fc-mediated effector functions such as antibody-dependent cellular cytotoxicity (ADCC) is one of the mechanisms by which stalk-specific antibodies contribute to protection in vivo.³⁰⁻³² To assess if antibodies mediating effector functions were induced by the various vaccine candidates, an established in vitro ADCC reporter assay was performed.³³ Pooled sera of mHA and cHA vaccinated mice induced ADCC reporter activity on MDCK cells infected with HK2014 and HK1968 viruses to comparable levels (FIGS. 16D, 16E), whereby the inclusion of adjuvant further boosted the detected activity. By contrast, the QIV did not elicit detectable levels of antibodies with ADCC reporter activity. Therefore, both the mHA and cHA vaccines were capable of eliciting ADCC reporter activity, which is likely attributable to the stalk-specific IgG both vaccines elicited.³⁴

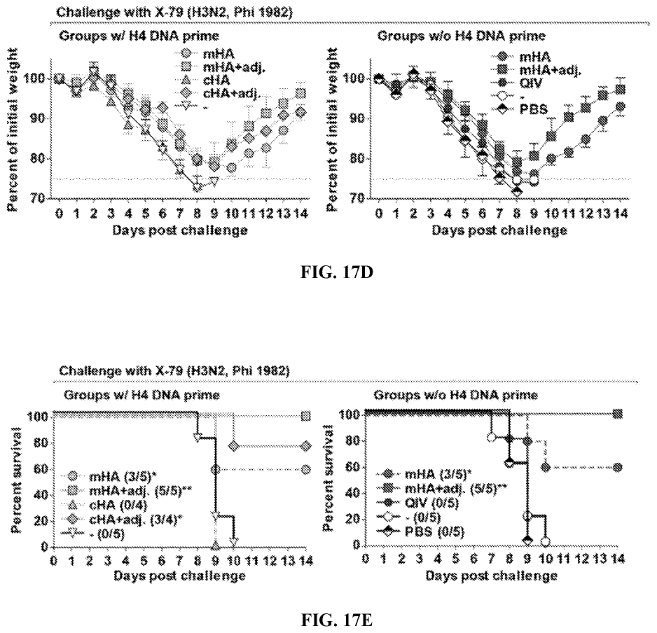

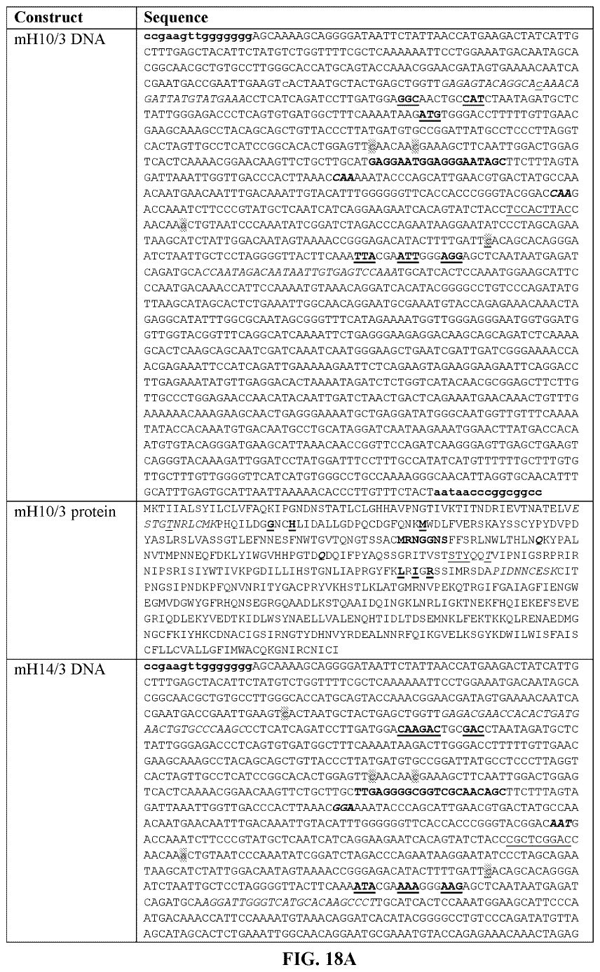

Antibodies elicited by the mosaic and the chimeric HA vaccines protect mice from lethal challenge with drifted H3N2 viruses. Next, the ability of the antibodies induced by the various vaccines to confer protection against lethal challenge with influenza viruses in vivo in a mouse model was determined. Groups of 4-5 naïve mice received 200 μL per individual of pooled sera intraperitoneally and were challenged two hours later with five 50% murine lethal doses (mLD₅₀) of either X-31 or X-79 challenge viruses (FIG. 17A). The challenge viruses are reassortant viruses expressing the HA and neuraminidase (NA) of HK1968 (X-31) or HA and NA of A/Philippines/2/1982 (X-79) and the internal proteins of PR8. Mice were observed daily for weight loss and mortality for 14 days post-challenge. In addition to pooled sera from the nine groups of mice described above, a tenth group receiving PBS instead of serum was included as an additional control. Of note, all mice challenged with X-31 virus showed substantial weight loss (FIG. 17B). All mice in the control groups (DNA priming only, naïve and PBS) as well as in the QIV group succumbed to the infection by day 9 post-infection, however, most animals in the mHA vaccine groups without adjuvant (75% and 80% survival with or without DNA prime, respectively) and with adjuvant (80% and 100% survival with or without DNA prime, respectively) survived (FIG. 17C). Similarly, the majority of mice in the cHA vaccine groups (80% survival irrespective of adjuvant) survived. Surviving mice regained weight to levels comparable to the initial weight by days 12-14. Comparable to X-31, all mice challenged with X-79 virus showed a substantial degree of weight loss (FIG. 17D) and mice in the control groups and the QIV group succumbed by day 10 post-infection (FIG. 17E). The majority of mice in the mHA groups (60% survival unadjuvanted and 100% adjuvanted, irrespective of the DNA prime immunization) survived and regained weight after day 10 post-infection. Most animals in the adjuvanted cHA group survived (75% survival), however, the unadjuvanted cHA group showed 0% survival.

In summary, sera elicited by the mHA and cHA vaccines induced significant protection against challenge with two different heterologous H3N2 viruses in mice, whereas sera induced by QIV did not confer a significant level of protection.

### 6.3.3 DISCUSSION

The results in this example demonstrate that mHA-based vaccines allow for the generation of broad antibody-mediated immunity against drifted H3N2 viruses in vivo. The mHA constructs were based on the recent H3N2 vaccine strain A/Hong Kong/4801/2014. Inactivated cHA expressing viruses afford broad protection against H3N2 viruses and mHA vaccines elicit comparable levels of cross-reactive and broadly protective antibodies.

When directly comparing the cHA to the mHA vaccines, the amount of total IgG elicited against drifted H3 hemagglutinins as well as stalk-specific IgG and ADCC-active antibodies were found to be similar. However, the mHA vaccine induced higher levels of total IgG against the HA of HK2014. Without being bound by any theory, this is likely due to head-specific antibodies. This was confirmed by the fact that the mHA vaccine, but not the cHA vaccine, elicited measurable signals in HI and MNT assays, indicative of functional head-specific antibodies. The mHA vaccine showed comparable protective effects to the cHA vaccine against X-31 virus that displays HA and NA of HK1968, and was more effective against Phi 1982 virus, perhaps due to additional head-specific antibodies that contributed to protection but may not be detectable by HI or MNT assays in vitro. When adjuvanted with AddaVax, an oil-in-water adjuvant similar to MF59 that is used in commercial seasonal influenza virus vaccines for the elderly³⁵, the mHA vaccine induced HI titers of 1:80 against HK2014 and 1:40 against Perth 2009, titers considered to be protective in adult humans²⁹. Therefore, the mHA vaccine described here may be at least as efficient as the cHA vaccine against drifted H3N2 strains, due to the induction of broad, anti-stalk antibodies, with the additional advantage of inducing head-specific antibodies.

The mHA vaccine induced cross-reactive antibodies against other group 2 HAs at comparable levels to the cHA vaccine, indicating additional protection against possible pandemics caused by, for instance, H7N9 viruses.³⁶ The mosaic vaccine approach may be used to manufacture inactivated influenza vaccine (IIV) or live-attenuated influenza vaccine (LAIV) preparations with existing infrastructure.

### 6.3.4 REFERENCES CITED IN, E.G., EXAMPLE 3

1. Krammer, F. et al. Influenza. Nat. Rev. Dis. Primers 4, 3 (2018).
2. Gerdil, C. et al. The annual production cycle for influenza vaccine. Vaccine 21, 1776-1779 (2003).
3. Krammer, F. & Palese, P. Universal influenza virus vaccines that target the conserved hemagglutinin stalk and conserved sites in the head domain. J. Infect. Dis. (Epub ahead of print) (2019).
4. Tricco, A. C. et al. Comparing influenza vaccine efficacy against mismatched and matched strains: a systematic review and meta-analysis. BMC Med. 11, 153 (2013).
5. Zost, S. J. et al. Contemporary H3N2 influenza viruses have a glycosylation site that alters binding of antibodies elicited by egg-adapted vaccine strains. Proc. Natl. Acad. Sci. USA 114, 12578-12583 (2017).
6. Gavigan, P. & McCullers, J. A. Influenza: annual seasonal severity. Curr. Opin. Pediatr. 31, 112-118 (2019).
7. Turbelin, C. et al. Age distribution of influenza like illness cases during post-pandemic A (H3N2): comparison with the twelve previous seasons, in France. PLoS One 8, e65919 (2013).
8. Rambaut, A. et al. The genomic and epidemiological dynamics of human influenza A virus. Nature 453, 615-619 (2008).
9. Khiabanian, H., Farrell, G. M., St George, K. & Rabadan, R. Differences in patient age distribution between influenza A subtypes. PLoS One 4, e6832 (2009).
10. Olson, D. R. et al. Monitoring the impact of influenza by age: emergency department fever and respiratory complaint surveillance in New York City. PLoS Med. 4, e247 (2007).
11. Wu, N. C. & Wilson, I. A. Structural insights into the design of novel anti-influenza therapies. Nat. Struct. Mol. Biol. 25, 115-121 (2012).
12. Wrammert, J. et al. Rapid cloning of high-affinity human monoclonal antibodies against influenza virus. Nature 453, 667-671 (2008).
13. Krammer, F. The human antibody response to influenza A virus infection and vaccination. Nat. Rev. Immunol. [Epub ahead of print] (2019).
14. Gerhard, W., Yewdell, J., Frankel, M. E. & Webster, R. Antigenic structure of influenza virus haemagglutinin defined by hybridoma antibodies. Nature 290, 713-717 (1981).
15. Wilson, I. A., Skehel, J. J. & Wiley, D. C. Structure of the haemagglutinin membrane glycoprotein of influenza virus at 3 A resolution. Nature 289, 366-373 (1981).
16. Wiley, D. C., Wilson, I. A. & Skehel, J. J. Structural identification of the antibody-binding sites of Hong Kong influenza haemagglutinin and their involvement in antigenic variation. Nature 289, 373-378 (1981).
17. Chen, C. J. et al. Influenza A Viruses Expressing Intra- or Intergroup Chimeric Hemagglutinins. J. Virol. 90, 3789-3793 (2016).
18. Krammer, F., Pica, N., Hai, R., Margine, I. & Palese, P. Chimeric hemagglutinin influenza virus vaccine constructs elicit broadly protective stalk-specific antibodies. J. Virol. 87, 6542-6550 (2013).
19. Margine, I. et al. Hemagglutinin stalk-based universal vaccine constructs protect against group 2 influenza A viruses. J. Virol. 87, 10435-10446 (2013).
20. Nachbagauer, R. et al. Hemagglutinin stalk immunity reduces influenza virus replication and transmission in ferrets. J. Virol. 90, 3268-3273 (2015).
21. Krammer, F. et al. Assessment of influenza virus hemagglutinin stalk-based immunity in ferrets. J. Virol. 88, 3432-3442 (2014).
22. Nachbagauer, R. et al. A universal influenza virus vaccine candidate confers protection against pandemic H1N1 infection in preclinical ferret studies. NPJ Vaccines 2, 26 (2017).
23. Ermler, M. E. et al. Chimeric Hemagglutinin Constructs Induce Broad Protection against Influenza B Virus Challenge in the Mouse Model. J. Virol. 91, e00286-17 (2017).
24. Sun, W. et al. Development of Influenza B Universal Vaccine Candidates using the Mosaic” Hemagglutinin Approach. J. Virol. (Epub ahead of print) (2019).
25. Broecker, F. et al. Immunodominance of antigenic site B in the hemagglutinin of the current H3N2 influenza virus in humans and mice. J. Virol. 92, e01100-18 (2018).
26. Lee, P. S. et al. Receptor mimicry by antibody F045-092 facilitates universal binding to the H3 subtype of influenza virus. Nat. Commun. 5, 3614 (2014).
27. Pettersen, E. F. et al. UCSF Chimera—a visualization system for exploratory research and analysis. J. Comput. Chem. 25, 1605-1612 (2004).
28. Tan, G. S. et al. Characterization of a broadly neutralizing monoclonal antibody that targets the fusion domain of group 2 influenza A virus hemagglutinin. J. Virol. 88, 13580-13592 (2014).
29. Cox, R. J. Correlates of protection to influenza virus, where do we go from here? Hum. Vaccin. Immunother. 9, 405-408 (2013).
30. Jacobsen, H. et al. Influenza Virus Hemagglutinin Stalk-Specific Antibodies in Human Serum are a Surrogate Marker for In Vivo Protection in a Serum Transfer Mouse Challenge Model. mBio 8, e01463-17 (2017).
31. Mullarkey, C. E. et al. Broadly Neutralizing Hemagglutinin Stalk-Specific Antibodies Induce Potent Phagocytosis of Immune Complexes by Neutrophils in an Fc-Dependent Manner. mBio 7, e01624-16 (2016).
32. Leon, P. E. et al. Optimal activation of Fc-mediated effector functions by influenza virus hemagglutinin antibodies requires two points of contact. Proc. Natl. Acad. Sci. USA 113, E5944-E5951 (2016).
33. Bailey, M. J., Broecker, F., Leon, P. E. & Tan, G. S. A Method to Assess Fc-mediated Effector Functions Induced by Influenza Hemagglutinin Specific Antibodies. J. Vis. Exp. 132, e56256 (2018).
34. Mullarkey C. E. et al. Broadly Neutralizing Hemagglutinin Stalk-Specific Antibodies Induce Potent Phagocytosis of Immune Complexes by Neutrophils in an Fc-Dependent Manner. mBio 7, e01624-16 (2016).
35. Domnich, A. et al. Effectiveness of MF59-adjuvanted seasonal influenza vaccine in the elderly: A systematic review and meta-analysis. Vaccine 35, 513-520 (2017).
36. Gao, R. et al. Human infection with a novel avian-origin influenza A (H7N9) virus. N. Engl. J. Med. 368, 1888-1897 (2013).
37. Larkin, M. A. et al. Clustal W and Clustal X version 2.0. Bioinformatics 23, 2947-2948 (2007).
38. Fulton, B. O., Sun, W., Heaton, N. S. & Palese, P. The influenza B virus hemagglutinin head domain is less tolerant to transposon mutagenesis than that of the influenza A virus. J. Virol. 92, e00754-18 (2018).
39. Hai, R. et al. Influenza viruses expressing chimeric hemagglutinins: globular head and stalk domains derived from different subtypes. J. Virol. 86, 5774-5781 (2012).
40. He, W. et al. Alveolar macrophages are critical for broadly-reactive antibody-mediated protection against influenza A virus in mice. Nat. Commun. 8, 846 (2017).
41. Krammer, F. A carboxy-terminal trimerization domain stabilizes conformational epitopes on the stalk domain of soluble recombinant hemagglutinin substrates. PLoS One 7, e43603 (2012).
42. Amanat, F., Meade, P., Strohmeier, S. & Krammer, F. Cross-reactive antibodies binding to H4 hemagglutinin protect against lethal H4N6 influenza virus challenge in the mouse model. Emerg. Microbes Infect. 8, 155-168 (2019).
43. Stadlbauer, D., Amanat, F., Strohmeier, S., Nachbagauer, R. & Krammer, F. Cross-reactive mouse monoclonal antibodies raised against the hemagglutinin of A/Shanghai/1/2013 (H7N9) protect against novel H7 virus isolates in the mouse model. Emerg. Microbes Infect. 7, 110 (2018).
44. Dreyfus, C. et al. Highly conserved protective epitopes on influenza B viruses. Science 337, 1343-1348 (2012).
45. Chromikova, V., Zaragoza, M. A. & Krammer F. Generation of a serum free CHO DG44 cell line stably producing a broadly protective anti-influenza virus monoclonal antibody. PLoS One 12, e0183315 (2017).
46. Benne, C. A. et al. Comparison of neutralizing and hemagglutination-inhibiting antibody responses to influenza A virus vaccination of human immunodeficiency virus-infected individuals. Clin. Diagn. Lab. Immunol. 5, 114-117 (1998).

### 7. EMBODIMENTS

Provided herein are the following exemplary embodiments:

1. A mosaic influenza virus hemagglutinin (HA) polypeptide comprising an influenza A virus HA ectodomain of a group 1 influenza A virus strain HA, wherein the HA ectodomain comprises an HA stem domain of the group 1 influenza A virus strain HA and an HA globular head domain of the group 1 influenza A virus strain HA, wherein the HA globular head domain of the group 1 influenza A virus strain HA has been engineered to comprise four or all of the following:
a. 1, 2, 3, 4, 5, 6, 7, 8, 9, 10, 11, 12 or more amino acid substitutions within the Sa antigenic site or a corresponding hypervariable antigenic site of the globular head domain of the group 1 influenza A virus strain HA;
b. 1, 2, 3, 4, 5, 6, 7, 8, 9, 10, 11 or more amino acid substitutions within the Sb antigenic site or a corresponding hypervariable antigenic site of the globular head domain of the group 1 influenza A virus strain HA;
c. 1, 2, 3, 4, 5, 6, 7, 8, 9, 10 or more amino acid substitutions within the Ca1 antigenic site or a corresponding hypervariable antigenic site of the globular head domain of the group 1 influenza A virus strain HA;
d. 1, 2, 3, 4, 5, 6, 7 or more amino acid substitutions within the Ca2 antigenic site or a corresponding hypervariable antigenic site of the globular head domain of the group 1 influenza A virus strain HA; and
e. 1, 2, 3, 4, 5 or more amino acid substitutions within the Cb antigenic site or a corresponding hypervariable antigenic site of the globular head domain of the group 1 influenza A virus strain HA.

2. A mosaic influenza virus hemagglutinin (HA) polypeptide comprising an HA ectodomain of a first group 1 influenza A virus strain HA, wherein the HA ectodomain comprises an HA stem domain of the first group 1 influenza A virus strain HA and an HA globular head domain of the first group 1 influenza A virus strain HA, wherein the HA globular head domain of the first group 1 influenza A virus strain has been engineered to comprise four or all of the following:
a. 1, 2, 3, 4, 5, 6, 7, 8, 9, 10, 11, 12 or more amino acid substitutions within the Sa antigenic site or a corresponding hypervariable antigenic site of the globular head domain of the first group 1 influenza A virus strain HA, wherein the amino acid substitutions substitute 1, 2, 3, 4, 5, 6, 7, 8, 9, 10, 11, 12 or more amino acid residues within the Sa antigenic site or the corresponding hypervariable antigenic site of the globular head domain of the first group 1 influenza A virus strain HA with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain;
b. 1, 2, 3, 4, 5, 6, 7, 8, 9, 10, 11 or more amino acid substitutions within the Sb antigenic site or a corresponding hypervariable antigenic site of the globular head domain of the first group 1 influenza A virus strain HA, wherein the amino acid substitutions substitute 1, 2, 3, 4, 5, 6, 7, 8, 9, 10, 11 or more amino acid residues within the Sb antigenic site or the corresponding hypervariable antigenic site of the globular head domain of the first group 1 influenza A virus strain HA with amino acid residues found in a corresponding region of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain;
c. 1, 2, 3, 4, 5, 6, 7, 8, 9, 10 or more amino acid substitutions within the Ca1 antigenic site or a corresponding hypervariable antigenic site of the globular head domain of the first group 1 influenza A virus, strain HA, wherein the amino acid substitutions substitute 1, 2, 3, 4, 5, 6, 7, 8, 9, 10 or more amino acid residues within the Ca1 antigenic site or the corresponding hypervariable antigenic site of the globular head domain of the first group 1 influenza A virus strain HA with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain;
d. 1, 2, 3, 4, 5, 6, 7 or more amino acid substitutions within the Ca2 antigenic site or a corresponding hypervariable antigenic site of the globular head domain of the first group 1 influenza A virus strain HA, wherein the amino acid substitutions substitute 1, 2, 3, 4, 5, 6, 7 or more amino acid residues within the Ca2 antigenic site or the corresponding hypervariable antigenic site of the globular head domain of the first group 1 influenza A virus strain HA with amino acid residues found in a corresponding region of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain; and
e. 1, 2, 3, 4, 5 or more amino acid substitutions within the Cb antigenic site or a corresponding hypervariable antigenic site of the globular head domain of the first group 1 influenza A virus strain, wherein the amino acid substitutions substitute 1, 2, 3, 4, 5 or more amino acid residues within the Cb antigenic site or the corresponding hypervariable antigenic site of the globular head domain of the first group 1 influenza A virus strain with amino acid residues found in a corresponding region of an HA globular head domain of: (1) a group 2 influenza A virus HA, (2) a group 1 influenza A virus HA of a different subtype or a different strain than the first group 1 influenza A virus strain, or (3) a combination of group 1 influenza A virus HAs of different subtypes or different strains than the first group 1 influenza A virus strain.

3. The mosaic influenza virus HA polypeptide of embodiment 2, wherein the corresponding region of the HA globular head domain is of a group 1 influenza A virus HA of a different subtype than the first group 1 influenza A virus strain.

4. The mosaic influenza virus HA polypeptide of embodiment 3, wherein the different subtype is an H5 subtype.

5. The mosaic influenza virus HA polypeptide of embodiment 4, wherein the H5 subtype is A/Vietnam/1203/2004.

6. The mosaic influenza virus HA polypeptide of embodiment 3, wherein the different subtype is an H13 subtype.

7. The mosaic influenza virus HA polypeptide of embodiment 6, wherein the H13 is A/black headed gull/Sweden/1/1999

8. The mosaic influenza virus HA polypeptide of embodiment 2, wherein the corresponding region of the HA globular head domain is of a combination of group 1 influenza A virus HAs of different subtypes than the first group 1 influenza A virus strain.

9. The mosaic influenza virus HA polypeptide of embodiment 8, wherein the different subtypes are H5 and H13 subtypes.

10. The mosaic influenza virus HA polypeptide of embodiment 9, wherein the H5 subtype is A/Vietnam/1203/2004 and the H13 subtype is A/black headed gull/Sweden/1/1999.

11. The mosaic influenza virus HA polypeptide of any one of embodiments 1 to 10, wherein the mosaic influenza virus HA polypeptide further comprises the transmembrane and cytoplasmic domains of the first group 1 influenza A virus strain HA.

12. The mosaic influenza virus HA polypeptide of any one of embodiments 1 to 11, wherein the first group 1 influenza A virus is an H1 subtype.

13. The mosaic influenza virus HA polypeptide of embodiment 12, wherein the H1 subtype is A/Michigan/45/2015.

14. A mosaic influenza virus HA polypeptide comprising an HA ectodomain of influenza A/Michigan/45/2015 virus HA, wherein the HA ectodomain comprises the influenza A/Michigan/45/2015 virus HA stem domain and HA globular head domain, wherein the globular head domain has been engineered to comprises one, two, three, four or all of the following amino acid sequence substitutions:
a. the amino acid sequences PN, KKGNS (SEQ ID NO: 1), and PKLNQS (SEQ ID NO: 2) in the HA globular head domain Sa antigenic site of influenza A/Michigan/45/2015 virus HA have been substituted with the following amino acid sequences PS, KKNST (SEQ ID NO: 3), and PTIKRS (SEQ ID NO: 4), respectively;
b. the amino acid sequence TTADQQSLYQNA (SEQ ID NO: 5) in the HA globular head domain Sb antigenic site of influenza A/Michigan/45/2015 virus HA has been substituted with the following amino acid sequence DAAEQTKLYQNP (SEQ ID NO: 6);
c. the amino acid sequences INDKG (SEQ ID NO: 7), TSR, and EPG in the HA globular head domain Ca1 antigenic site of influenza A/Michigan/45/2015 virus HA have been substituted with the following amino acid sequences NNTTG (SEQ ID NO: 8), TSS, and HPG, respectively;
d. the amino acid sequences PHAGAK (SEQ ID NO: 9) and RD in the HA globular head domain Ca2 antigenic site of influenza A/Michigan/45/2015 virus HA have been substituted with the following amino acid sequences PYQGKS (SEQ ID NO:10) and ND, respectively; and
e. the amino acid sequence LSTASS (SEQ ID NO: 11) in the HA globular head domain Cb antigenic site of influenza A/Michigan/45/2015 virus HA have been substituted with the following amino acid sequence LNVPE (SEQ ID NO: 12).

15. The mosaic influenza virus HA polypeptide of embodiment 14, wherein the mosaic influenza virus HA polypeptide further comprises the transmembrane and cytoplasmic domains of the influenza A/Michigan/45/2015 virus HA.

16. A mosaic influenza virus HA polypeptide comprising an HA ectodomain of a first group 2 influenza A virus strain HA, wherein the HA ectodomain comprises an HA stem domain of the first group 2 influenza A virus strain HA and an HA globular head domain of the first group 2 influenza A virus strain HA, wherein the HA globular head domain of the first group 2 influenza A virus strain HA has been engineered to comprise four or all of the following:
a. 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the first group 2 influenza A virus strain HA;
b. 1, 2, 3, 4, 5, 6, 7, 8, 9, 10, 11, 12, 13, 14, 15, 16, 17 or more amino acid substitutions within the B antigenic site or a corresponding hypervariable antigenic site of the globular head domain of the first group 2 influenza A virus strain HA;
c. 1, 2, 3, 4, 5, 6, 7, 8, 9, 10, 11, 12, 13, 14, 15, 16, 17, 18 or more amino acid substitutions within the C antigenic site or a corresponding hypervariable antigenic site of the globular head domain of the first group 2 influenza A virus strain HA;
d. 1, 2, 3, 4, 5, 6, 7, 8, 9, 10, 11, 12, 13, 14, 15, 16, 17, 18 or more amino acid substitutions within the D antigenic site or a corresponding hypervariable antigenic site of the globular head domain of the first group 2 influenza A virus strain HA; and
e. 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the first group 2 influenza A virus strain HA.

17. A mosaic influenza virus HA polypeptide comprising an HA ectodomain of a first group 2 influenza A virus strain HA, wherein the HA ectodomain comprises an HA stem domain of the first group 2 influenza A virus strain HA and an HA globular head domain of the first group 2 influenza A virus HA strain HA, wherein the HA globular head domain of the first group 2 influenza A virus strain HA has been engineered to comprise four or all of the following:
a. 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the A antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18, 19, 20, 21, 22, 23, 24, 25, 26 or more amino acid residues within the A antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain;
b. 1, 2, 3, 4, 5, 6, 7, 8, 9, 10, 11, 12, 13, 14, 15, 16, 17 or more amino acid substitutions within the B antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17 or more amino acid residues within the B antigenic site or the corresponding hypervariable antigenic site of the globular head domain of the group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain;
c. 11, 2, 3, 4, 5, 6, 7, 8, 9, 10, 11, 12, 13, 14, 15, 16, 17, 18 or more amino acid substitutions within the C antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18 or more amino acid residues within the C antigenic site or the corresponding hypervariable antigenic site of the globular head domain of the first group 2 influenza A virus strain HA with amino acid residues found in the corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain;
d. 1, 2, 3, 4, 5, 6, 7, 8, 9, 10, 11, 12, 13, 14, 15, 16, 17, 18 or more amino acid substitutions within the D antigenic site or a corresponding hypervariable antigenic site of the globular head domain of the first group 2 influenza A virus strain HA, wherein the amino acid substitutions substitute 1, 2, 3, 4, 5, 6, 7, 8, 9, 10, 11, 12, 13, 14, 15, 16, 17, 18, or more amino acid residues within the D antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain; and
e. 1, 2, 3, 4, 5, 6, 7, 8, 9, 10, 11, 12, 13, 14, 15, 16, 17, 18, 19, 20, 21, 22, 23, 24, 25, 26 or more amino acid substitutions within the E antigenic site or a corresponding hypervariable antigenic site of the globular head domain of the group 2 influenza A virus strain HA, wherein the amino acid substitutions substitute 1, 2, 3, 4, 5, 6, 7, 8, 9, 10, 11, 12, 13, 14, 15, 16, 17, 18, 19, 20, 21, 22, 23, 24, 25, 26 or more amino acid residues within the E antigenic site or the corresponding hypervariable antigenic site of the globular head domain of the first group 2 influenza A virus strain HA with amino acid residues found in a corresponding region of an HA globular head domain of: (1) a group 1 influenza A virus HA, (2) a group 2 influenza A virus HA of a different subtype or a different strain than the first group 2 influenza A virus strain, or (3) a combination of group 2 influenza A virus HAs of different subtypes or different strains than the first group 2 influenza A virus strain.

18. The mosaic influenza virus HA polypeptide of embodiment 17, wherein the corresponding region of the HA globular head domain is of a group 2 influenza A virus HA of a different subtype than the first influenza A virus group 2 strain.

19. The mosaic influenza virus HA polypeptide of embodiment 18, wherein the different subtype is an H10 subtype or H14 subtype.

20. The mosaic influenza virus HA polypeptide of embodiment 19, wherein the H10 subtype is A/Jiangxi-Donghu/346-1/2013.

21. The mosaic influenza virus HA polypeptide of embodiment 19, wherein the H14 subtype is A/mallard/Gurjev/263/1982.

22. The mosaic influenza virus HA polypeptide of any one of embodiments 17 to 21, wherein the mosaic influenza virus HA polypeptide further comprises the transmembrane and cytoplasmic domains of the first group 2 influenza A virus strain HA.

23. The mosaic influenza virus HA polypeptide of any one of embodiments 17 to 22, wherein the first group 2 influenza virus strain is A/Hong Kong/4801/2014.

24. A mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprises one, two, three four or all of the following amino acid sequence substitutions:
a. the amino acid sequence NNESFNWT-GVTQNGTSSACIRRSSSS (SEQ ID NO:13) in the HA globular head domain A antigenic site of influenza A/Hong Kong/4801/2014 virus HA has been substituted with the following amino acid sequence NNESFNWTGVTQNGTSSACMRNGGNS (SEQ ID NO: 14);
b. the amino acid sequences THL-NYK (SEQ ID NO: 15) and GTDKDQIFLYAQ (SEQ ID NO: 16) in the HA globular head domain B antigenic site of influenza A/Hong Kong/4801/2014 virus HA have been substituted with the following amino acid sequences THL-NQK (SEQ ID NO: 17) and GTNQDQIFLYAQ (SEQ ID NO: 18), respectively;
c. the amino acid sequences QNSSIGEICDS (SEQ ID NO: 19) and PIG-KCKSE (SEQ ID NO: 20) in the HA globular head domain C antigenic site of influenza A/Hong Kong/4801/2014 virus HA have been substituted with the following amino acid sequences ESTGINRLCMK (SEQ ID NO: 21) and PIDNNCESK (SEQ ID NO: 22), respectively;
d. the amino acid sequence RITVSTKRSQQAVIPNIGS (SEQ ID NO: 23) in the HA globular head domain D antigenic site of influenza A/Hong Kong/4801/2014 virus HA has been substituted with the following amino acid sequence RITVSTSTYQQAVIPNIGS (SEQ ID NO: 25); and
e. the amino acid sequences ENCT (SEQ ID NO: 124), GFQNKKWDLFVERSKAY (SEQ ID NO: 27) and IRSGKS (SEQ ID NO:28) in the HA globular head domain E antigenic site of influenza A/Hong Kong/4801/2014 virus HA have been substituted with the following amino acid sequences GNCH (SEQ ID NO: 125), GFQNKMWDLFVERSKAY (SEQ ID NO: 29) and LRIGRS (SEQ ID NO: 24), respectively.

25. A mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise one, two, three, four or all of the following amino acid sequence substitutions:
(a) the amino acid sequence IRRSSSS (SEQ ID NO: 127) in the HA globular head domain A antigenic site of influenza A/Hong Kong/4801/2014 virus HA has been substituted with the following amino acid sequence MRNGGNS (SEQ ID NO: 128);
(b) the amino acid sequences THLNYK (SEQ ID NO: 15) and TDKDQIFPYA (SEQ ID NO: 130) the HA globular head domain B antigenic site of influenza A/Hong Kong/4801/2014 virus HA have been substituted with the following amino acid sequences THLNQK (SEQ ID NO: 17) and TDQDQIFPYA (SEQ ID NO: 131), respectively;
(c) the amino acid sequences QNSSIGEICDS (SEQ ID NO: 19) and G-KCKSE (SEQ ID NO: 132) in the HA globular head domain C antigenic site of influenza A/Hong Kong/4801/2014 virus HA have been substituted with the following amino acid sequences ESTGTNRLCMK (SEQ ID NO: 133) and DNNCESK (SEQ ID NO: 134), respectively;
(d) the amino acid sequence KRSQQA (SEQ ID NO: 135) in the HA globular head domain D antigenic site of influenza A/Hong Kong/4801/2014 virus HA has been substituted with the following amino acid sequence STYQQT (SEQ ID NO: 136); and
(e) the amino acid sequences ENCT (SEQ ID NO: 124), K and IRSGK (SEQ ID NO: 137) the HA globular head domain E antigenic site of influenza A/Hong Kong/4801/2014 virus HA have been substituted with the following amino acid sequences GNCH (SEQ ID NO: 125), M, and LRIGR (SEQ ID NO: 138), respectively.

26. A mosaic influenza virus HA polypeptide comprising an HA ectodomain of influenza A/Hong Kong/4801/2014 virus HA, wherein the HA ectodomain comprises the influenza A/Hong Kong/4801/2014 virus HA stem domain and HA globular head domain, wherein the globular head domain has been engineered to comprise one, two, three, four or all of the following amino acid sequence substitutions:
(a) the amino acid sequence NNESFNWT-GVTQNGTSSACIRRSSSS (SEQ ID NO: 13) in the HA globular head domain A antigenic site of influenza A/Hong Kong/4801/2014 virus HA has been substituted with the following amino acid sequence NNESFNWT-GVTQNGTSSACLRGGRNS (SEQ ID NO: 139);
(b) the amino acid sequences THL-NYK (SEQ ID NO: 15) and GTDKDQIFLYAQ (SEQ ID NO: 16) in the HA globular head domain B antigenic site of influenza A/Hong Kong/4801/2014 virus HA have been substituted with the following amino acid sequences THL-NGK (SEQ ID NO: 140) and GTDNDQIFLYAQ (SEQ ID NO: 141), respectively;
(c) the amino acid sequences QNSSIGEICDS (SEQ ID NO: 19) and PIG-KCKSE (SEQ ID NO: 20) in the HA globular head domain C antigenic site of influenza A/Hong Kong/4801/2014 virus HA have been substituted with the following amino acid sequences ETTNHTDECPK (SEQ ID NO: 142) and PIGKSCTSP (SEQ ID NO: 143), respectively;
(d) the amino acid sequence RITVSTKRSQQAVIPNIGS (SEQ ID NO: 23) in the HA globular head domain D antigenic site of influenza A/Hong Kong/4801/2014 virus HA has been substituted with the following amino acid sequence RITVSTRSDQQTVIPNIGS (SEQ ID NO: 144); and
(e) the amino acid sequences ENCT (SEQ ID NO: 124), GFQNKKWDLFVERSKAY (SEQ ID NO: 27) and IRSGKS (SEQ ID NO:28) in the HA globular head domain E antigenic site of influenza A/Hong Kong/4801/2014 virus HA have been substituted with the following amino acid sequences QNCD (SEQ ID NO: 145), GFQNKTWDLFVERSKAY (SEQ ID NO: 146) and IRKGRS (SEQ ID NO: 147), respectively.

27. The mosaic influenza virus HA polypeptide of any one of embodiments 24 to 26, wherein the mosaic influenza virus HA polypeptide further comprises the transmembrane and cytoplasmic domains of the influenza A/Hong Kong/4801/2014 virus HA.

28. A mosaic influenza virus HA polypeptide comprising the amino acid sequence set forth in FIG. 18A under mH10/3 (SEQ ID NO: 173).

29. A mosaic influenza virus HA polypeptide comprising the amino acid sequence set forth in FIG. 18B under mH14/3 (SEQ ID NO: 175) 30. A mosaic influenza virus HA polypeptide comprising the amino acid sequence set forth in SEQ ID NO: 30.

31. A mosaic influenza virus HA polypeptide comprising the amino acid sequence set forth in SEQ ID NO: 31.

32. A nucleic acid sequence comprising the nucleotide sequence encoding the mosaic influenza virus HA polypeptide of any one of embodiments 1 to 31.

33. The nucleic acid sequence of embodiment 32, which further comprises a nucleotide sequence encoding an influenza A virus signal sequence.

34. The nucleic acid sequence of embodiment 32 or 33, which further comprises a nucleotide sequence comprising the 5′ and 3′ non-coding regions of an influenza A virus.

35. An expression vector comprising the nucleic acid sequence of any one of embodiments 32 to 34.

36. A viral vector comprising the mosaic influenza virus HA polypeptide of any one of embodiments 1 to 31.

37. An influenza A virus comprising the mosaic influenza virus HA polypeptide of any one of embodiments 1 to 31.

38. An influenza A virus engineered to express the mosaic influenza HA polypeptide of any one of embodiments 1 to 31.

39. The influenza A virus of embodiment 37 or 38, wherein the influenza A virus is A/Puerto Rico/8/34 or an influenza A virus lacking the NS1 protein.

40. The influenza A virus of embodiment 37 or 38, wherein the influenza A virus is cold-adapted influenza A virus.

41. The influenza A virus of embodiment 40, wherein the cold-adapted influenza A virus is A/Ann Arbor/6/60 or A/Leningrad/134/17/57.

42. The influenza A virus of any one of embodiments 37 to 41, which is attenuated.

43. The influenza A virus of any one of embodiments 37 to 41, which is inactivated.

44. A virus-like particle comprising the mosaic influenza virus HA polypeptide of any one of embodiments 1 to 31.

45. A cell line expressing the mosaic influenza virus HA polypeptide of any one of embodiments 1 to 31.

46. A cell line comprising the influenza A virus of any one of embodiments 37 to 42.

47. An immunogenic composition comprising the mosaic influenza virus HA polypeptide of any one of embodiments 1 to 31.

48. An immunogenic composition comprising the viral vector of embodiment 36.

49. An immunogenic composition comprising the influenza A virus of any one of embodiments 37 to 43.

50. An immunogenic composition comprising the nucleic acid sequence of embodiment 32.

51. The immunogenic composition of embodiment 50, wherein the nucleic acid sequence is an RNA sequence.

52. The immunogenic composition of embodiment 50 or 51, wherein the composition further comprising a second nucleic acid sequence comprising a second nucleotide sequence encoding an influenza A virus neuraminidase (NA).

53. The immunogenic composition of embodiment 52, wherein the composition further comprises a third nucleic acid sequence comprising a third nucleotide sequence encoding an influenza A virus nucleoprotein (NP).

54. The immunogenic composition of embodiment 52, wherein the second nucleic acid sequence is an RNA sequence.

55. The immunogenic composition of embodiment 53, wherein the second and third nucleic acid sequences are RNA sequences.

56. An immunogenic composition comprising the virus-like particle of embodiment 44.

57. The immunogenic composition of any one of embodiments 47 to 56 further comprising an adjuvant.

58. A subunit vaccine comprising the mosaic influenza virus HA polypeptide of any one of embodiments 1 to 31.

59. A split vaccine comprising the mosaic influenza virus HA polypeptide of any one of embodiments 1 to 31.

60. The subunit vaccine of embodiment 58 further comprising an adjuvant.

61. The split vaccine of embodiment 59 further comprising an adjuvant.

62. A method for inducing an immune response against influenza A virus to a subject, comprising administering to the subject the immunogenic composition of any one of embodiments 47 to 57.

63. A method of immunizing a subject against influenza A virus, comprising administering to the subject the immunogenic composition of any one of embodiments 47 to 57.

64. A method for immunizing a subject against influenza A virus, comprising administering to the subject the subunit vaccine of embodiment 58 or 60.

65. A method of immunizing a subject against influenza A virus, comprising administering to the subject the split vaccine of embodiment 59 or 61.

66. A method for preventing an influenza virus disease in a subject, comprising administering to the subject the immunogenic composition of any one of embodiments 47 to 57.

67. A method for preventing an influenza virus disease in a subject, comprising administering to the subject the subunit vaccine of embodiment 58 or 60.

68. A method preventing an influenza virus disease in a subject, comprising administering to the subject the split vaccine of embodiment 59 or 61.

69. The method of any one of embodiments 62 to 68, wherein the subject is a human.

70. A method of determining a change in a subject's immune response to a first influenza A virus, comprising:
a. measuring hemagglutination inhibition in a series of wells containing red blood cells and either inactivated plasma or sera from the subject from a first time point or inactivated plasma or sera from the subject from a second time point, wherein each of the series of wells contains a different influenza A virus, wherein each of the different influenza A viruses comprises a different mosaic influenza virus HA polypeptide, wherein each mosaic influenza virus HA polypeptide comprises an HA ectodomain of the first influenza A virus HA, wherein the HA ectodomain comprises an HA stem domain of the first influenza A virus HA and an HA globular head domain of the first influenza A virus HA, and wherein the HA globular head domain of the first influenza A virus HA has been engineered to comprise amino acid substitutions in one, two, three, four or more of the antigenic sites; and
b. comparing the hemagglutination inhibition in each of the wells, wherein a difference in the inhibition of the hemagglutination in wells containing the plasma or sera from the first time point relative to the inhibition of hemagglutination in wells containing the plasma or sera from the second time point indicates a change in the subject's immune response to the first influenza A virus.

71. The method of embodiment 70, wherein the first time point is prior to vaccination with an influenza virus vaccine and the second time point is post-vaccination.

72. The method of embodiment 70, wherein the first time point is 6 months, 1 year, 2 years or more before the second time point.

73. The method of embodiment 70, 71 or 72, wherein the difference is an increase in inhibition of hemagglutination using inactivated plasma or sera from the second time point relative to the inhibition of hemagglutinin using inactivated plasma or sera from the first time point.

74. The method of embodiment 73, wherein the change in the subject's immune response to the first influenza A virus is an improvement.

75. The method of any one of embodiments 70 to 74, wherein the subject is a human.

### 8. EQUIVALENTS

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1.-61. (canceled)

62. A method for inducing an immune response against influenza A virus in a subject or immunizing a subject for the prevention of influenza A virus disease comprising administering to the subject an immunogenic composition comprising a mosaic influenza virus hemagglutinin (HA) polypeptide, wherein the mosaic influenza virus HA polypeptide comprises an HA ectodomain of a first group 2 influenza A virus strain HA, wherein the HA ectodomain comprises an HA stem domain of the first group 2 influenza A virus strain HA and an HA globular head domain of the first group 2 influenza A virus strain HA, wherein the HA globular head domain of the first group 2 influenza A virus strain HA comprises amino acid residue substitutions in antigenic sites A to E, and wherein:
(a) antigenic site A of the HA globular head domain of the first group 2 influenza A virus strain HA comprises 1, 2, 3, 4, 5, 6, 7, 8, 9, 10, 11, 12, 13, 14, or 15 amino acid residue substitutions, wherein 1, 2, 3, 4, 5, 6, 7, 8, 9, 10, 11, 12, 13, 14, or 15 of the amino acid residue substitutions are substitutions to amino acid residues found in the antigenic site A of an HA globular head domain of HA of a second group 2 influenza A virus, wherein the second group 2 influenza A virus is a different subtype or a different strain than the first group 2 influenza A virus strain;
(b) antigenic site B of the HA globular head domain of the first group 2 influenza A virus strain HA comprises 1, 2, 3, 4, 5, 6, 7, 8, 9, or 10 amino acid residue substitutions, wherein 1, 2, 3, 4, 5, 6, 7, 8, 9, or 10 of the amino acid residue substitutions are substitutions to amino acid residues found in the antigenic site B of an HA globular head domain of the second group 2 influenza A virus;
(c) antigenic site C of the HA globular head domain of the first group 2 influenza A virus strain HA comprises 1, 2, 3, 4, 5, 6, 7, 8, 9, 10, 11, 12, 13, 14, 15, 16, or 17 amino acid residue substitutions, wherein 1, 2, 3, 4, 5, 6, 7, 8, 9, 10, 11, 12, 13, 14, 15, 16, or 17 of the amino acid residue substitutions are substitutions to amino acid residues found in the antigenic site C of an HA globular head domain of the second group 2 influenza A virus;
(d) antigenic site D of the HA globular head domain of the first group 2 influenza A virus strain HA comprises 1, 2, 3, 4, 5, 6, 7, 8, 9, or 10 amino acid residue substitutions, wherein 1, 2, 3, 4, 5, 6, 7, 8, 9, or 10 of the amino acid residue substitutions are substitutions to amino acid residues found in the antigenic site D of an HA globular head domain of the second group 2 influenza A virus; and
(e) antigenic site E of the HA globular head domain of the first group 2 influenza A virus strain HA comprises 1, 2, 3, 4, 5, 6, 7, 8, 9, or 10 amino acid residue substitutions, wherein 1, 2, 3, 4, 5, 6, 7, 8, 9, or 10 of the amino acid residue substitutions are substitutions to amino acid residues found in the antigenic site E of an HA globular head domain of the second group 2 influenza A virus;
wherein antigenic sites A to E of the first group 2 influenza A virus strain HA and antigenic sites A to E of the second group 2 influenza A virus strain HA correspond to antigenic sites A to E of influenza virus A/Hong Kong/4801/2014 (H3) HA (SEQ ID NO: 171); and
wherein the mosaic influenza virus HA polypeptide comprises 0, 1, 2, 3, 4, or 5 amino acid residue substitutions outside of antigenic sites A to E of the HA globular head domain of the first group 2 influenza A virus strain HA.

63. A method for inducing an immune response against influenza A virus in a subject or immunizing a subject for the prevention of influenza A virus disease comprising administering to the subject an immunogenic composition comprising a mosaic influenza virus hemagglutinin (HA) polypeptide, wherein the mosaic influenza virus HA polypeptide comprises an HA ectodomain of a first group 2 influenza A virus strain HA, wherein the HA ectodomain comprises an HA stem domain of the first group 2 influenza A virus strain HA and an HA globular head domain of the first group 2 influenza A virus strain HA, wherein the HA globular head domain of the first group 2 influenza A virus strain HA comprises amino acid residue substitutions in antigenic sites A to E, and wherein:
(a) antigenic site A of the HA globular head domain of the first group 2 influenza A virus strain HA comprises 1, 2, 3, 4, 5, 6, 7, 8, 9, 10, 11, 12, 13, 14, or 15 amino acid residue substitutions, wherein 1, 2, 3, 4, 5, 6, 7, 8, 9, 10, 11, 12, 13, 14, or 15 of the amino acid residue substitutions are substitutions to amino acid residues found in the antigenic site A of an HA globular head domain of HA of a second group 2 influenza A virus, wherein the second group 2 influenza A virus is a different subtype or a different strain than the first group 2 influenza A virus strain;
(b) antigenic site B of the HA globular head domain of the first group 2 influenza A virus strain HA comprises 1, 2, 3, 4, 5, 6, 7, 8, 9, or 10 amino acid residue substitutions, wherein 1, 2, 3, 4, 5, 6, 7, 8, 9, or 10 of the amino acid residue substitutions are substitutions to amino acid residues found in the antigenic site B of an HA globular head domain of the second group 2 influenza A virus;
(c) antigenic site C of the HA globular head domain of the first group 2 influenza A virus strain HA comprises 1, 2, 3, 4, 5, 6, 7, 8, 9, 10, 11, 12, 13, 14, 15, 16, or 17 amino acid residue substitutions, wherein 1, 2, 3, 4, 5, 6, 7, 8, 9, 10, 11, 12, 13, 14, 15, 16, or 17 of the amino acid residue substitutions are substitutions to amino acid residues found in the antigenic site C of an HA globular head domain of the second group 2 influenza A virus;
(d) antigenic site D of the HA globular head domain of the first group 2 influenza A virus strain HA comprises 1, 2, 3, 4, 5, 6, 7, 8, 9, or 10 amino acid residue substitutions, wherein 1, 2, 3, 4, 5, 6, 7, 8, 9, or 10 of the amino acid residue substitutions are substitutions to amino acid residues found in the antigenic site D of an HA globular head domain of the second group 2 influenza A virus; and
(e) antigenic site E of the HA globular head domain of the first group 2 influenza A virus strain HA comprises 1, 2, 3, 4, 5, 6, 7, 8, 9, or 10 amino acid residue substitutions, wherein 1, 2, 3, 4, 5, 6, 7, 8, 9, or 10 of the amino acid residue substitutions are substitutions to amino acid residues found in the antigenic site E of an HA globular head domain of the second group 2 influenza A virus;
wherein antigenic sites A to E of the first group 2 influenza A virus strain HA and antigenic sites A to E of the second group 2 influenza A virus strain HA correspond to antigenic sites A to E of influenza virus A/Hong Kong/4801/2014 (H3) HA (SEQ ID NO: 171); and
wherein the mosaic influenza virus HA polypeptide comprises 0, 1, 2, 3, 4, or 5 amino acid residue substitutions outside of antigenic sites A to E of the globular head domain of the first group 2 influenza A virus strain HA; and wherein the second group 2 influenza A virus is an H10 subtype or H14 subtype.

64. A method for inducing an immune response against influenza A virus in a subject or immunizing a subject for the prevention of influenza A virus disease comprising administering to the subject an immunogenic composition comprising a mosaic influenza virus hemagglutinin (HA) polypeptide, wherein the mosaic HA polypeptide comprises an influenza A virus HA ectodomain of an influenza virus A/Hong Kong/4801/2014 (H3) HA, wherein the HA ectodomain comprises an influenza virus A/Hong Kong/4801/2014 (H3) HA stem domain and an influenza virus A/Hong Kong/4801/2014 HA globular head domain, wherein the influenza virus A/Hong Kong/4801/2014 HA globular head comprises amino acid residue substitutions in antigenic sites A to E, and wherein:
(a) the amino acid sequence IRRSSSS (SEQ ID NO: 127) in the antigenic site A of the influenza virus A/Hong Kong/4801/2014 HA globular head domain has been substituted with the amino acid sequence MRNGGNS (SEQ ID NO: 128);
(b) the amino acid sequences THLNYK (SEQ ID NO: 15) and TDKDQIFPYA (SEQ ID NO: 130) in the antigenic site B of the influenza virus A/Hong Kong/4801/2014 HA globular head domain have been substituted with the amino acid sequences THLNOK (SEQ ID NO: 17) and TDQDQIFPYA (SEQ ID NO: 131), respectively;
(c) the amino acid sequences ONSSIGEICDS (SEQ ID NO: 19) and G-KCKSE (SEQ ID NO: 132) in the antigenic site C of the influenza virus A/Hong Kong/4801/2014 HA globular head domain have been substituted with the amino acid sequences ESTGTNRLCMK (SEQ ID NO: 133) and DNNCESK (SEQ ID NO: 134), respectively;
(d) the amino acid sequence KRSQQA (SEQ ID NO: 135) in the antigenic site D of the influenza virus A/Hong Kong/4801/2014 HA globular head domain has been substituted with the amino acid sequence STYQQT (SEQ ID NO: 136); and
(e) the amino acid sequences ENCT (SEQ ID NO: 124), GFQNKKWDLFVERSKAY (SEQ ID NO: 27) and IRSGK (SEQ ID NO: 137) in the antigenic site E of the influenza virus A/Hong Kong/4801/2014 HA globular head domain have been substituted with the amino acid sequences GNCH (SEQ ID NO: 125), GFQNKMWDLFVERSKAY (SEQ ID NO: 29) and LRIGR (SEQ ID NO: 138), respectively, and
wherein the mosaic influenza virus HA polypeptide comprises 0, 1, 2, 3, 4, or 5 amino residue acid substitutions outside of antigenic sites A to E of the influenza virus A/Hong Kong/4801/2014 HA globular head domain.

65. A method for inducing an immune response against influenza A virus in a subject or immunizing a subject for the prevention of influenza A virus disease comprising administering to the subject an immunogenic composition comprising a mosaic influenza virus hemagglutinin (HA) polypeptide, wherein the mosaic influenza virus HA polypeptide comprises an influenza A virus HA ectodomain of an influenza virus A/Hong Kong/4801/2014 (H3) HA, wherein the HA ectodomain comprises an influenza virus A/Hong Kong/4801/2014 (H3) HA stem domain and an influenza virus A/Hong Kong/4801/2014 HA globular head domain, wherein the influenza virus A/Hong Kong/4801/2014 HA globular head comprises amino acid residue substitutions in antigenic sites A to E, and wherein:
(a) the amino acid sequence NNESFNWTGVTQNGTSSACIRRSSSS (SEQ ID NO: 13) in the antigenic site A of the influenza virus A/Hong Kong/4801/2014 HA globular head domain has been substituted with the amino acid sequence NNESFNWTGVTQNGTSSACMRNGGNS (SEQ ID NO: 14);
(b) the amino acid sequences THLNYK (SEQ ID NO: 15) and GTDKDQIFLYAQ (SEQ ID NO: 16) in the antigenic site B of the influenza virus A/Hong Kong/4801/2014 HA globular head domain have been substituted with the amino acid sequences THLNOK (SEQ ID NO: 17) and GTNQDQIFLYAQ (SEQ ID NO: 18), respectively;
(c) the amino acid sequences QNSSIGEICDS (SEQ ID NO: 19) and PIGKCKSE (SEQ ID NO: 20) in the antigenic site C of the influenza virus A/Hong Kong/4801/2014 HA globular head domain have been substituted with the amino acid sequences ESTGTNRLCMK (SEQ ID NO: 133) and PIDNNCESK (SEQ ID NO: 22), respectively;
(d) the amino acid sequence RITVSTKRSQQAVIPNIGS (SEQ ID NO: 23) in the antigenic site D of the influenza virus A/Hong Kong/4801/2014 HA globular head domain has been substituted with the amino acid sequence RITVSTSTYQQAVIPNIGS (SEQ ID NO: 25); and
(e) the amino acid sequences ENCT (SEQ ID NO: 124), GFQNKKWDLFVERSKAY (SEQ ID NO: 27) and IRSGKS (SEQ ID NO:28) in the antigenic site E of the influenza virus A/Hong Kong/4801/2014 HA globular head domain have been substituted with the amino acid sequences GNCH (SEQ ID NO: 125), GFQNKMWDLFVERSKAY (SEQ ID NO: 29) and LRIGRS (SEQ ID NO: 24), respectively, and
wherein the mosaic HA polypeptide comprises 0, 1, 2, 3, 4, or 5 amino acid residue substitutions outside of antigenic sites A to E of the influenza virus A/Hong Kong/4801/2014 HA globular head domain.

66.-68. (canceled)

69. The method of claim 62, wherein the subject is a human.

70.-75. (canceled)

76. The method of claim 62, wherein the first group 2 influenza A virus strain is a strain of an H3 subtype.

77. The method of claim 62, wherein the first group 2 influenza A virus strain of the H3 subtype is influenza virus A/Hong Kong/4801/2014 (H3).

78. The method of claim 62, wherein the mosaic influenza virus HA polypeptide further comprises: (i) the transmembrane and cytoplasmic domains of the first group 2 influenza A virus HA; or (ii) a trimerization domain.

79. The method of claim 63, wherein the first group 2 influenza A virus strain is a strain of an H3 subtype.

80. The method of claim 63, wherein the first group 2 influenza A virus strain of the H3 subtype is influenza virus A/Hong Kong/4801/2014 (H3).

81. The method of claim 63, wherein the H10 subtype is influenza virus A/Jiangxi-Donghu/346-1/2013 HA (H10).

82. The method of claim 63, wherein the H14 subtype is influenza virus A/mallard/Gurjev/263/1982 HA (H14).

83. The method of claim 79, wherein the H10 subtype is influenza virus A/Jiangxi-Donghu/346-1/2013 HA (H10).

84. The method of claim 79, wherein the H14 subtype is influenza virus A/mallard/Gurjev/263/1982 HA (H14).

85. The method of claim 80, wherein the H10 subtype is influenza virus A/Jiangxi-Donghu/346-1/2013 HA (H10).

86. The method of claim 80, wherein the H14 subtype is influenza virus A/mallard/Gurjev/263/1982 HA (H14).

87. The method of claim 63, wherein the mosaic influenza virus HA polypeptide further comprises: (i) the transmembrane and cytoplasmic domains of the first group 2 influenza A virus HA; or (ii) a trimerization domain.

88. The method of claim 63, wherein the subject is a human.

89. The method of claim 80, wherein the subject is a human.

90. The method of claim 81, wherein the subject is a human.

91. The method of claim 82, wherein the subject is a human.

92. The method of claim 85, wherein the subject is a human.

93. The method of claim 86, wherein the subject is a human.

94. The method of claim 64, wherein the mosaic influenza virus HA polypeptide further comprises: (i) the transmembrane and cytoplasmic domains of influenza virus A/Hong Kong/4801/2014 HA; or (ii) a trimerization domain.

95. The method of claim 64, wherein the subject is a human.

96. The method of claim 65, wherein the mosaic influenza virus HA polypeptide further comprises: (i) the transmembrane and cytoplasmic domains of influenza virus A/Hong Kong/4801/2014 HA; or (ii) a trimerization domain.

97. The method of claim 65, wherein the subject is a human.

98. A method for inducing an immune response against influenza A virus in a subject or immunizing a subject for the prevention of influenza A virus disease, comprising administering to the subject a mosaic influenza virus hemagglutinin (HA) polypeptide, wherein the mosaic influenza virus HA polypeptide comprises an influenza A virus HA ectodomain of an influenza virus A/Hong Kong/4801/2014 (H3) HA, wherein the HA ectodomain comprises an influenza virus A/Hong Kong/4801/2014 (H3) HA stem domain and an influenza virus A/Hong Kong/4801/2014 HA globular head domain, wherein the influenza virus A/Hong Kong/4801/2014 HA globular head comprises amino acid residue substitutions in antigenic sites A to E, and wherein:
(a) the amino acid sequence IRRSSSS (SEQ ID NO: 127) in the antigenic site A of the influenza virus A/Hong Kong/4801/2014 HA has been substituted with the amino acid sequence LRGGRNS (SEQ ID NO: 148);
(b) the amino acid sequences THLNYK (SEQ ID NO: 15) and TDKDQIFPYA (SEQ ID NO: 130) in the antigenic site B of the influenza virus A/Hong Kong/4801/2014 HA have been substituted with the amino acid sequences THLNGK (SEQ ID NO: 140) and TDNDQIFPYA (SEQ ID NO: 149), respectively;
(c) the amino acid sequences QNSSIGEICDS (SEQ ID NO: 19) and G-KCKSE (SEQ ID NO: 132) in the antigenic site C of the influenza virus A/Hong Kong/4801/2014 HA have been substituted with the amino acid sequences ETNHTDELCPS (SEQ ID NO: 150) and G-SCTSP (SEQ ID NO: 151), respectively;
(d) the amino acid sequence KRSQQA (SEQ ID NO: 135) in the antigenic site D of the influenza virus A/Hong Kong/4801/2014 HA has been substituted with the amino acid sequence RSDQQT (SEQ ID NO: 152); and
(e) the amino acid sequences ENCT (SEQ ID NO: 124), K83 and IRSGK (SEQ ID NO: 137) in the antigenic site E of the influenza virus A/Hong Kong/4801/2014 HA have been substituted with the amino acid sequences QNCD (SEQ ID NO: 145), 83T, and IRKGK (SEQ ID NO: 153), respectively; and
wherein the mosaic HA polypeptide comprises 0, 1, 2, 3, 4, or 5 amino acid residue substitutions outside of antigenic sites A to E of the influenza virus A/Hong Kong/4801/2014 HA globular head domain.

99. The method of claim 98, wherein the mosaic influenza virus HA polypeptide further comprises: (i) the transmembrane and cytoplasmic domains of influenza virus A/Hong Kong/4801/2014 HA; or (ii) a trimerization domain.

100. The method of claim 98, wherein the subject is a human.

101. A method for inducing an immune response against influenza A virus in a subject or immunizing a subject for the prevention of influenza A virus disease, comprising administering to the subject a mosaic influenza virus hemagglutinin (HA) polypeptide comprising:
(a) the amino acid sequence of SEQ ID NO: 173; SEQ ID NO: 31; or SEQ ID NO: 175; or
(b) the amino acid sequence of SEQ ID NO: 173 or SEQ ID NO: 175 without the signal peptide.

102. The method of claim 101, wherein the subject is a human.


SEQUENCE LISTING (179 sequences)

SEQ ID NO: 1 - protein, 5 aa, Influenza A virus
  source 1..5: note = amino acid sequence in Sa antigenic site of A/Michigan/45/2015
KKGNS

SEQ ID NO: 2 - protein, 6 aa, Influenza A virus
  source 1..6: note = amino acid sequence in Sa antigenic site of A/Michigan/45/2015
PKLNQS

SEQ ID NO: 3 - protein, 5 aa, synthetic construct
  source 1..5: note = amino acid sequence in mosaic Sa antigenic site
KKNST

SEQ ID NO: 4 - protein, 6 aa, synthetic construct
  source 1..6: note = amino acid sequence in mosaic Sa antigenic site
PTIKRS

SEQ ID NO: 5 - protein, 12 aa, Influenza A virus
  source 1..12: note = amino acid sequence in Sb antigenic site of A/Michigan/45/2015
TTADQQSLYQNA

SEQ ID NO: 6 - protein, 12 aa, synthetic construct
  source 1..12: note = amino acid sequence in mosaic Sb antigenic site
DAAEQTKLYQNP

SEQ ID NO: 7 - protein, 5 aa, Influenza A virus
  source 1..5: note = amino acid sequence in Ca1 antigenic site of A/Michigan/45/2015
INDKG

SEQ ID NO: 8 - protein, 5 aa, synthetic construct
  source 1..5: note = amino acid sequence in mosaic Ca1 antigenic site
NNTTG

SEQ ID NO: 9 - protein, 6 aa, Influenza A virus
  source 1..6: note = amino acid sequence in Ca2 antigenic site of A/Michigan/45/2015
PHAGAK

SEQ ID NO: 10 - protein, 6 aa, synthetic construct
  source 1..6: note = amino acid sequence in mosaic Ca2 antigenic site
PYQGKS

SEQ ID NO: 11 - protein, 6 aa, Influenza A virus
  source 1..6: note = amino acid sequence in Cb antigenic site of A/Michigan/45/2015
LSTASS

SEQ ID NO: 12 - protein, 5 aa, synthetic construct
  source 1..5: note = amino acid sequence in mosaic Cb antigenic site
LNVPE

SEQ ID NO: 13 - protein, 26 aa, Influenza A virus
  source 1..26: note = amino acid sequence in A antigenic site of A/Hong Kong/4801/2014
NNESFNWTGVTQNGTSSACIRRSSSS

SEQ ID NO: 14 - protein, 26 aa, synthetic construct
  source 1..26: note = amino acid sequence in mosaic A antigenic site
NNESFNWTGVTQNGTSSACMRNGGNS

SEQ ID NO: 15 - protein, 6 aa, Influenza A virus
  source 1..6: note = amino acid sequence in B antigenic site of A/Hong Kong/4801/2014
THLNYK

SEQ ID NO: 16 - protein, 12 aa, Influenza A virus
  source 1..12: note = amino acid sequence in B antigenic site of A/Hong Kong/4801/2014
GTDKDQIFLYAQ

SEQ ID NO: 17 - protein, 6 aa, synthetic construct
  source 1..6: note = amino acid sequence in mosaic B antigenic site
THLNQK

SEQ ID NO: 18 - protein, 12 aa, synthetic construct
  source 1..12: note = amino acid sequence in mosaic B antigenic site
GTNQDQIFLYAQ

SEQ ID NO: 19 - protein, 11 aa, Influenza A virus
  source 1..11: note = amino acid sequence in C antigenic site of A/Hong Kong/4801/2014
QNSSIGEICDS

SEQ ID NO: 20 - protein, 8 aa, Influenza A virus
  source 1..8: note = amino acid sequence in C antigenic site of A/Hong Kong/4801/2014
PIGKCKSE

SEQ ID NO: 21 - protein, 11 aa, synthetic construct
  source 1..11: note = amino acid sequence in mosaic C antigenic site
ESTGINRLCMK

SEQ ID NO: 22 - protein, 9 aa, synthetic construct
  source 1..9: note = amino acid sequence in mosaic C antigenic site
PIDNNCESK

SEQ ID NO: 23 - protein, 19 aa, Influenza A virus
  source 1..19: note = amino acid sequence in D antigenic site of A/Hong Kong/4801/2014
RITVSTKRSQQAVIPNIGS

SEQ ID NO: 24 - protein, 6 aa, synthetic construct
  source 1..6: note = amino acid sequence in mosaic E antigenic site
LRIGRS

SEQ ID NO: 25 - protein, 19 aa, synthetic construct
  source 1..19: note = amino acid sequence in mosaic D antigenic site
RITVSTSTYQQAVIPNIGS

SEQ ID NO: 26 - skipped

SEQ ID NO: 27 - protein, 17 aa, Influenza A virus
  source 1..17: note = amino acid sequence in E antigenic site of A/Hong Kong/4801/2014
GFQNKKWDLFVERSKAY

SEQ ID NO: 28 - protein, 6 aa, Influenza A virus
  source 1..6: note = amino acid sequence in E antigenic site of A/Hong Kong/4801/2014
IRSGKS

SEQ ID NO: 29 - protein, 17 aa, synthetic construct
  source 1..17: note = amino acid sequence in mosaic E antigenic site
GFQNKMWDLFVERSKAY

SEQ ID NO: 30 - protein, 548 aa, synthetic construct
  source 1..548: note = Mosaic influenza virus H1
DTLCIGYHANNSTDTVDTVLEKNVTVTHSVNLLEDKHNGKLCKLRGVAPLHLGKCNIAGWILGNPECESLNVPEWSYIVETSNSDNGTCYPGDFINYEELREQLSSVSSFERFEIFPKTSSWPSHDSNKGVTAACPYQGKSSFYKNLIWLVKKNSTYPTIKRSYNNTTGKEVLVLWGIHHPSDAAEQTKLYQNPDAYVFVGTSSYSKKFKPEIATRPKVNDQEGRMNYYWTLVHPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 31 - protein, 551 aa, synthetic construct
  source 1..551: note = Mosaic influenza virus H3
QKIPGNDNSTATLCLGHHAVPNGTIVKTITNDRIEVTNATELVESTGINRLCMKPHQILDGGNCHLIDALLGDPQCDGFQNKMWDLFVERSKAYSNCYPYDVPDYASLRSLVASSGTLEFNNESFNWTGVTQNGTSSACMRNGGNSFFSRLNWLTHLNQKYPALNVTMPNNEQFDKLYIWGVHHPGTNQDQIFLYAQSSGRITVSTSTYQQAVIPNIGSRPRIRDIPSRISIYWTIVKPGDILLINSTGNLIAPRGYFKLRIGRSSIMRSDAPIDNNCESKCITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 32 - protein, 17 aa, Influenza A virus
  source 1..17: note = Signal Sequence of A/Michigan/45/2015 HA
MKAILVVLLYTFTTANA

SEQ ID NO: 33 - protein, 514 aa, Influenza A virus
  source 1..514: note = Ectodomain of A/Michigan/45/2015 HA
DTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQIL

SEQ ID NO: 34 - protein, 327 aa, Influenza A virus
  source 1..327: note = HA1 Domain of A/Michigan/45/2015 HA
DTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSR

SEQ ID NO: 35 - protein, 222 aa, Influenza A virus
  source 1..222: note = HA2 Domain of A/Michigan/45/2015 HA
GLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 36 - protein, 282 aa, Influenza A virus
  source 1..282: note = Stem Domain of A/Michigan/45/2015 HA
DTLCIGYHANNSTDTVDTVLEKNVTVTHSVNLLEDKHNGKLCCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQIL

SEQ ID NO: 37 - protein, 232 aa, Influenza A virus
  source 1..232: note = Globular Head Domain of A/Michigan/45/2015 HA
KLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHD

SEQ ID NO: 38 - protein, 21 aa, Influenza A virus
  source 1..21: note = Transmembrane Domain of A/Michigan/45/2015 HA
AIYSTVASSLVLVVSLGAISF

SEQ ID NO: 39 - protein, 14 aa, Influenza A virus
  source 1..14: note = Cytoplasmic Domain of A/Michigan/45/2015 HA
WMCSNGSLQCRICI

SEQ ID NO: 40 - protein, 16 aa, Influenza A virus
  source 1..16: note = Signal Sequence of A/Hong Kong/4801/2014
MKTIIALSYILCLVFA

SEQ ID NO: 41 - protein, 515 aa, Influenza A virus
  source 1..515: note = Ectodomain of A/Hong Kong/4801/2014
QKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWI

SEQ ID NO: 42 - protein, 329 aa, Influenza A virus
  source 1..329: note = HA1 Domain of A/Hong Kong/4801/2014
QKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTR

SEQ ID NO: 43 - protein, 221 aa, Influenza A virus
  source 1..221: note = HA2 Domain of A/Hong Kong/4801/2014
GIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 44 - protein, 291 aa, Influenza A virus
  source 1..291: note = Stem Domain of A/Hong Kong/4801/2014
QKIPGNDNSTATLCLGHHAVPNGTIVKTITNDRIEVTNATELVQNSSIGEICCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWI

SEQ ID NO: 45 - protein, 224 aa, Influenza A virus
  source 1..224: note = Globular Head Domain of A/Hong Kong/4801/2014
DSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGK

SEQ ID NO: 46 - protein, 21 aa, Influenza A virus
  source 1..21: note = Transmembrane Domain of A/Hong Kong/4801/2014
LWISFAISCFLLCVALLGFIM

SEQ ID NO: 47 - protein, 14 aa, Influenza A virus
  source 1..14: note = Cytoplasmic Domain of A/Hong Kong/4801/2014
WACQKGNIRCNICI

SEQ ID NO: 48 - protein, 16 aa, Influenza A virus
  source 1..16: note = Signal Sequence of A/Jiangxi-Donghu/346-1/2013 HA
MYKIVVIIALLGAVKG

SEQ ID NO: 49 - protein, 510 aa, Influenza A virus
  source 1..510: note = Ectodomain of A/Jiangxi-Donghu/346-1/2013 HA
LDKICLGHHAVANGTIVKTLTNEQEEVTNATETVESTGINRLCMKGRKHKDLGNCHPIGMLIGTPACDLHLTGMWDTLIERENAIAYCYPGATVNVEALRQKIMESGGINKISTGFTYGSSINSAGTTRACMRNGGNSFYAELKWLVSKSKGQNFPQTTNTYRNTDTAEHLIMWGIHHPSSTQEKNDLYGTQSLSISVGSSTYRNNFVPVVGARPQVNGQSGRIDFHWTLVQPGDNITFSHNGGLIAPSRVSKLIGRGLGIQSDAPIDNNCESKCFWRGGSINTRLPFQNLSPRTVGQCPKYVNRRSLMLATGMRNVPELIQGRGLFGAIAGFLENGWEGMVDGWYGFRHQNAQGTGQAADYKSTQAAIDQITGKLNRLVEKTNTEFESIESEFSEIEHQIGNVINWTKDSITDIWTYQAELLVAMENQHTIDMADSEMLNLYERVRKQLRQNAEEDGKGCFEIYHACDDSCMESIRNNTYDHSQYREEALLNRLNINPVTLSSGYKDII

SEQ ID NO: 50 - protein, 324 aa, Influenza A virus
  source 1..324: note = HA1 Domain of A/Jiangxi-Donghu/346-1/2013 HA
LDKICLGHHAVANGTIVKTLTNEQEEVTNATETVESTGINRLCMKGRKHKDLGNCHPIGMLIGTPACDLHLTGMWDTLIERENAIAYCYPGATVNVEALRQKIMESGGINKISTGFTYGSSINSAGTTRACMRNGGNSFYAELKWLVSKSKGQNFPQTTNTYRNTDTAEHLIMWGIHHPSSTQEKNDLYGTQSLSISVGSSTYRNNFVPVVGARPQVNGQSGRIDFHWTLVQPGDNITFSHNGGLIAPSRVSKLIGRGLGIQSDAPIDNNCESKCFWRGGSINTRLPFQNLSPRTVGQCPKYVNRRSLMLATGMRNVPELIQGR

SEQ ID NO: 51 - protein, 221 aa, Influenza A virus
  source 1..221: note = HA2 Domain of A/Jiangxi-Donghu/346-1/2013 HA
GLFGAIAGFLENGWEGMVDGWYGFRHQNAQGTGQAADYKSTQAAIDQITGKLNRLVEKTNTEFESIESEFSEIEHQIGNVINWTKDSITDIWTYQAELLVAMENQHTIDMADSEMLNLYERVRKQLRQNAEEDGKGCFEIYHACDDSCMESIRNNTYDHSQYREEALLNRLNINPVTLSSGYKDIILWFSFGASCFVLLAVVMGLFFFCLKNGNMRCTICI

SEQ ID NO: 52 - protein, 283 aa, Influenza A virus
  source 1..283: note = Stem Domain of A/Jiangxi-Donghu/346-1/2013 HA
LDKICLGHHAVANGTIVKTLTNEQEEVTNATETVESTGINRLCCESKCFWRGGSINTRLPFQNLSPRTVGQCPKYVNRRSLMLATGMRNVPELIQGRGLFGAIAGFLENGWEGMVDGWYGFRHQNAQGTGQAADYKSTQAAIDQITGKLNRLVEKTNTEFESIESEFSEIEHQIGNVINWTKDSITDIWTYQAELLVAMENQHTIDMADSEMLNLYERVRKQLRQNAEEDGKGCFEIYHACDDSCMESIRNNTYDHSQYREEALLNRLNINPVTLSSGYKDII

SEQ ID NO: 53 - protein, 227 aa, Influenza A virus
  source 1..227: note = Globular Head Domain of A/Jiangxi-Donghu/346-1/2013 HA
MKGRKHKDLGNCHPIGMLIGTPACDLHLTGMWDTLIERENAIAYCYPGATVNVEALRQKIMESGGINKISTGFTYGSSINSAGTTRACMRNGGNSFYAELKWLVSKSKGQNFPQTTNTYRNTDTAEHLIMWGIHHPSSTQEKNDLYGTQSLSISVGSSTYRNNFVPVVGARPQVNGQSGRIDFHWTLVQPGDNITFSHNGGLIAPSRVSKLIGRGLGIQSDAPIDNN

SEQ ID NO: 54 - protein, 21 aa, Influenza A virus
  source 1..21: note = Transmembrane Domain of A/Jiangxi-Donghu/346-1/2013 HA
LWFSFGASCFVLLAVVMGLFF

SEQ ID NO: 55 - protein, 16 aa, Influenza A virus
  source 1..16: note = Signal Sequence of A/Vietnam/1203/04 HA
MEKIVLLFAIVSLVKS

SEQ ID NO: 56 - protein, 517 aa, Influenza A virus
  source 1..517: note = Ectodomain of A/Vietnam/1203/04 HA
DQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQIL

SEQ ID NO: 57 - protein, 330 aa, Influenza A virus
  source 1..330: note = HA1 Domain of A/Vietnam/1203/04 HA
DQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKR

SEQ ID NO: 58 - protein, 221 aa, Influenza A virus
  source 1..221: note = HA2 Domain of A/Vietnam/1203/04 HA
GLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSWMCSNGSLQCRICI

SEQ ID NO: 59 - protein, 286 aa, Influenza A virus
  source 1..286: note = Stem Domain of A/Vietnam/1203/04 HA
DQICIGYHANNSTEQVDTIMEKNVTVTHAQDILEKKHNGKLCCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQIL

SEQ ID NO: 60 - protein, 231 aa, Influenza A virus
  source 1..231: note = Globular Head Domain of A/Vietnam/1203/04 HA
DLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGN

SEQ ID NO: 61 - protein, 21 aa, Influenza A virus
  source 1..21: note = Transmembrane Domain of A/Vietnam/1203/04 HA
SIYSTVASSLALAIMVAGLSL

SEQ ID NO: 62 - protein, 14 aa, Influenza A virus
  source 1..14: note = Cytoplasmic Domain of A/Vietnam/1203/04 HA
WMCSNGSLQCRICI

SEQ ID NO: 63 - protein, 53 aa, Influenza A virus
  source 1..53: note = HA1 C-Terminal Stem Segment of A/Hong Kong/4801/2014 HA
CKSECITPNGSIPNDKPFQNVNRITYGACPRYVKHSTLKLATGMRNVPEKQTR

SEQ ID NO: 64 - protein, 52 aa, Influenza A virus
  source 1..52: note = HA1 N-Terminal Stem Segment of A/Hong Kong/4801/2014 HA
QKIPGNDNSTATLCLGHHAVPNGTIVKTITNDRIEVTNATELVQNSSIGEIC

SEQ ID NO: 65 - protein, 54 aa, Influenza A virus
  source 1..54: note = HA1 C-Terminal Stem Segment of A/Jiangxi-Donghu/346-1/2013 HA
CESKCFWRGGSINTRLPFQNLSPRTVGQCPKYVNRRSLMLATGMRNVPELIQGR

SEQ ID NO: 66 - protein, 43 aa, Influenza A virus
  source 1..43: note = HA1 N-Terminal Stem Segment of A/Jiangxi-Donghu/346-1/2013 HA
LDKICLGHHAVANGTIVKTLTNEQEEVTNATETVESTGINRLC

SEQ ID NO: 67 - protein, 53 aa, Influenza A virus
  source 1..53: note = HA1 C-Terminal Stem Segment of A/Michigan/45/2015 HA
CNTTCQTPEGAINTSLPFQNIHPITIGKCPKYVKSTKLRLATGLRNVPSIQSR

SEQ ID NO: 68 - protein, 42 aa, Influenza A virus
  source 1..42: note = HA1 N-Terminal Stem Segment of A/Michigan/45/2015 HA
DTLCIGYHANNSTDTVDTVLEKNVTVTHSVNLLEDKHNGKLC

SEQ ID NO: 69 - protein, 57 aa, Influenza A virus
  source 1..57: note = HA1 C-Terminal Stem Segment of A/Vietnam/1203/04 HA
CNTKCQTPMGAINSSMPFHNIHPLTIGECPKYVKSNRLVLATGLRNSPQRERRRKKR

SEQ ID NO: 70 - protein, 42 aa, Influenza A virus
  source 1..42: note = HA1 N-Terminal Stem Segment of A/Vietnam/1203/04 HA
DQICIGYHANNSTEQVDTIMEKNVTVTHAQDILEKKHNGKLC

SEQ ID NO: 71 - protein, 186 aa, Influenza A virus
  source 1..186: note = HA2 Stem Domain of A/Hong Kong/4801/2014 HA
GIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWI

SEQ ID NO: 72 - protein, 186 aa, Influenza A virus
  source 1..186: note = HA2 Stem Domain of A/Jiangxi-Donghu/346-1/2013 HA
GLFGAIAGFLENGWEGMVDGWYGFRHQNAQGTGQAADYKSTQAAIDQITGKLNRLVEKTNTEFESIESEFSEIEHQIGNVINWTKDSITDIWTYQAELLVAMENQHTIDMADSEMLNLYERVRKQLRQNAEEDGKGCFEIYHACDDSCMESIRNNTYDHSQYREEALLNRLNINPVTLSSGYKDII

SEQ ID NO: 73 - protein, 187 aa, Influenza A virus
  source 1..187: note = HA2 Stem Domain of A/Michigan/45/2015 HA
GLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQIL

SEQ ID NO: 74 - protein, 187 aa, Influenza A virus
  source 1..187: note = HA2 Stem Domain of A/Vietnam/1203/2004 HA
GLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQIL

SEQ ID NO: 75 - protein, 18 aa, Influenza A virus
  source 1..18: note = Signal Sequence of A/black headed gull/Sweden/1/1999 HA
MDIPVVAFLILTSTCVQA

SEQ ID NO: 76 - protein, 512 aa, Influenza A virus
  source 1..512: note = Ectodomain of A/black headed gull/Sweden/1/1999 HA
DRICVGYLSTNSSEKVDTLLENDVPVTSSVDLVETNHTGTYCSLGGISPVHLGDCSFEGWIVGNPACASNLGIREWSYLIEDPSAPHGLCYPGELDNNGELRHLFSGIRSFSRTELIAPTSWGAVNDGVSSACPDKGASSFYRNLVWFVKRGNQYPVIRGTYNNTTGRDVLVIWGIHHPVSTDEAKQLYVNNNPYTLVSTSSWSRKYNLETGTRPGYNGQKSWMKIYWYLMHPGESISFESNGGLLAPRYGYIIEEYGKGRIFQSRIRIAKCNTKCQTSVGGINTNKTFQNIERNALGDCPKYIKSGQLKLATGLRNVPAISNRGLFGAIAGFIEGGWPGLINGWYGFQHQNEQGVGMAADKESTQKAIDQITTKINNIIEKMNGNYDSIRGEFNQVEQRINMLADRIDDAVTDVWSYNAKLLVLLENDKTLDMHDANVRNLHDQVRRALKTNAIDEGNGCFELLHKCNDSCMETIRNGTYNHTEYEEESKLKRQEIEGIKLKSDDSVYKAL

SEQ ID NO: 77 - protein, 324 aa, Influenza A virus
  source 1..324: note = HA1 Domain of A/black headed gull/Sweden/1/1999 HA
DRICVGYLSTNSSEKVDTLLENDVPVTSSVDLVETNHTGTYCSLGGISPVHLGDCSFEGWIVGNPACASNLGIREWSYLIEDPSAPHGLCYPGELDNNGELRHLFSGIRSFSRTELIAPTSWGAVNDGVSSACPDKGASSFYRNLVWFVKRGNQYPVIRGTYNNTTGRDVLVIWGIHHPVSTDEAKQLYVNNNPYTLVSTSSWSRKYNLETGTRPGYNGQKSWMKIYWYLMHPGESISFESNGGLLAPRYGYIIEEYGKGRIFQSRIRIAKCNTKCQTSVGGINTNKTFQNIERNALGDCPKYIKSGQLKLATGLRNVPAISNR

SEQ ID NO: 78 - protein, 223 aa, Influenza A virus
  source 1..223: note = HA2 Domain of A/black headed gull/Sweden/1/1999 HA
GLFGAIAGFIEGGWPGLINGWYGFQHQNEQGVGMAADKESTQKAIDQITTKINNIIEKMNGNYDSIRGEFNQVEQRINMLADRIDDAVTDVWSYNAKLLVLLENDKTLDMHDANVRNLHDQVRRALKTNAIDEGNGCFELLHKCNDSCMETIRNGTYNHTEYEEESKLKRQEIEGIKLKSDDSVYKALSIYSCIASSIVLVGLILTFIMWACSSGNCRFNICI

SEQ ID NO: 79 - protein, 283 aa, Influenza A virus
  source 1..283: note = Stem Domain of A/black headed gull/Sweden/1/1999 HA
DRICVGYLSTNSSEKVDTLLENDVPVTSSVDLVETNHTGTYCCNTKCQTSVGGINTNKTFQNIERNALGDCPKYIKSGQLKLATGLRNVPAISNRGLFGAIAGFIEGGWPGLINGWYGFQHQNEQGVGMAADKESTQKAIDQITTKINNIIEKMNGNYDSIRGEFNQVEQRINMLADRIDDAVTDVWSYNAKLLVLLENDKTLDMHDANVRNLHDQVRRALKTNAIDEGNGCFELLHKCNDSCMETIRNGTYNHTEYEEESKLKRQEIEGIKLKSDDSVYKAL

SEQ ID NO: 80 - protein, 53 aa, Influenza A virus
  source 1..53: note = HA1 C-Terminal Stem Segment of A/black headed gull/Sweden/1/1999 HA
CNTKCQTSVGGINTNKTFQNIERNALGDCPKYIKSGQLKLATGLRNVPAISNR

SEQ ID NO: 81 - protein, 42 aa, Influenza A virus
  source 1..42: note = HA1 N-Terminal Stem Segment of A/black headed gull/Sweden/1/1999 HA
DRICVGYLSTNSSEKVDTLLENDVPVTSSVDLVETNHTGTYC

SEQ ID NO: 82 - protein, 229 aa, Influenza A virus
  source 1..229: note = Globular Head Domain of A/black headed gull/Sweden/1/1999 HA
SLGGISPVHLGDCSFEGWIVGNPACASNLGIREWSYLIEDPSAPHGLCYPGELDNNGELRHLFSGIRSFSRTELIAPTSWGAVNDGVSSACPDKGASSFYRNLVWFVKRGNQYPVIRGTYNNTTGRDVLVIWGIHHPVSTDEAKQLYVNNNPYTLVSTSSWSRKYNLETGTRPGYNGQKSWMKIYWYLMHPGESISFESNGGLLAPRYGYIIEEYGKGRIFQSRIRIAK

SEQ ID NO: 83 - protein, 21 aa, Influenza A virus
  source 1..21: note = Transmembrane Domain of A/black headed gull/Sweden/1/1999 HA
SIYSCIASSIVLVGLILTFIM

SEQ ID NO: 84 - protein, 14 aa, Influenza A virus
  source 1..14: note = Cytoplasmic Domain of A/black headed gull/Sweden/1/1999 HA
WACSSGNCRFNICI

SEQ ID NO: 85 - protein, 17 aa, Influenza A virus
  source 1..17: note = Exemplary influenza A HA subtype H1 signal peptide
MKANLLVLLCALAAADA

SEQ ID NO: 86 - protein, 15 aa, Influenza A virus
  source 1..15: note = Exemplary influenza A HA subtype H2 signal peptide
MAIIYLILLFTAVRG

SEQ ID NO: 87 - protein, 16 aa, Influenza A virus
  source 1..16: note = Exemplary influenza A HA subtype H3 signal peptide
MKTIIALSYIFCLALG

SEQ ID NO: 88 - protein, 188 aa, Influenza A virus
  source 1..188: note = HA2 Stem Domain of A/black headed gull/Sweden/1/1999 HA
GLFGAIAGFIEGGWPGLINGWYGFQHQNEQGVGMAADKESTQKAIDQITTKINNIIEKMNGNYDSIRGEFNQVEQRINMLADRIDDAVTDVWSYNAKLLVLLENDKTLDMHDANVRNLHDQVRRALKTNAIDEGNGCFELLHKCNDSCMETIRNGTYNHTEYEEESKLKRQEIEGIKLKSDDSVYKAL

SEQ ID NO: 89 - protein, 16 aa, Influenza A virus
  source 1..16: note = Exemplary influenza A HA subtype H4 and H5 signal peptide
MLSIVILFLLIAENSS

SEQ ID NO: 90 - protein, 16 aa, Influenza A virus
  source 1..16: note = Exemplary influenza A HA subtype H6 signal peptide
MIAIIVVAILATAGRS

SEQ ID NO: 91 - protein, 18 aa, Influenza A virus
  source 1..18: note = Exemplary influenza A HA subtype H7 signal peptide
MNTQILVFALVAVIPTNA

SEQ ID NO: 92 - protein, 16 aa, Influenza A virus
  source 1..16: note = Exemplary influenza A HA subtype H8 signal peptide
MEKFIAIATLASTNAY

SEQ ID NO: 93 - protein, 16 aa, Influenza A virus
  source 1..16: note = Exemplary influenza A HA subtype H9 signal peptide
METKAIIAALLMVTAA

SEQ ID NO: 94 - protein, 16 aa, Influenza A virus
  source 1..16: note = Exemplary influenza A HA subtype H10 signal peptide
MYKVVVIIALLGAVKG

SEQ ID NO: 95 - protein, 16 aa, Influenza A virus
  source 1..16: note = Exemplary influenza A HA subtype H11 signal peptide
MEKTLLFAAIFLCVKA

SEQ ID NO: 96 - protein, 17 aa, Influenza A virus
  source 1..17: note = Exemplary influenza A HA subtype H12 signal peptide
MEKFIILSTVLAASFAY

SEQ ID NO: 97 - protein, 18 aa, Influenza A virus
  source 1..18: note = Exemplary influenza A HA subtype H13 signal peptide
MALNVIATLTLISVCVHA

SEQ ID NO: 98 - protein, 17 aa, Influenza A virus
  source 1..17: note = Exemplary influenza A HA subtype H14 signal peptide
MIALILVALALSHTAYS

SEQ ID NO: 99 - protein, 18 aa, Influenza A virus
  source 1..18: note = Exemplary influenza A HA subtype H15 signal peptide
MNTQIIVILVLGLSMVKS

SEQ ID NO: 100 - protein, 19 aa, Influenza A virus
  source 1..19: note = Exemplary influenza A HA subtype H16 signal peptide
MMIKVLYFLIIVLGRYSKA

SEQ ID NO: 101 - DNA, 36 bp, synthetic construct
  source 1..36: mol_type = other DNA; note = Primer H3
ccgaagttgggggggagcaaaagcaggggataattc

SEQ ID NO: 102 - DNA, 44 bp, synthetic construct
  source 1..44: mol_type = other DNA; note = Primer H3-rev
ggccgccgggttattagtagaaacaagggtgtttttaattaatg

SEQ ID NO: 103 - protein, 565 aa, Influenza A virus
  source 1..565: note = Exemplary H1 HA
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 104 - protein, 563 aa, Influenza A virus
  source 1..563: note = Exemplary H2 HA
MAIIYLILLFTAVRGDQICIGYHSNNSTEKVDTILERNVTVTHAQNILEKTHNGKLCKLNGIPPLELGDCSIAGWLLGNPECDRLLTVPEWSYIMEKENPRNGLCYPGSFNDYEELKHLLSSVTHFEKVKILPKDRWTQHTTTGGSRACAVSGNPSFFRNMVWLTKKGSNYPIAKGSYNNTSGEQMLIIWGVHHPSNDETEQRTLYQNVGTYVSIGTSTLNKRSIPVIATRPKVNGQGGRMEFSWTILDIWDTINFESTGNLIAPEYGFRISKRGSSGIMKTEGTLENCETKCQTPLGAINTTLPFHNVHPLTIGECPKYVKSERLVLATGLRNVPQIESRGLFGAIAGFIEGGWQGMIDGWYGYHHSNDQGSGYAADKESTQKAIDGITNRVNSVIEKMNTQFEAVGKEFSNLEKRLENLNKKMEDGFLDVWTYNAELLVLMENERTLDFHDSNVKNLYDRVRMQLRDNAKELGNGCFEFYHKCDDECMNSVKNGTYDYPKYEEESKLNRNEIKGVKLSNMGVYQILAIYATVAGSLSLAIMIAGISLWMCSNGSLQCRICI

SEQ ID NO: 105 - protein, 566 aa, Influenza A virus
  source 1..566: note = Exemplary H3 HA
MKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGNGFFSRLNWLTKSGSTYPVLNVTMPNNDNFDKLYIWGVHHPSTNQEQTSLYVQESGRVTVSTRRSQQSIIPNIGSRPWVRGQSSRISIYWTIVKPGDVLVINSNGNLIAPRGYFKMRTGKSSIMSSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 106 - protein, 564 aa, Influenza A virus
  source 1..564: note = Exemplary H4 HA
MLSIVILFLLIAENSSQNYTGNPVICMGHHAVANGTMVKTLADDQVEVVTAQELVESQNLPELCPSPLRLVDGQTCDIINGALGSPGCDHLNGAEWDVFIERPNAVDTCYPFDVPEYQSLRSILANNGKFEFIAEEFQWNTVKQNGKSGACKRANVDDFFNRLNWLVKSDGNAYPLQNLTKINNGDYARLYIWGVHHPSTSTEQTNLYKNNPGRVTVSTKTSQTSVVPDIGSRPLVRGQSGRVSFYWTIVEPGDLIVFNTIGNLIAPRGHYKLNNQKKSTILNTAIPIGSCVSKCHTDKGSLSTTKPFQNISRIAVGDCPRYVKQGSLKLATGMRNIPEKASRGLFGAIAGFIENGWQGLIDGWYGFRHQNAEGTGTAADLKSTQAAIDQINGKLNRLIEKTNDKYHQIEKEFEQVEGRIQDLENYVEDTKIDLWSYNAELLVALENQHTIDVTDSEMNKLFERVRRQLRENAEDKGNGCFEIFHKCDNNCIESIRNGTYDHDIYRDEAINNRFQIQGVKLTQGYKDIILWISFSISCFLLVALLLAFILWACQNGNIRCQICI

SEQ ID NO: 107 - protein, 564 aa, Influenza A virus
  source 1..564: note = Exemplary H5 HA
MERIVLLLAIVSLVKSDQICIGYHANKSTKQVDTIMEKNVTVTHAQDILERTHNGKLCSLNGVKPLILRDCSVAGWLLGNPMCDEFLNLPEWLYIVEKDNPINSLCYPGDFNDYEELKYLLSSTNHFEKIRIIPRSSWSNHDASSGVSSACPYIGRSSFLRNVVWLIKKNNTYPTIKRSYNNTNQEDLLILWGIHHPNDAAEQTKLYQNPTTYVSVGTSTLNQRSIPEIATRPKVNGQSGRMEFFWTILKPNDAINFESNGNFIAPRYAYKIVKKGDSAIMKSGLAYGNCDTKCQTPVGEINSSMPFHNIHPHTIGECPKYVKSDRLVLATGLRNVPQRKKRGLFGAIAGFIEGGWQGMVDGWYGYHHSNEQGSGYAADKESTQKAIDGITNKVNSIIDKMNTRFEAVGKEFNNLERRVENLNKKMEDGFLDVWTYNVELLVLMENERTLDFHDSNVNNLYDKVRLQLKDNARELGNGCFEFYHKCDNECMESVRNGTYDYPQYSEEARLNREEISGVKLESMGVYQILSIYSTVASSLALAIMIAGLSFWMCSNGSLQCRICI

SEQ ID NO: 108 - protein, 566 aa, Influenza A virus
  source 1..566: note = Exemplary H6 HA
MIAIIVVAILATAGRSDKICIGYHANNSTTQIDTILEKNVTVTHSVELLENQKEERFCKILKKAPLDLKGCTIEGWILGNPQCDLLLGDQSWSYIVERPTAQNGICYPGVLNEVEELKALIGSGERVERFEMFPKSTWTGVDTSSGVTRACPYNSGSSFYRNLLWIIKTKSAAYSVIKGAYNNTGNQPILYFWGVHHPPDTNEQNTLYGSGDRYVRMGTESMNFAKSPEIAARPAVNGQRGRIDYYWSILKPGETLNVESNGNLIAPWYAFRFVSTSNKGAVFKSNLPIENCDATCQTVAGVLRTNKTFQNVSPLWIGECPKYVKSESLRLATGLRNVPQIETRGLFGAIAGFIEGGWTGMIDGWYGYHHENSQGSGYAADRESTQKAVDGITNKVNSIIDKMNTQFEAVDHEFSNLERRIDNLNKRMEDGFLDVWTYNAELLVLLENERTLDLHDANVKNLYERVKSQLRDNAMILGNGCFEFWHKCDDECMESVKNGTYDYPKYQDESKLNRQEIESVKLESLGVYQILAIYSTVSSSLVLVGLIIAVGLWMCSNGSMQCRICI

SEQ ID NO: 109 - protein, 563 aa, Influenza A virus
  source 1..563: note = Exemplary H7 HA
MNTQILVFALVAVIPTNADKICLGHHAVSNGTKVNTLTERGVEVVNATETVERTNIPKICSKGKRTTDLGQCGLLGTITGPPQCDQFLEFSADLIIERREGNDVCYPGKFVNEEALRQILRGSGGIDKETMGFTYSGIRTNGTTSACRRSGSSFYAEMEWLLSNTDNASFPQMTKSYKNTRRESALIVWGIHHSGSTTEQTKLYGSGNKLITVGSSKYHQSFVPSPGTRPQINGQSGRIDFHWLILDPNDTVTFSFNGAFIAPNRASFLRGKSMGIQSDVQVDANCEGECYHSGGTITSRLPFQNINSRAVGKCPRYVKQESLLLATGMKNVPEPSKKRKKRGLFGAIAGFIENGWEGLVDGWYGFRHQNAQGEGTAADYKSTQSAIDQITGKLNRLIEKTNQQFELIDNEFTEVEKQIGNLINWTKDSITEVWSYNAELIVAMENQHTIDLADSEMNRLYERVRKQLRENAEEDGTGCFEIFHKCDDDCMASIRNNTYDHSKYREEAMQNRIQIDPVKLSSGYKDVILWFSFGASCFLLLAIAMGLVFICVKNGNMRCTICI

SEQ ID NO: 110 - protein, 565 aa, Influenza A virus
  source 1..565: note = Exemplary H8 HA
MEKFIAIATLASTNAYDRICIGYQSNNSTDTVNTLIEQNVPVTQTMELVETEKHPAYCNTDLGAPLELRDCKIEAVIYGNPKCDIHLKDQGWSYIVERPSAPEGMCYPGSVENLEELRFVFSSAASYKRIRLFDYSRWNVTRSGTSKACNASTGGQSFYRSINWLTKKEPDTYDFNEGAYVNNEDGDIIFLWGIHHPPDTKEQTTLYKNANTLSSVTTNTINRSFQPNIGPRPLVRGQQGRMDYYWGILKRGETLKIRTNGNLIAPEFGYLLKGESYGRIIQNEDIPIGNCNTKCQTYAGAINSSKPFQNASRHYMGECPKYVKKASLRLAVGLRNTPSVEPRGLFGAIAGFIEGGWSGMIDGWYGFHHSNSEGTGMAADQKSTQEAIDKITNKVNNIVDKMNREFEVVNHEFSEVEKRINMINDKIDDQIEDLWAYNAELLVLLENQKTLDEHDSNVKNLFDEVKRRLSANAIDAGNGCFDILHKCDNECMETIKNGTYDHKEYEEEAKLERSKINGVKLEENTTYKILSIYSTVAASLCLAILIAGGLILGMQNGSCRCMFCI

SEQ ID NO: 111 - protein, 560 aa, Influenza A virus
  source 1..560: note = Exemplary H9 HA
METKAIIAALLMVTAANADKICIGYQSTNSTETVDTLTESNVPVTHTKELLHTEHNGMLCATDLGHPLILDTCTIEGLIYGNPSCDILLGGKEWSYIVERSSAVNGMCYPGNVENLEELRSLFSSAKSYKRIQIFPDKTWNVTYSGTSRACSNSFYRSMRWLTHKSNSYPFQNAHYTNNERENILFMWGIHHPPTDTEQTDLYKNADTTTSVTTEDINRTFKPVIGPRPLVNGQQGRIDYYWSVLKPGQTLRIRSNGNLIAPWYGHVLTGESHGRILKTDLNNGNCVVQCQTEKGGLNTTLPFHNISKYAFGNCPKYVGVKSLKLPVGLRNVPAVSSRGLFGAIAGFIEGGWPGLVAGWYGFQHSNDQGVGMAADKGSTQKAIDKITSKVNNIIDKMNKQYEVIDHEFNELEARLNMINNKIDDQIQDIWAYNAELLVLLENQKTLDEHDANVNNLYNKVKRALGSNAVEDGNGCFELYHKCDDQCMETIRNGTYDRQKYQEESRLERQKIEGVKLESEGTYKILTIYSTVASSLVLAMGFAAFLFWAMSNGSCRCNICI

SEQ ID NO: 112 - protein, 561 aa, Influenza A virus
  source 1..561: note = Exemplary H10 HA
MYKVVVIIALLGAVKGLDRICLGHHAVANGTIVKTLTNEQEEVTNATETVESTNLNKLCMKGRSYKDLGNCHPVGMLIGTPVCDPHLTGTWDTLIERENAIAHCYPGATINEEALRQKIMESGGISKMSTGFTYGSSITSAGTTKACMRNGGDSFYAELKWLVSKTKGQNFPQTTNTYRNTDTAEHLIIWGIHHPSSTQEKNDLYGTQSLSISVESSTYQNNFVPVVGARPQVNGQSGRIDFHWTLVQPGDNITFSDNGGLIAPSRVSKLTGRDLGIQSEALIDNSCESKCFWRGGSINTKLPFQNLSPRTVGQCPKYVNQRSLLLATGMRNVPEVVQGRGLFGAIAGFIENGWEGMVDGWYGFRHQNAQGTGQAADYKSTQAAIDQITGKLNRLIEKTNTEFESIESEFSETEHQIGNVINWTKDSITDIWTYNAELLVAMENQHTIDMADSEMLNLYERVRKQLRQNAEEDGKGCFEIYHTCDDSCMESIRNNTYDHSQYREEALLNRLNINPVKLSSGYKDIILWFSFGESCFVLLAVVMGLVFFCLKNGNMRCTICI

SEQ ID NO: 113 - protein, 565 aa, Influenza A virus
  source 1..565: note = Exemplary H11 HA
MEKTLLFAAIFLCVKADEICIGYLSNNSTDKVDTIIENNVTVTSSVELVETEHTGSFCSINGKQPISLGDCSFAGWILGNPMCDELIGKTSWSYIVEKPNPTNGICYPGTLESEEELRLKFSGVLEFNKFEVFTSNGWGAVNSGVGVTAACKFGGSNSFFRNMVWLIHQSGTYPVIKRTFNNTKGRDVLIVWGIHHPATLTEHQDLYKKDSSYVAVGSETYNRRFTPEINTRPRVNGQAGRMTFYWKIVKPGESITFESNGAFLAPRYAFEIVSVGNGKLFRSELNIESCSTKCQTEIGGINTNKSFHNVHRNTIGDCPKYVNVKSLKLATGPRNVPAIASRGLFGAIAGFIEGGWPGLINGWYGFQHRDEEGTGIAADKESTQKAIDQITSKVNNIVDRMNTNFESVQHEFSEIEERINQLSKHVDDSVVDIWSYNAQLLVLLENEKTLDLHDSNVRNLHEKVRRMLKDNAKDEGNGCFTFYHKCDNKCIERVRNGTYDHKEFEEESKINRQEIEGVKLDSSGNVYKILSIYSCIASSLVLAALIMGFMFWACSNGSCRCTICI

SEQ ID NO: 114 - protein, 564 aa, Influenza A virus
  source 1..564: note = Exemplary H12 HA
MEKFIILSTVLAASFAYDKICIGYQTNNSTETVNTLSEQNVPVTQVEELVHRGIDPILCGTELGSPLVLDDCSLEGLILGNPKCDLYLNGREWSYIVERPKEMEGVCYPGSIENQEELRSLFSSIKKYERVKMFDFTKWNVTYTGTSKACNNTSNQGSFYRSMRWLTLKSGQFPVQTDEYKNTRDSDIVFTWAIHHPPTSDEQVKLYKNPDTLSSVTTVEINRSFKPNIGPRPLVRGQQGRMDYYWAVLKPGQTVKIQTNGNLIAPEYGHLITGKSHGRILKNNLPMGQCVTECQLNEGVMNTSKPFQNTSKHYIGKCPKYIPSGSLKLAIGLRNVPQVQDRGLFGAIAGFIEGGWPGLVAGWYGFQHQNAEGTGIAADRDSTQRAIDNMQNKLNNVIDKMNKQFEVVNHEFSEVESRINMINSKIDDQITDIWAYNAELLVLLENQKTLDEHDANVRNLHDRVRRVLRENAIDTGDGCFEILHKCDNNCMDTIRNGTYNHKEYEEESKIERQKVNGVKLEENSTYKILSIYSSVASSLVLLLMIIGGFIFGCQNGNVRCTFCI

SEQ ID NO: 115 - protein, 566 aa, Influenza A virus
  source 1..566: note = Exemplary H13 HA
MALNVIATLTLISVCVHADRICVGYLSTNSSERVDTLLENGVPVTSSIDLIETNHTGTYCSLNGVSPVHLGDCSFEGWIVGNPACTSNFGIREWSYLIEDPAAPHGLCYPGELNNNGELRHLFSGIRSFSRTELIPPTSWGEVLDGTTSACRDNTGTNSFYRNLVWFIKKNTRYPVISKTYNNTTGRDVLVLWGIHHPVSVDETKTLYVNSDPYTLVSTKSWSEKYKLETGVRPGYNGQRSWMKIYWSLIHPGEMITFESNGGFLAPRYGYIIEEYGKGRIFQSRIRMSRCNTKCQTSVGGINTNRTFQNIDKNALGDCPKYIKSGQLKLATGLRNVPAISNRGLFGAIAGFIEGGWPGLINGWYGFQHQNEQGTGIAADKESTQKAIDQITTKINNIIDKMNGNYDSIRGEFNQVEKRINMLADRIDDAVTDIWSYNAKLLVLLENDKTLDMHDANVKNLHEQVRRELKDNAIDEGNGCFELLHKCNDSCMETIRNGTYDHTEYAEESKLKRQEIDGIKLKSEDNVYKALSIYSCIASSVVLVGLILSFIMWACSSGNCRFNVCI

SEQ ID NO: 116 - protein, 568 aa, Influenza A virus
  source 1..568: note = Exemplary H14 HA
MIALILVALALSHTAYSQITNGTTGNPIICLGHHAVENGTSVKTLTDNHVEVVSAKELVETNHTDELCPSPLKLVDGQDCHLINGALGSPGCDRLQDTTWDVFIERPTAVDTCYPFDVPDYQSLRSILASSGSLEFIAEQFTWNGVKVDGSSSACLRGGRNSFFSRLNWLTKATNGNYGPINVTKENTGSYVRLYLWGVHHPSSDNEQTDLYKVATGRVTVSTRSDQISIVPNIGSRPRVRNQSGRISIYWTLVNPGDSIIFNSIGNLIAPRGHYKISKSTKSTVLKSDKRIGSCTSPCLTDKGSIQSDKPFQNVSRIAIGNCPKYVKQGSLMLATGMRNIPGKQAKGLFGAIAGFIENGWQGLIDGWYGFRHQNAEGTGTAADLKSTQAAIDQINGKLNRLIEKTNEKYHQIEKEFEQVEGRIQDLEKYVEDTKIDLWSYNAELLVALENQHTIDVTDSEMNKLFERVRRQLRENAEDQGNGCFEIFHQCDNNCIESIRNGTYDHNIYRDEAINNRIKINPVTLTMGYKDIILWISFSMSCFVFVALILGFVLWACQNGNIRCQICI

SEQ ID NO: 117 - protein, 570 aa, Influenza A virus
  source 1..570: note = Exemplary H15 HA
MNTQIIVILVLGLSMVKSDKICLGHHAVANGTKVNTLTERGVEVVNATETVEITGIDKVCTKGKKAVDLGSCGILGTIIGPPQCDLHLEFKADLIIERRNSSDICYPGRFTNEEALRQIIRESGGIDKESMGFRYSGIRTDGATSACKRTVSSFYSEMKWLSSSMNNQVFPQLNQTYRNTRKEPALIVWGVHHSSSLDEQNKLYGTGNKLITVGSSKYQQSFSPSPGARPKVNGQAGRIDFHWMLLDPGDTVTFTFNGAFIAPDRATFLRSNAPSGIEYNGKSLGIQSDAQIDESCEGECFYSGGTINSPLPFQNIDSRAVGKCPRYVKQSSLPLALGMKNVPEKIRTRGLFGAIAGFIENGWEGLIDGWYGFRHQNAQGQGTAADYKSTQAAIDQITGKLNRLIEKTNKQFELIDNEFTEVEQQIGNVINWTRDSLTEIWSYNAELLVAMENQHTIDLADSEMNKLYERVRRQLRENAEEDGTGCFEIFHRCDDQCMESIRNNTYNHTEYRQEALQNRIMINPVKLSSGYKDVILWFSFGASCVMLLAIAMGLIFMCVKNGNLRCTICI

SEQ ID NO: 118 - protein, 565 aa, Influenza A virus
  source 1..565: note = Exemplary H16 HA
MMIKVLYFLIIVLGRYSKADKICIGYLSNNSSDTVDTLTENGVPVTSSVDLVETNHTGTYCSLNGISPIHLGDCSFEGWIVGNPSCATNINIREWSYLIEDPNAPNKFCYPGELDNNGELRHLFSGVNSFSRTELINPSKWGNVLDGVTASCLDRGASSFYRNLVWIVKKDEKYPVIKGDYNNTTGRDVLVLWGIHHPDTETTATNLYVNKNPYTLVSTKEWSKRYELEIGTRIGDGQRSWMKLYWHLMHPGERIMFESNGGLIAPRYGYIIEKYGTGRIFQSGVRMARCNTKCQTSLGGINTNKTFQNIERNALGDCPKYIKSGQLKLATGLRNVPSIGERGLFGAIAGFIEGGWPGLINGWYGFQHQNEQGTGIAADKASTQKAINEITTKINNIIEKMNGNYDSIRGEFNQVEKRINMLADRVDDAVTDIWSYNAKLLVLLENDRTLDLHDANVRNLHDQVKRALKSNAIDEGDGCFNLLHKCNDSCMETIRNGTYNHEDYREESQLKRQEIEGIKLKTEDNVYKVLSIYSCIASSIVLVGLILAFIMWACSNGSCRFNVCI

SEQ ID NO: 119 - protein, 564 aa, Influenza A virus
  source 1..564: note = Exemplary H17 HA
MELIVLLILLNPYTFVLGDRICIGYQANQNNQTVNTLLEQNVPVTGAQEILETNHNGKLCSLNGVPPLDLQSCTLAGWLLGNPNCDSLLEAEEWSYIKINESAPDDLCFPGNFENLQDLLLEMSGVQNFTKVKLFNPQSMTGVTTNNVDQTCPFEGKPSFYRNLNWIQGNSGLPFNIEIKNPTSNPLLLLWGIHNTKDAAQQRNLYGNDYSYTIFNFGEKSEEFRPEIGQRDEVKAHQDRIDYYWGSLPAQSTLRIESTGNLIAPEYGFYYKRKEGKGGLMKSKLPISDCSTKCQTPLGALNSTLPFQNVHQQTIGNCPKYVKATSLMLATGLRNNPQMEGRGLFGAIAGFIEGGWQGMIDGWYGYHHENQEGSGYAADKEATQKAVDAITNKVNSIIDKMNSQFESNIKEFNRLELRIQHLSDRVDDALLDIWSYNTELLVLLENERTLDFHDANVKNLFEKVKAQLKDNAIDEGNGCFLLLHKCNNSCMDDIKNGTYKYMDYREESHIEKQKIDGVKLTDYSRYYIMTLYSTIASSVVLGSLIIAAFLWGCQKGSIQCKICI

SEQ ID NO: 120 - protein, 6 aa, synthetic construct
  source 1..6: note = His tag
HHHHHH

SEQ ID NO: 121 - protein, 29 aa, synthetic construct
  source 1..29: note = Foldon amino acid domain sequence
GSGYIPEAPRDGQAYVRKDGEWVLLSTFL

SEQ ID NO: 122 - protein, 7 aa, synthetic construct
  source 1..7: note = Thrombin cleavage site amino acid sequence
LVPRGSP

SEQ ID NO: 123 - protein, 7 aa, synthetic construct
  source 1..7: note = Cleavage site recognized by Tobacco Etch Virus (TEV) protease
  VARIANT 7: note = Xaa is G or S
ENLYFQX

SEQ ID NO: 124 - protein, 4 aa, Influenza A virus
  source 1..4: note = amino acid sequence in E antigenic site of A/Hong Kong/4801/2014
ENCT

SEQ ID NO: 125 - protein, 4 aa, synthetic construct
  source 1..4: note = amino acid sequence in mosaic E antigenic site
GNCH

SEQ ID NO: 126 - protein, 14 aa, Influenza A virus
  source 1..14: note = Cytoplasmic Domain of A/Jiangxi-Donghu/346-1/2013 HA
FCLKNGNMRCTICI

SEQ ID NO: 127 - protein, 7 aa, synthetic construct
  source 1..7: note = amino acid sequence in A antigenic site of A/Hong Kong/4801/2014
IRRSSSS

SEQ ID NO: 128 - protein, 7 aa, synthetic construct
  source 1..7: note = amino acid sequence in mosaic A antigenic site
MRNGGNS

SEQ ID NO: 129 - skipped

SEQ ID NO: 130 - protein, 10 aa, Influenza A virus
  source 1..10: note = amino acid sequence in B antigenic site of A/Hong Kong/4801/2014
TDKDQIFPYA

SEQ ID NO: 131 - protein, 10 aa, synthetic construct
  source 1..10: note = amino acid sequence in mosaic B antigenic site
TDQDQIFPYA

SEQ ID NO: 132 - protein, 6 aa, Influenza A virus
  source 1..6: note = amino acid sequence in C antigenic site of A/Hong Kong/4801/2014
GKCKSE

SEQ ID NO: 133 - protein, 11 aa, synthetic construct
  source 1..11: note = amino acid sequence in mosaic C antigenic site
ESTGTNRLCMK

SEQ ID NO: 134 - protein, 7 aa, synthetic construct
  source 1..7: note = amino acid sequence in mosaic C antigenic site
DNNCESK

SEQ ID NO: 135 - protein, 6 aa, Influenza A virus
  source 1..6: note = amino acid sequence in D antigenic site of A/Hong Kong/4801/2014
KRSQQA

SEQ ID NO: 136 - protein, 6 aa, synthetic construct
  source 1..6: note = amino acid sequence in mosaic D antigenic site
STYQQT

SEQ ID NO: 137 - protein, 5 aa, Influenza A virus
  source 1..5: note = amino acid sequence in E antigenic site of A/Hong Kong/4801/2014
IRSGK

SEQ ID NO: 138 - protein, 5 aa, synthetic construct
  source 1..5: note = amino acid sequence in mosaic E antigenic site
LRIGR

SEQ ID NO: 139 - protein, 26 aa, synthetic construct
  source 1..26: note = amino acid sequence in mosaic A antigenic site
NNESFNWTGVTQNGTSSACLRGGRNS

SEQ ID NO: 140 - protein, 6 aa, synthetic construct
  source 1..6: note = amino acid sequence in mosaic B antigenic site
THLNGK

SEQ ID NO: 141 - protein, 12 aa, synthetic construct
  source 1..12: note = amino acid sequence in mosaic B antigenic site
GTDNDQIFLYAQ

SEQ ID NO: 142 - protein, 11 aa, synthetic construct
  source 1..11: note = amino acid sequence in mosaic C antigenic site
ETTNHTDECPK

SEQ ID NO: 143 - protein, 9 aa, synthetic construct
  source 1..9: note = amino acid sequence in mosaic C antigenic site
PIGKSCTSP

SEQ ID NO: 144 - protein, 19 aa, synthetic construct
  source 1..19: note = amino acid sequence in mosaic D antigenic site
RITVSTRSDQQTVIPNIGS

SEQ ID NO: 145 - protein, 4 aa, synthetic construct
  source 1..4: note = amino acid sequence in mosaic E antigenic site
QNCD

SEQ ID NO: 146 - protein, 17 aa, synthetic construct
  source 1..17: note = amino acid sequence in mosaic E antigenic site
GFQNKTWDLFVERSKAY

SEQ ID NO: 147 - protein, 6 aa, synthetic construct
  source 1..6: note = amino acid sequence in mosaic E antigenic site
IRKGRS

SEQ ID NO: 148 - protein, 7 aa, synthetic construct
  source 1..7: note = amino acid sequence in mosaic A antigenic site
LRGGRNS

SEQ ID NO: 149 - protein, 10 aa, synthetic construct
  source 1..10: note = amino acid sequence in mosaic B antigenic site
TDNDQIFYPA

SEQ ID NO: 150 - protein, 11 aa, synthetic construct
  source 1..11: note = amino acid sequence in mosaic C antigenic site
ETNHTDELCPS

SEQ ID NO: 151 - protein, 6 aa, synthetic construct
  source 1..6: note = amino acid sequence in mosaic C antigenic site
GSCTSP

SEQ ID NO: 152 - protein, 6 aa, synthetic construct
  source 1..6: note = amino acid sequence in mosaic D antigenic site
RSDQQT

SEQ ID NO: 153 - protein, 5 aa, synthetic construct
  source 1..5: note = amino acid sequence in mosaic E antigenic site
IRKGK

SEQ ID NO: 154 - DNA, 24 bp, synthetic construct
  source 1..24: mol_type = other DNA; note = pDZ_forward primer
tacagctcctgggcaacgtgctgg

SEQ ID NO: 155 - DNA, 23 bp, synthetic construct
  source 1..23: mol_type = other DNA; note = pDZ_reverse primer
aggtgtccgtgtcgcgcgtcgcc

SEQ ID NO: 156 - DNA, 24 bp, synthetic construct
  source 1..24: mol_type = other DNA; note = H3_forward primer
gggagcaaaagcaggggataattc

SEQ ID NO: 157 - DNA, 20 bp, synthetic construct
  source 1..20: mol_type = other DNA; note = H3_internal primer
tacccagcattgaacgtgac

SEQ ID NO: 158 - DNA, 37 bp, synthetic construct
  source 1..37: mol_type = other DNA; note = H3_reverse primer
gggttattagtagaaacaagggtgtttttaattaatg

SEQ ID NO: 159 - protein, 296 aa, Influenza A virus
  source 1..296: note = A/Hong Kong/4801/2014 (H3)
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSSCYPYDVPDYASLRSLVASSGTLEFNNESFNWTGVTQNGTSSACIRRSSSSFFSRLNWLTHLNYKYPALNVTMPNNEQFDKLYIWGVHHPGTDKDQIFPYAQSSGRITVSTKRSQQAVIPNIGSRPRIRNIPSRISIYWTIVKPGDILLINSTGNLIAPRGYFKIRSGKSSIMRSDAPIGKCKSE

SEQ ID NO: 160 - protein, 291 aa, Influenza A virus
  source 1..291: note = A/Jiangxi-Donghu/346-1/2013 (H10)
MYKIVVIITALLGAVKGLDKICLGHHAVANGTIVKTLTNEQEEVTNATETVESTGINRLCMKGRKHKDLGNCHPIGMLIGTPACDLHLTGMWDTLIERENAIAYCYPGATVNVEALRQKIMESGGINKISTGFTYGSSINSAGTTRACMRNGGNSFYAELKWLVSKSKGQNFPQTTNTYRNTDTAEHLIMWGIHHPSSTQEKNDLYGTQSLSISVGSSTYRNNFVPVVGARPQVNGQSGRIDFHWTLVQPGDNITFSHNGGLIAPSRVSKLIGRGLGIQSDAPIDNNCESK

SEQ ID NO: 161 - protein, 8 aa, Influenza A virus
  source 1..8: note = amino acid sequence in antigenic site B of H10 HA
VSKSKGQN

SEQ ID NO: 162 - protein, 10 aa, Influenza A virus
  source 1..10: note = amino acid sequence in antigenic site B of H3-delta B
TNQDQIFPYA

SEQ ID NO: 163 - protein, 10 aa, Influenza A virus
  source 1..10: note = amino acid sequence in antigenic site B of H10 HA
STQEKNDLYG

SEQ ID NO: 164 - protein, 11 aa, Influenza A virus
  source 1..11: note = amino acid sequence in antigenic site C of H10 HA
ETSGINRLCMK

SEQ ID NO: 165 - protein, 6 aa, Influenza A virus
  source 1..6: note = amino acid sequence in antigenic site D of H10 HA
STYRNN

SEQ ID NO: 166 - protein, 4 aa, Influenza A virus
  source 1..4: note = amino acid sequence in antigenic site E of H10 HA
LIGR

SEQ ID NO: 167 - protein, 547 aa, Influenza A virus
  source 1..547: note = A/black headed gull/Sweden/1/1999 H13 HA (H13N6)
DRICVGYLSTNSSEKVDTLLENDVPVTSSVDLVETNHTGTYCSLGGISPVHLGDCSFEGWIVGNPACASNLGIREWSYLIEDPSAPHGLCYPGELDNNGELRHLFSGIRSFSRTELIAPTSWGAVNDGVSSACPDKGASSFYRNLVWFVKRGNQYPVIRGTYNNTTGRDVLVIWGIHHPVSTDEAKQLYVNNNPYTLVSTSSWSRKYNLETGTRPGYNGQKSWMKIYWYLMHPGESISFESNGGLLAPRYGYIIEEYGKGRIFQSRIRIAKCNTKCQTSVGGINTNKTFQNIERNALGDCPKYIKSGQLKLATGLRNVPAISNRGLFGAIAGFIEGGWPGLINGWYGFQHQNEQGVGMAADKESTQKAIDQITTKINNIIEKMNGNYDSIRGEFNQVEQRINMLADRIDDAVTDVWSYNAKLLVLLENDKTLDMHDANVRNLHDQVRRALKTNAIDEGNGCFELLHKCNDSCMETIRNGTYNHTEYEEESKLKRQEIEGIKLKSDDSVYKALSIYSCIASSIVLVGLILTFIMWACSSGNCRFNICI

SEQ ID NO: 168 - protein, 552 aa, Influenza A virus
  source 1..552: note = A/Vietnam/1203/2004 H5 HA(H5N1)
DQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 169 - protein, 549 aa, Influenza A virus
  source 1..549: note = A/Michigan/45/2015 H1 HA (H1N2)
DTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 170 - protein, 545 aa, Influenza A virus
  source 1..545: note = A/Jiangxi-Donghu/346-1/2013 H10 HA (H10N8)
LDKICLGHHAVANGTIVKTLTNEQEEVTNATETVESTGINRLCMKGRKHKDLGNCHPIGMLIGTPACDLHLTGMWDTLIERENAIAYCYPGATVNVEALRQKIMESGGINKISTGFTYGSSINSAGTTRACMRNGGNSFYAELKWLVSKSKGQNFPQTTNTYRNTDTAEHLIMWGIHHPSSTQEKNDLYGTQSLSISVGSSTYRNNFVPVVGARPQVNGQSGRIDFHWTLVQPGDNITFSHNGGLIAPSRVSKLIGRGLGIQSDAPIDNNCESKCFWRGGSINTRLPFQNLSPRTVGQCPKYVNRRSLMLATGMRNVPELIQGRGLFGAIAGFLENGWEGMVDGWYGFRHQNAQGTGQAADYKSTQAAIDQITGKLNRLVEKTNTEFESIESEFSEIEHQIGNVINWTKDSITDIWTYQAELLVAMENQHTIDMADSEMLNLYERVRKQLRQNAEEDGKGCFEIYHACDDSCMESIRNNTYDHSQYREEALLNRLNINPVTLSSGYKDIILWFSFGASCFVLLAVVMGLFFFCLKNGNMRCTICI

SEQ ID NO: 171 - protein, 550 aa, Influenza A virus
  source 1..550: note = A/Hong Kong/4801/2014 H3 HA (H3N2)
QKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 172 - DNA, 1795 bp, synthetic construct
  source 1..1795: mol_type = other DNA; note = mH10/3 DNA
ccgaagttgggggggagcaaaagcaggggataattctattaaccatgaagactatcattgctttgagctacattctatgtctggttttcgctcaaaaaattcctggaaatgacaatagcacggcaacgctgtgccttgggcaccatgcagtaccaaacggaacgatagtgaaaacaatcacgaatgaccgaattgaagtcactaatgctactgagctggttgagagtacaggcacaaacagattatgtatgaaacctcatcagatccttgatggaggcaactgccatctaatagatgctctattgggagaccctcagtgtgatggctttcaaaataagatgtgggacctttttgttgaacgaagcaaagcctacagcagctgttacccttatgatgtgccggattatgcctcccttaggtcactagttgcctcatccggcacactggagttcaacaacgaaagcttcaattggactggagtcactcaaaacggaacaagttctgcttgcatgaggaatggagggaatagcttctttagtagattaaattggttgacccacttaaaccaaaaatacccagcattgaacgtgactatgccaaacaatgaacaatttgacaaattgtacatttggggggttcaccacccgggtacggaccaagaccaaatcttcccgtatgctcaatcatcaggaagaatcacagtatctacctccacttaccaacaaactgtaatcccaaatatcggatctagacccagaataaggaatatccctagcagaataagcatctattggacaatagtaaaaccgggagacatacttttgattcacagcacagggaatctaattgctcctaggggttacttcaaattacgaattgggaggagctcaataatgagatcagatgcaccaatagacaataattgtgagtccaaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactaataacccggcggcc

SEQ ID NO: 173 - protein, 567 aa, synthetic construct
  source 1..567: note = mH10/3 protein
MKTIIALSYILCLVFAQKIPGNDNSTATLCLGHHAVPNGTIVKTITNDRIEVTNATELVESTGTNRLCMKPHQILDGGNCHLIDALLGDPQCDGFQNKMWDLFVERSKAYSSCYPYDVPDYASLRSLVASSGTLEFNNESFNWTGVTQNGTSSACMRNGGNSFFSRLNWLTHLNQKYPALNVTMPNNEQFDKLYIWGVHHPGTDQDQIFPYAQSSGRITVSTSTYQQTVIPNIGSRPRIRNIPSRISIYWTIVKPGDILLIHSTGNLIAPRGYFKLRIGRSSIMRSDAPIDNNCESKCITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 174 - DNA, 1792 bp, synthetic construct
  source 1..1792: mol_type = other DNA; note = mH14/3 DNA
ccgaagttgggggggagcaaaagcaggggataattctattaaccatgaagactatcattgctttgagctacattctatgtctggttttcgctcaaaaaattcctggaaatgacaatagcacggcaacgctgtgccttgggcaccatgcagtaccaaacggaacgatagtgaaaacaatcacgaatgaccgaattgaagtcactaatgctactgagctggttgagacgaaccacactgatgaactgtgcccaagccctcatcagatccttgatggacaagactgcgacctaatagatgctctattgggagaccctcagtgtgatggctttcaaaataagacttgggacctttttgttgaacgaagcaaagcctacagcagctgttacccttatgatgtgccggattatgcctcccttaggtcactagttgcctcatccggcacactggagttcaacaacgaaagcttcaattggactggagtcactcaaaacggaacaagttctgcttgcttgaggggcggtcgcaacagcttctttagtagattaaattggttgacccacttaaacggaaaatacccagcattgaacgtgactatgccaaacaatgaacaatttgacaaattgtacatttggggggttcaccacccgggtacggacaatgaccaaatcttcccgtatgctcaatcatcaggaagaatcacagtatctacccgctcggaccaacaaactgtaatcccaaatatcggatctagacccagaataaggaatatccctagcagaataagcatctattggacaatagtaaaaccgggagacatacttttgattcacagcacagggaatctaattgctcctaggggttacttcaaaatacgaaaagggaagagctcaataatgagatcagatgcaaggattgggtcatgcacaagcccttgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactaataacccggcggcc

SEQ ID NO: 175 - protein, 566 aa, synthetic construct
  source 1..566: note = mH14/3 protein
MKTIIALSYILCLVFAQKIPGNDNSTATLCLGHHAVPNGTIVKTITNDRIEVTNATELVETNHTDELCPSPHQILDGQDCDLIDALLGDPQCDGFQNKTWDLFVERSKAYSSCYPYDVPDYASLRSLVASSGTLEFNNESFNWTGVTQNGTSSACLRGGRNSFFSRLNWLTHLNGKYPALNVTMPNNEQFDKLYIWGVHHPGTDNDQIFPYAQSSGRITVSTRSDQQTVIPNIGSRPRIRNIPSRISIYWTIVKPGDILLIHSTGNLIAPRGYFKIRKGKSSIMRSDARIGSCTSPCITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 176 - DNA, 1801 bp, synthetic construct
  source 1..1801: mol_type = other DNA; note = cH10/3 DNA
ccgaagttgggggggagcaaaagcaggggataattctattaaccatgaagactatcattgctttgagctacattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttgagagtacagtcataaacagattatgtatgaaaggaagaaaacataaagacctgggcaactgccatccaatagggatgctaatagggactccagcttgtgatctgcaccttacagggatgtgggacactctcattgaacgagagaatgctattgcttactgctaccctggagctactgtaaatgtagaagcactaaggcagaagataatggagagtggagggatcaacaagataagcactggcttcacttatggatcttccataaactcggccgggaccactagagcgtgcatgaggaatggagggaatagcttttatgcagagcttaagtggctggtatcaaagagcaaaggacaaaacttccctcagaccacgaacacttacagaaatacagacacggctgaacacctcataatgtggggaattcatcacccttctagcactcaagagaagaatgatctatatggaacacaatcactgtccatatcagtcgggagttccacttaccggaacaattttgttccggttgttggagcaagacctcaggtcaatggacaaagtggcagaattgattttcactggacactagtacagccaggtgacaacatcaccttctcacacaatgggggcctgatagcaccgagccgagttagcaaattaattgggaggggattgggaatccaatcagacgcaccaatagacaataattgtgagtccaaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactaataacccggcggcc

SEQ ID NO: 177 - protein, 569 aa, synthetic construct
  source 1..569: note = cH10/3 protein
MKTIIALSYILCLVFAQKIPGNDNSTATLCLGHHAVPNGTIVKTITNDRIEVTNATELVESTGINRLCMKGRKHKDLGNCHPIGMLIGTPACDLHLTGMWDTLIERENAIAYCYPGATVNVEALRQKIMESGGINKISTGFTYGSSINSAGTTRACMRNGGNSFYAELKWLVSKSKGQNFPQTTNTYRNTDTAEHLIMWGIHHPSSTQEKNDLYGTQSLSISVGSSTYRNNFVPVVGARPQVNGQSGRIDFHWTLVQPGDNITFSHNGGLIAPSRVSKLIGRGLGIQSDAPIDNNCESKCITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 178 - DNA, 1798 bp, synthetic construct
  source 1..1798: mol_type = other DNA; note = cH14/3 DNA
ccgaagttgggggggagcaaaagcaggggataattctattaaccatgaagactatcattgctttgagctacattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttgagacgaaccacactgatgaactgtgcccaagccccttgaagcttgtcgacgggcaagactgcgacctcatcaatggtgcattggggagtccaggctgtgaccgtttgcaggacaccacttgggatgtcttcattgaaaggcccactgcagtagacacatgttatccattcgacgtcccagattaccagagtctcagaagcatcctagcaagcagtgggagtttggagttcatcgccgaacaattcacctggaatggtgtcaaagttgacggatcaagcagtgcttgtttgaggggcggtcgcaacagcttcttctcccgactaaactggctaaccaaagcaacaaatggaaactatggacctattaacgtcactaaagaaaatacgggctcttatgtcaggctctatctctggggagtgcatcacccatcaagcgataatgagcaaacggatctctacaaggtggcaacagggagagtaacagtatctacccgctcggaccaaatcagtattgttcccaatataggaagtagaccgagggtaaggaatcagagcggcaggataagcatctactggaccctagtaaacccaggggactccatcattttcaacagtattgggaatttgattgcaccaagaggccactacaaaataagcaaatctactaagagcacagtgcttaaaagtgacaaaaggattgggtcatgcacaagcccttgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctactaataacccggcggcc

SEQ ID NO: 179 - protein, 568 aa, synthetic construct
  source 1..568: note = cH14/3 protein
MKTIIALSYILCLVFAQKIPGNDNSTATLCLGHHAVPNGTIVKTITNDRIEVTNATELVETNHTDELCPSPLKLVDGQDCDLINGALGSPGCDRLQDTTWDVFIERPTAVDTCYPFDVPDYQSLRSILASSGSLEFIAEQFTWNGVKVDGSSSACLRGGRNSFFSRLNWLTKATNGNYGPINVTKENTGSYVRLYLWGVHHPSSDNEQTDLYKVATGRVTVSTRSDQISIVPNIGSRPRVRNQSGRISIYWTLVNPGDSIIFNSIGNLIAPRGHYKISKSTKSTVLKSDKRIGSCTSPCITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI